(19) United States
(12) Patent Application Publication
(10) Pub. No.: US 2019/0316209 A1
(43) Pub. Date: Oct. 17, 2019

(54) Multi-Assay Prediction Model for Cancer Detection

(71) Applicant: GRAIL, Inc., Menlo Park, CA (US)

(72) Inventors: Earl Hubbell, Palo Alto, CA (US); Samuel S. Gross, Sunnyvale, CA (US); Darya Filippova, Sunnyvale, CA (US); Ling Shen, Redwood City, CA (US); Oliver Claude Venn, San Francisco, CA (US); Alexander Weaver Blocker, Mountain View, CA (US); Nan Zhang, Fremont, CA (US); Tara Maddala, Sunnyvale, CA (US); Alex Aravanis, San Mateo, CA (US); Qinwen Liu, Fremont, CA (US); Anton Valouev, Palo Alto, CA (US); Virgil Nicula, Cupertino, CA (US)

(21) Appl. No.: 16/384,784

(22) Filed: Apr. 15, 2019

Related U.S. Application Data
(60) Provisional application No. 62/657,635, filed on Apr. 13, 2018.
(60) Provisional application No. 62/679,738, filed on Jun. 1, 2018.

(51) Int. Cl. C12Q 1/6886; G16H 50/30; G16H 50/20; C12Q 1/6806; C40B 40/06; C12Q 1/6869

(52) U.S. Cl. CPC C12Q 1/6886; G16H 50/30; G16H 50/20; C12Q 1/6806; C40B 40/06; C12Q 2600/158; C12Q 2600/112; C12Q 2600/156; C12Q 2600/118; C12Q 2600/154; C12Q 1/6869

(57) ABSTRACT

A predictive cancer model generates a cancer prediction for an individual of interest by analyzing values of one or more types of features that are derived from cfDNA obtained from the individual. Specifically, cfDNA from the individual is sequenced to generate sequence reads using one or more physical assays, examples of which include a small variant sequencing assay, whole genome sequencing assay, and methylation sequencing assay. The sequence reads of the physical assays are processed through corresponding computational analyses to generate each of small variant features, whole genome features, and methylation features. The values of features can be provided to a predictive cancer model that generates a cancer prediction. In some embodiments, the values of different types of features can be separately provided into different predictive models. Each separate predictive model can output a score that can serve as input into an overall model that outputs the cancer prediction.

### CROSS-REFERENCE TO RELATED APPLICATIONS

This application claims the benefit of priority to U.S. Provisional Application No. 62/657,635, filed Apr. 13, 2018, and U.S. Provisional Application No. 62/679,738 filed Jun. 1, 2018, both of which are incorporated herein by reference in their entirety for all purposes.

### BACKGROUND

This disclosure generally relates to identification of cancer in a patient, and more specifically to performing a physical assay on a test sample obtained from the patient, as well as statistical analysis of the results of the physical assay.

Analysis of circulating cell-free nucleotides, such as cell-free DNA (cfDNA) or cell-free RNA (cfRNA), using next generation sequencing (NGS) is recognized as a valuable tool for detection and diagnosis of cancer. Analyzing cfDNA can be advantageous in comparison to traditional tumor biopsy methods; however, identifying cancer-indicative signals in tumor-derived cfDNA faces distinct challenges, especially for purposes such as early detection of cancer where the cancer-indicative signals are not yet pronounced. As one example, it may be difficult to achieve the necessary sequencing depth of tumor-derived fragments. As another example, errors introduced during sample preparation and sequencing can make accurate identification cancer-indicative signals difficult. The combination of these various challenges stand in the way of accurately predicting, with sufficient sensitivity and specificity, characteristics of cancer in a subject through the use of cfDNA obtained from the subject.

### SUMMARY

Embodiments of the invention provide for a method of generating a cancer prediction, such as a presence or absence of cancer, for an individual based on cfDNA in a test sample obtained from the individual. Specifically, cfDNA from the individual is sequenced to generate sequence reads using one or more sequencing assays, also referred to herein as physical assays, examples of which include a small variant sequencing assay, whole genome sequencing assay, and methylation sequencing assay. The sequence reads of the sequencing assays are processed through corresponding computational analyses, also hereafter referred to any one of computational pipelines, computational assessments, and computational analyses. Each computational analysis identifies values of features of sequence reads that are informative for generating a cancer prediction while accounting for interfering signals (e.g., noise). As an example, small variant features (e.g., features derived from sequence reads that were generated by a small variant sequencing assay) can include a total number of somatic variants. As another example, whole genome features (e.g., features derived from sequence reads that were generated by a whole genome sequencing assay) can include a total number of copy number aberrations. As yet another example, methylation features (e.g., features derived from sequence reads that were generated by a methylation sequencing assay) can include a total number hypermethylated or hypomethylated regions. Additional features that are not derived from sequencing-based approaches, such as baseline features that can refer to clinical symptoms and patient information, can be further generated and analyzed.

In some embodiments, one, two, three, or all four of the types of features (e.g., small variant features, whole genome features, methylation features, and baseline features) can be provided to a single predictive cancer model that generates a cancer prediction. In some embodiments, the values of different types of features can be separately provided into different predictive models. Each separate predictive model can output a score that then serves as input into an overall model that outputs the cancer prediction.

Embodiments disclosed herein describe a method for detecting the presence of cancer in a subject, the method comprising: obtaining sequencing data generated from a plurality of cell-free nucleic acids in a test sample from the subject, wherein the sequencing data comprises a plurality of sequence reads determined from the plurality of cell-free nucleic acids; analyzing, using a suitable programed computer, the plurality of sequence reads to identify two or more sequencing based features; and detecting the presence of cancer based on the analysis of the two or more features.

Embodiments disclosed herein further describe a method for detecting the presence of cancer in an asymptomatic subject, the method comprising: obtaining sequencing data generated from a plurality of cell-free nucleic acids in a test sample from an asymptomatic subject; analyzing, using a suitable programed computer, the sequencing data to identify two or more sequencing based features; detecting the presence of cancer based on the analysis of the two or more features.

Embodiments disclosed herein further describe a method for detecting the presence of cancer in an asymptomatic subject, the method comprising: obtaining sequencing data generated from a plurality of cell-free nucleic acids in a test sample from an asymptomatic subject; analyzing, using a suitable programed computer, the sequencing data to identify two or more sequencing based features; detecting the presence of cancer based on the analysis of the two or more features.

In some embodiments, the method detects three or more different types of cancer. In some embodiments, the method detects five or more different types of cancer. In some embodiments, the method detects ten or more different types of cancer. In some embodiments, the method detects twenty or more different types of cancer. In some embodiments, the two or more different types of cancer are selected from breast cancer, lung cancer, prostate cancer, colorectal cancer, renal cancer, uterine cancer, pancreas cancer, esophageal cancer, lymphoma, head and neck cancer, ovarian cancer, hepatobiliary cancer, melanoma, cervical cancer, multiple myeloma, leukemia, thyroid cancer, bladder cancer, gastric cancer, anorectal cancer, and any combination thereof.

In some embodiments, the cell-free nucleic acids comprise cell-free DNA (cfDNA). In some embodiments, the sequence reads are generated from a next generation sequencing (NGS) procedure. In some embodiments, the sequence reads are generated from a massively parallel sequencing procedure using sequencing-by-synthesis. In some embodiments, the cell-free nucleic acids includes cf-DNA from white blood cells.

In some embodiments, the two or more features are derived from: a methylation sequencing assay on the plurality of cell-free nucleic acids in the test sample; a whole genome sequencing assay on the plurality of cell-free nucleic acids in the test sample; and/or a small variant sequencing assay on the plurality of cell-free nucleic acids in the test sample.

In some embodiments, the methylation sequencing assay is a whole genome bisulfite sequencing assay. In some embodiments, the methylation sequencing assay is a targeted bisulfite sequencing assay. In some embodiments, detecting the presence of cancer is based on the analysis of two or more features determined from the methylation sequencing assay. In some embodiments, the methylation sequencing assay features comprise one or more of a quantity of hypomethylated counts, quantity of hypermethylated counts, presence or absence of abnormally methylated fragments at CpG sites, hypomethylation score per CpG site, hypermethylation score per CpG site, rankings based on hypermethylation scores, and rankings based on hypomethylation scores.

In some embodiments, detecting the presence of cancer is based on the analysis of two or more features determined from the whole genome sequencing assay. In some embodiments, the whole genome sequencing assay features comprise one or more of characteristics of bins across the genome either a cfDNA sample or a gDNA sample, characteristics of segments across the genome from either a cfDNA sample or a gDNA sample, presence of one or more copy number aberrations, and reduced dimensionality features. In some embodiments, the method further comprising obtaining sequence data of genomic DNA from one of more white blood cells of the subject.

In some embodiments, the small variant sequencing assay is a targeted sequencing assay, and wherein the sequence data is derived from a targeted panel of genes. In some embodiments, detecting the presence of cancer based on the analysis of two or more features determined from the small variant sequencing assay. In some embodiments, the small variant sequencing assay features comprise one or more of a total number of somatic variants, a total number of nonsynonymous variants, total number of synonymous variants, a presence/absence of somatic variants per gene, a presence/absence of somatic variants for particular genes that are known to be associated with cancer, an allele frequency of a somatic variant per gene, order statistics according to AF of somatic variants, and classification of somatic variants that are known to be associated with cancer based on their allele frequency.

In some embodiments, the analysis further comprises one or more baseline features, and wherein the baseline feature comprises a polygenic risk score or clinical features of an individual, the clinical features comprising one or more of age, behavior, family history, symptoms, anatomical observations, and penetrant germline cancer carrier.

In some embodiments, the detected cancer is breast cancer, lung cancer, colorectal cancer, ovarian cancer, uterine cancer, melanoma, renal cancer, pancreatic cancer, thyroid cancer, gastric cancer, hepatobiliary cancer, esophageal cancer, prostate cancer, lymphoma, multiple myeloma, head and neck cancer, bladder cancer, cervical cancer, or any combination thereof.

In some embodiments, the analysis further comprises detecting the presence of one or more viral-derived nucleic acids in the test sample and wherein the detection of cancer is based, in part, on detection of the one or more viral nucleic acids. In some embodiments, the one or more viral-derived nucleic acids are selected from the group consisting of human papillomavirus, Epstein-Barr virus, hepatitis B, hepatitis C, and any combination thereof.

In some embodiments, the test sample is a blood, plasma, serum, urine, cerebrospinal fluid, fecal matter, saliva, pleural fluid, pericardial fluid, cervical swab, saliva, or peritoneal fluid sample.

In some embodiments, the predictive cancer model is one of a regression predictor, a random forest predictor, a gradient boosting machine, a Näive Bayes classifier, a neural network, or a XGBoost model.

### BRIEF DESCRIPTION OF THE DRAWINGS

FIG. 1A depicts an overall flow process for generating a cancer prediction based on features derived from a cfDNA sample obtained from an individual, in accordance with an embodiment.

FIG. 1B-1D each depicts an overall flow diagram for determining a cancer prediction using at least a cfDNA sample obtained from an individual, in accordance with an embodiment.

FIG. 2 depicts a flow process 104 of a method for performing a sequencing assay to generate sequence reads, in accordance with an embodiment.

FIG. 3A is an example flow process 300 for performing a data workflow to analyze sequence reads generated by a small variant sequencing assay, in accordance with an embodiment.

FIG. 3B is flowchart of a method for processing candidate variants using different types of filters and models according to one embodiment.

FIG. 3C is a diagram of an application of a Bayesian hierarchical model according to one embodiment.

FIG. 3D shows dependencies between parameters and sub-models of a Bayesian hierarchical model for determining true single nucleotide variants according to one embodiment.

FIG. 3E shows dependencies between parameters and sub-models of a Bayesian hierarchical model for determining true insertions or deletions according to one embodiment.

FIGS. 3F-3G illustrate diagrams associated with a Bayesian hierarchical model according to one embodiment.

FIG. 3H is a diagram of determining parameters by fitting a Bayesian hierarchical model according to one embodiment.

FIG. 3I is a diagram of using parameters from a Bayesian hierarchical model to determine a likelihood of a false positive according to one embodiment.

FIG. 3J is flowchart 315 of a method for training a Bayesian hierarchical model according to one embodiment.

FIG. 3K is flowchart 325 of a method for scoring candidate variants of a given nucleotide mutation according to one embodiment.

FIG. 3L is flowchart 335 of a method for using a joint model to process cell free nucleic acid samples and genomic nucleic acid samples according to one embodiment.

FIG. 3M is a diagram of an application of a joint model according to one embodiment.

FIG. 3N is a diagram of observed counts of variants in samples from healthy individuals according to one embodiment.

FIG. 3O is a diagram of example parameters for a joint model according to one embodiment.

FIGS. 3R-3S are diagrams of variant calls determined by a joint model according to one embodiment.

FIG. 3T is a diagram of probability densities determined by a joint model according to one embodiment.

FIG. 3U is a diagram of sensitivity and specificity of a joint model according to one embodiment.

FIG. 3V is a diagram of a set of genes detected from small variant sequencing assays using a joint model according to one embodiment.

FIG. 3W is a diagram of length distributions of the set of genes shown in FIG. 17 detected from small variant sequencing assays using the joint model according to one embodiment.

FIG. 3X is a diagram of another set of genes detected from small variant sequencing assays using a joint model according to one embodiment.

FIG. 3Y is flowchart 350 of a method for tuning a joint model to process cell free nucleic acid samples and genomic nucleic acid samples according to one embodiment.

FIG. 3Z is a table of example counts of candidate variants of cfDNA samples according to an embodiment.

FIG. 3AA is a table of example counts of candidate variants of cfDNA samples from healthy individuals according to one embodiment.

FIG. 3AB is a diagram of candidate variants plotted based on ratio of cfDNA and gDNA according to one embodiment.

FIG. 4A depicts a process 400 of generating an artifact distribution and a non-artifact distribution using training variants according to one embodiment.

FIG. 4B depicts sequence reads that are categorized in an artifact training data category according to one embodiment.

FIG. 4C depicts sequence reads that are categorized in the non-artifact training data category according to one embodiment.

FIG. 4D depicts sequence reads that are categorized in the reference allele training data category according to one embodiment.

FIG. 4E is an example depiction of a process for extracting a statistical distance from edge feature according to one embodiment.

FIG. 4F is an example depiction of a process for extracting a significance score feature according to one embodiment.

FIG. 4G is an example depiction of a process for extracting an allele fraction feature according to one embodiment.

FIGS. 4H and 4I depict example distributions used for identifying edge variants according to various embodiments.

FIG. 4J depicts a block diagram flow process for determining a sample-specific predicted rate according to one embodiment.

FIG. 4K depicts the application of an edge variant prediction model for identifying edge variants according to one embodiment.

FIG. 4L depicts a flow process 452 of identifying and reporting edge variants detected from a sample according to one embodiment.

FIGS. 4M-4O each depict the features of example training variants that are categorized in one of the artifact or non-artifact categories according to various embodiments.

FIG. 4P depicts the identification of edge variants across various subject samples according to one embodiment.

FIG. 4Q depicts concordant variants called in both solid tumor and in cfDNA following the removal of edge variants using different edge filters as a fraction of the variants called in cfDNA according to one embodiment.

FIG. 4R depicts concordant variants called in both solid tumor and in cfDNA following the removal of edge variants using different edge filters as a fraction of the variants called in solid tumor according to one embodiment.

FIG. 4S is a table describing individuals of a sample set for a cell free genome study according to one embodiment.

FIG. 4T is a chart indicating types of cancers associated with the sample set for the cell free genome study of FIG. 4S according to one embodiment.

FIG. 4U is another table describing the sample set for the cell free genome study of FIG. 4S according to one embodiment.

FIG. 4V shows diagrams of example counts of called variants determined using one or more types of filters and models according to one embodiment.

FIG. 4W is a diagram of example quality scores of samples known to have breast cancer according to one embodiment.

FIG. 4X is a diagram of example counts of called variants for samples known to have various types of cancer and at different stages of cancer according to one embodiment.

FIG. 4Y is a diagram of example counts of called variants for samples known to have early or late stage cancer according to one embodiment.

FIG. 4Z is another diagram of example counts of called variants for samples known to have early or late stage cancer according to one embodiment.

FIG. 5A depicts an example flow process of two different workflows for determining whole genome features, in accordance with an embodiment.

FIG. 5B depicts an example flow process that describes the analysis for identifying characteristics of bins and segments derived from cfDNA and gDNA samples, in accordance with an embodiment.

FIG. 5C is an example depiction of sequence reads in relation to bins of a reference genome, in accordance with an embodiment.

FIG. 5E and FIG. 5F depicts bin scores across bins of a genome for a cfDNA sample and a gDNA sample, respectively, that are obtained from a breast cancer subject.

FIG. 5G and FIG. 5H depicts bin scores across bins of a genome determined from a cfDNA sample and a gDNA sample, respectively, that are obtained from a non-cancer individual.

FIG. 5I and FIG. 5J depicts bin scores across bins of a genome determined from a cfDNA sample and a gDNA sample, respectively, that are obtained from a non-cancer individual.

FIG. 6A illustrates a flow process for determining a classification score by reducing the dimensionality of high dimensionality data, in accordance with an embodiment.

FIG. 6B depicts a sample process for analyzing data to reduce data dimensionality, in accordance with an embodiment.

FIG. 6C depicts a process for analyzing data from a test sample based on information learned from data with reduced dimensionality, in accordance with an embodiment.

FIG. 6D depicts a sample process for data analysis in accordance with an embodiment.

FIG. 6E depicts a table comparing the current method (classification score) with a previous known segmentation method (z-score).

FIG. 6F depicts the improved predictive power of using the classification score method can be observed for all types of cancer.

FIG. 7A is a flowchart describing a process for identifying anomalously methylated fragments from a subject, according to an embodiment.

FIG. 7B is an illustration of an example p-value score calculation, according to an embodiment.

FIG. 7C is a flowchart describing a process of training a classifier based on methylation status of fragments, according to an embodiment.

FIGS. 7D-7F are graphs showing the cancer log-odds ratio determined for various cancers across different stages of cancer.

FIG. 8A depicts a flow process for determining baseline features that can be used to stratify a patient, in accordance with an embodiment.

FIG. 8B depicts the performance of models based on different combinations of baseline features.

FIG. 9A depicts the experimental parameters of the CCGA study.

FIG. 9B depicts the experimental details (e.g., gene panel, sequencing depth, etc.) used to determine values of features for each respective predictive cancer model.

FIG. 9C depicts a receiver operating characteristic (ROC) curve of the specificity and sensitivity of a predictive cancer model that predicts the presence of cancer using small variant features, whole genomes features, and methylation features, in accordance with the embodiment shown in FIG. 1B.

FIG. 10A depicts a receiver operating characteristic (ROC) curve of the specificity and sensitivity of a predictive cancer model that predicts the presence of cancer using a first set of small variant features.

FIG. 10B depicts a ROC curve of the specificity and sensitivity of a predictive cancer model that predicts the presence of cancer using a second set of small variant features.

FIG. 10OC depicts a ROC curve of the specificity and sensitivity of a predictive cancer model that predicts the presence of cancer using a third set of small variant features.

FIG. 10D depicts a ROC curve of the specificity and sensitivity of a predictive cancer model that predicts the presence of cancer using a set of whole genome features.

FIG. 10OE depicts a ROC curve of the specificity and sensitivity of a predictive cancer model that predicts the presence of cancer using a first set of methylation features.

FIG. 10OF depicts a ROC curve of the specificity and sensitivity of a predictive cancer model that predicts the presence of cancer using a second set of methylation features.

FIG. 10G depicts a ROC curve of the specificity and sensitivity of a predictive cancer model that predicts the presence of cancer using a third set of methylation features.

FIG. 10H depicts the performance of each of the single-assay predictive cancer models (e.g., predictive cancer models applied for features derived following performance of each of the small variant sequencing assay, whole genome sequencing assay, and methylation sequencing assay).

FIG. 10I depicts the performance of predictive cancer models for different types of cancer across different stages. As shown in FIG. 10I, the different cancer types analyzed are divided into two groups those with a high 5 year mortality rate (225%) and those with a low 5 year mortality rate (<25%).

FIGS. 10J-10OL each depicts the performance of additional predictive cancer models (in addition to the small variant (referred to in each of FIGS. 10OJ-10L as the nonsyn variant), WG, and methylation predictive cancer models shown in FIG. 10I) for different types of invasive cancers.

FIG. 10M depicts the performance of predictive cancer models for different stages of colorectal cancer.

FIG. 10ON depicts the performance of additional predictive cancer models for different stages of colorectal cancer.

FIG. 10O depicts the performance of predictive cancer models for different stages and different types of breast cancer.

FIG. 10P depicts the performance of predictive cancer models for different stages and different types of lung cancer.

FIGS. 10Q-10R depict ROC curve plots for a multi-stage model including a first model generating a cancer prediction using small variant features, a second model generating a cancer prediction using WGS features, and a combined model generating a cancer prediction using the cancer predictions of the first model and the second model.

FIG. 10S depicts a comparison of the sensitivity as a function of sensitivity for the first model, the second model, and the combination model depicted in FIGS. 10Q-R. FIGS. 10T-10U depict ROC curve plots for a multi-stage model including a first model generating a cancer prediction using WGS features, a second model generating a cancer prediction using methylation features, and a combined model generating a cancer prediction using the cancer predictions of the first model and the second model.

FIGS. 10V-X depict ROC curve plots for a multi-stage model including a first model generating a cancer prediction using baseline features, a second model generating a cancer prediction using methylation features, and a combined model generating a cancer prediction using the cancer predictions of the first model and the second model in high-signal cancers, lung cancer, and HR− cancer, respectively.

FIG. 11A depicts a ROC curve of the specificity and sensitivity of a two-stage predictive cancer model that predicts the presence of cancer.

FIG. 12A depicts a ROC curve of the specificity, sensitivity, and area under the curve (AUC) for a single-stage predictive cancer model including small variant features comprising maximum allele frequency (MAF) of non-synonymous variant within genes included in a targeted gene panel.

FIG. 12B depicts a ROC curve of the specificity, sensitivity, and area under the curve (AUC) for a single-stage predictive cancer model including small variant features comprising order statistics within genes included in a targeted gene panel.

FIG. 12C depicts a ROC curve of the specificity, sensitivity, and area under the curve (AUC) for a single-stage predictive cancer model including methylation features.

FIG. 12D. depicts a ROC curve of the specificity, sensitivity, and area under the curve (AUC) for a single-stage predictive cancer model including small variant and methylation features.

FIG. 12E depicts a ROC curve of the specificity, sensitivity, and area under the curve (AUC) for a single-stage predictive cancer model including whole genome sequencing (WGS) features.

FIG. 12F depicts a ROC curve of the specificity, sensitivity, and area under the curve (AUC) for a single-stage predictive cancer model including small variant and WGS features.

FIG. 12G depicts a ROC curve of the specificity, sensitivity, and area under the curve (AUC) for a single-stage predictive cancer model including methylation and WGS features.

FIG. 12H depicts a ROC curve of the specificity, sensitivity, and area under the curve (AUC) for a single-stage predictive cancer model including small variant, methylation and WGS features.

FIG. 12I depicts a ROC curve of the specificity, sensitivity, and area under the curve (AUC) for a single-stage predictive cancer model including baseline features.

FIG. 12J depicts a ROC curve of the specificity, sensitivity, and area under the curve (AUC) for a single-stage predictive cancer model including baseline and WGS features.

FIG. 12K depicts a ROC curve of the specificity, sensitivity, and area under the curve (AUC) for a single-stage predictive cancer model including baseline and methylation features.

FIG. 12L depicts a ROC curve of the specificity, sensitivity, and area under the curve (AUC) for a single-stage predictive cancer model including baseline, small variant and methylation features.

FIG. 12M depicts a ROC curve of the specificity, sensitivity, and area under the curve (AUC) for a single-stage predictive cancer model including baseline and WGS features.

FIG. 12N depicts a ROC curve of the specificity, sensitivity, and area under the curve (AUC) for a single-stage predictive cancer model including baseline, small variant and WGS features.

FIG. 12O depicts a ROC curve of the specificity, sensitivity, and area under the curve (AUC) for a single-stage predictive cancer model including baseline, methylation and WGS features.

FIG. 12P depicts a ROC curve of the specificity, sensitivity, and area under the curve (AUC) for a single-stage predictive cancer model including baseline, small variant, methylation and WGS features.

### DETAILED DESCRIPTION

The figures and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. For example, a letter after a reference numeral, such as “predictive cancer model 170A,” indicates that the text refers specifically to the element having that particular reference numeral. A reference numeral in the text without a following letter, such as “predictive cancer model 170,” refers to any or all of the elements in the figures bearing that reference numeral (e.g. “predictive cancer model 170” in the text refers to reference numerals “predictive cancer model 170A” and/or “predictive cancer model 170B” in the figures).

The term “individual” refers to a human individual. The term “healthy individual” refers to an individual presumed to not have a cancer or disease. The term “subject” refers to an individual who is known to have, or potentially has, a cancer or disease.

The term “sequence reads” refers to nucleotide sequences read from a sample obtained from an individual. Sequence reads can be obtained through various methods known in the art.

The term “read segment” or “read” refers to any nucleotide sequences including sequence reads obtained from an individual and/or nucleotide sequences derived from the initial sequence read from a sample obtained from an individual.

The term “single nucleotide variant” or “SNV” refers to a substitution of one nucleotide to a different nucleotide at a position (e.g., site) of a nucleotide sequence, e.g., a sequence read from an individual. A substitution from a first nucleobase X to a second nucleobase Y may be denoted as “X>Y.” For example, a cytosine to thymine SNV may be denoted as “C>T.”

The term “indel” refers to any insertion or deletion of one or more base pairs having a length and a position (which may also be referred to as an anchor position) in a sequence read. An insertion corresponds to a positive length, while a deletion corresponds to a negative length.

The term “mutation” refers to one or more SNVs or indels.

The term “true” or “true positive” refers to a mutation that indicates real biology, for example, presence of a potential cancer, disease, or germline mutation in an individual. True positives are tumor-derived mutations and are not caused by mutations naturally occurring in healthy individuals (e.g., recurrent mutations) or other sources of artifacts such as process errors during assay preparation of nucleic acid samples.

The term “false positive” refers to a mutation incorrectly determined to be a true positive.

The term “cell free nucleic acid,” “cell free DNA,” or “cfDNA” refers to nucleic acid fragments that circulate in an individual's body (e.g., bloodstream) and originate from one or more healthy cells and/or from one or more cancer cells. Additionally cfDNA may come from other sources such as viruses, fetuses, etc.

The term “genomic nucleic acid,” “genomic DNA,” or “gDNA” refers to nucleic acid including chromosomal DNA that originates from one or more healthy (e.g., non-tumor) cells. In various embodiments, gDNA can be extracted from a cell derived from a blood cell lineage, such as a white blood cell.

The term “circulating tumor DNA” or “ctDNA” refers to nucleic acid fragments that originate from tumor cells or other types of cancer cells, which may be released into an individual's bloodstream as result of biological processes such as apoptosis or necrosis of dying cells or actively released by viable tumor cells.

The term “alternative allele” or “ALT” refers to an allele having one or more mutations relative to a reference allele, e.g., corresponding to a known gene.

The term “sequencing depth” or “depth” refers to a total number of read segments from a sample obtained from an individual.

The term “alternate depth” or “AD” refers to a number of read segments in a sample that support an ALT, e.g., include mutations of the ALT.

The term “reference depth” refers to a number of read segments in a sample that include a reference allele at a candidate variant location.

The term “variant” or “true variant” refers to a mutated nucleotide base at a position in the genome. Such a variant can lead to the development and/or progression of cancer in an individual.

The term “candidate variant,” “called variant,” or “putative variant” refers to one or more detected nucleotide variants of a nucleotide sequence, for example, at a position in the genome that is determined to be mutated. Generally, a nucleotide base is deemed a called variant based on the presence of an alternative allele on sequence reads obtained from a sample, where the sequence reads each cross over the position in the genome. The source of a candidate variant may initially be unknown or uncertain. During processing, candidate variants may be associated with an expected source such as gDNA (e.g., blood-derived) or cells impacted by cancer (e.g., tumor-derived). Additionally, candidate variants may be called as true positives.

The term “copy number aberrations” or “CNAs” refers to changes in copy number in somatic tumor cells. For example, CNAs can refer to copy number changes in a solid tumor.

The term “copy number variations” or “CNVs” refers to changes in copy number changes that derive from germline cells or from somatic copy number changes in non-tumor cells. For example, CNVs can refer to copy number changes in white blood cells that can arise due to clonal hematopoiesis.

The term “copy number event” refers to one or both of a copy number aberration and a copy number variation.

### 1. Generating a Cancer Prediction

1.1. Overall Process Flow

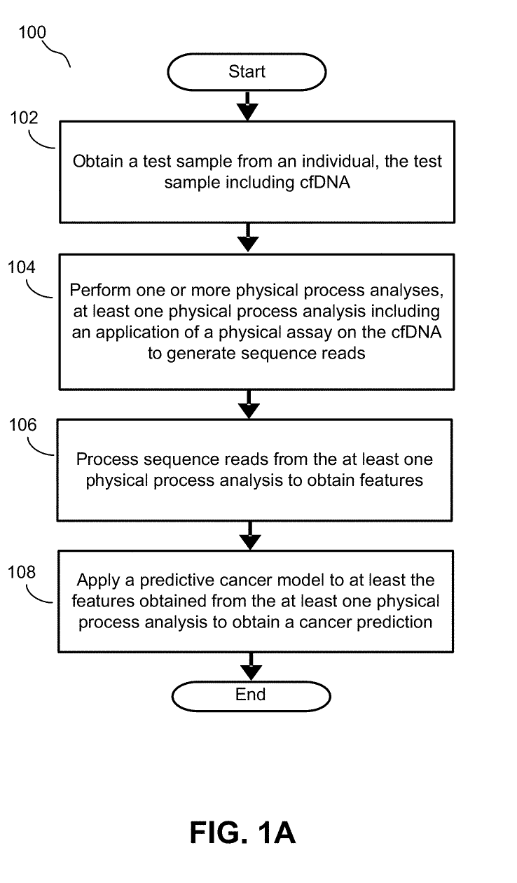

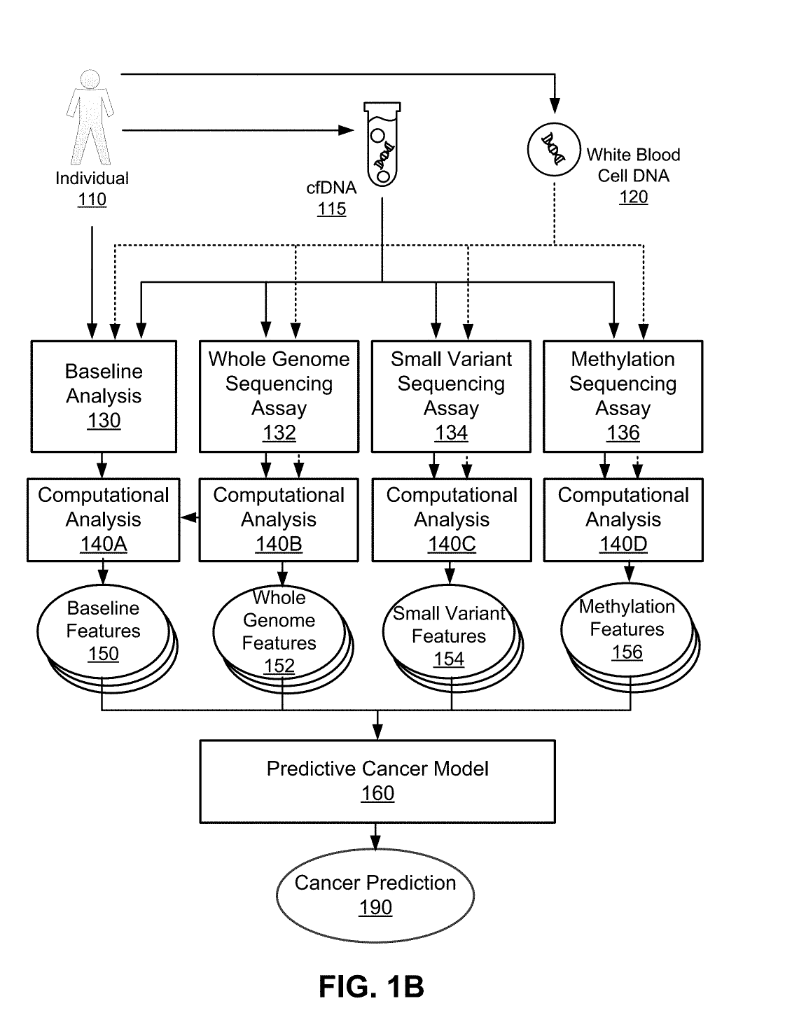

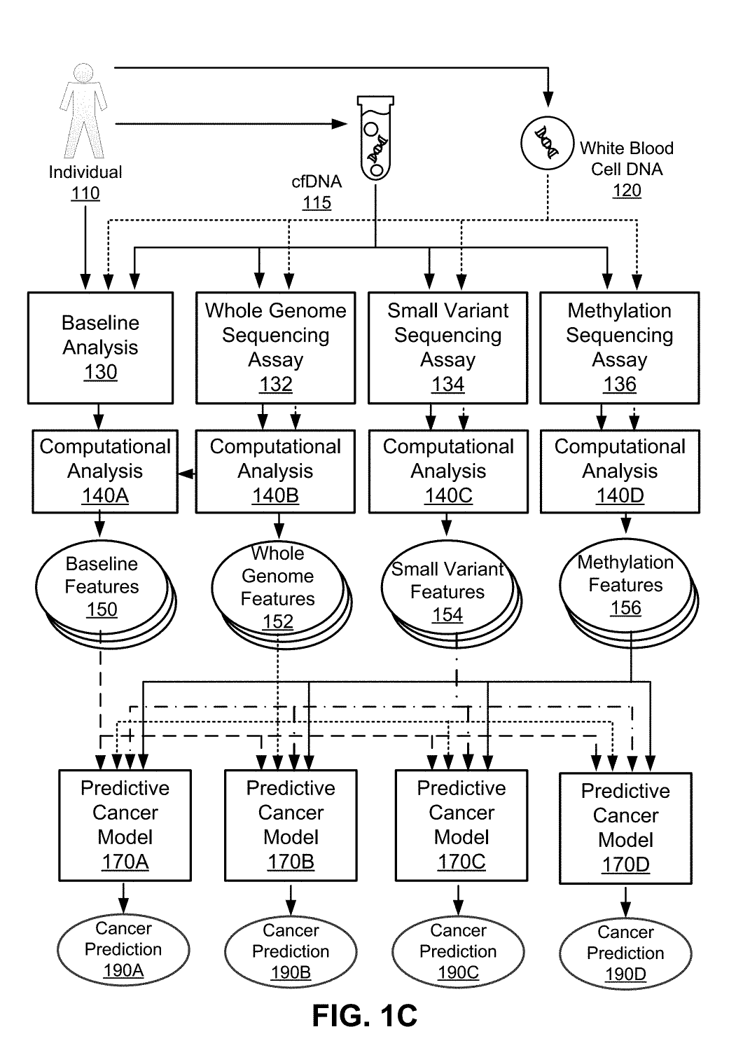

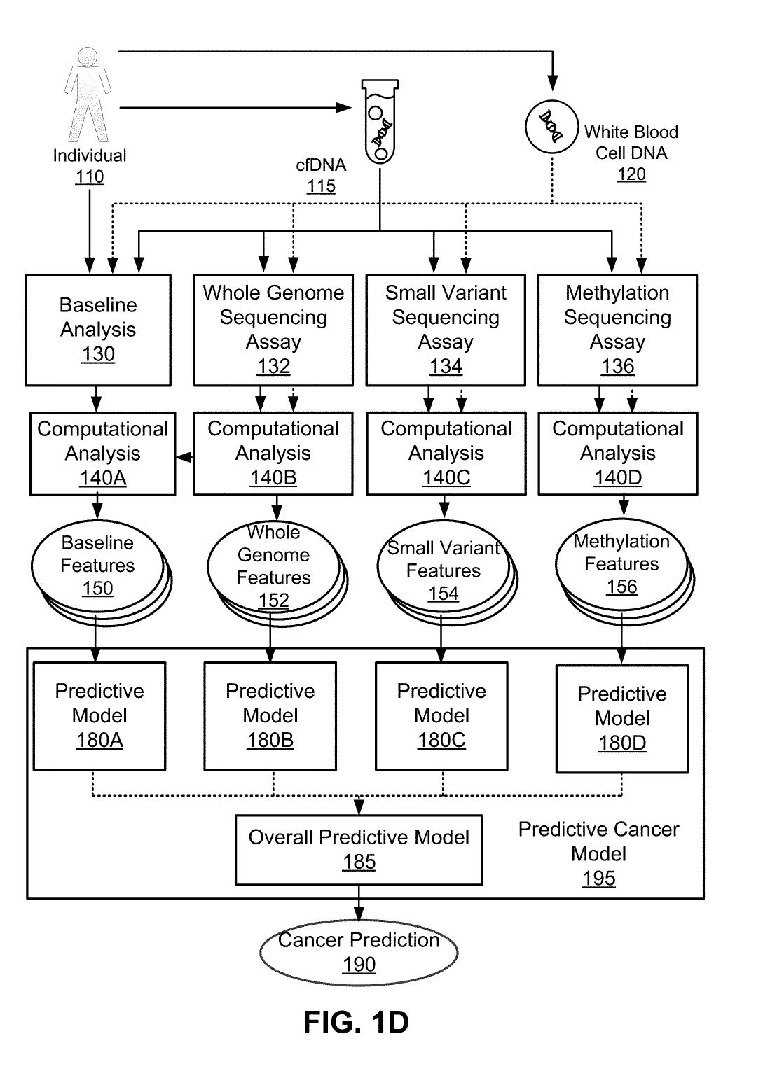

FIG. 1A depicts an overall flow process 100 for generating a cancer prediction based on features derived from a cfDNA sample obtained from an individual, in accordance with an embodiment. Further reference will be made to FIGS. 1B-1D, each of which depicts an overall flow diagram for determining a cancer prediction using at least a cfDNA sample obtained from an individual, in accordance with an embodiment.

At step 102, the test sample is obtained from the individual. Generally, samples may be from healthy subjects, subjects known to have or suspected of having cancer, or subjects where no prior information is known (e.g., asymptomatic subjects). The test sample may be a sample selected from the group consisting of blood, plasma, serum, urine, fecal, and saliva samples. Alternatively, the test sample may comprise a sample selected from the group consisting of whole blood, a blood fraction, a tissue biopsy, pleural fluid, pericardial fluid, cerebral spinal fluid, and peritoneal fluid.

As shown in each of FIGS. 1B-1D, a test sample may include cfDNA 115. In various embodiments, a test sample may include genomic DNA (gDNA). An example of a source of gDNA, as shown in FIGS. 1B-1D, is white blood cell (WBC) DNA 120.

At step 104, one or more physical process analyses are performed, at least one physical process analysis including a sequencing-based assay on cfDNA 115 to generate sequence reads. Referring to FIGS. 1B-1D, examples of a physical process analysis can be a baseline analysis 130 of the individual 110 or a sequencing-based assay on cfDNA 115 such as the performance of a whole genome sequencing assay 132, a small variant sequencing assay 134, or a methylation sequencing assay 136.

A baseline analysis 130 of the individual 110 can include a clinical analysis of the individual 110 and can be performed by a physician or a medical professional. In some embodiments, the baseline analysis 130 can include an analysis of germline changes detectable in the cfDNA 115 of the individual 110. In some embodiments, the baseline analysis 130 can perform the analysis of germline changes with additional information such as an identification of upregulated or downregulated genes. In other embodiments, the baseline analysis include analysis of clinical features (e.g., known risk factors for cancer, such as, a subject's age, race, body mass index (BMI), smoking history, alcohol intake, and/or family cancer history). Such additional information can be provided by a computational analysis, such as computational analysis 140B as depicted in FIGS. 1B-1D. The baseline analysis 130 is described in further detail below.

As used hereafter, a small variant sequencing assay refers to a physical assay that generates sequence reads, typically through targeted gene sequencing panels that can be used to determine small variants, examples of which include single nucleotide variants (SNVs) and/or insertions or deletions. Alternatively, as one of skill in the art would appreciate, assessment of small variants may also be done using a whole genome sequencing approach or a whole exome sequencing approach.

As used hereafter, a whole genome sequencing assay refers to a physical assay that generates sequence reads for a whole genome or a substantial portion of the whole genome which can be used to determine large variations such as copy number variations or copy number aberrations. Such a physical assay may employ whole genome sequencing techniques or whole exome sequencing techniques.

As used hereafter, a methylation sequencing assay refers to a physical assay that generates sequence reads which can be used to determine the methylation status of a plurality of CpG sites, or methylation patterns, across the genome. An example of such a methylation sequencing assay can include the bisulfite treatment of cfDNA for conversion of unmethylated cytosines (e.g., CpG sites) to uracil (e.g., using EZ DNA Methylation-Gold or an EZ DNA Methylation-Lightning kit (available from Zymo Research Corp)). Alternatively, an enzymatic conversion step (e.g., using a cytosine deaminase (such as APOBEC-Seq (available from NEBiolabs))) may be used for conversion of unmethylated cytosines to uracils. Following conversion, the converted cfDNA molecules can be sequenced through a whole genome sequencing process or a targeted gene sequencing panel and sequence reads used to assess methylation status at a plurality of CpG sites. Methylation-based sequencing approaches are known in the art (e.g., see US 2014/0080715 and U.S. Ser. No. 16/352,602, which are incorporated herein by reference). In another embodiment, DNA methylation may occur in cytosines in other contexts, for example CHG and CHH, where H is adenine, cytosine or thymine. Cytosine methylation in the form of 5-hydroxymethylcytosine may also assessed (see, e.g., WO 2010/037001 and WO 2011/127136, which are incorporated herein by reference), and features thereof, using the methods and procedures disclosed herein. In some embodiments, a methylation sequencing assay need not perform a base conversion step to determine methylation status of CpG sites across the genome. For example, such methylation sequencing assays can include PacBio sequencing or Oxford Nanopore sequencing.

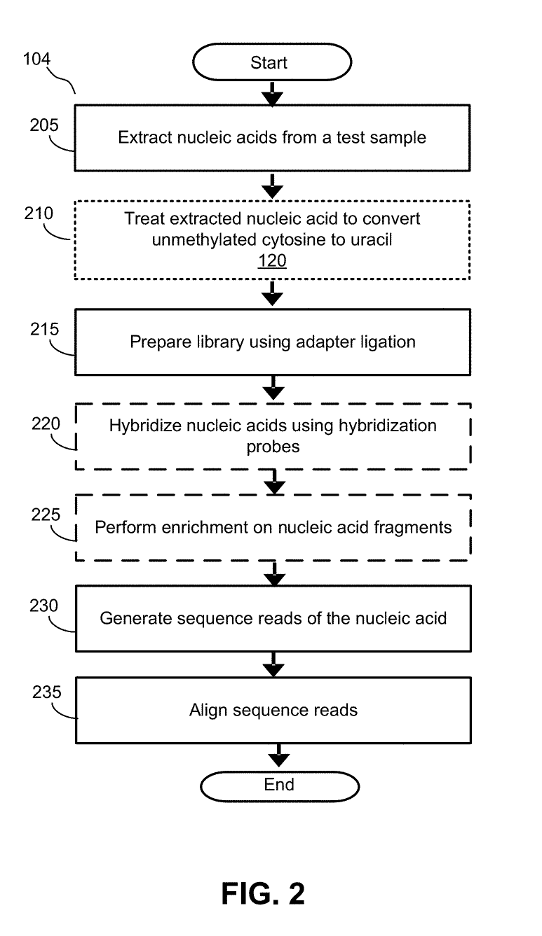

Each of the whole genome sequencing assay 132, small variant sequencing assay 134, and methylation sequencing assay 136 is performed on the cfDNA 115 to generate sequence reads of the cfDNA 115. In various embodiments, each of the whole genome sequencing assay 132, small variant sequencing assay 134, and methylation sequencing assay 136 are further performed on the WBC DNA 120 to generate sequence reads of the WBC DNA 120. The process steps performed in each of the whole genome sequencing assay 132, small variant sequencing assay 134, and methylation sequencing assay 136 is described in further detail in relation to FIG. 2.

At step 106, the sequence reads generated as a result of performing the sequencing-based assay are processed to determine values for features. Features, generally, are types of information obtainable from physical assays and/or computational analyses that may be used in predicting cancer in an individual. Generally, any given predictive model for identifying cancer in an individual includes one or more features as constituent components of the model. For any given patient or sample, a feature will have a value that is determined from the physical and/or computational analysis. These values are input into the predictive model to generate an output of the model.

Sequence reads are processed by applying a computational analysis. Generally, each computational analysis 140 represents an algorithm that is executable by a processor of a computer, hereafter referred to as a processing system. Therefore, each computational analysis analyzes sequence reads and outputs value features based on the sequence reads. Each computational analysis is specific for a given sequencing-based assay and therefore, each computational analysis outputs a particular type of feature that is specific for the sequencing-based assay.

As shown in FIGS. 1B-1D, sequence reads generated from application of a whole genome sequencing assay 132 are processed using computational analysis 140B, otherwise referred to as a whole genome computational analysis. The computational analysis 140B outputs whole genome features 152. Sequence reads generated from application of a small variant sequencing assay are processed using a computational analysis 140C, otherwise referred to as a small variant computational analysis. The computational analysis 140C outputs small variant feature 154. Sequence reads generated from application of a methylation sequencing assay are processed using computational analysis 140D, otherwise referred to as a methylation computational analysis. The computational analysis 140C outputs methylation features 156. Additionally, computational analysis 140A analyzes information from the baseline analysis 130 and outputs baseline features 150.

At step 108, a predictive cancer model is applied to the features to generate a cancer prediction for the individual 110. Examples of a cancer prediction include a presence or absence of cancer, a tissue of origin of cancer, a severity, stage, a grade of cancer, a cancer sub-type, a treatment decision, and a likelihood of response to a treatment. In various embodiments, the cancer prediction output by the predictive cancer model is a score, such as a likelihood or probability that indicates one or more of: a presence or absence of cancer, a tissue of origin of cancer, a severity, stage, a grade of cancer, a cancer sub-type, a treatment decision, and a likelihood of response to a treatment.

Generally, any such scores may either be singular, such as the presence or absence of cancer generally, presence/absence of a particular type of cancer. Alternatively, such scores may be plural, such that the output of the predictive cancer model may, for example, be a score representing the presence/absence of each of a number of types of cancer, a score representing the severity/grade of each of a number of types of cancer, a score representing the likelihood that particular cfDNA originated in each of a number of types of tissue, and so on. For clarity of description, the output of the predictive cancer model is generally referred to as a set of scores, the set comprising one or more scores depending upon what the predictive cancer model is configured to determine.

The predictive cancer model can be differently structured based on the particular features of the predictive cancer model. For example, the predictive cancer model can include one, two, three, or four different types of features, such as the baseline features 150, whole genome features 152, small variant features 154, and methylation features 156. In some embodiments, there may be four separate predictive cancer models, each structured to include one type of feature. In some embodiments, the predictive cancer model is a two-stage model that includes a first set of sub-models that each include a type of feature and a second sub-model that analyzes the outputs of the first set of sub-models to determine a cancer prediction. Each particularly structured predictive cancer model is described hereafter in relation to a processing workflow that generates values of one or more types of features that the predictive cancer model receives. As used hereafter, a workflow process refers to the performance of the physical process analysis, computational analysis, and application of a predictive cancer model.

In various embodiments, values of features output from different computational analyses are input into a single predictive cancer model to generate a cancer prediction. For example, referring to FIG. 1B, values of each of baseline features 150, whole genome features 152, small variant features 154, and methylation features 156 can be compiled (e.g., into a feature vector) and provided as input to a predictive cancer model 160. The predictive cancer model 160 outputs the cancer prediction 190 based on the provided features.

In various embodiments, a single workflow process is performed to generate a single cancer prediction 190 without a need for performing other workflow processes. Therefore, values of features output from a single computational analysis are input into a single predictive cancer model to generate a cancer prediction. For example, referring to FIG. 1C, to generate cancer prediction 190A, an individual 110, cfDNA 115, and/or WBC DNA 120 are analyzed using a baseline analysis 132, computational analysis 140B that analyzes the output of the baseline analysis 130 to obtain values of baseline features 150, and the values of baseline features 150 are provided as input to the predictive cancer model 170A. As another example, to generate cancer prediction 190B, cfDNA 115 and/or WBC DNA 120 are analyzed using a whole genome sequencing assay 132, computational analysis 140B that analyzes the sequence reads generated by the whole genome sequencing assay 132 to obtain values of whole genome features 152, and the values of whole genome features 152 are provided as input to the predictive cancer model 170B. As yet another example, to generate cancer prediction 190C, cfDNA 115 and/or WBC DNA 120 are analyzed using a small variant sequencing assay 134, computational analysis 140C that analyzes the sequence reads generated by the small variant sequencing assay 134 to obtain values of small variant features 154, and the values of small variant features 154 are provided as input to the predictive cancer model 170C. As yet another example, to generate cancer prediction 190D, cfDNA 115 and/or WBC DNA 120 are analyzed using a methylation sequencing assay 136, computational analysis 140D that analyzes the sequence reads generated by the methylation sequencing assay 136 to obtain values of methylation features 156, and the values of methylation features 156 are provided as input to the predictive cancer model 170D.

In various embodiments, a predictive cancer model (e.g., any one of predictive cancer models 170A-D) can generate a cancer prediction 190A-D based on values of two types of features (e.g., two features selected from baseline features 150, whole genome features 152, small variant features 154, and methylation features 156). In some embodiments, a predictive cancer model can generate a cancer prediction 190A-D based on values of three types of features (e.g., three features selected from baseline features 150, whole genome features 152, small variant features 154, and methylation features 156).

In various embodiments, a two stage predictive cancer model is applied to the features to generate a cancer prediction. For example, at a first stage, the values of features output from each computational analysis are separately input into individual sub-models. At a second stage, the output of each individual sub-model is provided as input into an overall sub-model to generate a cancer prediction. FIG. 1D depicts an example of a two stage predictive cancer model 195. Here, baseline features 150 are provided as input to predictive model 180A, whole genome features 152 are provided as input to predictive model 180B, small variant features 154 are provided as input to predictive model 180C, and methylation features 156 are provided as input to predictive model 180D. The output of each of predictive models 180A, 180B, 180C, and 180D can serve as input into the overall predictive model 185. In various embodiments, the output of each of predictive models 180A, 180B, 180C, and 180D is one or more scores. Therefore, the overall predictive model 185 generates a cancer prediction 190 based on the one or more scores.

Although FIG. 1D depicts that the output of four separate prediction models 180A, 180B, 180C, and 180D are provided as input to the overall predictive model 185, in various embodiments, additional or fewer prediction models may be involved in provided an input to the overall predictive model 185. For example, in some embodiments, one, two, or three of the four predictive models 180A, 180B, 180C, and 180D output information that is provided as input to the overall predictive model 185.

In various embodiments, the number of scores output by each of the predictive models 180A, 180B, 180C, and 180D may differ. For example, predictive model 180B may output one set of scores (hereafter referred to a “WGS score”), predictive model 180C may output a set of two scores (hereafter referred to as “variant gene score” and “Order score”), and predictive model 180C may output a set of three scores (hereafter referred to as “MSUM score,” “WGBS score,” and “Binary score”).

In each of the different embodiments of the predictive cancer model (e.g., predictive cancer model 160 in FIG. 1B, predictive cancer models 170A-D, or predictive cancer model 195 in FIG. 1D), each predictive cancer model can be one of a decision tree, an ensemble (e.g., bagging, boosting, random forest), gradient boosting machine, linear regression, Naïve Bayes, neural network, XGBoost, or logistic regression. Each predictive cancer model includes learned weights for the features that are adjusted during training. The term weights is used generically here to represent the learned quantity associated with any given feature of a model, regardless of which particular machine learning technique is used.

During training, training data is processed to generate values for features that are used to train the weights of the predictive cancer model. As an example, training data can include cfDNA and/or WBC DNA obtained from training samples, as well as an output label. For example, the output label can be indication as to whether the individual is known to be cancerous or known to be devoid of cancer (e.g., healthy), an indication of a cancer tissue of origin, or an indication of a severity of the cancer. Depending on the particular embodiment shown in FIGS. 1B-D, the predictive cancer model receives the values for one or more of the features obtained from one or more the physical assays and computational analyses relevant to the model to be trained. Depending on the differences between the scores output by the model-in-training and the output labels of the training data, the weights of the predictive cancer model are optimized enable the predictive cancer model to make more accurate predictions. In various embodiments, a predictive cancer model may be a non-parametric model (e.g., k-nearest neighbors) and therefore, the predictive cancer model can be trained to make more accurately make predictions without having to optimize parameters.

The trained predictive cancer model can be stored and subsequently retrieved when needed, for example, during deployment in step 108 of FIG. 1A.

1.2. Physical Assays

FIG. 2 is flowchart of a method for performing a physical assay (e.g., a sequencing assay) to generate sequence reads, in accordance with an embodiment. Here, the flowchart depicts step 104 of FIG. 1A in more detail. The method 104 includes, but is not limited to, the following steps. For example, any step of the method 104 may comprise a quantitation sub-step for quality control or other laboratory assay procedures known to one skilled in the art.

Generally, various sub-combinations of the steps (e.g., steps 205-235) are performed for each of the whole genome sequencing assay, small variant sequencing assay, and methylation sequencing assay. Specifically, steps 205, 215, 230, and 235 are performed for the whole genome sequencing assay. Steps 205 and 215-235 are performed for the small variant sequencing assay. In some embodiments, each of steps 205-235 are performed for the methylation sequencing assay. For example, a methylation sequencing assay that employs a targeted gene panel bisulfite sequencing employs each of steps 205-235. In some embodiments, steps 205-215 and 230-235 are performed for the methylation sequencing assay. For example, a methylation sequencing assay that employs whole genome bisulfite sequencing need not perform steps 220 and 225.

At step 205, nucleic acids (DNA or RNA) are extracted from a test sample. In the present disclosure, DNA and RNA may be used interchangeably unless otherwise indicated. That is, the following embodiments for using error source information in variant calling and quality control may be applicable to both DNA and RNA types of nucleic acid sequences.

However, the examples described herein may focus on DNA for purposes of clarity and explanation. In various embodiments, DNA (e.g., cfDNA) is extracted from the test sample through a purification process. In general, any known method in the art can be used for purifying DNA. For example, nucleic acids can be isolated by pelleting and/or precipitating the nucleic acids in a tube. The extracted nucleic acids may include cfDNA or it may include gDNA, such as WBC DNA.

In step 210, the cfDNA fragments are treated to convert unmethylated cytosines to uracils. In one embodiment, the method uses a bisulfite treatment of the DNA which converts the unmethylated cytosines to uracils without converting the methylated cytosines. For example, a commercial kit such as the EZ DNA METHYLATION—Gold, EZ DNA METHYLATION—Direct or an EZ DNA METHYLATION—Lightning kit (available from Zymo Research Corp, Irvine, Calif.) is used for the bisulfite conversion. In another embodiment, the conversion of unmethylated cytosines to uracils is accomplished using an enzymatic reaction. For example, the conversion can use a commercially available kit for conversion of unmethylated cytosines to uracils, such as APOBEC-Seq (NEBiolabs, Ipswich, Mass.).

At step 215, a sequencing library is prepared. During library preparation, adapters, for example, include one or more sequencing oligonucleotides for use in subsequent cluster generation and/or sequencing (e.g., known P5 and P7 sequences for used in sequencing by synthesis (SBS) (Illumina, San Diego, Calif.)) are ligated to the ends of the nucleic acid fragments through adapter ligation. In one embodiment, unique molecular identifiers (UMI) are added to the extracted nucleic acids during adapter ligation. The UMIs are short nucleic acid sequences (e.g., 4-10 base pairs) that are added to ends of nucleic acids during adapter ligation. In some embodiments, UMIs are degenerate base pairs that serve as a unique tag that can be used to identify sequence reads obtained from nucleic acids. As described later, the UMIs can be further replicated along with the attached nucleic acids during amplification, which provides a way to identify sequence reads that originate from the same original nucleic acid segment in downstream analysis.

In step 220, hybridization probes are used to enrich a sequencing library for a selected set of nucleic acids. Hybridization probes can be designed to target and hybridize with targeted nucleic acid sequences to pull down and enrich targeted nucleic acid fragments that may be informative for the presence or absence of cancer (or disease), cancer status, or a cancer classification (e.g., cancer type or tissue of origin). In accordance with this step, a plurality of hybridization pull down probes can be used for a given target sequence or gene. The probes can range in length from about 40 to about 160 base pairs (bp), from about 60 to about 120 bp, or from about 70 bp to about 100 bp. In one embodiment, the probes cover overlapping portions of the target region or gene. For targeted gene panel sequencing, the hybridization probes are designed to target and pull down nucleic acid fragments that derive from specific gene sequences that are included in the targeted gene panel. For whole exome sequencing, the hybridization probes are designed to target and pull down nucleic acid fragments that derive from exon sequences in a reference genome. As one of skill in the art would readily appreciate, other known means in the art for targeted enrichment of nucleic acids may be used.

After a hybridization step 220, the hybridized nucleic acid fragments are enriched 225. For example, the hybridized nucleic acid fragments can be captured and amplified using PCR. The target sequences can be enriched to obtain enriched sequences that can be subsequently sequenced. This improves the sequencing depth of sequence reads.

In step 230, the nucleic acids are sequenced to generate sequence reads. Sequence reads may be acquired by known means in the art. For example, a number of techniques and platforms obtain sequence reads directly from millions of individual nucleic acid (e.g., DNA such as cfDNA or gDNA) molecules in parallel. Such techniques can be suitable for performing any of targeted gene panel sequencing, whole exome sequencing, whole genome sequencing, targeted gene panel bisulfite sequencing, and whole genome bisulfite sequencing.

As a first example, sequencing-by-synthesis technologies rely on the detection of fluorescent nucleotides as they are incorporated into a nascent strand of DNA that is complementary to the template being sequenced. In one method, oligonucleotides 30-50 bases in length are covalently anchored at the 5′ end to glass cover slips. These anchored strands perform two functions. First, they act as capture sites for the target template strands if the templates are configured with capture tails complementary to the surface-bound oligonucleotides. They also act as primers for the template directed primer extension that forms the basis of the sequence reading. The capture primers function as a fixed position site for sequence determination using multiple cycles of synthesis, detection, and chemical cleavage of the dye-linker to remove the dye. Each cycle consists of adding the polymerase/labeled nucleotide mixture, rinsing, imaging and cleavage of dye.

In an alternative method, polymerase is modified with a fluorescent donor molecule and immobilized on a glass slide, while each nucleotide is color-coded with an acceptor fluorescent moiety attached to a gamma-phosphate. The system detects the interaction between a fluorescently-tagged polymerase and a fluorescently modified nucleotide as the nucleotide becomes incorporated into the de novo chain.

Any suitable sequencing-by-synthesis platform can be used to identify mutations. Sequencing-by-synthesis platforms include the Genome Sequencers from Roche/454 Life Sciences, the GENOME ANALYZER from Illumina/SOLEXA, the SOLID system from Applied BioSystems, and the HELISCOPE system from Helicos Biosciences. Sequencing-by-synthesis platforms have also been described by Pacific BioSciences and VisiGen Biotechnologies. In some embodiments, a plurality of nucleic acid molecules being sequenced is bound to a support (e.g., solid support). To immobilize the nucleic acid on a support, a capture sequence/universal priming site can be added at the 3′ and/or 5′ end of the template. The nucleic acids can be bound to the support by hybridizing the capture sequence to a complementary sequence covalently attached to the support. The capture sequence (also referred to as a universal capture sequence) is a nucleic acid sequence complementary to a sequence attached to a support that may dually serve as a universal primer.

As an alternative to a capture sequence, a member of a coupling pair (such as, e.g., antibody/antigen, receptor/ligand, or the avidin-biotin pair) can be linked to each fragment to be captured on a surface coated with a respective second member of that coupling pair. Subsequent to the capture, the sequence can be analyzed, for example, by single molecule detection/sequencing, including template-dependent sequencing-by-synthesis. In sequencing-by-synthesis, the surface-bound molecule is exposed to a plurality of labeled nucleotide triphosphates in the presence of polymerase. The sequence of the template is determined by the order of labeled nucleotides incorporated into the 3′ end of the growing chain. This can be done in real time or can be done in a step-and-repeat mode. For real-time analysis, different optical labels to each nucleotide can be incorporated and multiple lasers can be utilized for stimulation of incorporated nucleotides.

Massively parallel sequencing or next generation sequencing (NGS) techniques include synthesis technology, pyrosequencing, ion semiconductor technology, single-molecule real-time sequencing, sequencing by ligation, nanopore sequencing, or paired-end sequencing. Examples of massively parallel sequencing platforms are the Illumina HISEQ or MISEQ, ION PERSONAL GENOME MACHINE, the PACBIO RSII sequencer or SEQUEL System, Qiagen's GENEREADER, and the Oxford MINION. Additional similar current massively parallel sequencing technologies can be used, as well as future generations of these technologies.

At step 230, the sequence reads may be aligned to a reference genome using known methods in the art to determine alignment position information. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene.

In various embodiments, a sequence read is comprised of a read pair denoted as R₁ and R₂. For example, the first read R₁ may be sequenced from a first end of a nucleic acid fragment whereas the second read R₂ may be sequenced from the second end of the nucleic acid fragment. Therefore, nucleotide base pairs of the first read R₁ and second read R₂ may be aligned consistently (e.g., in opposite orientations) with nucleotide bases of the reference genome. Alignment position information derived from the read pair R₁ and R₂ may include a beginning position in the reference genome that corresponds to an end of a first read (e.g., R₁) and an end position in the reference genome that corresponds to an end of a second read (e.g., R₂). In other words, the beginning position and end position in the reference genome represent the likely location within the reference genome to which the nucleic acid fragment corresponds. An output file having SAM (sequence alignment map) format or BAM (binary alignment map) format may be generated and output for further analysis.

Following step 235, the aligned sequence reads are processed using a computational analysis, such as computational analysis 140B, 140C, or 140D as described above and shown in FIG. 1D. Each of the small variant computational analysis 140C, whole genome computation assay 140B, methylation computational analysis 140D, and baseline computational analysis are described in further detail below.

### 2. Small Variant Computational Analysis

2.1. Small Variant Features

The small variant computational analysis 140C receives sequence reads generated by the small variant sequencing assay 134 and determines values of small variant features 154 based on the sequence reads. As previously described, the small variant sequencing assay may be a sequencing-based assay that generates sequence reads, typically through targeted gene sequencing panels that can be used to determine small variants, examples of which include single nucleotide variants (SNVs) and/or insertions or deletions. Alternatively, as one of skill in the art would appreciate, assessment of small variants may also be done using a whole genome sequencing approach or a whole exome sequencing approach. Examples of small variant features 154 include any of: a total number of somatic variants in a subject's cfDNA, a total number of non-synonymous variants, total number of synonymous variants, a presence/absence of somatic variants per gene in a gene panel, a presence/absence of somatic variants for particular genes that are known to be associated with cancer, an allele frequency (AF) of a somatic variant per gene in a gene panel, a maximum AF of a somatic variant per gene in a gene panel, an AF of a somatic variant per category as designated by a publicly available database, such as oncoKB, and a ranked order of somatic variants according to the AF of somatic variants.

Generally, the feature values for the small variant features 154 are predicated on the accurate identification of somatic variants that may be indicative of cancer in the individual. The small variant computational analysis 140C identifies candidate variants and from amongst the candidate variants, differentiates between somatic variants likely present in the genome of the individual and false positive variants that are unlikely to be predictive of cancer in the individual. More specifically, the small variant computational analysis 140C identifies candidate variants present in cfDNA that are likely to be derived from a somatic source in view of interfering signals such as noise and/or variants that can be attributed to a genomic source (e.g., from gDNA or WBC DNA). Additionally candidate variants can be filtered to remove false positive variants that may arise due to an artifact and therefore are not indicative of cancer in the individual. As an example, false positive variants may be variants detected at or near the edge of sequence reads, which arise due to spontaneous cytosine deamination and end repair errors. Thus, somatic variants, and features thereof, that remain following the filtering out of false positive variants can be used to determine the small variant features.

For the feature of the total number of somatic variants, the small variant computational analysis 140C totals the identified somatic variants across the genome, or gene panel. Thus, for a cfDNA sample obtained from an individual, the feature of the total number of somatic variants is represented as a single, numerical value of the total number of somatic variants identified in the cfDNA of the sample.

For the feature of the total number of nonsynonymous variants, the small variant computational analysis 140C may further filter the identified somatic variants to identify the somatic variants that are nonsynonymous variants. As is well known in the art, a non-synonymous variant of a nucleic acid sequence results in a change in the amino acid sequence of a protein associated with the nucleic acid sequence. For instance, non-synonymous variants may alter one or more phenotypes of an individual or cause (or leave more vulnerable) the individual to develop cancer, cancerous cells, or other types of diseases. Therefore, the small variant computation analysis 140C determines that a candidate variant would result in a non-synonymous variant by determining that a modification to one or more nucleobases of a trinucleotide would cause a different amino acid to be produced based on the modified trinucleotide. A feature value for the total number of nonsynonymous variants is determined by summating the identified nonsynonymous variants across the genome. Thus, for a cfDNA sample obtained from an individual, the feature of the total number of nonsynonymous variants is represented as a single, numerical value.

For the feature of the total number of synonymous variants, synonymous variants represent other somatic variants that are not categorized as nonsynonymous variants. In other words, the small variant computational analysis 140C can perform the filtering of identified somatic variants, as described in relation to nonsynonymous variants, and identify the synonymous variants across the genome, or gene panel. Thus, for a cfDNA sample obtained from an individual, the feature of the total number of synonymous variants is represented as a single numerical value.

The feature of a presence/absence of somatic variants per gene can involve multiple feature values for a cfDNA sample. For example, a targeted gene panel may include 500 genes in the panel and therefore, the small variant computational analysis 140C can generate 500 feature values, each feature value representing either a presence or absence of somatic variants for a gene in the panel. As an example, if a somatic variant is present in the gene, then the value of the feature is 1. Conversely, if a somatic variant is not present in the gene, then the value of the feature is 0. In general, any size gene panel may be used. For example, the gene panel may comprise 100, 200, 500, 1000, 2000, 10,000 or more genes targets across the genome. In other embodiment, the gene panel may comprise from about 50 to about 10,000 gene targets, from about 100 to about 2,000 gene targets, or from about 200 to about 1,000 gene targets.

For the feature of presence/absence of somatic variants for particular genes that are known to be associated with cancer, the particular genes known to be associated with cancer can be accessed from a public database such as OncoKB. Examples of genes known to be associated with cancer include p53, LRP1B, and KRAS. Each gene known to be associated with cancer can be associated with a feature value, such as a 1 (indicating that a somatic variant is present in the gene) or a 0 (indicating that a somatic variant is not present in the gene).

The AF of a somatic variant per gene (e.g., in a gene panel) refers to the frequency of one or more somatic variants in the sequence reads. Generally, this feature is represented by one feature value per gene of a gene panel or per gene across the genome. The value of this feature can be a statistical value of AFs of somatic variants of the gene. In various embodiments, this feature refers to the one somatic variant in the gene with the maximum AF. In some embodiments, this feature refers to the average AF of somatic variants of the gene. Therefore, for a targeted gene panel of 500 genes, there are 500 feature values that represent the AF of a somatic variant per gene (e.g., in a gene panel).

The AF of a somatic variant per category as designated by a publicly available database, such as oncoKB. For example, oncoKB categorizes genes in one of four different categories. In one embodiment, the AF of a somatic variant per category is a maximum AF of a somatic variant across the genes in the category. In one embodiment, the AF of a somatic variant per category is a mean AF across somatic variants across the genes in the category.

The ranked order of somatic variants according to the AF of somatic variants refers to the top N allele frequencies of somatic variants. In general, the value of a variant allele frequency can be between 0 to 1, where a variant allele frequency of 0 indicates no sequence reads that possess the alternate allele at the position and where a variant allele frequency of 1 indicates that all sequence reads possess the alternate allele at the position. In other embodiments, other ranges and/or values of variant allele frequencies can be used. In various embodiments, the ranked order feature is independent of the somatic variants themselves and instead, is only represented by the values of the top Nvariant allele frequencies. An example of the ranked order feature for the top 5 allele frequencies can be represented as: [0.1, 0.08, 0.05, 0.03, 0.02] which indicates that the 5 highest allele frequencies, independent of the somatic variants, range from 0.02 up to 0.1.

2.2. Small Variant Computational Analysis Process Overview

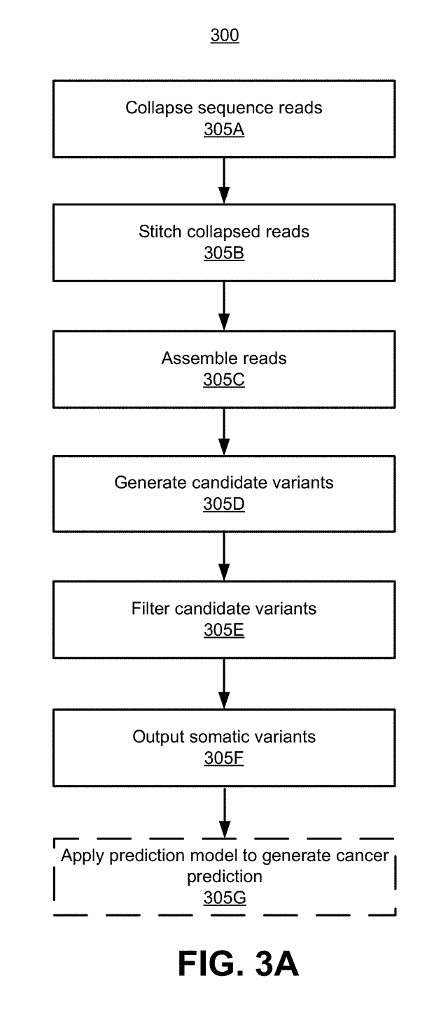

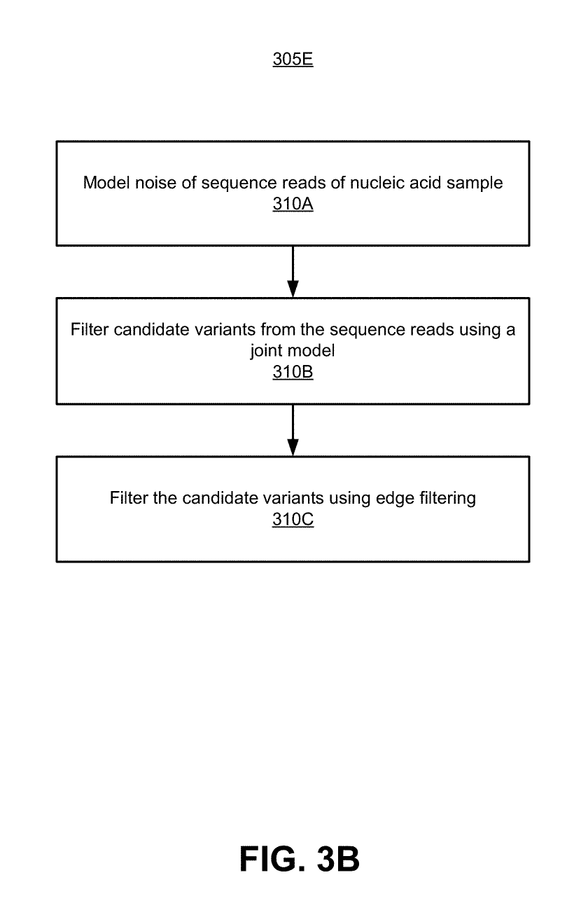

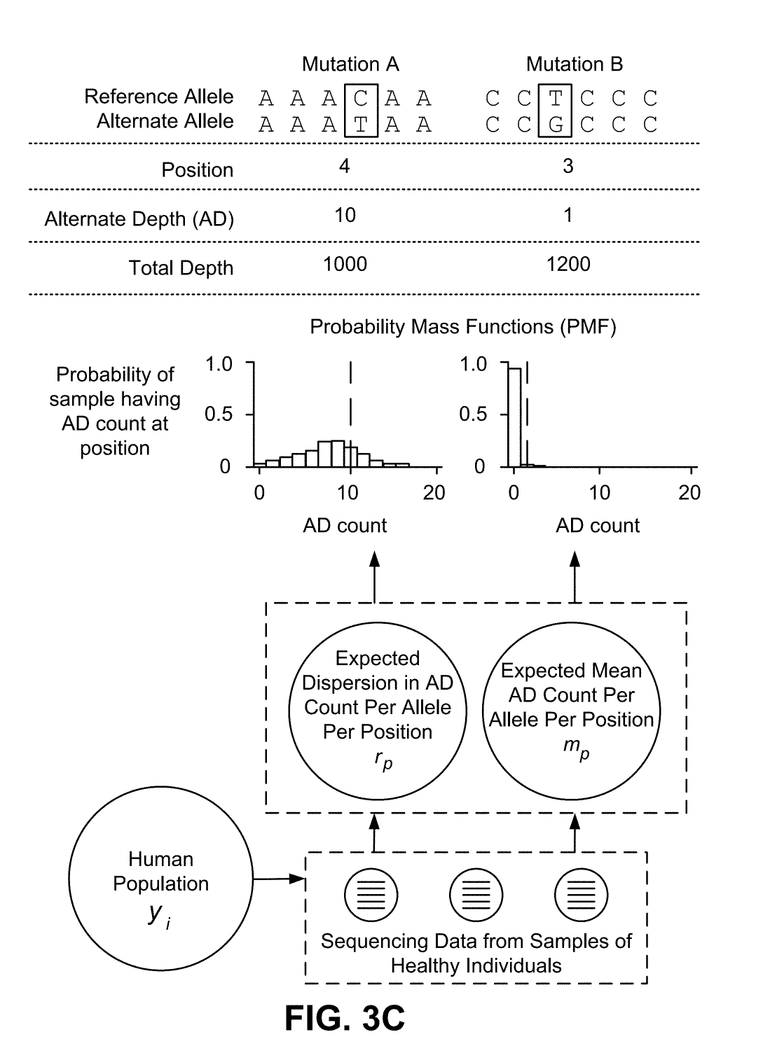

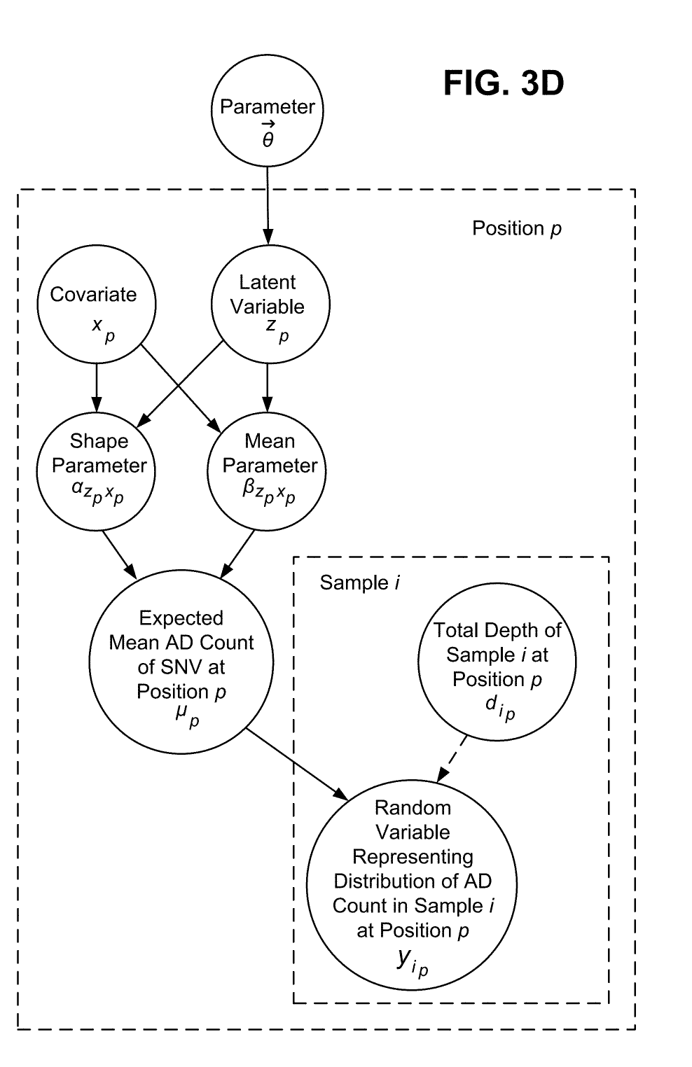

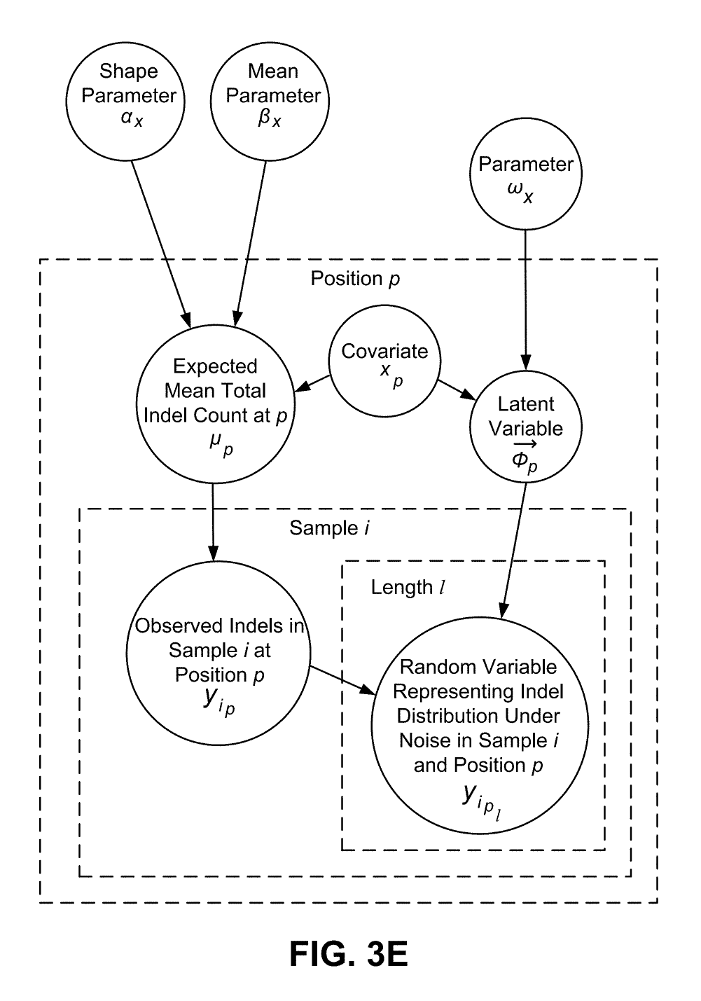

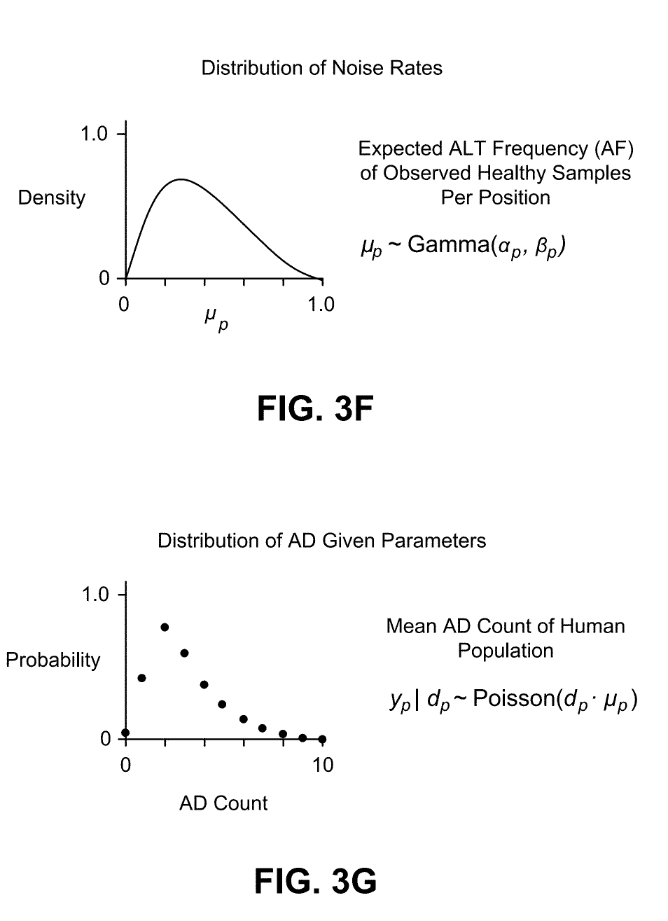

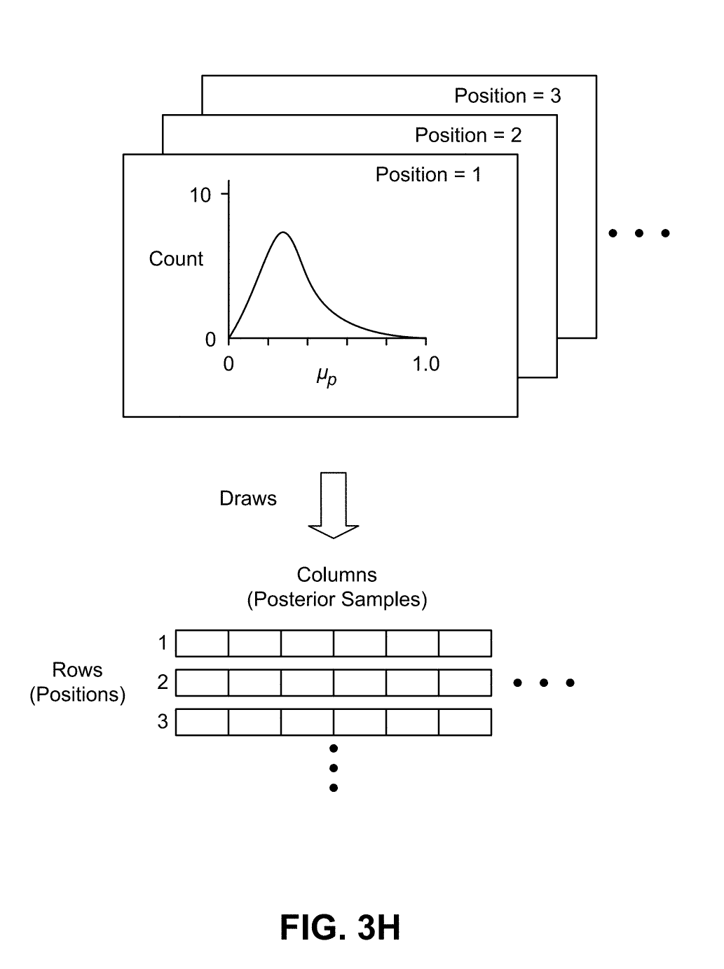

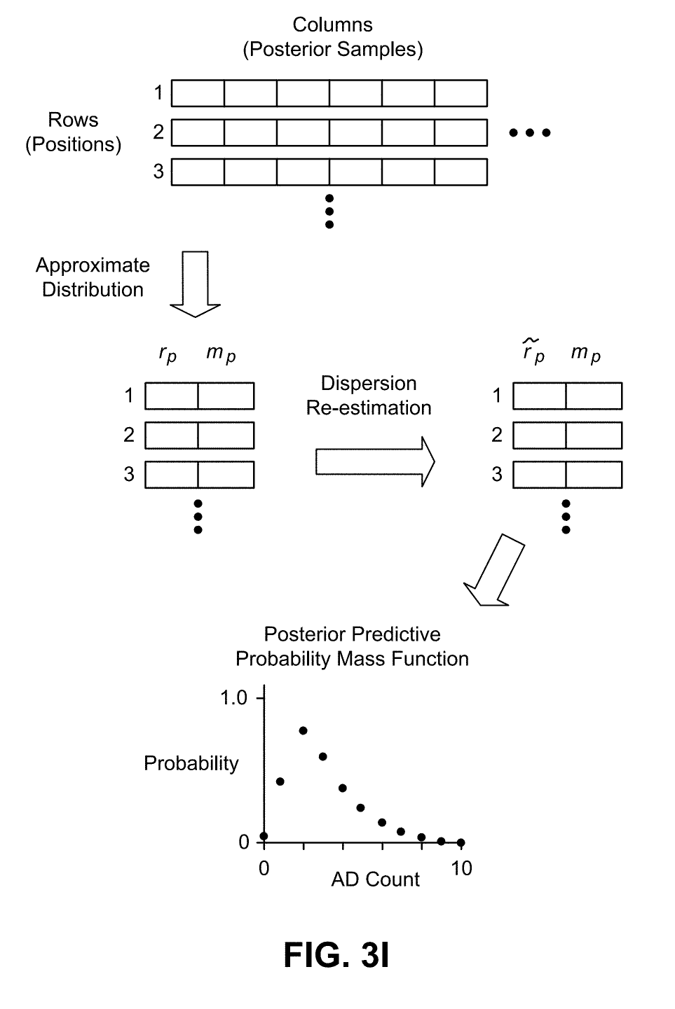

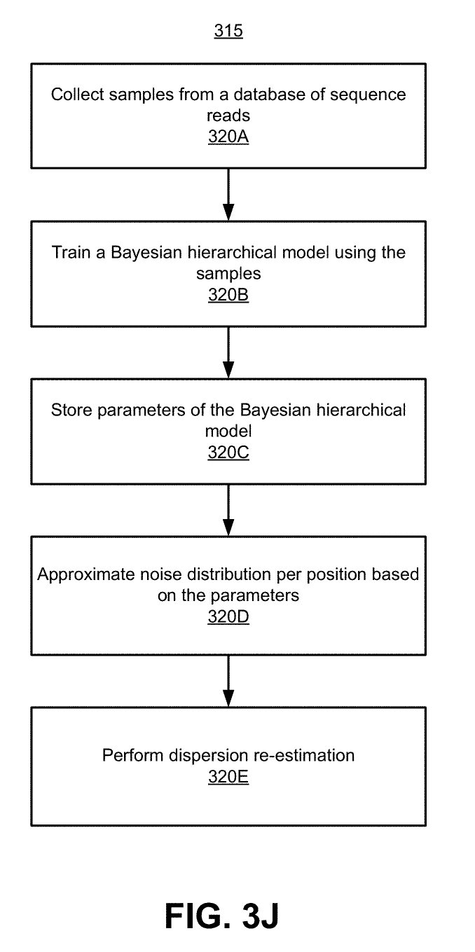

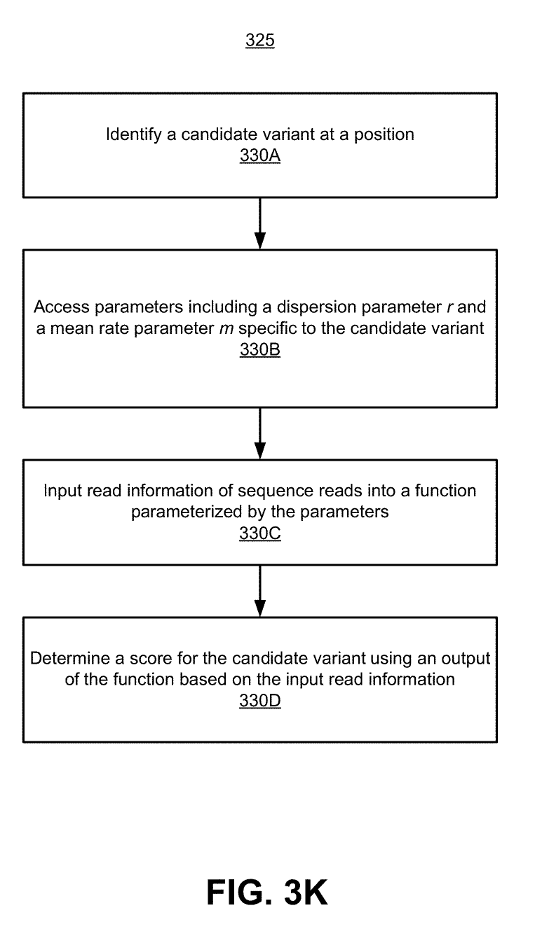

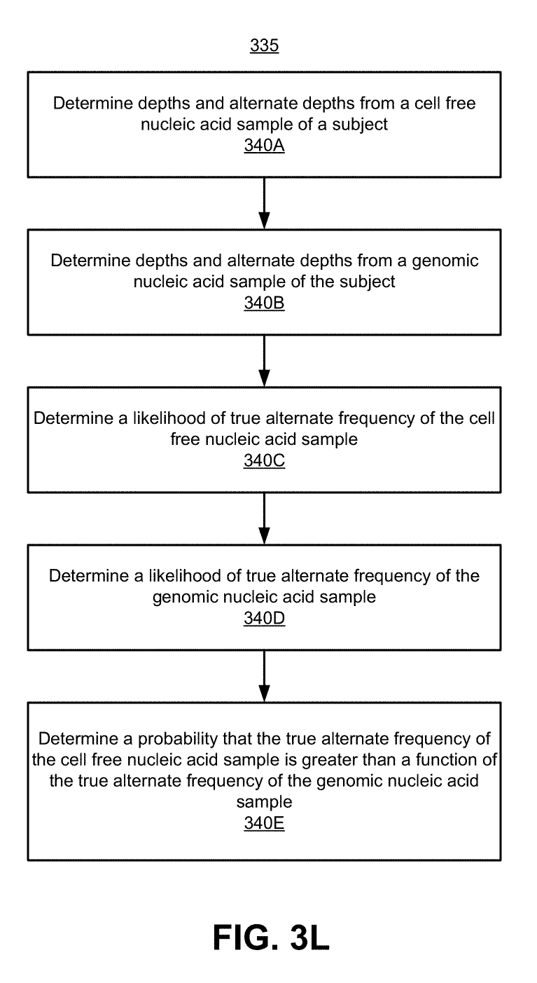

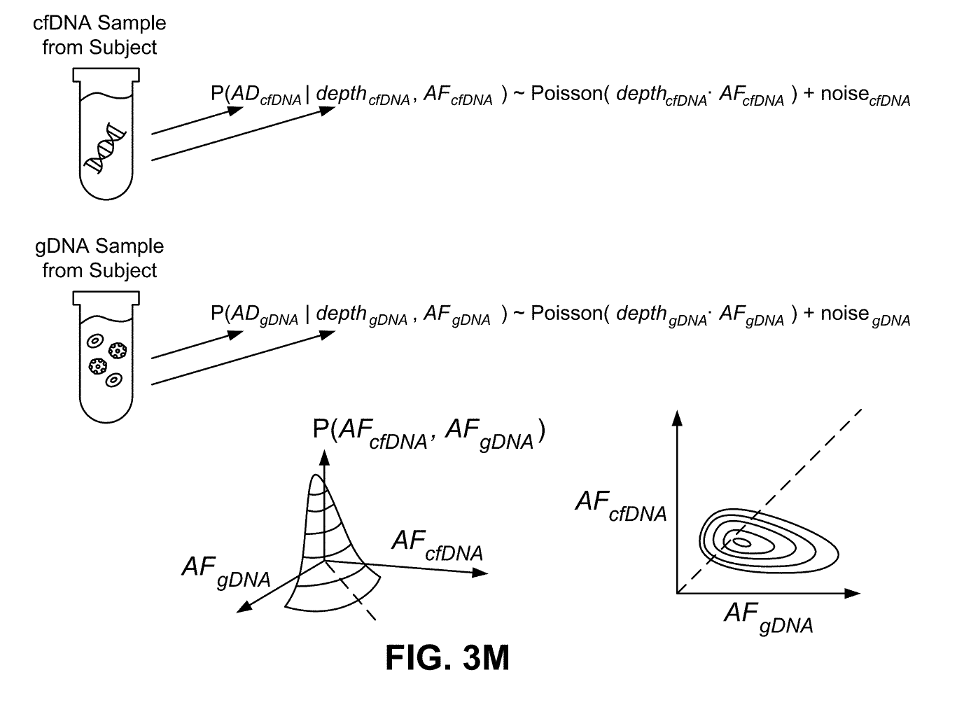

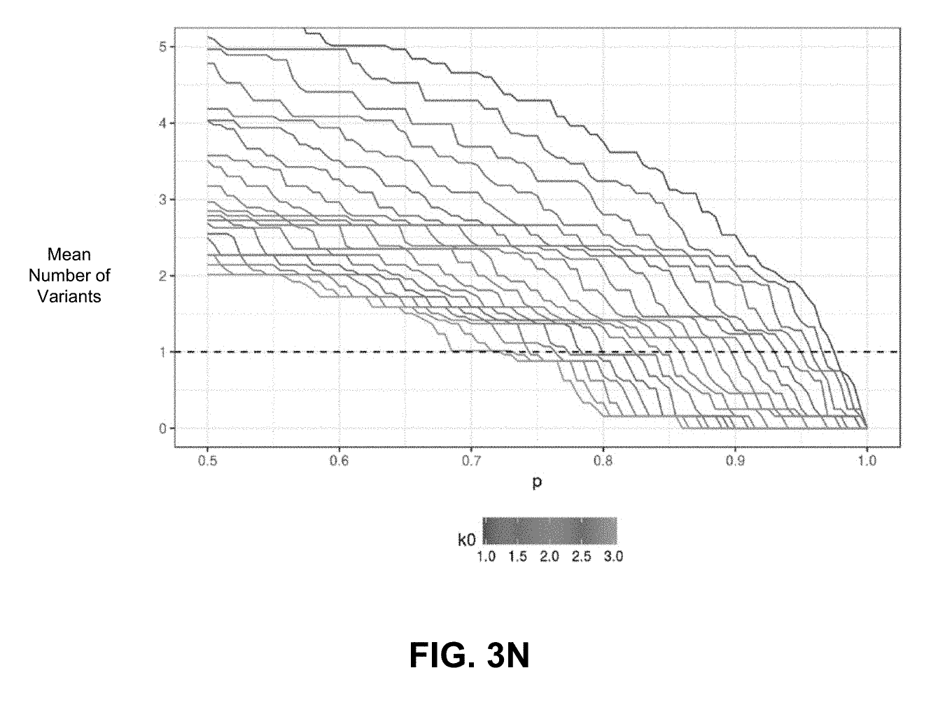

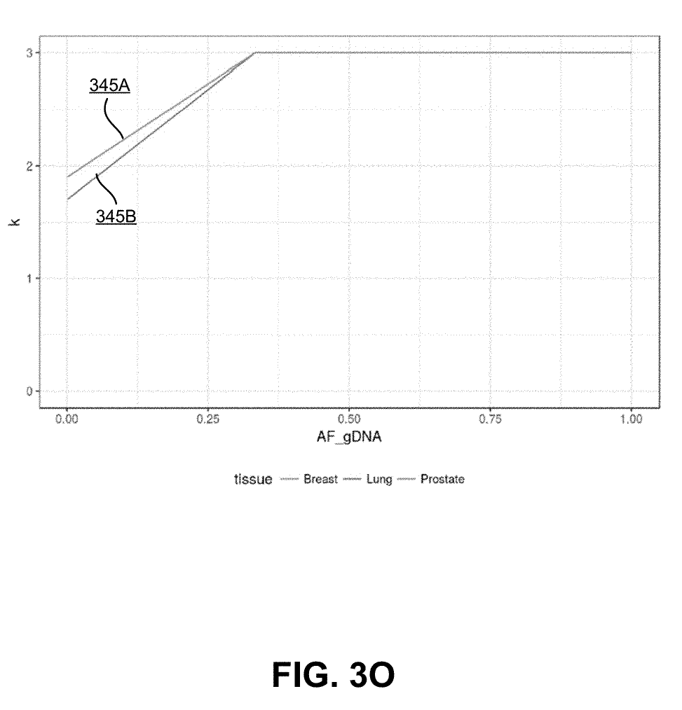

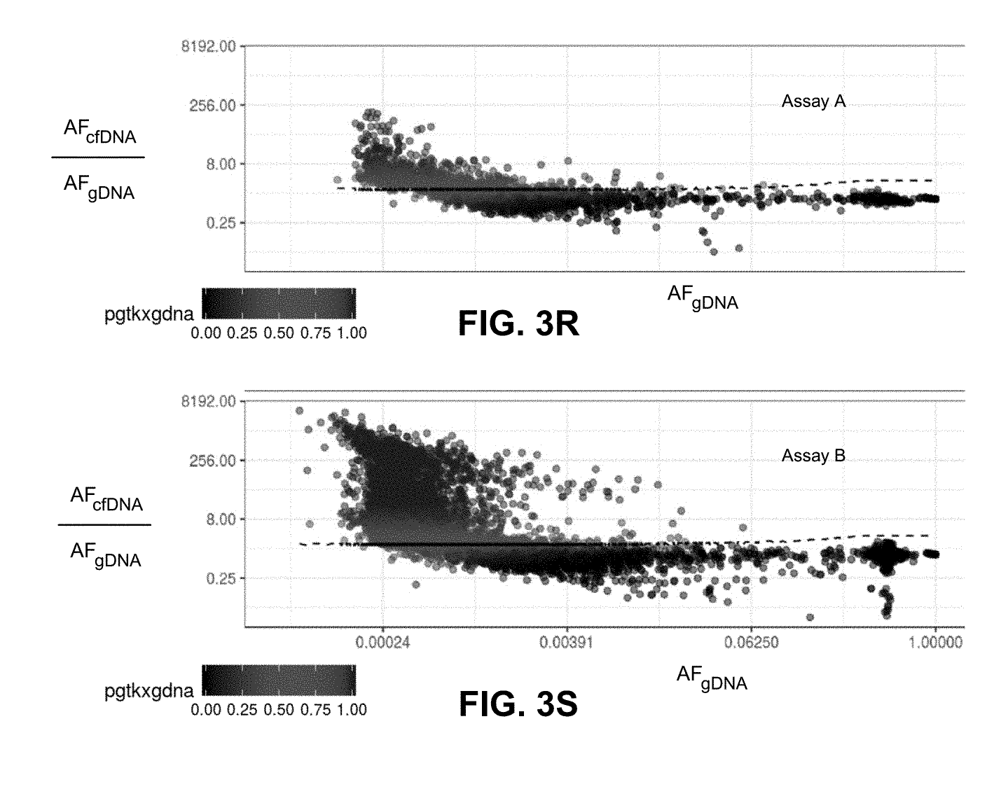

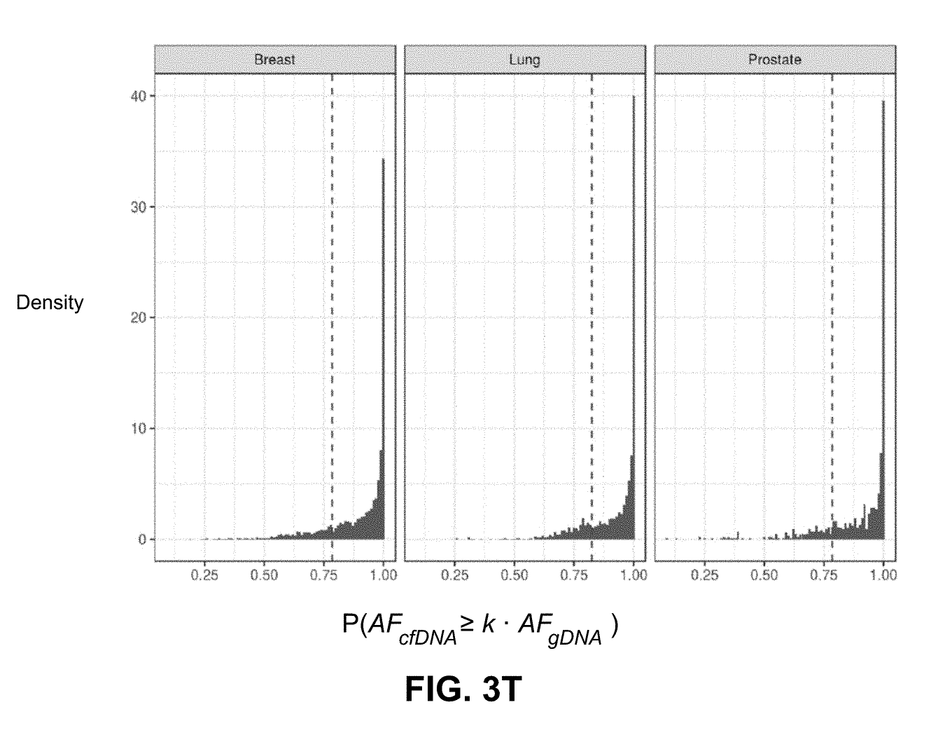

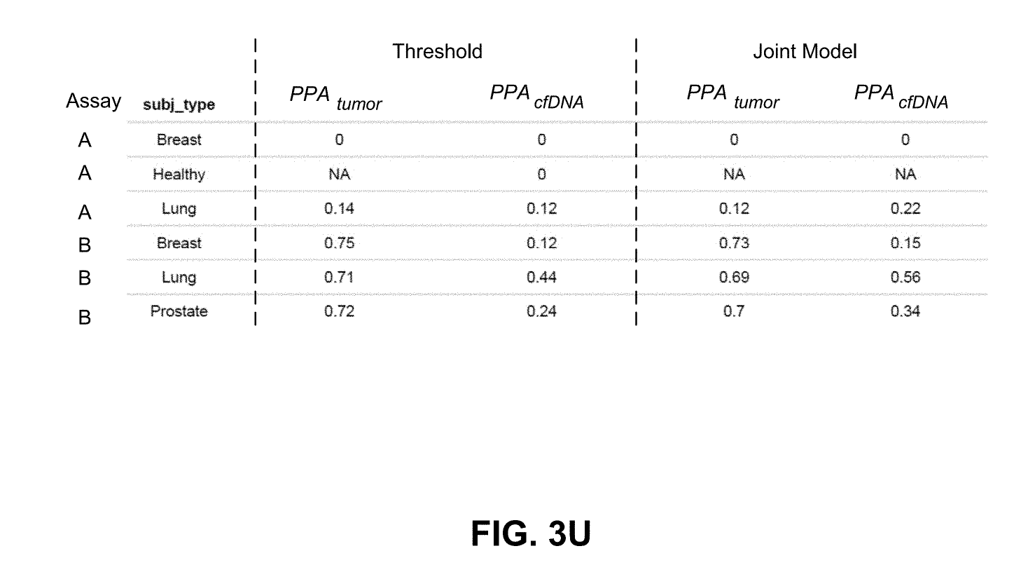

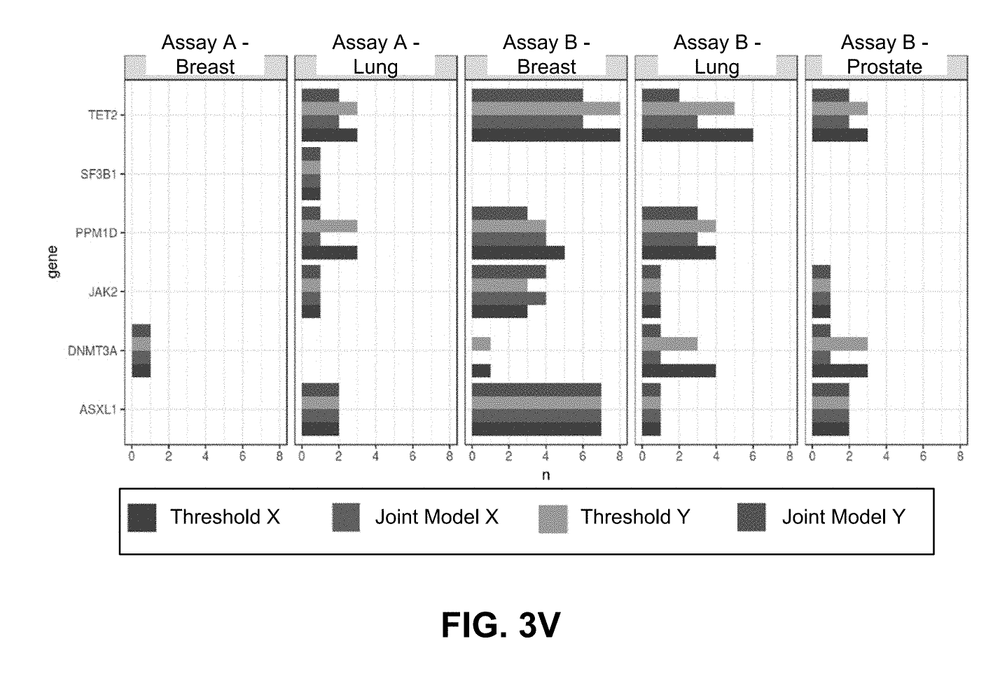

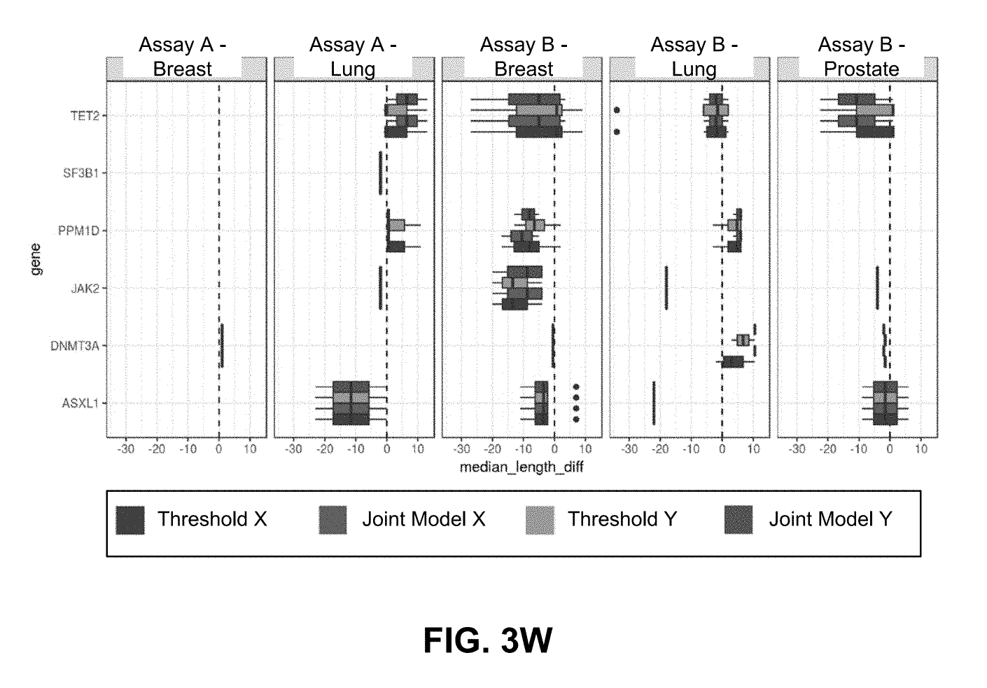

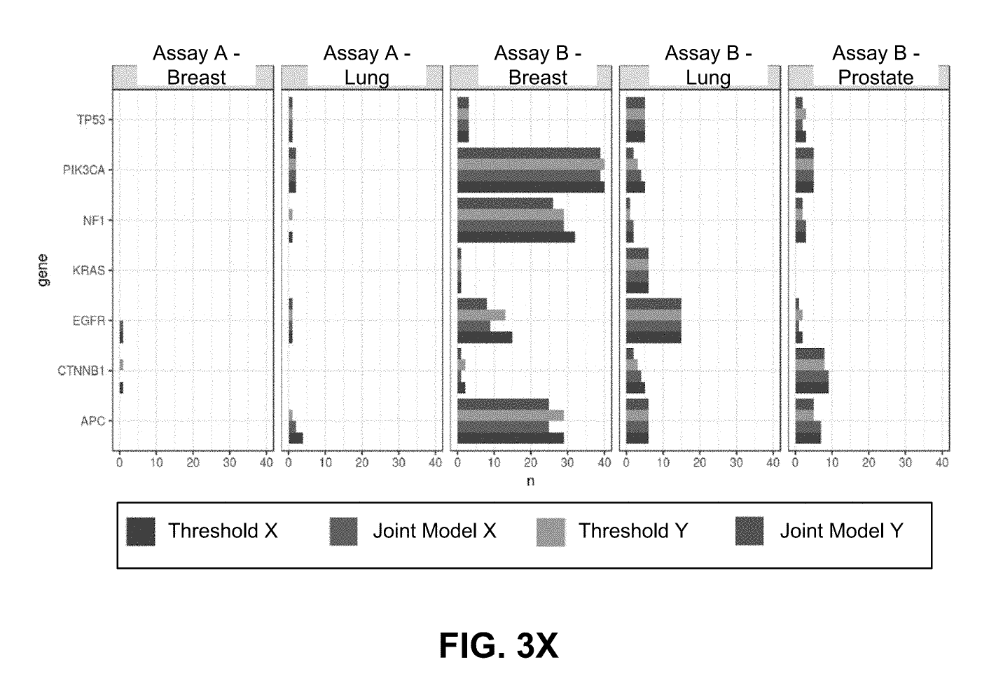

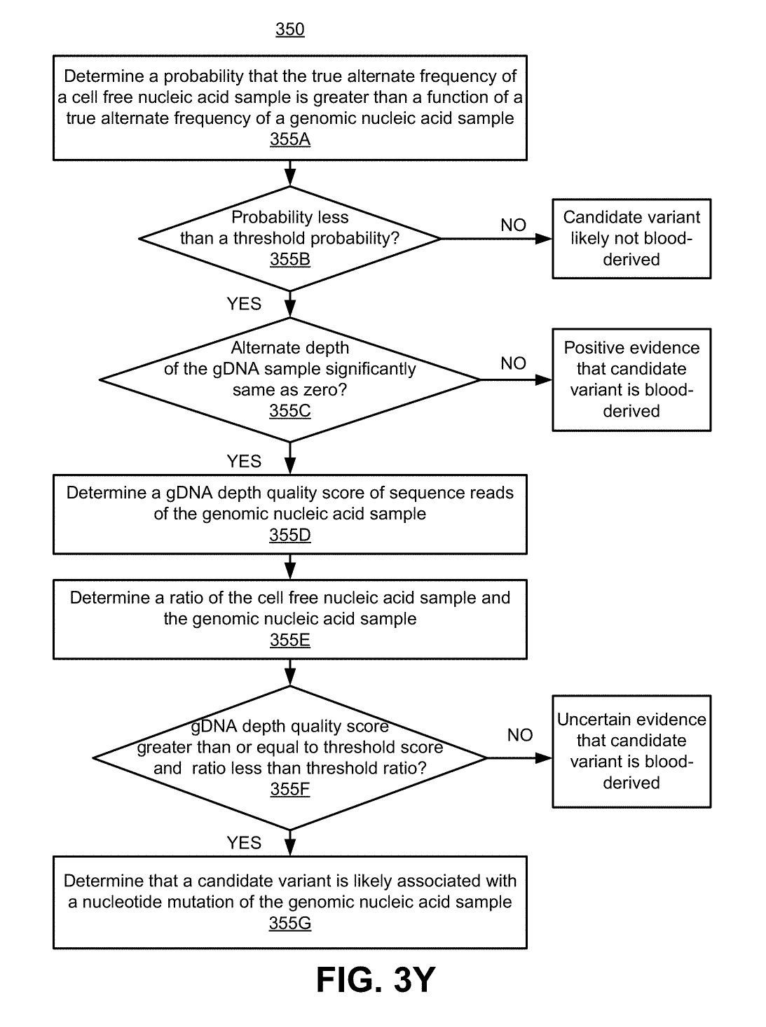

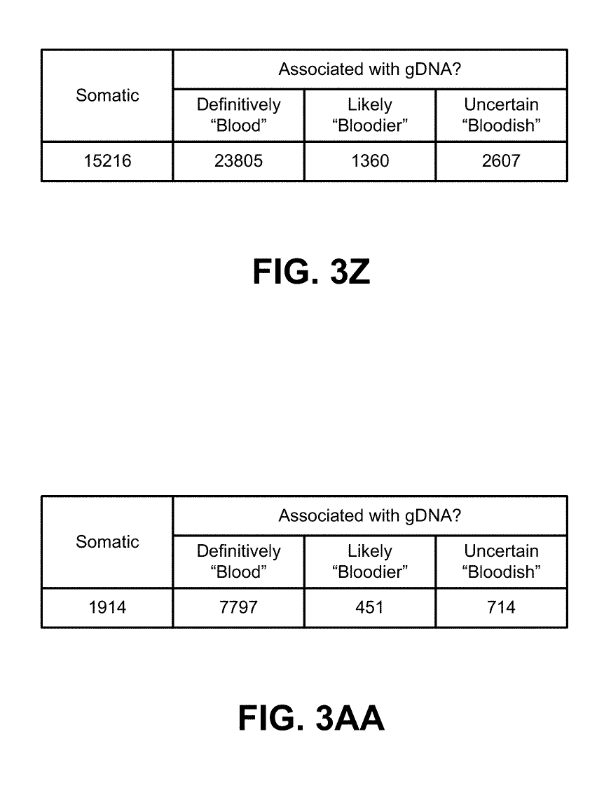

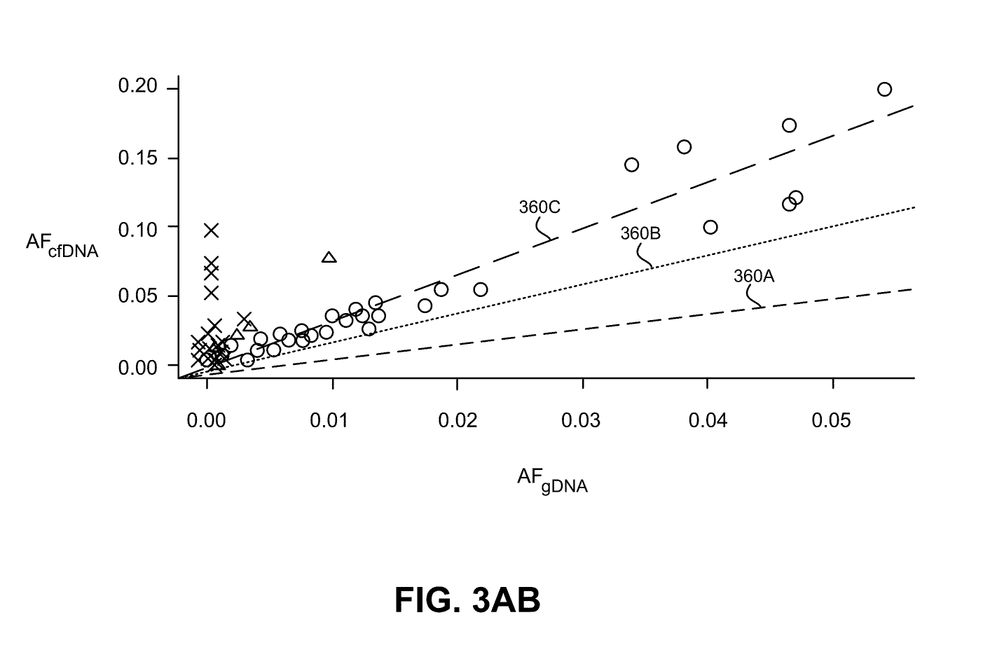

A processing system, such as a processor of a computer, executes the code for performing the small variant computational analysis 140C. FIG. 3A is flowchart of a method 300 for determining somatic variants from sequence reads, in accordance with an embodiment. At step 305A, the processing system collapses aligned sequence reads. In one embodiment, collapsing sequence reads includes using UMIs, and optionally alignment position information from sequencing data of an output file to collapse multiple sequence reads into a consensus sequence for determining the most likely sequence of a nucleic acid fragment or a portion thereof. The unique sequence tag can be from about 4 to 20 nucleic acids in length. Since the UMIs are replicated with the ligated nucleic acid fragments through enrichment and PCR, the sequence processor 205 may determine that certain sequence reads originated from the same molecule in a nucleic acid sample. In some embodiments, sequence reads that have the same or similar alignment position information (e.g., beginning and end positions within a threshold offset) and include a common UMI are collapsed, and the processing system generates a collapsed read (also referred to herein as a consensus read) to represent the nucleic acid fragment. The processing system designates a consensus read as “duplex” if the corresponding pair of collapsed reads have a common UMI, which indicates that both positive and negative strands of the originating nucleic acid molecule is captured; otherwise, the collapsed read is designated “non-duplex.” In some embodiments, the processing system may perform other types of error correction on sequence reads as an alternative to, or in addition to, collapsing sequence reads.

At step 305B, the processing system stitches the collapsed reads based on the corresponding alignment position information. In some embodiments, the processing system compares alignment position information between a first sequence read and a second sequence read to determine whether nucleotide base pairs of the first and second sequence reads overlap in the reference genome. In one use case, responsive to determining that an overlap (e.g., of a given number of nucleotide bases) between the first and second sequence reads is greater than a threshold length (e.g., threshold number of nucleotide bases), the processing system designates the first and second sequence reads as “stitched”; otherwise, the collapsed reads are designated “unstitched.” In some embodiments, a first and second sequence read are stitched if the overlap is greater than the threshold length and if the overlap is not a sliding overlap. For example, a sliding overlap may include a homopolymer run (e.g., a single repeating nucleotide base), a dinucleotide run (e.g., two-nucleotide base sequence), or a trinucleotide run (e.g., three-nucleotide base sequence), where the homopolymer run, dinucleotide run, or trinucleotide run has at least a threshold length of base pairs.

At step 305C, the processing system assembles reads into paths. In some embodiments, the processing system assembles reads to generate a directed graph, for example, a de Bruijn graph, for a target region (e.g., a gene). Unidirectional edges of the directed graph represent sequences of k nucleotide bases (also referred to herein as “k-mers”) in the target region, and the edges are connected by vertices (or nodes). The processing system aligns collapsed reads to a directed graph such that any of the collapsed reads may be represented in order by a subset of the edges and corresponding vertices.

In some embodiments, the processing system determines sets of parameters describing directed graphs and processes directed graphs. Additionally, the set of parameters may include a count of successfully aligned k-mers from collapsed reads to a k-mer represented by a node or edge in the directed graph. The processing system stores directed graphs and corresponding sets of parameters, which may be retrieved to update graphs or generate new graphs. For instance, the processing system may generate a compressed version of a directed graph (e.g., or modify an existing graph) based on the set of parameters. In one use case, in order to filter out data of a directed graph having lower levels of importance, the processing system removes (e.g., “trims” or “prunes”) nodes or edges having a count less than a threshold value, and maintains nodes or edges having counts greater than or equal to the threshold value.

At step 305D, the processing system generates candidate variants from the assembled paths. In one embodiment, the processing system generates the candidate variants by comparing a directed graph (which may have been compressed by pruning edges or nodes in step 305B) to a reference sequence of a target region of a genome. The processing system may align edges of the directed graph to the reference sequence, and records the genomic positions of mismatched edges and mismatched nucleotide bases adjacent to the edges as the locations of candidate variants. In some embodiments, the genomic positions of mismatched edges and mismatched nucleotide bases to the left and right of edges are recorded as the locations of called variants. Additionally, the processing system may generate candidate variants based on the sequencing depth of a target region. In particular, the processing system may be more confident in identifying variants in target regions that have greater sequencing depth, for example, because a greater number of sequence reads help to resolve (e.g., using redundancies) mismatches or other base pair variations between sequences.

In one embodiment, the processing system generates candidate variants using a model to determine expected noise rates for sequence reads from a subject. The model may be a Bayesian hierarchical model, though in some embodiments, the processing system uses one or more different types of models. Moreover, a Bayesian hierarchical model may be one of many possible model architectures that may be used to generate candidate variants and which are related to each other in that they all model position-specific noise information in order to improve the sensitivity/specificity of variant calling. More specifically, the processing system trains the model using samples from healthy individuals to model the expected noise rates per position of sequence reads.

Further, multiple different models may be stored in a database or retrieved for application post-training. For example, a first model is trained to model SNV noise rates and a second model is trained to model insertion deletion noise rates. Further, the processing system may use parameters of the model to determine a likelihood of one or more true positives in a sequence read. The processing system may determine a quality score (e.g., on a logarithmic scale) based on the likelihood. For example, the quality score is a Phred quality score Q=−10·log₁₀ P, where P is the likelihood of an incorrect candidate variant call (e.g., a false positive). Other models, such as a joint model, may use output of one or more Bayesian hierarchical models to determine expected noise of nucleotide mutations in sequence reads of different samples.

At step 305E, the processing system filters the candidate variants using one or more types of models or filters. For example, the processing system scores the candidate variants using a joint model, edge variant prediction model, or corresponding likelihoods of true positives or quality scores.

Reference is now made to FIG. 3B, which is flowchart of step 305E shown in FIG. 3A for processing candidate variants using different types of filters and models, in accordance with an embodiment. At step 310A, the processing system models noise of sequence reads of a nucleic acid sample, e.g., a cfDNA sample. The model may be a Bayesian hierarchical model as previously described, which approximates expected noise distribution per position of the sequence reads. At step 310B, the processing system filters candidate variants from the sequence reads using a joint model. In some embodiments, the processing system uses the joint model to determine whether a given candidate variant observed in the cfDNA sample is likely associated with a nucleotide mutation of a corresponding gDNA sample (e.g., from white blood cells).

At step 310C, the processing system filters the candidate variants using edge filtering. In particular, the processing system may apply an edge filter that predicts the likelihood that a candidate variant is a false positive, also referred to as an edge variant, in view of the variants observed in the sample e.g., cfDNA sample. The term edge variant refers to a mutation located near an edge of a sequence read, for example, within a threshold distance of nucleotide bases from the edge of the sequence read. Specifically, given the collection of candidate variants for a sample, the edge filter may perform a likelihood estimation to determine a predicted rate of edge variants in the sample. Given certain conditions of the sample, the predicted rate may best explain the observed collection of candidate variants for the sample in view of two distributions. One distribution describes features of known edge variants whereas another trained distribution describes features of known non-edge variants. The predicted rate is a sample-specific parameter that controls how aggressively the sample is analyzed to identify and filter edge from the sample. Edge variants of the sample are filtered and removed, leaving non-edge variants for subsequent consideration. The term non-edge variant refers to a candidate variant that is not determined to be resulting from an artifact process, e.g., using an edge variant filtering method described herein. In some scenarios, a non-edge variant may not be a true variant (e.g., mutation in the genome) as the non-edge variant could arise due to a different reason as opposed to one or more artifact processes.

Returning to FIG. 3A, at step 305F, the processing system outputs the filtered candidate variants (e.g., somatic variants). Here, the filtered candidate variants can then be used to determine the small variant features that were described above.

At step 305G, optionally, the small variant features derived from the somatic variants can be used to generate a cancer prediction. For example, a prediction model, such as predictive cancer model 170C shown in FIG. 1C, can be applied to the small variant features. In other words, the prediction model (e.g., predictive cancer model 170C) can serve as a single-assay prediction model that outputs a cancer prediction 190C using only small variant features 154.

2.2.1. Example Noise Models

FIG. 3C is a diagram of an application of a Bayesian hierarchical model according to one embodiment. Mutation A and Mutation B are shown as examples for purposes of explanation. In the embodiment of FIG. 3C, Mutations A and B are represented as SNVs, though in other embodiments, the following description is also applicable to indels or other types of mutations. Mutation A is a C>T mutation at position 4 of a first reference allele from a first sample. The first sample has a first alternate depth (AD) of 10 and a first total depth of 1000. Mutation B is a T>G mutation at position 3 of a second reference allele from a second sample. The second sample has a second AD of 1 and a second total depth of 1200. Based merely on AD (or AF), Mutation A may appear to be a true positive, while Mutation B may appear to be a false positive because the AD (or AF) of the former is greater than that of the latter. However, Mutations A and B may have different relative levels of noise rates per allele and/or per position of the allele. In fact, Mutation A may be a false positive and Mutation B may be a true positive, once the relative noise levels of these different positions are accounted for. The models described herein model this noise for appropriate identification of true positives accordingly.

The probability mass functions (PMFs) illustrated in FIG. 3C indicate the probability (or likelihood) of a sample from a subject having a given AD count at a position. Using sequencing data from samples of healthy individuals, the processing system trains a model from which the PDFs for healthy samples may be derived. In particular, the PDFs are based on mₚ, which models the expected mean AD count per allele per position in normal tissue (e.g., of a healthy individual), and rₚ, which models the expected variation (e.g., dispersion) in this AD count. Stated differently, mₚ and/or rₚ represent a baseline level of noise, on a per position per allele basis, in the sequencing data for normal tissue.

Using the example of FIG. 3C to further illustrate, samples from the healthy individuals represent a subset of the human population modeled by yᵢ, where i is the index of the healthy individual in the training set. Assuming for sake of example, the model has already been trained, PDFs produced by the model visually illustrate the likelihood of the measured ADs for each mutation, and therefore provide an indication of which are true positives and which are false positives. The example PDF on the left of FIG. 3C associated with Mutation A indicates that the probability of the first sample having an AD count of 10 for the mutation at position 4 is approximately 20%. Additionally, the example PDF on the right associated with Mutation B indicates that the probability of the second sample having an AD count of 1 for the mutation at position 3 is approximately 1% (note: the PDFs of FIG. 3C are not exactly to scale). Thus, the noise rates corresponding to these probabilities of the PDFs indicate that Mutation A is more likely to occur than Mutation B, despite Mutation B having a lower AD and AF. Thus, in this example, Mutation B may be the true positive and Mutation A may be the false positive. Accordingly, the processing system may perform improved variant calling by using the model to distinguish true positives from false positives at a more accurate rate, and further provide numerical confidence as to these likelihoods.

FIG. 3D shows dependencies between parameters and sub-models of a Bayesian hierarchical model for determining true single nucleotide variants according to one embodiment. In the example shown in FIG. 3D, \vec{θ} represents the vector of weights assigned to each mixture component. The vector \vec{θ} takes on values within the simplex in K dimensions and may be learned or updated via posterior sampling during training. It may be given a uniform prior on said simplex for such training. The mixture component to which a position p belongs may be modeled by latent variable zₚ using one or more different multinomial distributions:

zₚ˜Multinom(\vec{θ})

Together, the latent variable zₚ, the vector of mixture components \vec{θ}, α, and β allow the model for μ, that is, a sub-model of the Bayesian hierarchical model, to have parameters that “pool” knowledge about noise, that is they represent similarity in noise characteristics across multiple positions. Thus, positions of sequence reads may be pooled or grouped into latent classes by the model. Also advantageously, samples of any of these “pooled” positions can help train these shared parameters. A benefit of this is that the processing system may determine a model of noise in healthy samples even if there is little to no direct evidence of alternative alleles having been observed for a given position previously (e.g., in the healthy tissue samples used to train the model).

The covariate xₚ (e.g., a predictor) encodes known contextual information regarding position p which may include, but is not limited to, information such as trinucleotide context, mappability, segmental duplication, or other information associated with sequence reads. Trinucleotide context may be based on a reference allele and may be assigned numerical (e.g., integer) representation. For instance, “AAA” is assigned 1, “ACA” is assigned 2, “AGA” is assigned 3, etc. Mappability represents a level of uniqueness of alignment of a read to a particular target region of a genome. For example, mappability is calculated as the inverse of the number of position(s) where the sequence read will uniquely map. Segmental duplications correspond to long nucleic acid sequences (e.g., having a length greater than approximately 1000 base pairs) that are nearly identical (e.g., greater than 90% match) and occur in multiple locations in a genome as result of natural duplication events (e.g., not associated with a cancer or disease).

The expected mean AD count of a SNV at position p is modeled by the parameter μₚ. For sake of clarity in this description, the terms μₚ and yₚ refer to the position specific sub-models of the Bayesian hierarchical model. In one embodiment, μₚ is modeled as a Gamma-distributed random variable having shape parameter α_{z}_{ₚ}_{,x}_{ₚ} and mean parameter β_{z}_{ₚ}_{,x}_{ₚ}:

μₚ˜Gamma(α_{z}_{ₚ}_{,x}_{ₚ},β_{z}_{ₚ})

In other embodiments, other functions may be used to represent μₚ, examples of which include but are not limited to: a log-normal distribution with log-mean γ_{z}_{ₚ} and log-standard-deviation σ_{z}_{ₚ}_{,x}_{ₚ}, a Weibull distribution, a power law, an exponentially-modulated power law, or a mixture of the preceding.

In the example shown in FIG. 3D, the shape and mean parameters are each dependent on the covariate xₚ and the latent variable zₚ, though in other embodiments, the dependencies may be different based on various degrees of information pooling during training. For instance, the model may alternately be structured so that α_{z}_{ₚ} depends on the latent variable but not the covariate. The distribution of AD count of the SNV at position p in a human population sample i (of a healthy individual) is modeled by the random variable yᵢ_{ₚ}. In one embodiment, the distribution is a Poisson distribution given a depth dᵢₚ of the sample at the position:

yᵢ_{ₚ}|dᵢ_{ₚ}˜Poisson(dᵢ_{ₚ}·μₚ)

In other embodiments, other functions may be used to represent yᵢ_{ₚ}, examples of which include but are not limited to: negative binomial, Conway-Maxwell-Poisson distribution, zeta distribution, and zero-inflated Poisson.

FIG. 3E shows dependencies between parameters and sub-models of a Bayesian hierarchical model for determining true insertions or deletions according to one embodiment. In contrast to the SNV model shown in FIG. 3D, the model for indels shown in FIG. 3E includes different levels of hierarchy. The covariate xₚ encodes known features at position p and may include, e.g., a distance to a homopolymer, distance to a RepeatMasker repeat, or other information associated with previously observed sequence reads. Latent variable \vec{ϕₚ} may be modeled by a Dirichlet distribution based on parameters of vector \vec{ω}ₓ, which represent indel length distributions at a position and may be based on the covariate. In some embodiments, \vec{ω}ₓ is also shared across positions (\vec{ω}_{x,p}) that share the same covariate value(s). Thus for example, the latent variable may represent information such as that homopolymer indels occur at positions 1, 2, 3, etc. base pairs from the anchor position, while trinucleotide indels occur at positions 3, 6, 9, etc. from the anchor position.

The expected mean total indel count at position p is modeled by the distribution μₚ. In some embodiments, the distribution is based on the covariate and has a Gamma distribution having shape parameter αₓ_{ₚ} and mean parameter βₓ_{ₚ}ₓ_{ₚ}:

μₚ˜Gamma(αₓ_{_{p,}}βₓ_{ₚ}_{z}_{ₚ})

In other embodiments, other functions may be used to represent p, examples of which include but are not limited to: negative binomial, Conway-Maxwell-Poisson distribution, zeta distribution, and zero-inflated Poisson.

The observed indels at position p in a human population sample i (of a healthy individual) is modeled by the distribution yᵢ_{ₚ}. Similar to example in FIG. 3D, in some embodiments, the distribution of indel intensity is a Poisson distribution given a depth dᵢ_{ₚ} of the sample at the position:

yᵢ_{ₚ}|dᵢ_{ₚ}˜Poisson(dᵢ_{_{P}}·μₚ)

In other embodiments, other functions may be used to represent yᵢ_{ₚ}, examples of which include but are not limited to: negative binomial, Conway-Maxwell-Poisson distribution, zeta distribution, and zero-inflated Poisson.

Due to the fact that indels may be of varying lengths, an additional length parameter is present in the indel model that is not present in the model for SNVs. As a consequence, the example model shown in FIG. 3E has an additional hierarchical level (e.g., another sub-model), which is again not present in the SNV models discussed above. The observed count of indels of length l (e.g., up to 100 or more base pairs of insertion or deletion) at position p in sample i is modeled by the random variable yᵢ_{ₚᵢ}, which represents the indel distribution under noise conditional on parameters. The distribution may be a multinomial given indel intensity yᵢ_{ₚ} of the sample and the distribution of indel lengths \vec{ϕₚ} at the position:

\vec{y}ᵢ_{ₚᵢ}|yᵢ_{ₚ},\vec{ϕₚ}˜Multinom(yᵢ_{ₚ},\vec{ϕₚ})

In other embodiments, a Dirichlet-Multinomial function or other types of models may be used to represent yᵢ_{ₚᵢ}.

By architecting the model in this manner, the processing system may decouple learning of indel intensity (i.e., noise rate) from learning of indel length distribution. Independently determining inferences for an expectation for whether an indel will occur in healthy samples and expectation for the length of the indel at a position may improve the sensitivity of the model. For example, the length distribution may be more stable relative to the indel intensity at a number of positions or regions in the genome, or vice versa.

FIGS. 3F and 3G illustrate diagrams associated with a Bayesian hierarchical model according to one embodiment. The graph shown in FIG. 3F depicts the distribution μₚ of noise rates, i.e., likelihood (or intensity) of SNVs or indels for a given position as characterized by a model. The continuous distribution represents the expected AF μₚ of non-cancer or non-disease mutations (e.g., mutations naturally occurring in healthy tissue) based on training data of observed healthy samples from healthy individuals (e.g., retrieved from the sequence database 210). Though not shown in FIG. 3F, in some embodiments, the shape and mean parameters of μₚ may be based on other variables such as the covariate xₚ or latent variable zₚ. The graph shown in FIG. 3G depicts the distribution of AD at a given position for a sample of a subject, given parameters of the sample such as sequencing depth dₚ at the given position. The discrete probabilities for a draw of μₚ are determined based on the predicted true mean AD count of the human population based on the expected mean distribution pp.

FIG. 3H is a diagram of an example process for determining parameters by fitting a Bayesian hierarchical model according to one embodiment. To train a model, the processing system samples iteratively from a posterior distribution of expected noise rates (e.g., the graph shown in FIG. 3G) for each position of a set of positions. The processing system may use Markov chain Monte Carlo (MCMC) methods for sampling, e.g., a Metropolis-Hastings (MH) algorithm, custom MH algorithms, Gibbs sampling algorithm, Hamiltonian mechanics-based sampling, random sampling, among other sampling algorithms. During Bayesian Inference training, parameters are drawn from the joint posterior distribution to iteratively update all (or some) parameters and latent variables of the model (e.g., θ, zₚ, α_{z}_{ₚ}_{,x}_{ₚ}, β_{z}_{ₚ}_{,x}_{ₚ}, μₚ, etc.).

In one embodiment, the processing system performs model fitting by storing draws of μₚ, the expected mean counts of AF per position and per sample. The model is trained or fitted through posterior sampling, as previously described. In an embodiment, the draws of μₚ are stored in a matrix data structure having a row per position of the set of positions sampled and a column per draw from the joint posterior (e.g., of all parameters conditional on the observed data). The number of rows R may be greater than 6 million and the number of columns for N iterations of samples may be in the thousands. In other embodiments, the row and column designations are different than the embodiment shown in FIG. 3H, e.g., each row represents a draw from a posterior sample, and each column represents a sampled position (e.g., transpose of the matrix example shown in FIG. 3H).

FIG. 3I is a diagram of using parameters from a Bayesian hierarchical model to determine a likelihood of a false positive according to one embodiment. The processing system may reduce the R rows-by-N column matrix shown in FIG. 3H into an R rows-by-2 column matrix illustrated in FIG. 3I. In one embodiment, the processing system determines a dispersion parameter rₚ (e.g., shape parameter) and mean parameter mₚ (which may also be referred to as a mean rate parameter mₚ) per position across the posterior samples μₚ. The dispersion parameter rₚ may be determined as

$${r}_{p} = \frac{{m}_{p}^{2}}{{v}_{p}^{2}} ,$$

where mₚ and vₚ are the mean and variance of the sampled values of μₚ at the position, respectively. Those of skill in the art will appreciate that other functions for determining rₚ may also be used such as a maximum likelihood estimate.

The processing system may also perform dispersion re-estimation of the dispersion parameters in the reduced matrix, given the mean parameters. In one embodiment, following Bayesian training and posterior approximation, the processing system performs dispersion re-estimation by retraining for the dispersion parameters [symbol] based on a negative binomial maximum likelihood estimator per position. The mean parameter may remain fixed during retraining. In one embodiment, the processing system determines the dispersion parameters r′ₚ at each position for the original AD counts of the training data (e.g., yᵢ_{ₚ} and dᵢ_{ₚ} based on healthy samples). The processing system determines \tilde{r}ₚ=max(rₚ, r′ₚ), and stores \tilde{r}ₚ in the reduced matrix. Those of skill in the art will appreciate that other functions for determining rₚ may also be used, such as a method of moments estimator, posterior mean, or posterior mode.

During application of trained models, the processing system may access the dispersion (e.g., shape) parameters \tilde{r}ₚ and mean parameters mₚ to determine a function parameterized by \tilde{r}ₚ and mₚ. The function may be used to determine a posterior predictive probability mass function (or probability density function) for a new sample of a subject. Based on the predicted probability of a certain AD count at a given position, the processing system may account for site-specific noise rates per position of sequence reads when detecting true positives from samples.

Referring back to the example use case described with respect to FIG. 3C, the PDFs shown for Mutations A and B may be determined using the parameters from the reduced matrix of FIG. 3I. The posterior predictive probability mass functions may be used to determine the probability of samples for Mutations A or B having an AD count at certain position.

2.3. Example Process Flows for Noise Models

FIG. 3J is flowchart of a method 315 for training a Bayesian hierarchical model according to one embodiment. In step 320A, the processing system collects samples, e.g., training data, from a database of sequence reads. In step 320B, the processing system trains the Bayesian hierarchical model using the samples using a Markov Chain Monte Carlo method. During training, the model may keep or reject sequence reads conditional on the training data. The processing system may exclude sequence reads of healthy individuals that have less than a threshold depth value or that have an AF greater than a threshold frequency in order to remove suspected germline mutations that are not indicative of target noise in sequence reads. In other embodiments, the processing system may determine which positions are likely to contain germline variants and selectively exclude such positions using thresholds like the above. In one embodiment, the processing system may identify such positions as having a small mean absolute deviation of AFs from germline frequencies (e.g., 0, ½, and 1).

The Bayesian hierarchical model may update parameters simultaneously for multiple (or all) positions included in the model. Additionally, the model may be trained to model expected noise for each ALT. For instance, a model for SNVs may perform a training process four or more times to update parameters (e.g., one-to-one substitutions) for mutations of each of the A, T, C, and G bases to each of the other three bases. In step 320C, the processing system stores parameters of the Bayesian hierarchical model (e.g., ensemble parameters output by the Markov Chain Monte Carlo method). In step 320D, the processing system approximates the noise distribution (e.g., represented by a dispersion parameter and a mean parameter) per position based on the parameters. In step 320E, the processing system performs dispersion re-estimation (e.g., maximum likelihood estimation) using original AD counts from the samples (e.g., training data) used to train the Bayesian hierarchical model.

FIG. 3K is flowchart of a method 325 for determining a likelihood of a false positive according to one embodiment. At step 330A, the processing system identifies a candidate variant, e.g., at a position p of a sequence read, from a set of sequence reads, which may be sequenced from a cfDNA sample obtained from an individual. At step 330B, the processing system accesses parameters, e.g., dispersion and mean rate parameters

[symbol]

and mₚ, respectively, specific to the candidate variant, which may be based on the position p of the candidate variant. The parameters may be derived using a model, e.g., a Bayesian hierarchical model representing a posterior predictive distribution with an observed depth of a given sequence read and a mean parameter μₚ at the position p as input. In an embodiment, the mean parameter μₚ is a gamma distribution encoding a noise level of nucleotide mutations with respect to the position p for a training sample.

At step 330C, the processing system inputs read information (e.g., AD or AF) of the set of sequence reads into a function (e.g., based on a negative binomial) parameterized by the parameters, e.g., [symbol] and mₚ. At step 330D, the processing system determines a score for the candidate variant (e.g., at the position p) using an output of the function based on the input read information. The score may indicate a likelihood of seeing an allele count for a given sample (e.g., from a subject) that is greater than or equal to a determined allele count of the candidate variant (e.g., determined by the model and output of the function). The processing system may convert the likelihood into a Phred-scaled score. In some embodiments, the processing system uses the likelihood to determine false positive mutations responsive to determining that the likelihood is less than a threshold value. In some embodiments, the processing system uses the function to determine that a sample of sequence reads includes at least a threshold count of alleles corresponding to a gene found in sequence reads from a tumor biopsy of an individual. In some embodiments, the processing system may perform weighting based on quality scores, use the candidate variants and quality scores for false discovery methods, annotate putative calls with quality scores, or provision to subsequent systems.

The processing system may use functions encoding noise levels of nucleotide mutations with respect to a given training sample for downstream analysis. In some embodiments, the processing system uses the aforementioned negative binomial function parameterized by the dispersion and mean rate parameters

[symbol]

and mₚ to determine expected noise for a particular nucleic acid position within a sample, e.g., cfDNA or gDNA. Moreover, the processing system may derive the parameters by training a Bayesian hierarchical model using training data associated with the particular nucleic acid sample. The embodiments below describe another type of model referred to herein as a joint model, which may use output of a Bayesian hierarchical model.

2.4. Example Joint Model

FIG. 3L is flowchart of a method 335 for using a joint model to process cell free nucleic acid (e.g., cfDNA) samples and genomic nucleic acid (e.g., gDNA) samples according to one embodiment. Other examples of a joint model found in the art may also be similarly employed (see, e.g., U.S. Ser. No. 16/201,912, which is incorporated by reference herein). The joint model may be independent of positions of nucleic acids of cfDNA and gDNA. The method 335 may be performed in conjunction with the methods 315 and/or 325 shown in FIGS. 3J and 3K. For example, the methods 315 and 325 are performed to determine noise of nucleotide mutations with respect to cfDNA and gDNA samples of training data from health samples. FIG. 3M is a diagram of an application of a joint model according to one embodiment. Steps of the method 335 are described below with reference to FIG. 3M.

In step 340A, the processing system determines depths and ADs for the various positions of nucleic acids from the sequence reads obtained from a cfDNA sample of a subject. The cfDNA sample may be collected from a sample of blood plasma from the subject.

In step 340B, the processing system determines depths and ADs for the various positions of nucleic acids from the sequence reads obtained from a gDNA of the same subject. The gDNA may be collected from white blood cells or a tumor biopsy from the subject.

In step 340C, a joint model determines a likelihood of a “true” AF of the cfDNA sample of the subject by modeling the observed ADs for cfDNA. In one embodiment, the joint model uses a Poisson distribution function, parameterized by the depths observed from the sequence reads of cfDNA and the true AF of the cfDNA sample, to model the probability of observing a given AD in cfDNA of the subject (also shown in FIG. 3M). The product of the depth and the true AF may be the rate parameter of the Poisson distribution function, which represents the mean expected AF of cfDNA.

P(AD_{cfDNA}|depth_{cfDNA},AF_{cfDNA})˜Poisson(depth_{cfDNA}·AF_{cfDNA})+noise_{cjDNA}

The noise component noise_{cfDNA} is further described below. In other embodiments, other functions may be used to represent AD_{cfDNA}, examples of which include but are not limited to: negative binomial, Conway-Maxwell-Poisson distribution, zeta distribution, and zero-inflated Poisson.

In step 340D, the joint model determines a likelihood of a “true” AF of the gDNA sample of the subject by modeling the observed ADs for gDNA. In one embodiment, the joint model uses a Poisson distribution function parameterized by the depths observed from the sequence reads of gDNA and the true AF of the gDNA sample to model the probability of observing a given AD in gDNA of the subject (also shown in FIG. 3M). The joint model may use a same function for modeling the likelihoods of true AF of gDNA and cfDNA, though the parameter values differ based on the values observed from the corresponding sample of the subject.

P(AD_{gDNA}|depth_{gDNA},AF_{gDNA})˜Poisson(depth_{gDNA}·AF_{gDNA})+noise_{gDNA}

The noise component noise_{gDNA} is further described below. In other embodiments, other functions may be used to represent AD_{gDNA}, examples of which include but are not limited to: negative binomial, Conway-Maxwell-Poisson distribution, zeta distribution, and zero-inflated Poisson.

Since the true AF of cfDNA, as well as the true AF of gDNA, are inherent properties of the biology of a particular subject, it may not necessarily be practical to determine an exact value of the true AF from either source. Moreover, various sources of noise also introduce uncertainty into the estimated values of the true AF. Accordingly, the joint model uses numerical approximation to determine the posterior distributions of true AF conditional on the observed data (e.g., depths and ADs) from a subject and corresponding noise parameters:

P(AF_{cfDNA}|depth_{cfDNA},AD_{cfDNA}, [symbol] _{cfDNA},mₚ_{_{cfDNA}})

P(AF_{gDNA}|depth_{gDNA},AD_{gDNA}, [symbol] _{gDNA},mₚ_{_{gDNA}})

The joint model determines the posterior distributions using Bayes' theorem with a prior, for example, a uniform distribution. The priors used for cfDNA and gDNA may be the same (e.g., a uniform distribution ranging from 0 to 1) and independent from each other.

In an embodiment, the joint model determines the posterior distribution of true AF of cfDNA using a likelihood function by varying the parameter, true AF of cfDNA, given a fixed set of observed data from the sample of cfDNA. Additionally, the joint model determines the posterior distribution of true AF of gDNA using another likelihood function by varying the parameter, true AF of gDNA, given a fixed set of observed data from the sample of gDNA. For both cfDNA and gDNA, the joint model numerically approximates the output posterior distribution by fitting a negative binomial (NB):

$$P ( AF \vert depth , AD ) ∝ {\sum }_{i = 0}^{AD} \frac{{{e}^{- AF \cdot depth} ( AF \cdot depth )}^{i}}{i !} \cdot NB ( AD - i , size = r , μ = m \cdot depth )$$

In an embodiment, the joint model performs numerical approximation using the following parameters for the negative binomial, which may provide an improvement in computational speed:

P(AF|depth,AD)∂NB(AD,size=r,μ=m·depth)

Where

m=AF+m

r=r·m²/m²

Since the observed data is different between cfDNA and gDNA, the parameters determined for the negative binomial of cfDNA will vary from those determined for the negative binomial of gDNA.

In step 340E, the processing system determines, using the likelihoods, a probability that the true AF of the cfDNA sample is greater than a function of the true AF of the gDNA sample. The function may include one or more parameters, for example, empirically-determined k and p values and described with additional detail with reference to FIGS. 3N-3O. The probability represents a confidence level that at least some nucleotide mutations from the sequence reads of cfDNA are not found in sequence reads of reference tissue. The processing system may provide this information to other processes for downstream analysis. For instance, a high probability indicates that nucleotide mutations from a subject's sequence reads of cfDNA and that are not found in sequence reads of gDNA may have originated from a tumor or other source of cancer within the subject. In contrast, low probability indicates that nucleotide mutations observed in cfDNA likely did not originate from potential cancer cells or other diseased cells of the subject. Instead, the nucleotide mutations may be attributed to naturally occurring mutations in healthy individuals, due to factors such as germline mutations, clonal hematopoiesis (unique mutations that form subpopulations of blood cell DNA), mosaicism, chemotherapy or mutagenic treatments, technical artifacts, among others.

In an embodiment, the processing system determines that the posterior probability satisfies a chosen criteria based on the one or more parameters (e.g., k and p described below). The distributions of variants are conditionally independent given the sequences of the cfDNA and gDNA. That is, the processing system presumes that ALTs and noise present in one of the cfDNA or gDNA sample is not influenced by those of the other sample, and vice versa. Thus, the processing system considers the probabilities of the expected distributions of AD as independent events in determining the probability of observing both a certain true AF of cfDNA and a certain true AF of gDNA, given the observed data and noise parameters from both sources:

[symbol]

P(AF_{cfDNA},AF_{gDNA}|depth,AD, [symbol] ,mₚ)∂

P(AF_{cfDNA}|depth_{cfDNA},AD_{cfDNA}, [symbol] _{cfDNA},mₚ_{_{cfDNA}}).

P(AF_{gfDNA}|depth_{gDNA},AD_{gDNA}, [symbol] _{gDNA},mₚ_{_{gDNA}})

In the example 3D plot in FIG. 3M, the probability P(AF_{cfDNA},AF_{gDNA}) is plotted as a 3D contour for pairs of AF_{cfDNA} and AF_{gDNA} values. The example 2D slice of the 3D contour plot along the AF_{cfDNA} and AF_{gDNA} axis illustrates that the volume of the contour plot is skewed toward greater values of AF_{gDNA} relative to the values of AF_{cfDNA}. In other embodiments, the contour plot may be skewed differently or have a different form than the example shown in FIG. 3M. To numerically approximate the joint likelihood, the processing system may calculate the volume defined by the 3D contour of P(AF_{cfDNA}, AF_{gDNA}) and a boundary line illustrated by the dotted line shown in the plots of FIG. 3M. The processing system determines the slope of the boundary line according to the k parameter value, and the boundary line intersects the origin point. The k parameter value may account for a margin of error in the determined true AF. Particularly, the margin of error may cover naturally occurring mutations in healthy individuals such as germline mutations, clonal hematopoiesis, loss of heterozygosity (further described below with reference to FIG. 3O), and other sources as described above. Since the 3D contour is split by the boundary line, at least a portion of variants detected from the cfDNA sample may potentially be attributed to variants detected from the gDNA sample, while another portion of the variants may potentially be attributed to a tumor or other source of cancer.

In an embodiment, the processing system determines that a given criteria is satisfied by the posterior probability by determining the portion of the joint likelihood that satisfies the given criteria. The given criteria may be based on the k and p parameter, where p represents a threshold probability for comparison. For example, the processing system determines the posterior probability that true AF of cfDNA is greater than or equal to the true AF of gDNA multiplied by k, and whether the posterior probability is greater than p:

$$P ( {AF}_{cfDNA} \geq k \cdot {AF}_{gDNA} ) > p , where$$

$$P ( {AF}_{cfDNA} \geq k \cdot {AF}_{gDNA} ) = {\int }_{0}^{1} {\int }_{k \cdot {AF}_{gDNA}}^{1} {f}_{cfDNA} ( {AF}_{cfDNA} ) \cdot {f}_{gDNA} ( {AF}_{gDNA} ) {dAF}_{cfDNA} {dAF}_{gDNA} = {\int }_{0}^{1} {f}_{gDNA} ( {AF}_{gDNA} ) [ {\int }_{k \cdot {AF}_{gDNA}}^{1} {f}_{cfDNA} ( {AF}_{cfDNA} ) \cdot {dAF}_{cfDNA} ] {dAF}_{gDNA} = {\int }_{0}^{1} {f}_{gDNA} ( {AF}_{gDNA} ) ( 1 - {F}_{cfDNA} ( k \cdot {AF}_{gDNA} ) ) {dAF}_{gDNA}$$

As shown in the above equations, the processing system determines a cumulative sum F_{cfDNA} of the likelihood of the true AF of cfDNA. Furthermore, the processing system integrates over the likelihood function of the true AF of gDNA. In another embodiment, the seq processing system may determine the cumulative sum for the likelihood of the true AF of gDNA, and integrates over the likelihood function of the true AF of cfDNA. By calculating the cumulative sum of one of the two likelihoods (e.g., building a cumulative distribution function), instead of computing a double integral over both likelihoods for cfDNA and gDNA, the processing system reduces the computational resources (expressed in terms of compute time or other similar metrics) required to determine whether the joint likelihood satisfies the criteria and may also increase precision of the calculation of the posterior probability.

To account for noise in the estimated values of the true AF introduced by noise in the cfDNA and gDNA samples, the joint model may use other models (e.g., Bayesian hierarchical model) of the processing system previously described with respect to FIGS. 3C-3I. In an embodiment, the noise components shown in the above equations for P(AD_{cfDNA}|depth_{cfDNA},AF_{cfDNA}) and P(AD_{gDNA}|depth_{gDNA},AF_{gDNA}) are determined using Bayesian hierarchical models, which may be specific to a candidate variant (e.g., SNV or indel). Moreover, the Bayesian hierarchical models may cover candidate variants over a range of specific positions of nucleotide mutations or indel lengths.

In one example, the joint model uses a function parameterized by cfDNA-specific parameters to determine a noise level for the true AF of cfDNA. The cfDNA-specific parameters may be derived using a Bayesian hierarchical model trained with a set of cfDNA samples, e.g., from healthy individuals. In addition, the joint model uses another function parameterized by gDNA-specific parameters to determine a noise level for the true AF of gDNA. The gDNA-specific parameters may be derived using another Bayesian hierarchical model trained with a set of gDNA samples, e.g., from the same healthy individuals. In an embodiment, the functions are negative binomial functions having a mean parameter m and dispersion parameter \vec{r}, and may also depend on the observed depths of sequence reads from the training samples:

noise_{cfDNA}=NB(m_{cfDNA}·depth_{cfDNA},\tilde{r}_{cfDNA})

noise_{gDNA}=NB(m_{gDNA}·depth_{gDNA},\tilde{r}_{gDNA})

In other embodiments, the processing system may use a different type of function and types of parameters for cfDNA and/or gDNA. Since the cfDNA-specific parameters and gDNA-specific parameters are derived using different sets of training data, the parameters may be different from each other and particular to the respective type of nucleic acid sample. For instance, cfDNA samples may have greater variation in AF than gDNA samples, and thus \tilde{r}_{cfDNA} may be greater than \tilde{r}_{gDNA}.

2.5. Examples for Joint Models

The example results shown in the following figures were determined by the processing system using one or more trained models, which may include joint models and Bayesian hierarchical models. For purposes of comparison, some example results were determined using an empirical threshold or a simple model and are referred to as “empirical threshold” examples and denoted as “threshold” in the figures; these example results were not obtained using one of the trained models. In various embodiments, the results were generated using one of a number of example targeted sequencing assays, including “targeted sequencing assay A” and “targeted sequencing assay B,” also referred to herein and in the figures as “Assay A” and “Assay B,” respectively.

In an example process performed for a targeted sequencing assay, two tubes of whole blood were drawn into Streck BCTs from healthy individuals (self-reported as no cancer diagnosis). After plasma was separated from the whole blood, it was stored at −80° C. Upon assay processing, cfDNA was extracted and pooled from two tubes of plasma. Corielle genomic DNA (gDNA) were fragmented to a mean size of 180 base pairs (bp) and then sized selected to a tighter distribution using magnetic beads. The library preparation protocol was optimized for low input cell free DNA (cfDNA) and sheared gDNA. Unique molecular identifiers (UMIs) were incorporated into the DNA molecules during adapter ligation. Flowcell clustering adapter sequences and dual sample indices were then incorporated at library preparation amplification with PCR. Libraries were enriched using a targeted capture panel.

Target DNA molecules were first captured using biotinlyated single-stranded DNA hybridization probes and then enriched using magnetic streptavidin beads. Non-target molecules were removed using subsequent wash steps. The HiSeq X Reagent Kit v2.5 (Illumina; San Diego, Calif.) was used for flowcell clustering and sequencing. Four libraries per flowcell were multiplexed. Dual indexing primer mix was included to enable dual sample indexing reads. The read lengths were set to 150, 150, 8, and 8, respectively for read 1, read 2, index read 1, and index read 2. The first 6 base reads in read 1 and read 2 are the UMI sequences.

FIG. 3N is a diagram of observed counts of variants in samples from healthy individuals according to one embodiment. Each data point corresponds to a position (across a range of nucleic acid positions) of a given one of the individuals. The parameters k and p used by the joint model for joint likelihood computations may be selected empirically (e.g., to tune sensitivity thresholds) by cross-validating with sets of cfDNA and gDNA samples from healthy individuals and/or samples known to have cancer. The example results shown in FIG. 3N were obtained with Assay B and using blood plasma samples for the cfDNA and white blood cell samples for the gDNA. For given parameter values for k (“k0” as shown in FIG. 3N) and p, the diagram plots a mean number of variants, which represents a computed upper confidence bound (UCB) of false positives for the corresponding sample. The diagram indicates that the number of false positives decreases as the value of p increases. In addition, the plotted curves have greater numbers of false positives for lower values of k, e.g., closer to 1.0. The dotted line indicates a target of one variant, though the empirical results show that the mean number of false positives mostly fall within the range of 1-5 variants, for k values between 1.0 and 5.0, and p values between 0.5 and 1.0.

The selection of parameters may involve a tradeoff between a target sensitivity (e.g., adjusted using k and p) and target error (e.g., the upper confidence bound). For given pairs of k and p values, the corresponding mean number of false positives may be similar in value, though the sensitivity values may exhibit greater variance. In some embodiments, the sensitivity is measured using percent positive agreement (PPA) values for tumor, in contrast to PPA for cfDNA, which may be used a measurement of specificity:

$${PPA}_{tumor} = \frac{tumor + cfDNA}{tumor}$$

$${PPA}_{cfDNA} = \frac{tumor + cfDNA}{cfDNA}$$

In the above equations, “tumor” represents the number of mean variant calls from a ctDNA sample using a set of parameters, and “cfDNA” represents the number of mean variant calls from the corresponding cfDNA sample using the same set of parameters.

In an embodiment, cross-validation is performed to estimate the expected fit of the joint model to sequence reads (for a given type of tissue) that are different from the sequence reads used to train the joint model. For example, the sequence reads may be obtained from tissues having lung, prostate, and breast cancer, etc. To avoid or reduce the extent of overfitting the joint model for any given type of cancer tissue, parameter values derived using samples of a set of types of cancer tissue are used to assess statistical results of other samples known to have a different type of cancer tissue. For instance, parameter values for lung and prostate cancer tissue are applied to a sample having breast cancer tissue. In some embodiments, one or more lowest k values from the lung and prostate cancer tissue data that maximizes the sensitivity is selected to be applied to the breast cancer sample. Parameter values may also be selected using other constraints such as a threshold deviation from a target mean number of false positives, or 95% UCB of at most 3 per sample. The processing system may cycle through multiple types of tissue to cross validate sets of cancer-specific parameters.

FIG. 3O is a diagram of example parameters for a joint model according to one embodiment. The parameter values for k may be determined as a function of AF observed in gDNA samples, and may vary based on a particular type of cancer tissue, e.g., breast, lung, or prostate as illustrated. The curve 345A represents parameter values for breast and prostate cancer tissue, and the curve 345B represents parameters values for lung cancer tissue. Although the examples thus far describe k and p generally and with reference to implementations where these parameters are fixed, in practice k and p may vary as any function of AF observed in the gDNA sample. In the example shown in the FIG. 3O, the function is a hinge loss function with a hinge value (or lower threshold value), e.g., one-third. Specifically, the function specifies that k equal a predetermined upper threshold, e.g., 3, for AF_{gDNA} values greater than or equal to the hinge value. For AF_{gDNA} values less than the hinge value, the corresponding k values modulates with AF_{gDNA}. The example of FIG. 3O specifically illustrates that the k values for AF_{gDNA} values less than one-third may be proportional to AF_{gDNA} according to a coefficient (e.g., slope in the case of a linear relationship), which may vary between types of cancer tissue. In other embodiments, the joint model can use another type of loss function such as square loss, logistic loss, cross entropy loss, etc.

The joint model may alter k according to a hinge loss function or another function to guard against non-tumor or disease related effects where a fixed value for k would not accurately capture and categorize those events. The hinge loss function example is particularly targeted at handling loss of heterozygosity (LOH) events. LOH events are germline mutations that occur when a copy of a gene is lost from one of an individual's parents. LOH events may contribute to significant portions of observed AF of a gDNA sample. By capping the k values to the predetermined upper threshold of the hinge loss function, the joint model may achieve greater sensitivity for detecting true positives in most sequence reads while also controlling the number of false positives that would otherwise be flagged as true positives due to the presence of LOH. In other embodiments, k and p may be selected based on training data specific to a given application of interest, e.g., having a target population or sequencing assay.

In some embodiments, the joint model takes into account both the AF of a gDNA sample and a quality score of the gDNA sample to guard against underweighting low AF candidate variants. The quality score for a noise model may be used to estimate the probability of error on a Phred scale. Additionally, the joint model may use a modified piecewise function for the hinge function. For example, the piecewise function includes two or more additive components. One component is a linear function based on the AF of the gDNA sample, and another component is an exponential function based on the quality score of the gDNA sample. Given a quality score threshold and a maximum AF scaling factor kₘₐₓ, the joint model determines, using the exponential component of the piecewise function:

$${k}_{ma x} \cdot P ( not error ) = \frac{1 - P ( error )}{{P ( error )}_{m i n}}$$

In the above calculation, P(not error) is the probability that an allele of the gDNA sample is not an error, P(error) is the probability that the allele of the gDNA sample is an error, and P(error)ₘᵢₙ is a minimum probability of error. A minimum threshold for error rate may be empirically determined as the intersecting point for the quality score densities between the likely somatic and likely germline candidate variants of alleles of the gDNA sample.

2.5.1. Example Variant Calls of Joint Model

FIGS. 3R-3S are diagrams of variant calls determined by a joint model according to one embodiment. The example results shown in FIG. 3R were obtained using targeted sequencing assay A and samples known to be affected by early stage cancer. The example results shown in FIG. 3S were obtained using targeted sequencing assay B and samples known to be affected by late stage cancer. The plots in FIGS. 3R-3S share a common x-axis representing observed AF for gDNA. Further, the plots indicate that a variance of the ratio of observed AF of samples of cfDNA and gDNA is greater for late stage cancer than for early stage cancer. The variant caller 240 determines the posterior probability P(AF_{cfDNA}≥k·AF_{gDNA}) for pairs of AF_{cfDNA} and AF_{gDNA} data points, where the gradients of the plots represent the range of probabilities. Each data point represents a candidate cfDNA variant (e.g., for a given nucleic acid position) in an individual, and the plots include data points for multiple individuals in a data set. In the embodiment illustrated, the posterior probability is closer to 1.0 for ratios greater than 8.00 and AF_{gDNA} values less than 0.00391, while the posterior probability is closer to 0.0 for ratios approaching 0.25.

FIG. 3T is a diagram of probability densities determined by a joint model according to one embodiment. The example results shown in FIG. 3T were determined using sequence reads from breast, lung, and prostate tissue samples with an observed AF of gDNA that equals 0. FIG. 3T illustrates some general points about the joint model, regardless of the specific implementation. In such cases where no ALTs are observed (AF_{gDNA}=0), or where low numbers of ALTs are observed in gDNA, the processing system may have a low confidence level regarding the source of ALTs observed in the corresponding cfDNA samples. These situations may occur due to background noise or low depth of the gDNA sample. Since the processing system may not necessarily detect all of the ALTs of the gDNA sample, sequence reads of the cfDNA may still include false positives even when observed AF_{gDNA}=0. Additionally, the joint model models AF_{gDNA} as a distribution with noise, so the true AF_{gDNA} may be modeled as a distribution over non-zero values of likelihood. As a consequence, in these conditions the processing system may filter out ALTs observed in cfDNA samples due to the low confidence of the source of the ALTs, for example, where it is uncertain whether the observed ALTs originated from gDNA or from cancer or diseased cells. In an embodiment, the processing system filters out data points having a probability less than a threshold probability, as illustrated by the dotted line in FIG. 3T.

FIG. 3U is a diagram of sensitivity and specificity of a joint model according to one embodiment. The processing system determines the sensitivity (e.g., PPAₜᵤₘₒᵣ) and specificity (e.g., PPA_{cfDNA}) measurements using assays A and B and with healthy samples, as well as samples known to have breast, lung, and prostate cancer. In comparison to the example results obtained using an empirical threshold, the example results obtained using the joint model show a slight decrease in sensitivity, e.g., 0.14 decreased to 0.12 for PPAₜᵤₘₒᵣ or of assay A using lung tissue samples. However, the joint model results show a greater increase in specificity, e.g., 0.12 increased to 0.22 for PPA_{cfDNA} of assay A using lung tissue samples.

FIG. 3V is a diagram of a set of genes detected from targeted sequencing assays using a joint model according to one embodiment. The set includes genes that are commonly mutated during clonal hematopoiesis. The processing system determines the results using assays A and B and samples known to have breast, lung, and prostate cancer. The tests “Threshold X” and “Joint Model X” do not include nonsynonymous mutations, while the tests “Threshold Y” and “Joint Model Y” do include nonsynonymous mutations. The example results obtained using the joint model 225 reduces the counts of detected germline mutations from samples of various types of tissue, in comparison to the counts detected using an empirical threshold. For instance, as illustrated by the graph for assay B with lung cancer, “Threshold X” and “Threshold Y” results in counts of 5 and 6 detected TET2 genes, respectively. The “Joint Model X” and “Joint Model Y” results in counts of 2 and 3 detected TET2 genes, respectively, which indicates that the joint model 225 provides improved sensitivity.

FIG. 3W is a diagram of length distributions of the set of genes shown in FIG. 3V detected from targeted sequencing assays using a joint model according to one embodiment. Generally, nucleic acid fragments that originate from tumor or diseased cells have shorter lengths (e.g., of nucleotides) than those that originate from reference alleles. As shown in the box plot results for assay B with the breast cancer sample, the median differences in length between detected ALTs and reference alleles for the TET2 gene are approximately zero for both “Threshold X” and “Threshold Y.” In contrast, the median differences in length between detected ALTs and reference alleles for the TET2 gene are approximately −5 for both “Joint Model X” and “Joint Model Y.” Thus, the processing system may determine with greater confidence that the detected ALTs potentially originated from a tumor or diseased cell, instead of a reference allele. Moreover, the example results indicate that the joint model can perform variant calls of short fragments of sequence reads in samples with varying noise levels.

FIG. 3X is a diagram of another set of genes detected from targeted sequencing assays using a joint model according to one embodiment. The example results indicate that the sensitivity for detecting driver genes of the joint model is comparable to that of filters that do not use a model. That is, the joint model does not significantly over filter the detected driver genes relative to the results obtained using an empirical threshold.

2.6. Example Tuning of Joint Model

FIG. 3Y is flowchart of a method 350 for tuning a joint model to process cell free nucleic acid (e.g., cfDNA) samples and genomic nucleic acid (e.g., gDNA) samples according to one embodiment. The method 350 may be performed in conjunction with the methods 315, 325, and/or 335 shown in FIGS. 3J-3L, or another similar method. For example, the method 350 is performed using a joint model to determine the probability for step 355A of the method 350. The examples described with respect to FIGS. 3Y, 3Z, and 3AA reference blood (e.g., white blood cells) of a subject as the source of the gDNA sample, though it should be noted that in other embodiments, the gDNA may be from a different type of biological sample. The processing system may implement at least a portion of the method 350 as a decision tree to filter or process candidate variants in the cfDNA sample. For instance, the processing system determines whether a candidate variant is likely associated or not with the gDNA sample, or if an association is uncertain. An association may indicate that the variant can be accounted for by a mutation in the gDNA sample (e.g., due to factors such as germline mutations, clonal hematopoiesis, artifacts, edge variants, human leukocyte antigens such as HLA-A, etc.) and thus likely not tumor-derived and not indicative of cancer or disease. The method 350 may include different or additional steps than those described in conjunction with FIG. 3Y in some embodiments or perform steps in different orders than the order described in conjunction with FIG. 3Y.

In step 355A, the processing system determines a probability that the true alternate frequency of a cfDNA sample is greater than a function of a true alternate frequency of a gDNA sample. Step 355A may correspond to previously described step 340E of the method 335 shown in FIG. 3L.

In step 355B, the processing system determines whether the probability is less than a threshold probability. As an example, the threshold probability may be 0.8, however in practice the threshold probability may be any value between 0.5 and 0.999 (e.g., determined based on a desired filtering stringency), static or dynamic, vary by gene and/or set by position, or other macro factors, etc. Responsive to determining that the probability is greater than or equal to the threshold probability, the processing system determines that the candidate variant is likely not associated with the gDNA sample such as a blood draw including white blood cells of a subject, i.e., not blood-derived. For example, the candidate variant is typically not present in sequence reads of the gDNA sample for a healthy individual. Accordingly, the processing system may call the candidate variant as a true positive that potentially associated with cancer or disease, e.g., potentially tumor-derived.

In step 355C, the processing system determines whether the alternate depth of the gDNA sample is significantly the same as or different than zero. For instance, the processing system performs an assessment using a quality score of the candidate variant using a noise model. The processing system may also compare the alternate depth against a threshold depth, e.g., determining whether the alternate depth is less than or equal to a threshold depth. As an example, the threshold depth may be 0 or 1 reads. Responsive to determining that the alternate depth of the gDNA sample is significantly different than zero, the processing system determines that there is positive evidence that the candidate variant is associated with nucleotide mutations not caused by cancer or disease. For instance, the candidate variant is blood-derived based on mutations that may typically occur in sequence reads of healthy white blood cells.

Responsive to determining that the alternate depth of the gDNA sample is not significantly nonzero, the processing system determines that the candidate variant is possibly associated with the gDNA sample, but does not make a determination of a source of the candidate variant. In other words, the processing system may be uncertain about whether the candidate variant is blood-derived or tumor-derived. In some embodiments, the processing system may select one of multiple threshold depths for comparison with the alternate depth. The selection may be based on a type of processed sample, noise level, confidence level, or other factors.

In step 355D, the processing system determines a gDNA depth quality score of sequence reads of the gDNA sample. In an embodiment, the processing system calculates the gDNA depth quality score using the an alternate depth of the gDNA sample, where C is a predetermined constant (e.g., 2) to smooth the gDNA depth quality score using a weak prior, which avoids divide-by-zero computations:

$${AD}_{gDNA} depth quality score = \frac{{AD}_{gDNA}}{\sqrt{{AD}_{gDNA} + C}}$$

In step 355E, the processing system determines a ratio of sequence reads of the gDNA sample. The ratio may represent the observed cfDNA frequency and observed gDNA frequency in the processed samples. In an embodiment, the processing system calculates the ratio using the depths and alternate depths of the cfDNA sample and gDNA sample:

$$ratio = \frac{( {AD}_{cfDNA} + {C}_{1} ) ( {depth}_{gDNA} + {C}_{2} )}{( {AD}_{gDNA} + {C}_{3} ) ( {depth}_{cfDNA} + {C}_{4} )}$$

The processing system may use the predetermined constants C₁, C₂, C₃, and C₄ to smooth the ratio by a weak prior. As examples, the constants may be: C₁=2, C₂=4, C₃=2, and C₄=4. Thus, the processing system may avoid a divide-by-zero computation if one of the depths or alternate depths in the ratio denominator equals zero. Thus, the processing system may use the predetermined constants to steer the ratio to a certain value, e.g., 1 or 0.5.

In step 355F, the processing system determines whether the gDNA depth quality score is greater than or equal to a threshold score (e.g., 1) and whether the ratio is less than a threshold ratio (e.g., 6). Responsive to determining that the gDNA depth quality score is less than the threshold score or that the ratio is greater than or equal to the threshold ratio, the processing system determines that there is uncertain evidence regarding association of the candidate variant with the gDNA sample. Stated differently, the processing system may be uncertain about whether the candidate variant is blood-derived or tumor-derived because the candidate variant appears “bloodish” but there is not definitive evidence that a corresponding mutation is found in healthy blood cells.

In step 355G, responsive to determining that the gDNA depth quality score is greater than or equal to the threshold score and that the ratio is less than the threshold ratio, the processing system determines that the candidate variant is likely associated with a nucleotide mutation of the gDNA sample. In other words, the processing system determines that although there is not definitive evidence that a corresponding mutation is found in healthy blood cells, the candidate variant appears bloodier than normal.

Thus, the processing system may use the ratio and gDNA depth quality score to tune the joint model to provide greater granularity in determining whether certain candidate variants should be filtered out as false positives (e.g., initially predicted as tumor-derived, but actually blood-derived), true positives, or uncertain due to insufficient evidence or confidence to classify into either category. For example, based on the result of the method 350, the processing system may modify one or more of the parameters (e.g., k parameter) for a hinge loss function of the joint model. In some embodiments, the processing system uses one or more steps of the method 350 to assign candidate variants to different categories, for instance, “definitively,” “likely,” or “uncertain” association with gDNA (e.g., as shown in FIGS. 3Z and 3AA).

In various embodiments, the processing system processes candidate variants using one or more filters in addition to the steps described with reference to the flowchart of the method 350 shown in FIG. 3Y. The processing system may implement the filters in a sequence as part of a decision tree, where the processing system continues to check criteria of the filters until a given candidate variant “exits” the decision tree, e.g., because the given candidate variant is filtered upon satisfying at least one of the criteria. A filtered candidate variant may indicate that the candidate variant can be accounted for by a source or cause of mutations naturally occurring in healthy individuals (e.g., associated with white blood cell gDNA) or due to process errors.

In some embodiments, the processing system filters candidate variants of sequence reads of a cfDNA sample responsive to determining that there is no quality score for the sequence reads. The processing system may determine quality scores for candidate variants using a noise model. The processing system may determine the quality scores with no base alignment. In some embodiments, the quality score may be missing for some samples or candidate variants due to a lack of training data for the joint model or poor training data that fails to produce useful parameters for a given candidate variant. For instance, high noise levels in sequence reads may lead to unavailability of useful training data. The processing system may tune specificity and selectivity of the joint model based on whether a single variant is processed or if the processing system is controlling for a targeted panel. As other examples, the processing system filters a candidate variant responsive to determining that the candidate variant is an edge variant artifact, has less than a threshold cfDNA depth (e.g., 200 sequence reads), has less than a threshold cfDNA quality score (e.g., 60), or corresponds to human leukocyte antigens (HLA), e.g., HLA-A. Since sequences associated with HLA-A may be difficult to align, the processing system may perform a custom filtering or variant calling process for sequences in these regions.

In some embodiments, the processing system filters candidate variants determined to be associated with germline mutations. The processing system may determine that a candidate variant is germline by determining that the candidate variant occurs at an appropriate frequency corresponding to a given germline mutation event and is present at a particular one or more positions (e.g., in a nucleotide sequence) known to be associated with germline events. Additionally, the processing system may determine a point estimate of gDNA frequency, where C is a constant (e.g., 0.5):

$${point}_{afDNA} = \frac{{AD}_{gDNA}}{{depth}_{gDNA} + C}$$

The processing system may determine that a candidate variant is germline responsive to determining that point_{afDNA} is greater than a threshold point estimate threshold (e.g., 0.3). In some embodiments, the processing system filters candidate variants responsive to determining that a number of variants associated with local sequence repetitions is greater than a threshold value. For example, an “AAAAAA” or “ATATATAT” local sequence repetition may be result of a polymerase slip that causes an increase in local error rates.

FIG. 3Z is a table of example counts of candidate variants of cfDNA samples according one an embodiment. The example data in FIGS. 3Z, 3AA, and 3AB were generated using sequence reads obtained from a sample set of individuals. The cfDNA samples include samples from individuals known to have cancer or another type of disease. In the example shown FIG. 3Z, the processing system uses the method 350 of FIG. Y to determine that 23805 of the candidate variants are “definitively” associated with gDNA (e.g., accounted for by germline mutations or clonal hematopoiesis in blood) and that 1360 of the candidate variants are likely associated with gDNA (e.g., “bloodier” or greater than a threshold confidence level). Thus, the processing system may filter out these candidate variants from the joint model or another pipeline, e.g., such that these candidate variants are classified as blood-derived. The processing system may determine to neither categorize the count of 2607 uncertain (e.g., bloodish) candidate variants as tumor-derived nor blood-derived. Thus, by tuning the joint model, for example, using the gDNA ratio and gDNA depth quality score from the method 350, the processing system improves granularity (e.g., different levels of confidence) in classifying sources of candidate variants. FIG. 3AA is a table of example counts of candidate variants of cfDNA samples from healthy individuals according to an embodiment. The example counts shown in FIGS. 3Z and 3AA were determined by the processing system using a threshold depth of 200 reads, threshold quality score of 60 (e.g., on a Phred scale), quality score at the corresponding position having a mean squared deviation from germline mutation frequency threshold of 0.005, threshold point estimate of gDNA frequency of 0.3, threshold artifact recurrence rate of 0.05, threshold local sequence repetition count of 7, threshold probability (e.g., that the true alternate frequency of a cfDNA sample is greater than a function of a true alternate frequency of a gDNA sample) of 0.8, threshold gDNA depth of 0, threshold gDNA depth quality score of 1, and threshold gDNA sample ratio of 6. Furthermore, the processing system 200 filtered out candidate variants with no quality score, somatic variants, and HLA-A regions.

FIG. 3AB is a diagram of candidate variants plotted based on ratio of cfDNA and gDNA according to one embodiment. For each of a number of plotted candidate variants of a subject, the x-axis value represents the AF observed in gDNA samples and the y-axis represents the AF observed in a corresponding cfDNA sample of the subject. The example shown in FIG. 3AB includes candidate variants passed by a joint model using a hinge function such as the curve 345A or curve 345B illustrated in FIG. 3O. For this example data and the recited parameters above, the processing system 200 determines that the cluster of candidate variants depicted as cross marks toward the left of the plot, which have a relatively higher AF_{cfDNA} to AF_{gDNA} ratio, are likely to be not associated with nucleotide mutations naturally occurring in white blood cells, and thus predicted as tumor-derived. The dotted line 360A is a reference line representing a 1:1 AF_{cfDNA} to AF_{gDNA} ratio. A hinge function is represented by the dotted graphic 360B, which may not necessarily be a line (e.g., may include multiple segments connected at one or more hinges). The cluster of candidate variants depicted as circles have relatively lower AF_{cfDNA} to AF_{gDNA} ratios, but were still passed by the joint model when using the hinge function represented by 360B (e.g., because several of the candidate variants are plotted above 360B. However, some of these candidate variants may actually be associated with gDNA, e.g., blood-derived, and should be filtered out instead of being called as tumor-derived. The dotted line 360C is a regression line determined using robust fit regression on the clusters of data points depicted in the cross marks. By tuning the hinge function using the regression line 360C, the joint model can filter out more of the candidate variants that may actually be blood-derived. In some embodiments, 360A-C each intersect the origin (0, 0). The processing system determines that there is uncertain evidence as to whether the cluster of candidate variants depicted as triangles (located generally between the clusters of cross marks and circle-type candidate variants) are blood or tumor-derived.

To improve the accuracy of catching these candidate variants, the processing system may use the filters as described above with reference to FIG. 3Y. Further, the processing system may tune the joint model by using more aggressive parameters for a hinge function under certain conditions. For example, the processing system uses a greater probability threshold (e.g., for step 355B of the method 350 shown in FIG. 3Y) responsive to determining that the AD of the gDNA sample is greater than a threshold depth (e.g., 0), which is supportive evidence of nucleotide mutations in blood of healthy samples. In some embodiments, the processing system determines a modified hinge function (or another type of function for classifying true and false positives) using the greater probability threshold. For instance, the modified function may have a sharper cutoff (e.g., relative to the curves 345A and 345B of FIG. 3O) that would filter out at least some candidate variants of the cluster along the dotted diagonal lines in FIG. 3AB. The processing system may also tune the modified function using the gDNA sample quality score or ratio as determined in steps 355D and 355E of the method 350, respectively.

2.7. Example Edge Filtering

Edge filtering is performed (e.g., step 310C shown in FIG. 3B) to filter out candidate variants that may be false positives due to their proximity to the edge of sequence reads.

2.7.1. Example Training Distributions of Features from Artifact and Non-Edge Variants

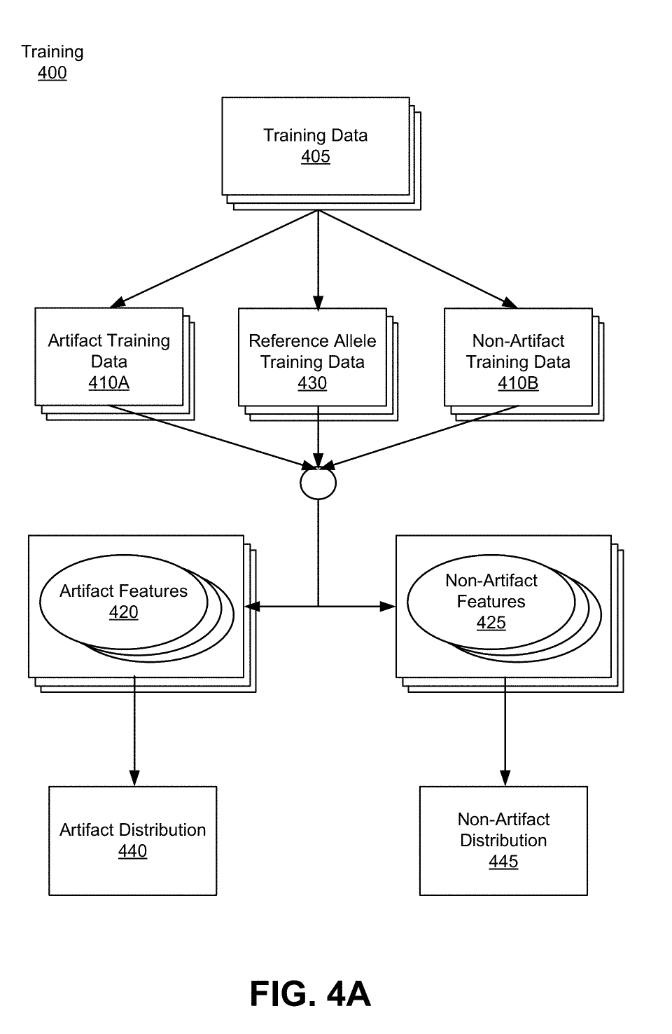

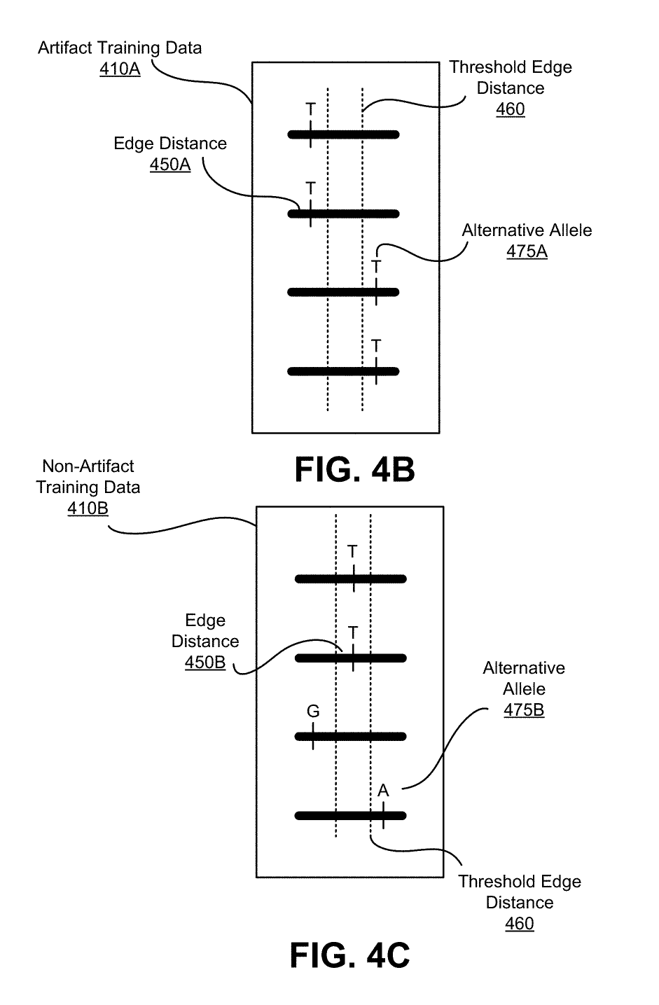

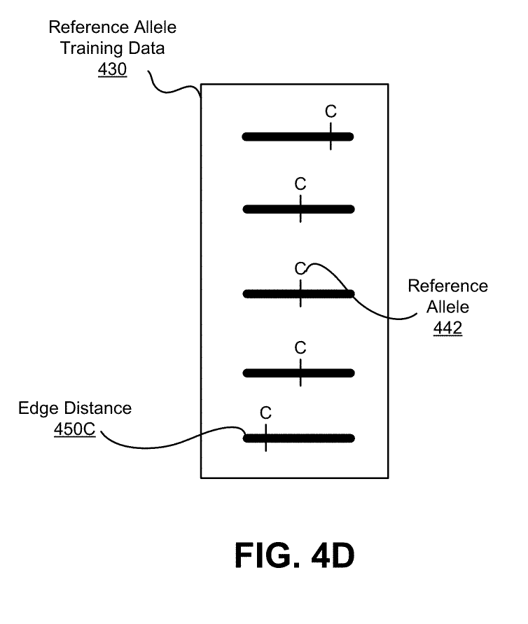

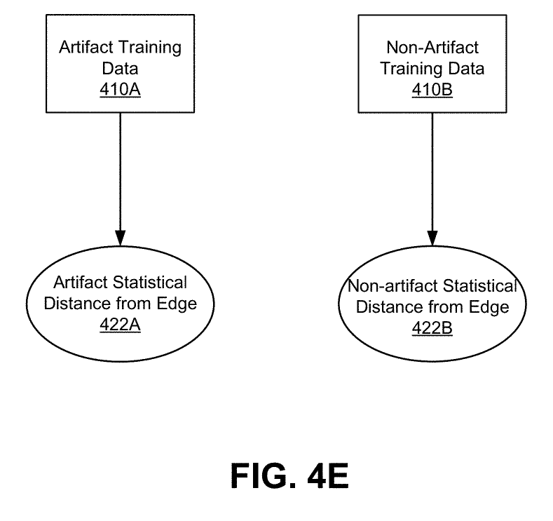

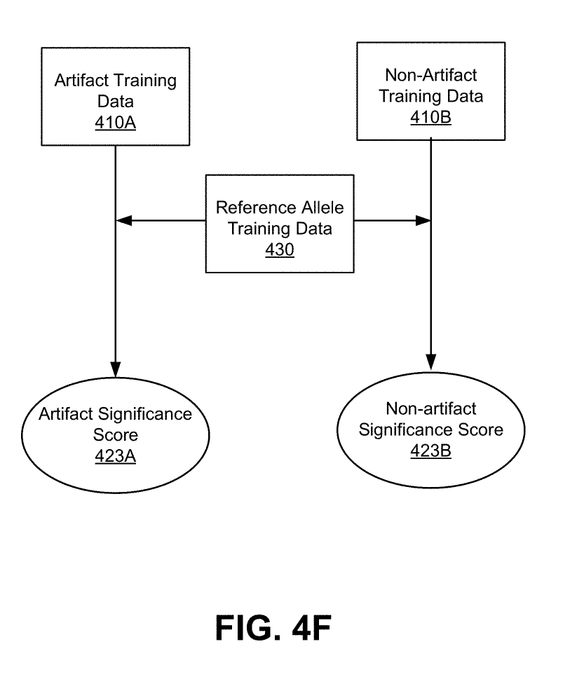

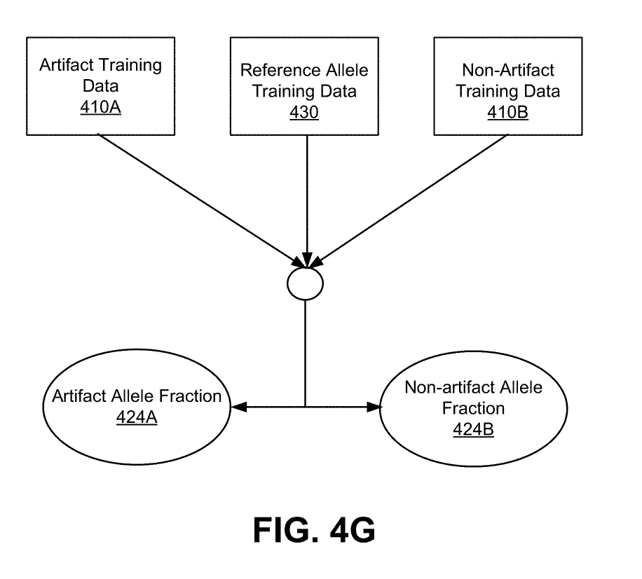

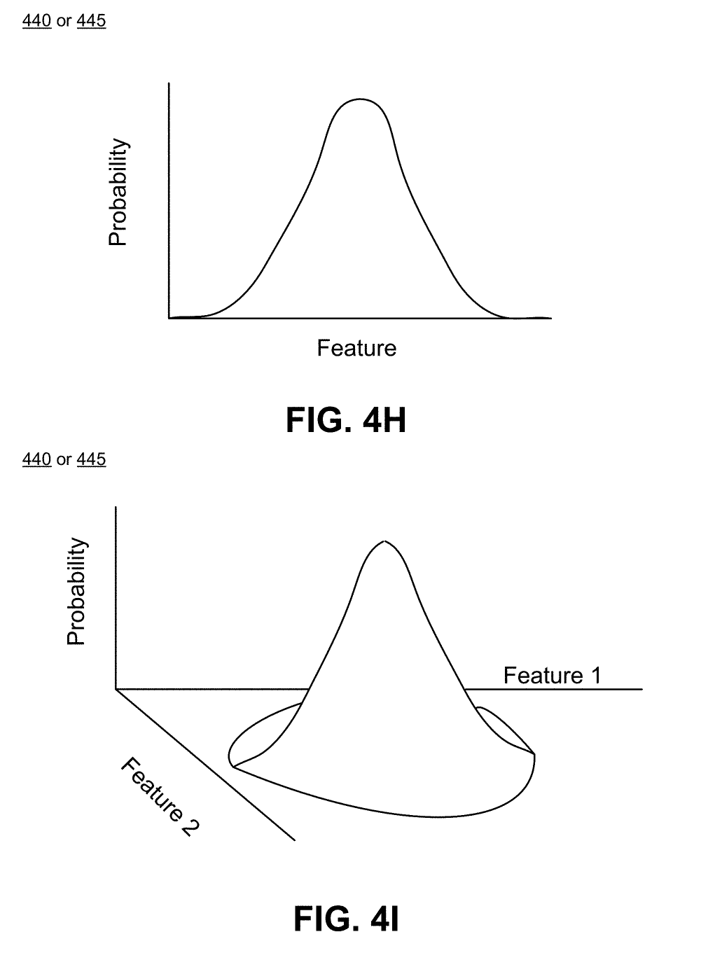

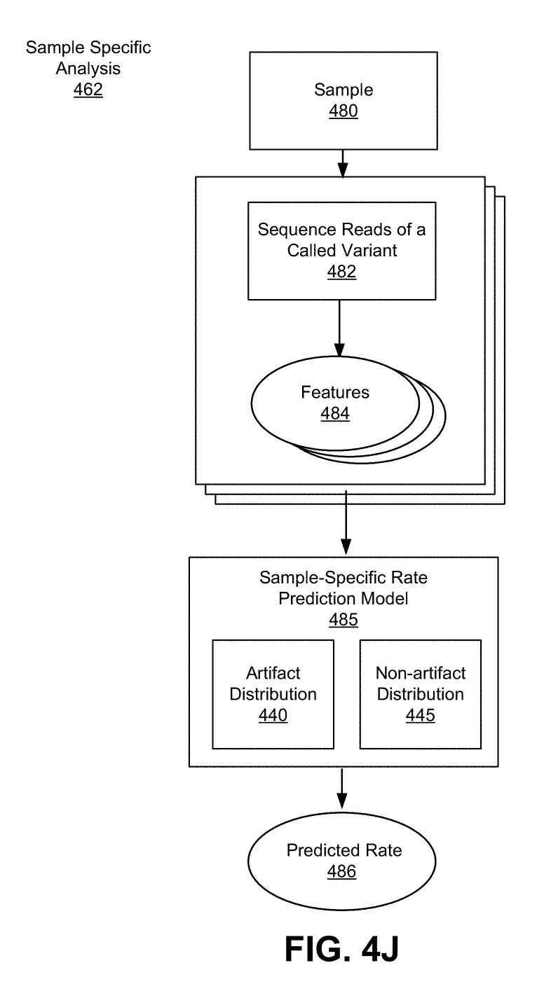

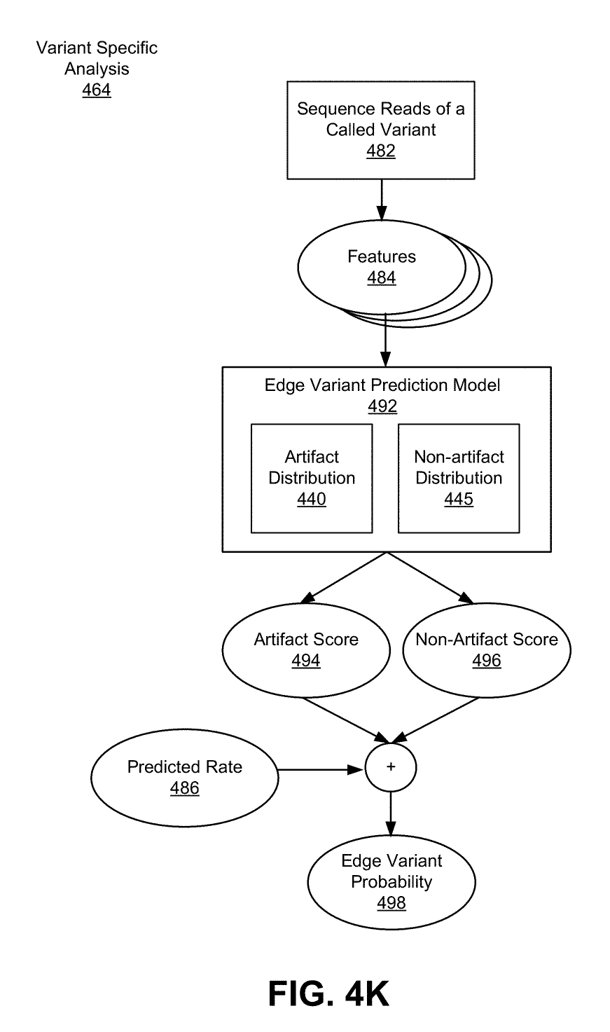

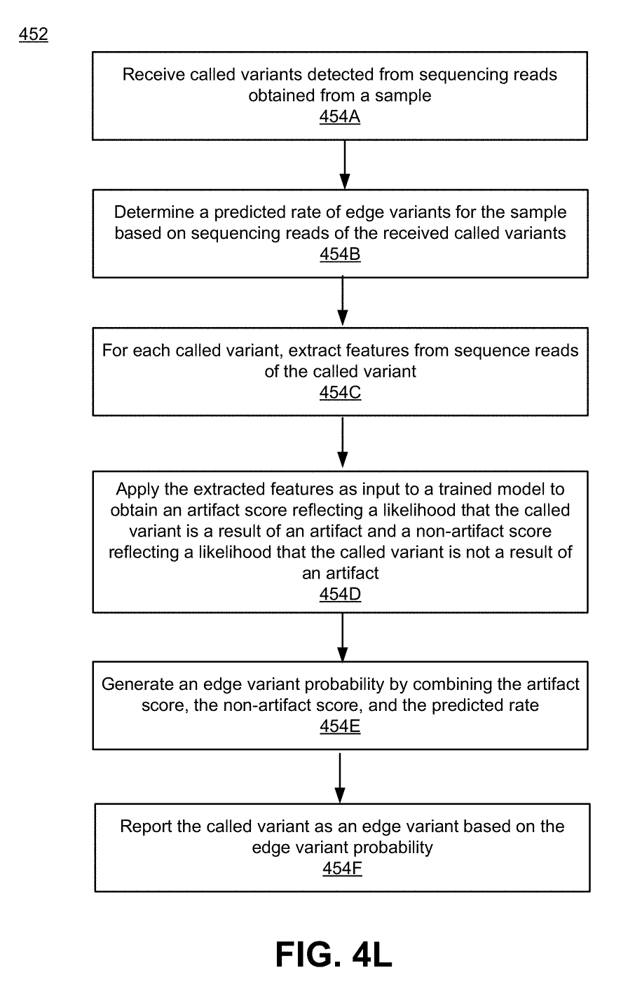

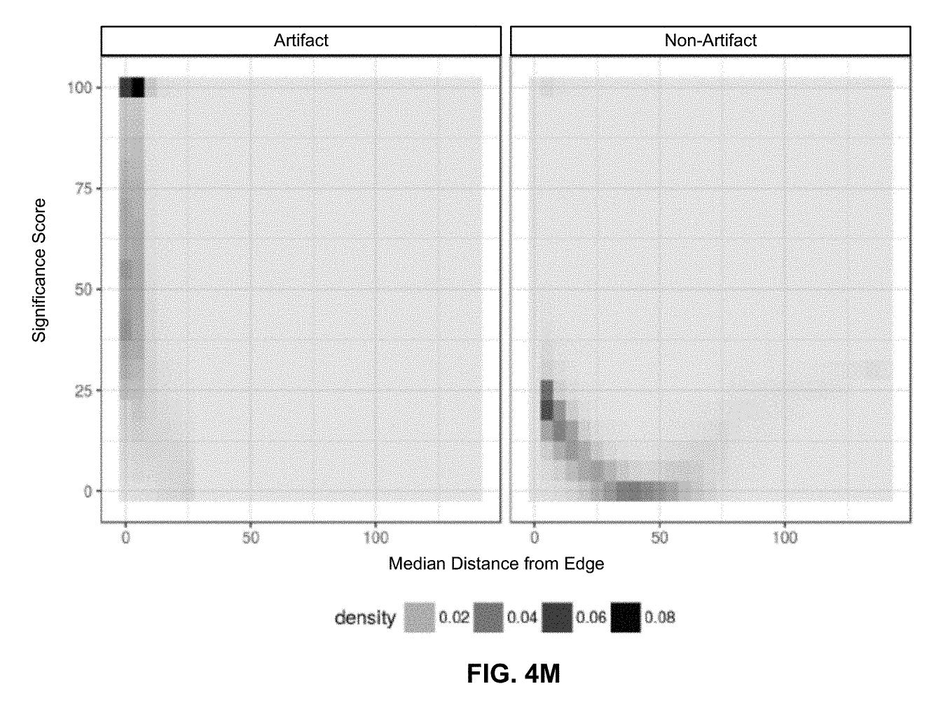

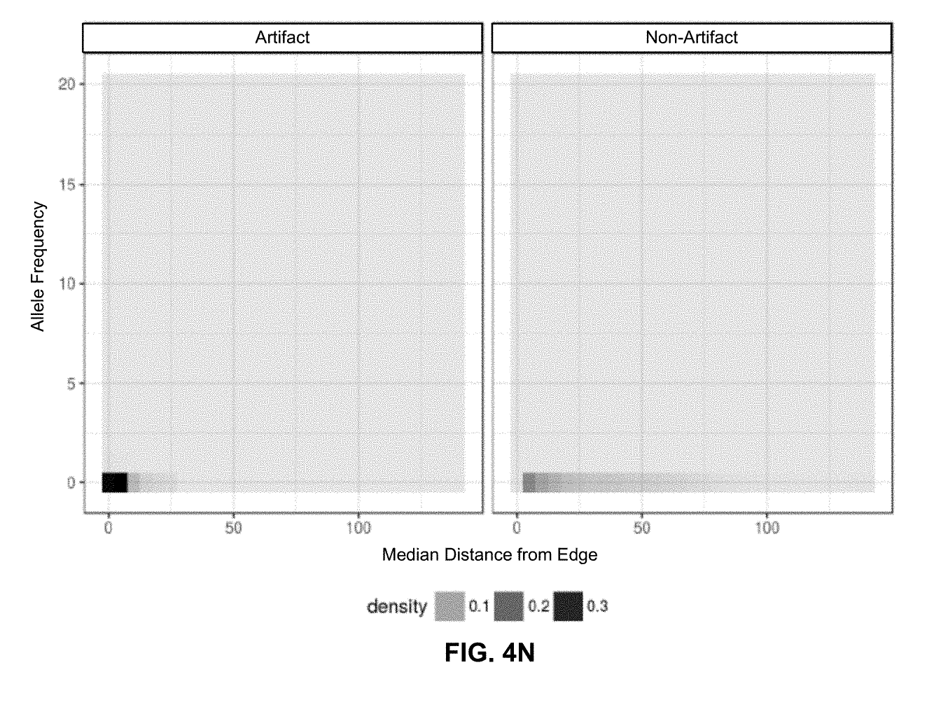

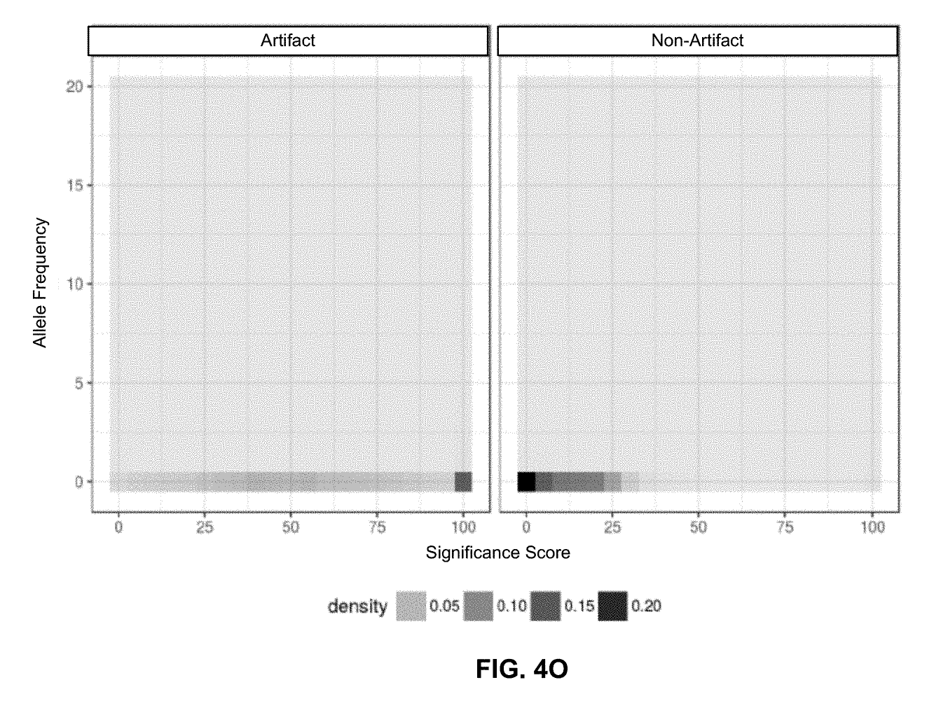

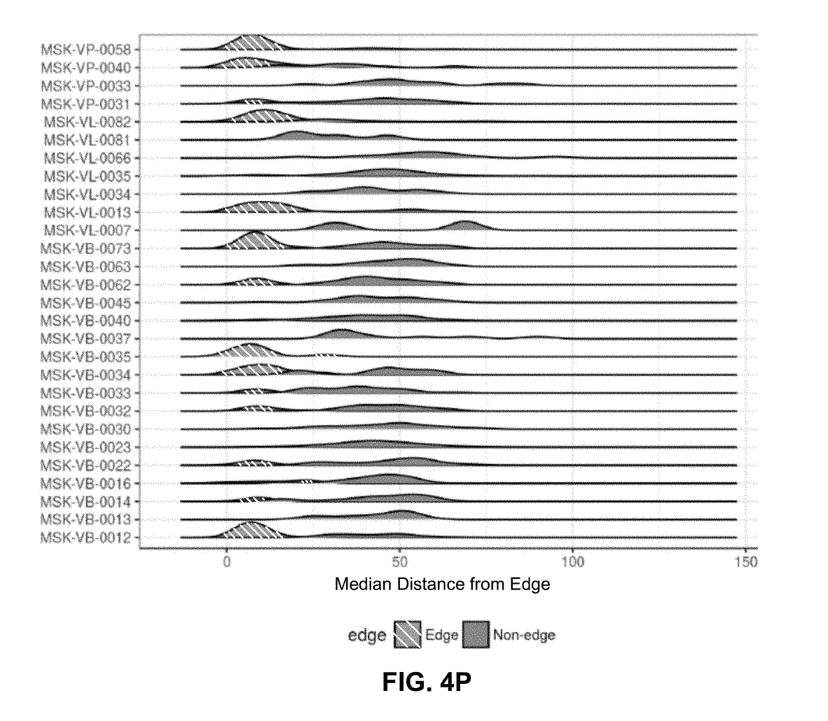

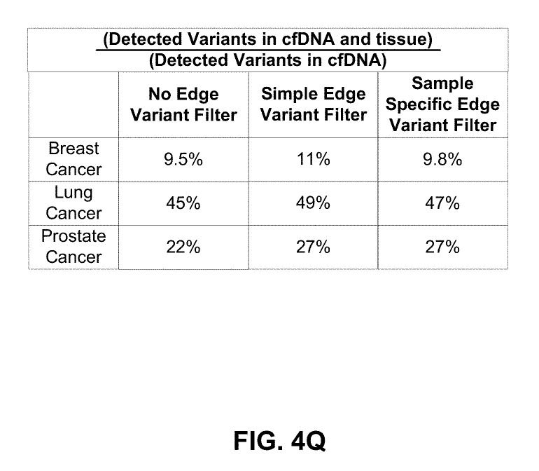

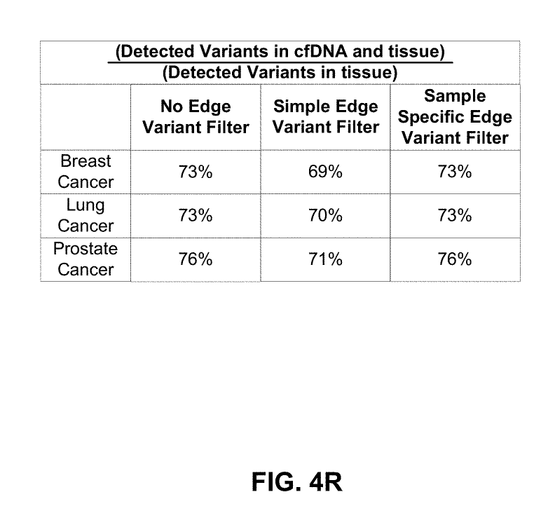

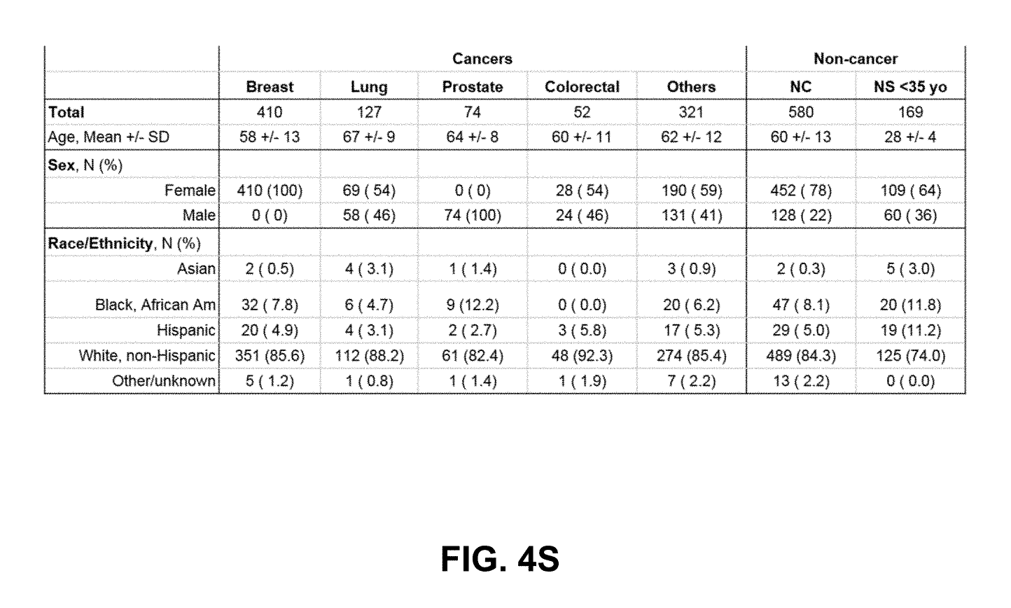

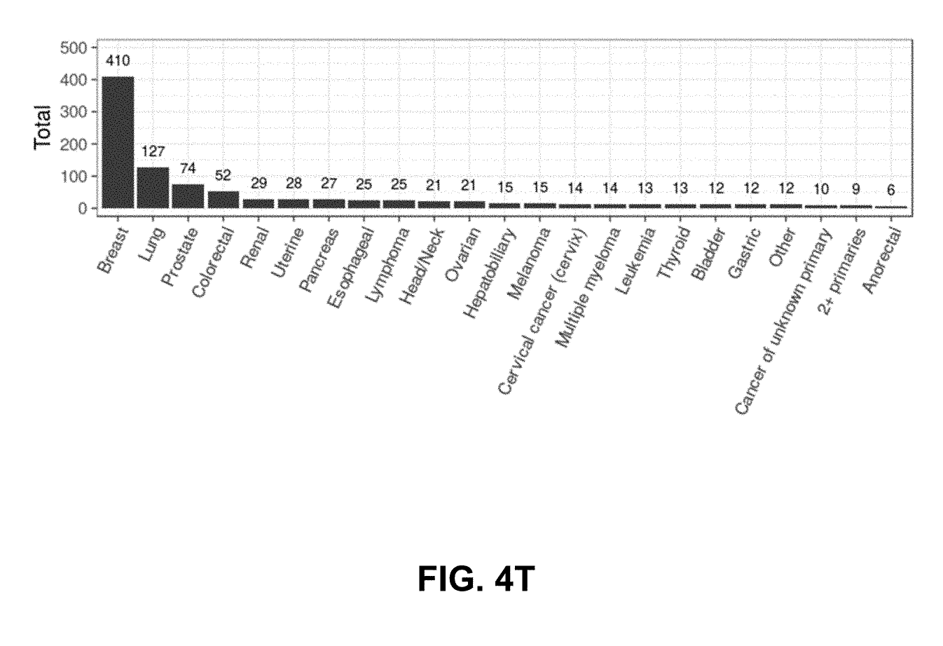

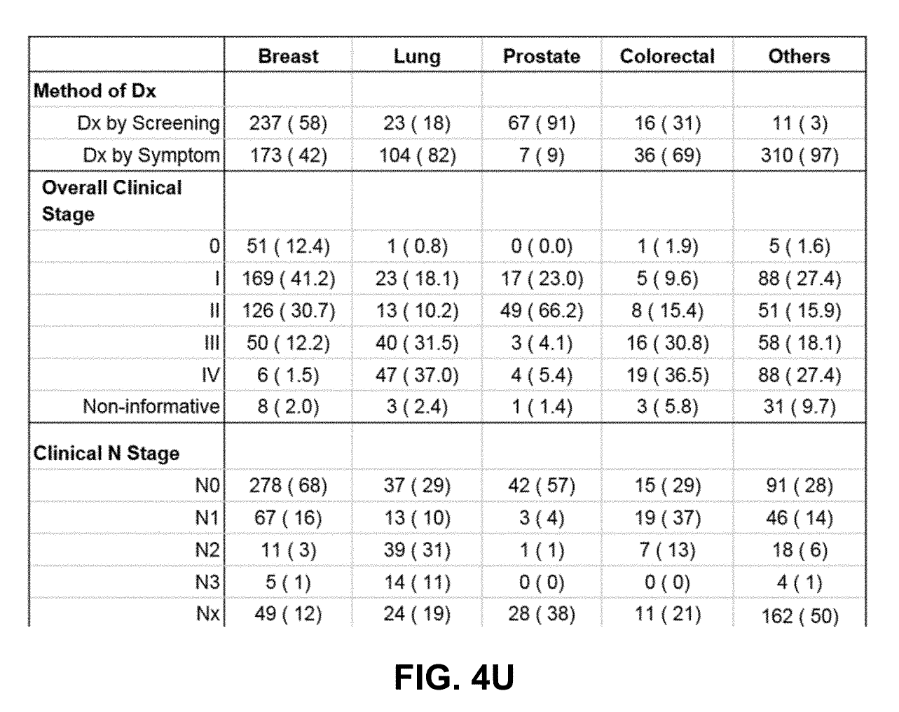

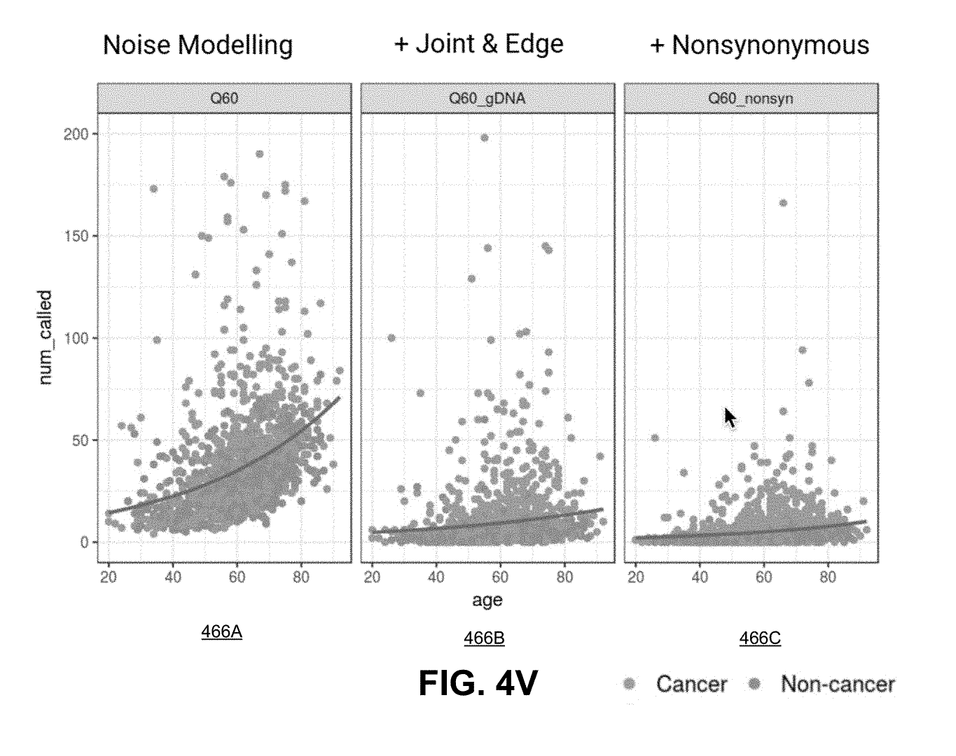

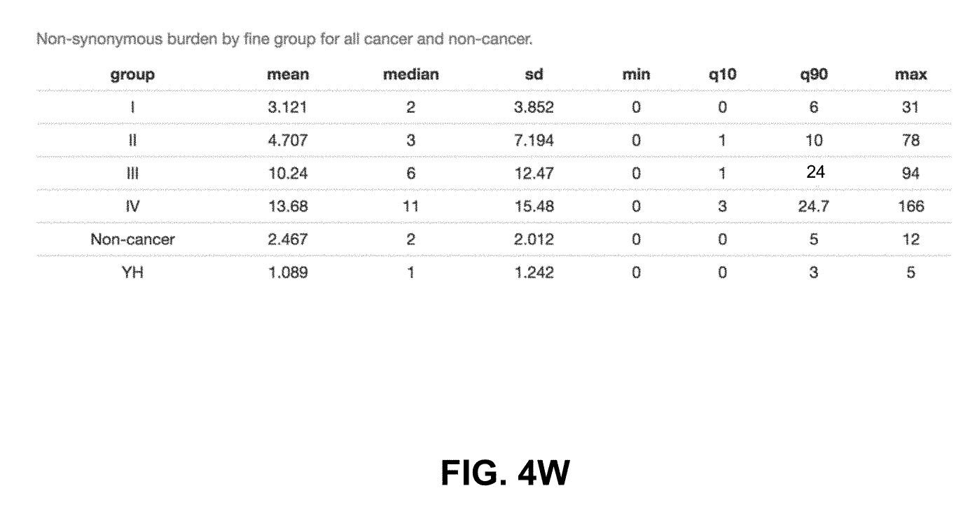

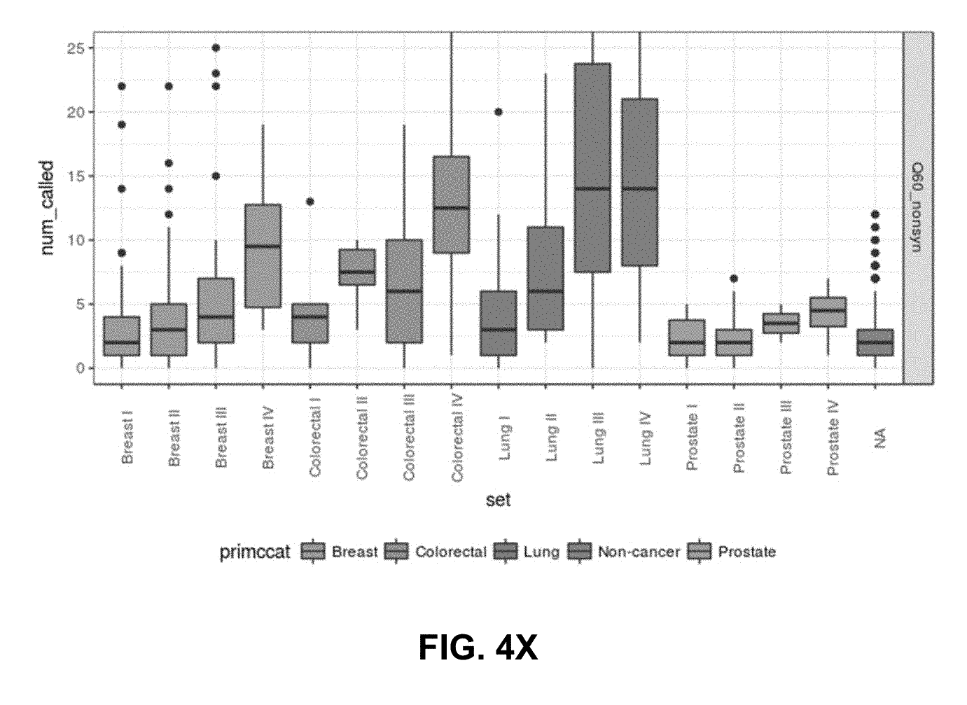

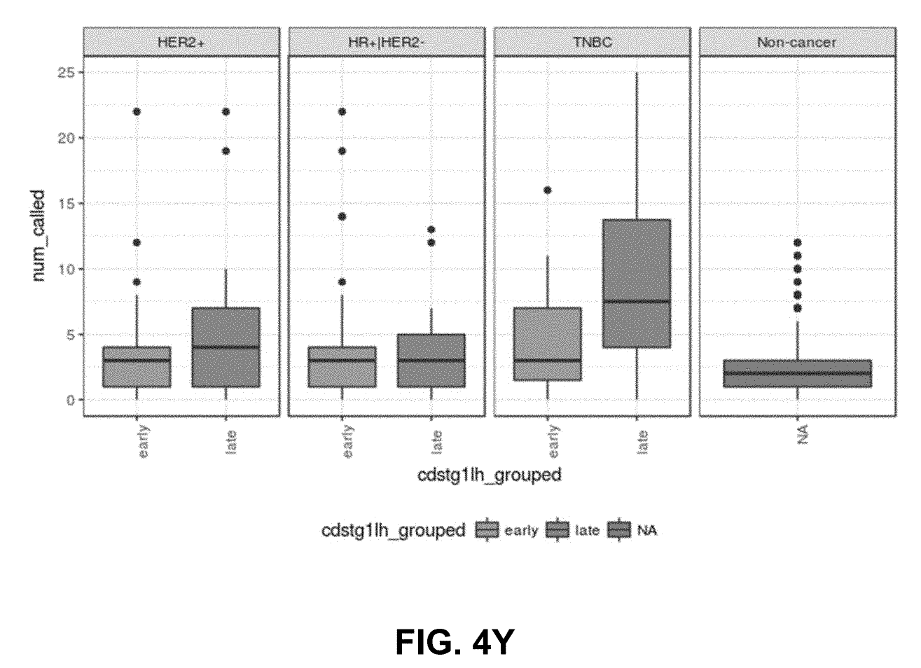

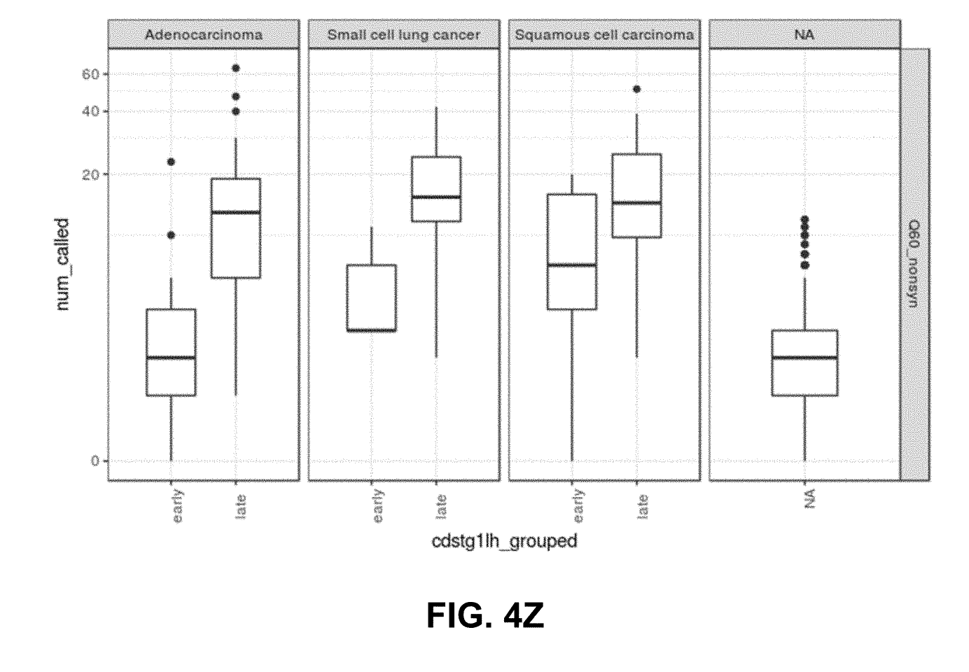

FIG. 4A depicts a process of generating an artifact distribution and a non-artifact distribution using training variants according to one embodiment. The edge filter generates the artifact distribution 440 and non-artifact distribution 445 during a training process 400 using training data 405 from previous samples (e.g., training samples). Once generated, the artifact distribution 440 and non-artifact distribution 445 can each be stored (e.g., in the model database 215) for subsequent retrieval at a needed time.

Training data 405 includes various sequence reads. Sequence reads in the training data 405 can correspond to various positions on the genome. In various embodiments, sequence reads in the training data 405 are obtained from more than one training sample.

The edge filter categorizes sequence reads in the training data 405 into one of an artifact training data 410A category, reference allele training data 430 category, or non-artifact training data 410B category. In various embodiments, sequence reads in the training data 405 can also be categorized into a no result or a no classification category responsive to determining that the sequence reads do not satisfy the criteria to be placed in any of the artifact training data 410A category, reference allele training data 430 category, or non-artifact training data 410B category.

As shown in FIG. 4A, there may be multiple groups of artifact training data 410A, multiple groups of reference allele training data 430, and multiple groups of non-artifact training data 410B. Generally, sequence reads that are in a group cross over (overlap) a common position in the genome. In various embodiments, sequence reads in a group derive from a single training sample (e.g., a training sample obtained from a single individual) and cross over the common position in the genome. For example, given sequence reads from M different training samples obtained from M different individuals, there can be M different groups each including sequence reads from one of the M different training samples. Although the subsequent description refers to groups of sequence reads that cross over a common position on the genome, the description can be further expanded to other groups of sequence reads that cross over other positions on the genome.

Sequence reads that correspond to a common position on the genome include: 1) sequencing reads that include a nucleotide base at the position that is different from the reference allele (e.g., an ALT) and 2) sequencing reads that include a nucleotide base at the position that matches the reference allele. The edge filter categorizes sequence reads that include an ALT into one of the artifact training data 410A or non-artifact training data 410B. Specifically, sequence reads that satisfy one or more criteria are categorized as artifact training data 410A. The criteria can be a combination of a type of mutation of the ALT and a location of the ALT on the sequence read. Referring to an example of a type of mutation, sequence reads categorized as artifact training data include an alternative allele that is either a cytosine to thymine (C>T) nucleotide base substitution or a guanine to adenine (G>A) nucleotide base substitution. Referring to an example of the location of the alternative allele, the alternative allele is less than a threshold number of base pairs from an edge of a sequence read. In one implementation, the threshold number of base pairs is 25 nucleotide base pairs, however, the threshold number may vary by implementation.

FIG. 4B depicts sequence reads that are categorized in an artifact training data 410A category according to one embodiment. Additionally, each of the sequence reads satisfy one or more criteria. For example, each sequence read includes an alternative allele 475A that is a C>T nucleotide base substitution. Additionally, the alternative allele 2375A on each sequence read is located at an edge distance 450A that is less than a threshold edge distance 460.

Sequence reads with an alternative allele that are categorized into the non-artifact training data 410B category are all other sequence reads with an alternative allele that do not satisfy the criteria of being categorized as artifact training data 410A. For example, any sequence read that includes an alternative allele that is not one of a C>T or G>A nucleotide base substitution is categorized as a non-edge training variant. As another example, irrespective of the type of nucleotide mutation, any sequence read that includes an alternative allele that is located greater than a threshold number of base pairs from an edge of a sequence read is categorized as non-artifact training data 410B. In one implementation, the threshold number of base pairs is 25 nucleotide base pairs, however, the threshold number may vary by implementation.

FIG. 4C depicts sequence reads that are categorized in the non-artifact training data 410B category according to one embodiment. Here, each of the sequence reads includes an alternative allele 475B that does not satisfy both criteria. For example, each alternative allele 475B can either be a non C>T or non G>A nucleotide base substitution, irrespective of the location of the alternative allele 475B. As another example, each alternative allele 475B is a C>T or G>A nucleotide base substitution, but is located with an edge distance 450B that is greater than the threshold edge distance 460.

Referring now to the reference allele training data 430 category, sequence reads that include the reference allele are categorized in the reference allele training data 430 category. FIG. 4D depicts sequence reads corresponding to the same position in the genome that are categorized in the reference allele training data 430 category according to one embodiment. As an example, the sequence reads shown in FIG. 4D each include the reference allele 442 (which matches the cytosine nucleotide base 162 shown in FIG. 1B). Additionally, these sequence reads that include the reference allele 442 are categorized in the reference allele training data 430 irrespective of the edge distance 450C between the reference allele and the edge of the sequence read.

Returning to FIG. 4A, the edge filter extracts features from groups of sequencing reads categorized in each of the artifact training data 410A, non-artifact training data 410B, and reference allele training data 430. Each group of sequencing reads corresponds to the same position in the genome. Specifically, artifact features 420 and non-artifact features 425 are extracted from sequence reads in one, two, or all three of the artifact training data 410A, non-artifact training data 410B, and reference allele training data 430. Examples of artifact features 420 and non-artifact features 425 include a statistical distance from edge feature, a significance score feature, and an allele fraction feature. Each of these features are described in further detail below in relation to FIGS. 4E-4G.

FIG. 4E is an example depiction of a process for extracting a statistical distance from edge feature according to one embodiment. Here, the edge filter extracts the artifact and non-artifact statistical distance from edge 422A and 422B features from a group of sequence reads in the artifact training data 410A and a group of sequence reads in the non-artifact training data 410B, respectively. Each statistical distance from edge 422A and 422B feature can represent one of a mean, median, or mode of the distance (e.g., number of nucleotide base pairs) between alternative alleles 475 on sequence reads and the corresponding edge of sequence reads. More specifically, artifact statistical distance from edge 422A represents the combination of edge distances 450A (see FIG. 4B) across sequence reads in a group of the artifact training data 410A. Similarly, non-artifact statistical distance from edge 422B represents the combination of edge distances 450B (see FIG. 4C) across sequence reads in a group of the artifact training data 410B.

FIG. 4F is an example depiction of a process for extracting a significance score feature according to one embodiment. The edge filter extracts the artifact significance score 423A feature from a combination of a group of sequence reads in the artifact training data 410A and a group of sequence reads in the reference allele training data 430. Similarly, the edge filter extracts non-artifact significance score 423B feature from a combination of a group of sequence reads in the non-artifact training data 410B and a group of sequence reads in the reference allele training data 430. Generally, the groups of sequence reads from the artifact training data 410A, non-artifact training data 410B, and reference allele training data 430 correspond to a common position on the genome. Therefore, for each position, there can be an artifact significance score 423A and a non-artifact significance score 423B for that position. Although the subsequent description refers to the process of extracting an artifact significance score 423A, the same description applies to the process of extracting a non-artifact significance score 423B.

The artifact significance score 423A feature is a representation of whether the location of alternative alleles 475A (e.g., in terms of distance from edge of a sequence read or another measure) on a group of sequence reads in the artifact training data 410A is sufficiently different, to a statistically significant degree, from the location of reference alleles 442 on a group of sequencing reads in the reference allele training data 430. Specifically, artifact significance score 423A is a comparison between edge distances 450A of alternative alleles 475A (see FIG. 4B) in the artifact training data 410A and edge distances 450C of reference alleles 442 (see FIG. 4D) in the reference allele training data 430.

In various embodiments, the edge filter performs a statistical significance test for the comparison between edge distances. As one example, the statistical significance test is a Wilcoxon rank-sum test. Here, the edge filter assigns each sequence read in the artifact training data 410A and each sequence read in the reference allele training data 430 a rank depending on the magnitude of each edge distance 450A and 450C, respectively. For example, a sequence read that has the greatest edge distance 450A or 450C can be assigned the highest rank (e.g., rank=1), the sequence read that has the second greatest edge distance 450A or 450C can be assigned the second highest rank (e.g., rank=2), and so on. The edge filter compares the median rank of sequence reads in the artifact training data 410A to the median rank of sequence reads in the reference allele training data 430 to determine whether the locations of alternative alleles 475 in the artifact training data 410A significantly differ from locations of reference alleles 442 in the reference allele training data 430A. As an example, the comparison between the median ranks can yield a p-value, which represents a statistical significance score as to whether the median ranks are significantly different. In various embodiments, the artifact significance score 423A is represented by a Phred score, which can be expressed as:

Phred Score=−10 log₁₀ P

where P is the p-value score. Altogether, a low artifact significance score 423A signifies that the difference in median ranks is not statistically significant whereas a high artifact significance score 423A signifies that the difference in median ranks is statistically significant.

FIG. 4G is an example depiction of a process for extracting an allele fraction feature according to one embodiment. The allele fraction feature refers to the allele fraction of alternative alleles 475A or 475B. Specifically, artifact allele fraction 424A refers to the allele fraction of alternative allele 475A (see FIG. 4B) whereas non-artifact allele fraction 424B refers to the allele fraction of alternative allele 475B (see FIG. 4C). The allele fraction represents the fraction of sequence reads corresponding to the position in the genome that includes the alternative allele. For example, there may be X total sequence reads in the artifact training data 410A that include the alternative allele 475A. There may also be Y total sequence reads in the non-artifact training data 410B that include the alternative allele 475B. Additionally, there may be Z total sequence reads in the reference allele training data 430 with the reference allele. Therefore, the artifact allele fraction 424A of the alternative allele 475A can be denoted as

$$Allele Fraction ( 424 A ) = \frac{X}{X + Y + Z} .$$

Additionally, the non-artifact allele fraction 424B of the alternative allele 475B can be denoted as

$$Allele Fraction ( 424 B ) = \frac{Y}{X + Y + Z} .$$

Returning to FIG. 4A, the edge filter compiles extracted artifact features 420 from groups of sequence reads across various positions of the genome to generate an artifact distribution 440. Additionally, the edge filter compiles extracted non-artifact features 425 from groups of sequence reads across various positions of the genome to generate a non-artifact distribution 445. FIG. 4A depicts one particular embodiment where three different features 420A are used to generate an artifact distribution 440 and three different features 420B are used to generate a non-artifact distribution 445. In other embodiments, fewer or more of each type of feature 420A or 420B are used to generate an artifact distribution 440 or non-artifact distribution 445.

FIGS. 4H and 4I depict example distributions used for identifying edge variants, according to various embodiments. Specifically, FIG. 4H depicts a distribution 440 or 445 generated from one type of artifact feature 420 or non-artifact feature 425. Although FIG. 4G depicts a normal distribution for sake of illustration, in practice, distributions 440 and 445 will vary depending on the values of the feature 420 or 425.

In another embodiment, the edge filter may use multiple artifact features 420 or non-artifact features 425 to generate a single distribution 440 or 445. For example, FIG. 4I depicts a distribution 440 or 445 generated from two types of artifact features 420 or two types of non-artifact features 425. Here, the distribution 440 or 445 describes a relationship between a first feature and a second feature. In further embodiments, a distribution 440 or 445 can represent relationships between three or more types of artifact features 420 or non-artifact features 425.

2.7.2. Example Determining of a Sample-Specific Rate for Identifying Edge Variants

FIG. 4J depicts a block diagram flow process 462 for determining a sample-specific predicted rate according to one embodiment. Generally, the edge filter conducts a sample-wide analysis of called variants in the sample 480 to determine the predicted rate 486 that is specific for the sample 480. In other words, the process 462 shown in FIG. 4J can be conducted once for each sample 480.

Sequence reads of a called variant 482 are obtained from a sample 480. Generally, the sequence reads of a called variant 482 refer to a group of sequence reads that cross over the position in the genome to which the called variant corresponds.

For each called variant, the edge filter extracts features 484 from the sequence reads of the called variant 482. Each feature 484 extracted from sequence reads of a called variant 482 can be a statistical distance from edge of alternative alleles in the sequence reads, an allele fraction of an alternative allele, a significance score, another type of feature, or some combination thereof. The edge filter applies values of features 484 extracted across called variants of the sample 480 as input to a sample-specific rate prediction model 485 that determines a predicted rate 486 for the sample 480. The predicted rate 486 for the sample 480 refers to an estimated proportion of called variants that are edge variants. In various embodiments, the predicted rate 486 is a value between 0 and 1, e.g., inclusive.

As shown in FIG. 4J, the sample-specific rate prediction model 485 uses both the previously generated artifact distribution 440 and non-artifact distribution 445. The sample-specific rate prediction model 485 determines the predicted rate 486 by analyzing the features 484 extracted from sequence reads of called variants in the sample 480 in view of the artifact distribution 440 and non-artifact distribution 445. As an example, the sample-specific rate prediction model 485 performs a goodness of fit to determine a predicted rate 486 that explains the observed features 484, given the artifact distribution 440 and non-artifact distribution 445. In one implementation, the sample-specific rate prediction model 485 performs a maximum likelihood estimation to estimate the predicted rate 486 that maximizes the likelihood of observing the features 484 in view of the artifact distribution 440 and non-artifact distribution 445. However, other implementations may use other processes.

In one embodiment, the likelihood equation for the estimation can be expressed as:

L(w|x)=w*(L(x)d₁)+(1−w)*(L(x|d₂)

where w is the predicted rate 486, x represents the features 484, d₁ represents the artifact distribution 440, and d₂ represents the non-artifact distribution 445. In other words, L(w|x) is the weighted sum of a likelihood of observing the features 484 in view of the artifact distribution 440 and a likelihood of observing the features 484 in view of the non-artifact distribution 445. Therefore, the maximum likelihood estimation determines the predicted rate 486 (e.g., rate w) that maximizes this overall likelihood given a certain set of conditions.

As shown in FIG. 4J, the edge filter can extract multiple features 484 from sequence reads of a called variant 482 and provide the features 484 to the rate prediction model 485. For example, there may be three types of features (e.g., statistical distance from edge of alternative alleles in the sequence reads, an allele fraction of an alternative allele, or a significance score). Generalizing further, assuming that n different types of features 484 (e.g., x₁, x₂, . . . xₙ) are provided to the rate prediction model 485, L(w|x) can be expressed as:

L(w|x₁,x₂ . . . xₙ)=w*(Π₁ⁿL(xₙ)|d₁)+(1−w)*(Π₁ⁿL(xₙ)|d₂) (2)

Altogether, responsive to determining that the distribution of features 484 extracted from sequence reads of the called variants in the sample 480 are more similar to the artifact distribution 440 than the non-artifact distribution 445, the rate prediction model 485 determines a high predicted rate 486, which indicates that a high estimated proportion of called variants are likely edge variants. Alternatively, responsive the distribution of features 484 extracted from sequence reads of variants in the sample 480 are more similar to the non-artifact distribution 445 than the artifact distribution 440, the rate prediction model 485 determines a low predicted rate 486, which indicates that a low estimated proportion of called variants are likely edge variants. As discussed below, the predicted rate 486 can be used to control for the level of “aggressiveness” in which edge variants are identified in a sample. Therefore, a sample that is assigned a high predicted rate 486 can be aggressively filtered (e.g., using broader criteria to filter out a greater number of possible edge variants) whereas a sample that is assigned a low predicted rate 486 can be less aggressively filtered.

2.7.3. Example Variant Specific Analysis for Identifying an Edge Variant

FIG. 4K depicts the application of an edge variant prediction model 492 for identifying edge variants according to one embodiment. In a variant specific analysis 464, the edge filter analyzes sequence reads of a called variant 482 to determine whether the called variant is an edge variant. The process depicted in FIG. 4K can be conducted for each called variant or a subset of called variants that were detected for a single sample 480.

In one embodiment, the edge filter filters called variants based on a type of mutation of the called variant. Here, a called variant that is not of the C>T or G>A mutation type can be automatically characterized as a non-edge variant. Alternatively, any called variant that is of the C>T or G>A is further analyzed in the subsequent steps described hereafter.

As shown in FIG. 4K, the edge filter extracts features 484 from the sequence reads of a called variant 482. The extracted features 484 of the sequence reads of a called variant 482 can be the same features 484 extracted from the sequence reads of a called variant 482 as shown in FIG. 4J. Namely, the features 484 can be one or more of: a statistical distance from edge of alternative alleles in the sequence reads, an allele fraction of an alternative allele, or a significance score, among other types of features.

The edge filter provides values of the extracted features 484 as input to the edge variant prediction model 492. As shown in FIG. 4K, the edge variant prediction model 492 uses both the previously generated artifact distribution 440 and the non-artifact distribution 445. The edge variant prediction model 492 generates multiple scores, such as an artifact score 494 that represents a likelihood that the called variant is an edge variant as well as a non-artifact score 496 that represents a likelihood that the called variant is a non-edge variant.

Specifically, the edge variant prediction model 492 determines the probability of observing the features 484 of the sequence reads of a called variant 482 in view of the artifact distribution 440 and the non-artifact distribution 445. In one embodiment, the edge variant prediction model 492 determines the artifact score 494 by analyzing the features 484 in view of the artifact distribution 440 and determines the non-artifact score 496 by analyzing the features 484 in view of the non-artifact distribution 445.

As a visual example, referring back to the example distribution shown in FIG. 4H, the edge variant prediction model 492 identifies a probability based on where along the x-axis a feature 484 falls. In this example, the identified probability can be the score, such as the artifact score 494 or non-artifact score 496, outputted by the edge variant prediction model 492.

As shown in FIG. 4K, the edge filter combines the artifact score 494 and non-artifact score 496 with the sample-specific predicted rate 486 (as described in FIG. 4J). The combination yields the edge variant probability 498, which represents the likelihood that the called variant is a result of a processing artifact.

In one embodiment, edge variant probability 498 can be expressed as the posterior probability of the called variant being an edge variant in view of the features 484 extracted from sequence reads of the called variant 482. The combination of the artifact score 494, the non-artifact score 496, and the sample-specific predicted rate 486 can be expressed as:

$$Edge Variant Probability = \frac{w ⋆ artifact score}{( w ⋆ artifact score ) + ( 1 - w ) ⋆ ( nonartifact score )} \qquad ( 3 )$$

The edge filter may compare the edge variant probability 498 against a threshold value. Responsive to determining that the edge variant probability 498 is greater than the threshold value, the edge filter determines that the called variant is an edge variant. Responsive to determining that the edge variant probability 498 is less than the threshold value, the edge filter determines that the called variant is a non-edge variant.

2.7.4. Example Variant Specific Analysis for Identifying an Edge Variant

FIG. 4L depicts a flow process 452 of identifying and reporting edge variants detected from a sample according to one embodiment. Called variants from various sequencing reads are received 454A from a sample. A sample-specific predicted rate is determined 454B for the sample based on sequencing reads of the called variants from the sample. As one example, a predicted rate is determined by performing a maximum likelihood estimation. Here, the predicted rate is a parameter value that maximizes (e.g., given certain conditions) the likelihood of observing features 484 of sequence reads of the called variants in view of previously generated distributions.

For each called variant, one or more features 484 are extracted 454C from the sequence reads of the variant. Values of the extracted features 484 are applied 454D as input to a trained model to obtain an artifact score 494. The artifact score 494 represents a likelihood that the called variant is an edge variant (e.g., result of a processing artifact). The trained model further outputs a non-artifact score 496 which represents a likelihood that the called variant is a non-edge variant (e.g., not a result of a processing artifact).

For each called variant, an edge variant probability 498 is generated 498 by combining the artifact score 494 for the called variant, non-artifact score 496 for the called variant, and the sample-specific predicted rate 486. Based on the edge variant probability 498, the called variant can be reported 454E as an edge variant (e.g., variants that were called as a result of a processing artifact).

2.7.5. Examples of Edge Filtering

The following examples are put forth so as to provide those of ordinary skill in the art with a complete disclosure and description of how to make and use the disclosed embodiments, and are not intended to limit the scope of what is regarded as the invention. Efforts have been made to ensure accuracy with respect to the numbers used (e.g. amounts, temperature, concentrations, etc.) but some experimental errors and deviations should be allowed for. It will be appreciated by those of skill in the art that, in light of the present disclosure, numerous modifications and changes can be made in the particular embodiments exemplified without departing from the intended scope of the invention.

2.7.5.1. Categorizing Artifact and Clean Training Samples

FIGS. 4M, 4N, and 4O each depict the features of example training variants that are categorized in one of the artifact or non-artifact categories according to various embodiments. The examples shown in FIGS. 4M, 4N, and 4O include artifact distributions and non-artifact distributions determined using the process 400 shown in FIG. 4A. cfDNA samples were obtained from subjects with one of breast cancer, lung cancer, or prostate cancer through a blood draw. The sample set includes at least 50 subjects for each type of cancer (breast, lung, and prostate cancer). For all participating subjects, blood was drawn contemporaneously within six weeks of (prior to or after) biopsy.

The edge filter categorizes sequence reads that include an alternative allele for a particular site on the genome into artifact and non-artifact groups, as is described below. Additionally, the sequence reads that include the reference allele for the particular site on the genome are included as reference allele data to be later used to determine features of the sequence reads.

The edge filter categorizes sequence reads that include the alternative allele into the artifact or non-artifact category based on two criteria. A first criteria includes a threshold distance of 25 nucleotide base pairs. Therefore, sequence reads categorized in the artifact category include an alternative allele that is within 25 nucleotide base pairs from the edge of the sequence read. A second criteria is a type of nucleotide base mutation. Specifically, sequence reads categorized into the artifact category include an alternative allele that is one of a C>T or G>A mutation. The edge filter categorizes sequence reads that include an alternative allele that does not satisfy these two criteria into the non-artifact category.

The edge filter extracts features from sequence reads of a called variant, including sequence reads that include an alternative allele as well as sequence reads that include the reference allele. Here, the three types of features extracted include: 1) median distance of alternative alleles from the edge of the sequence read, 2) allele fraction of the alternative allele, and 3) significance score. The three types of extracted features are compiled and used to generate the artifact distributions and non-artifact distributions shown in FIGS. 4M-O.

FIGS. 4M-O each show an artifact distribution (left) and non-artifact distribution (right). Each distribution depicts a relationship between two features extracted from sequencing reads that are categorized as artifact training data or non-artifact training data. Specifically, FIG. 4M depicts a relationship between the significance score and median distance from edge. FIG. 4N depicts a relationship between the distribution of the allele fraction and median distance from edge. FIG. 4O depicts a relationship between the distribution of the allele fraction and significance score.

Several trends are observed across the artifact distributions and non-artifact distributions shown in FIGS. 4M-O. Notably, edge variants in the artifact category tend to have high significance scores (e.g., high concentration of edge variants at a significance score of 100 as shown in FIG. 4M and FIG. 4O) whereas non-edge variants in the non-artifact category tend to have far lower significance scores. Additionally, a lower median distance from the edge is correlated with a higher concentration of edge variants. For example, both FIG. 4M and FIG. 26N depict a higher concentration of edge variants with an alternative allele at or near a median distance of zero nucleotide bases from an edge as opposed to a median distance of 25 nucleotide bases from an edge. Of note, a large number of non-edge variants also include an alternative allele that is within 25 nucleotide bases from the edge of a sequencing read (see FIG. 4M and FIG. 4N). This indicates that there is a population of non C>T and non G>A nucleotide base substitutions that are identified as called variants.

FIG. 4P depicts the identification of edge variants and non-edge variants across various subject samples according to one embodiment. FIG. 4P includes data from various subject samples. Specifically, FIG. 4P depicts distributions of identified edge variants and non-edge variants of subject samples (the y-axis) as a function of the median distance from the edge of sequencing reads (the x-axis). Identified edge variants are shown with diagonal hatching patterns whereas non-edge variants are shown without pattern (e.g., solid color).

FIG. 4P demonstrates that for each subject sample, the filtering method of the edge filter can differently identify edge variants and non-edge variants. For example, MSK-VP-0082 (e.g., the fifth sample from the top) includes a large number of edge variants that exhibit a median distance from the edge between 10 and 25 nucleotide base pairs. In addition, MSK-VP-VL-0081 (e.g., the sixth sample from the top) includes a significant number of non-edge variants that exhibit a median distance from the edge between 10 and 25 nucleotide base pairs. This sample-specific filtering enables more accurate identification and removal of edge variants in comparison to filters that employ the same filtering method across all samples. Examples of non-sample specific filters can employ a fixed cutoff based on a feature such as allele frequency such that if the allele frequency of an alternative allele is greater than a fixed threshold amount, then the called variant corresponding to the alternative allele is categorized as an edge variant.

FIG. 4Q depicts concordant variants called in both solid tumor and in cfDNA following the removal of edge variants using different edge filters as a fraction of the variants called in cfDNA according to one embodiment. FIG. 4R depicts concordant variants called in both solid tumor and in cfDNA following the removal of edge variants using different edge filters as a fraction of the variants called in solid tumor according to one embodiment. In particular, both FIG. 4Q and FIG. 4R depict concordance numbers that vary depending on the edge variant filter that is applied (e.g., no edge variant filter, simple edge variant filter, or sample-specific edge variant filter).

For the datasets shown in FIG. 4Q and FIG. 4R, samples were obtained from subjects and processed using the assay process described above. Candidate variants included in an initial set have not yet undergone further filtering to remove edge variants.

In two separate scenarios, these candidate variants in the initial set were further filtered by the edge filter to identify and remove edge variants. A first scenario included the application of a first filter, hereafter referred to as a simple edge variant filter. The simple edge variant filter removes called variants that exhibit a median distance from edges of sequence reads that fall below a threshold distance. Here, the threshold distance is determined based on the location of edge variants in training sequence reads that are categorized in the artifact training data category. Specifically, the threshold distance is expressed as the summation of the median distance of the edge variants from edges of sequence reads and the median absolute deviation of the median distance of the edge variants from edges of sequence reads. The simple edge variant filter is a simple indiscriminate filter that removes all variants that satisfy this threshold distance criteria. The second filter refers to the edge filtering process. Here, the sample specific edge variant filter identifies edge variants while considering the distribution of called variants observed for the sample.

The non-edge variants that remain after removing edge variants using either the simple edge variant filter or the sample specific edge variant filter are retained for analysis in comparison to a conventional method. As referred to hereafter, the conventional method refers to the identification of genomic variations from solid tumor samples using a conventional process, specifically the Memorial Sloan Kettering Integrated Mutation Profiling of Actionable Cancer Targets (MSK-IMPACT) Pipeline (Cheng, D., et al, Memorial Sloan Kettering-Integrated Mutation Profiling ofActionable Cancer Targets (MSK-IMPACT), A Hybridization Capture-Based Next-Generation Sequencing Clinical Assay for Solid Tumor Molecular Oncology, Journal of Molecular Diagnostics, 17(3), p. 251-264).

Here, called variants that are both non-edge variants and detected by the conventional method are referred to as concordant variants.

FIG. 4Q depicts the concordant variants detected in a cfDNA sample following the application of an edge filter (or non-application of an edge filter) and called variants detected in solid tumor tissue as a fraction of the non-edge variants detected in cfDNA. This proportion can be expressed as:

$$\frac{\begin{matrix}( True Variants detected in cf DNA ) ⋂ \\ ( Variants detected in solid tumor tissue )\end{matrix}}{( True Variants detected in cf DNA )}$$

FIG. 4R depicts the concordant variants detected in a cfDNA sample following the application of an edge filter (or non-application of an edge filter) and called variants detected in solid tumor tissue as a fraction of the called variants detected in solid tumor tissue. This proportion can be expressed as:

$$\frac{\begin{matrix}( True Variants detected in cf DNA ) ⋂ \\ ( Variants detected in solid tumor tissue )\end{matrix}}{( True Variants detected in solid tumor tissue )}$$

The percentage of concordant variants shown in FIG. 4Q and FIG. 4R depict several trends of interest. A significantly greater percentage of concordant variants is shown in FIG. 4R in comparison to the percentage of concordant variants depicted in FIG. 4Q. As an example, the percentage of concordant variants detected in breast cancer as a fraction of called variants detected solely in cfDNA is 9.8%, which is significantly lower than the 73% of concordant variants detected in breast cancer as a fraction of called variants detected in solid tumor tissue. This indicates that the identification of non-edge variants in cfDNA samples (irrespective of type of cancer) achieves higher sensitivity in comparison to the conventional method that calls variants in solid tumor tissue.

Referring to the simple edge variant filter in FIG. 4Q, the application of the simple edge variant filter increases the specificity of the called variants. For example, in comparison to the no edge variant filter, the application of the simple edge variant filter increases the specificity of called variants detected in breast cancer (e.g., 9.5% to 11%), in lung cancer (e.g., 45% to 49%), and in prostate cancer (e.g., 22% to 27%). However, this increase in specificity comes at a cost of sensitivity, as shown in FIG. 4R. In comparison to the no edge variant filter, the application of the simple edge variant filter decreases the sensitivity of called variants detected in breast cancer (e.g., 73% to 69%), in lung cancer (e.g., 73% to 70%), and in prostate cancer (e.g., 76% to 71%).

Comparatively, the application of a sample specific edge variant filter improves specificity without sacrificing sensitivity. As shown in FIG. 4Q, in comparison to the no edge variant filter, the application of the sample-specific edge variant filter increases the specificity of called variants detected in breast cancer (e.g., 9.5% to 9.8%), in lung cancer (e.g., 45% to 47%), and in prostate cancer (e.g., 22% to 27%). Additionally, as shown in FIG. 4R, in comparison to the no edge variant filter, the application of the sample-specific edge variant filter maintains the sensitivity of called variants detected in breast cancer (e.g., maintained at 73%), in lung cancer (e.g., maintained at 73%), and in prostate cancer (e.g., maintained at 76%).

2.8. Examples of Combined Filtering and Scoring

The example data in the following FIGS. 4S-Z were generated using sequence reads obtained from a sample set of individuals of a cell free genome study and processed using one or more of the methods described herein (e.g., noise modeling, joint modeling, edge filtering, non-synonymous filtering, etc.). The sample set includes healthy individuals from which blood samples (e.g., cfDNA) were obtained. Additionally, the sample set includes individuals known to have at least one type of cancer, from which blood samples and tissue samples (e.g., tumor or gDNA) were obtained. The data was collected from individuals over approximately 140 centers in the United States of America and Canada.

FIG. 4S is a table describing individuals of a sample set for a cell free genome study according to one embodiment. The sample set includes samples known to have at least breast, lung, prostate, colorectal, and other types of cancer. The demographic data (e.g., age, gender, and ethnicity) of the individuals are also shown in FIG. 4S. FIG. 4T is a chart indicating types of cancers associated with the sample set for the cell free genome study of FIG. 4S according to one embodiment. As shown in FIG. 4T, the circulating cell-free genome atlas (CCGA) sample set included the following cancer types: breast, lung, prostate, colorectal, renal, uterine, pancreas, esophageal, lymphoma, head and neck, ovarian, hepatobiliary, melanoma, cervical, multiple myeloma, leukemia, thyroid, bladder, gastric, and anorectal. FIG. 4U is another table describing the sample set for the cell free genome study of FIG. 4S according to one embodiment. Particularly, the table shows counts of the samples known to have cancer organized based on clinical stages of the cancers.

FIG. 4V shows diagrams of example counts of called variants determined using one or more types of filters and models according to one embodiment. Each of the diagrams includes data points of the sample set plotted on an x-axis representing age of the corresponding individual and a y-axis representing a number of called variants after processing by the processing system. Diagram 466A includes results from processing sequence reads of the sample set using noise modeling. Diagram 466B includes results from processing sequence reads of the sample set using joint modeling and edge filtering in addition to the noise modeling. Diagram 466C includes results from processing sequence reads of the sample set using non-synonymous filtering in addition to the joint modeling, edge filtering, and noise modeling.

As illustrated by the progression of diagrams, the number of called variants generally decreases as the extent of filtering increases. Thus, the examples suggest that that non-synonymous filtering, joint modeling, edge filtering, and noise modeling by the processing system can successfully identify and remove a notable amount of false positives. Accordingly, the processing system provides for a more accurate variant caller that mitigates influence from various sources of noise or artifacts. Targeted assays analyzing cfDNA from blood samples using the disclosed methods may be able to capture tumor relevant biology. A slight proportional correlation may be observed in the diagrams between the count of called variants and age of the individuals (e.g., more evident in diagram 466A). Moreover, there are greater counts of called variants for cancer samples than non-cancer samples as expected.

FIG. 4W is a table of example counts of called variants for samples known to have various types of cancer and at different stages of cancer according to one embodiment. FIG. 4X is a diagram of example counts of called variants for samples known to have various types of cancer and at different stages of cancer according to one embodiment. As shown by the box plots for samples known to have breast, colorectal, lung, or prostate cancers, the median number of called variants tend to increase as the stages of cancer increase from I to IV, and the number for non-cancer samples is relatively lower compared to those of the cancer samples.

FIG. 4Y is a diagram of example counts of called variants for samples known to have early or late stage cancer according to one embodiment. FIG. 4Z is another diagram of example counts of called variants for samples known to have early or late stage cancer according to one embodiment. In particular, FIG. 4Y and FIG. 4Z show called variants of sequence reads from cdstg1lh_grouped genes associated with breast cancer (e.g., HER2+, HR+|HER2−, TNBC) and lung cancer (e.g., Adenocarcinoma, small cell lung cancer, and squamous cell carcinoma), respectively. FIGS. 4Y-4Z show a trend where the number of called variants tend to increase as the cancers progress from early stage to late stage. The example data indicates that the processing system can detect different subtypes or variants of sequences in genes. Additionally, the number for non-cancer samples is relatively lower compared to those of the cancer samples.

### 3. Whole Genome Computational Analysis

3.1. Whole Genome Features

Referring briefly again to FIGS. 1B-1D, the whole genome computational analysis 140C receives sequence reads generated by the whole genome sequencing assay 132 and determines values of whole genome features 152 based on the sequence reads. Examples of whole genome features 152 include any of: characteristics of a bin determined for each of a plurality of bins across the genome (e.g., bin scores and bin variance of bins across the genome from a cfDNA sample and/or bin scores and bin variance of bins across the genome from a gDNA sample), characteristics of segments across the genome (e.g., segment scores and segment variance of segments across the genome from a cfDNA sample and/or segment scores and segment variance of segments across the genome from a gDNA sample), total number of copy number aberrations, presence of copy number aberrations per chromosome (or portion of a chromosome such as a chromosome arm), and reduced dimensionality features.

Generally, bin scores and segment scores represent a normalized sequence read count. Specifically, bin scores and segment scores represent a total number of sequence reads categorized in a bin or segment that is normalized based on a total number of expected sequence reads in the bin or segment (e.g., expected based on training data).

In some embodiments, the total number of copy number aberrations is a single, numerical feature value for a sample. For example, the presence of copy number aberrations per chromosome can be a value of 0 or 1 that indicates whether a copy number aberration is located on a particular chromosome. Here, copy number aberrations is predicated on a workflow that can accurately different copy number aberrations from other copy number events, such as a copy number variation that arises in non-somatic source (e.g., arises in blood). An example of the copy number aberration detection workflow is described below.

Reduced dimensionality features refer to features that have been reduced to a lower dimensionality space (in comparison to data of original sequence reads) while still representing the main characteristics of the original sequence reads. As an example, bin scores of bins across the genome can be reduced to a lower dimensionality space and represented by reduced dimensionality features. Reduced dimensionality features can be generated through a dimensionality reduction process such as principal component analysis (PCA).

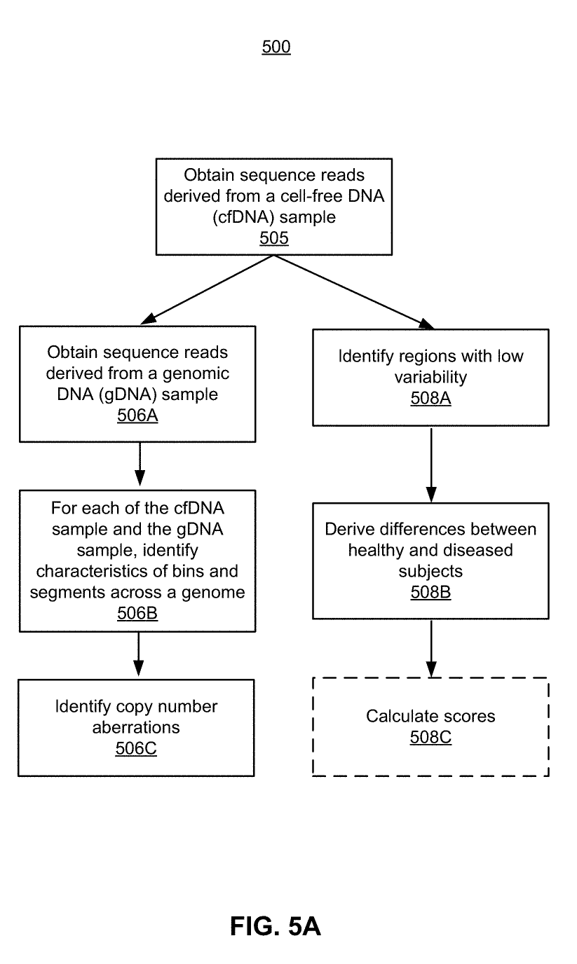

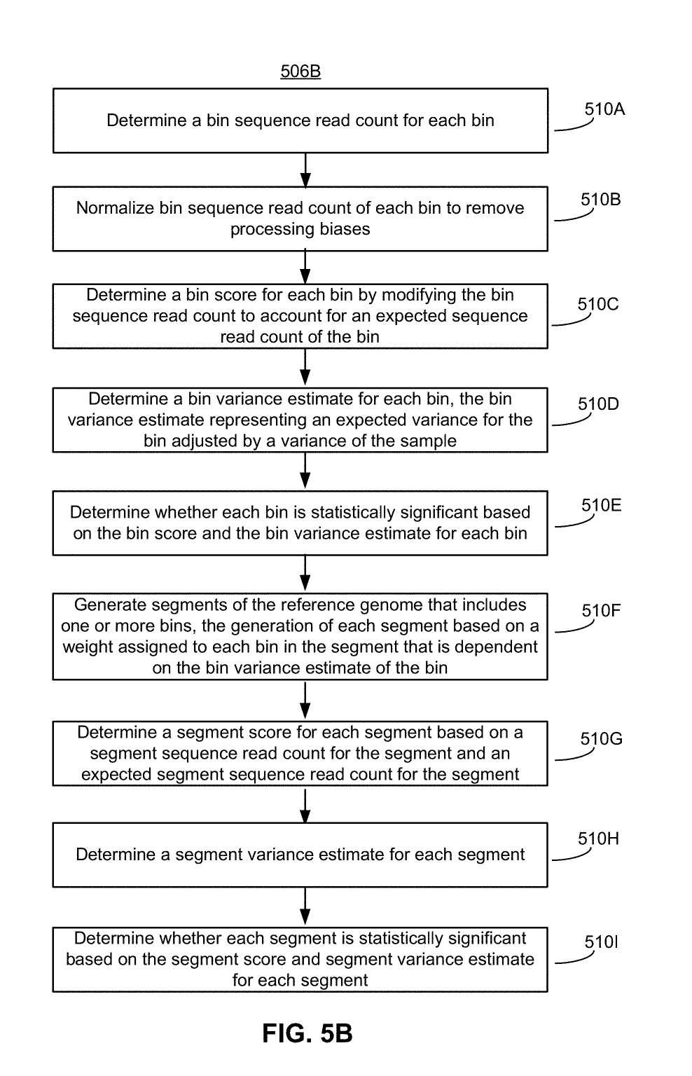

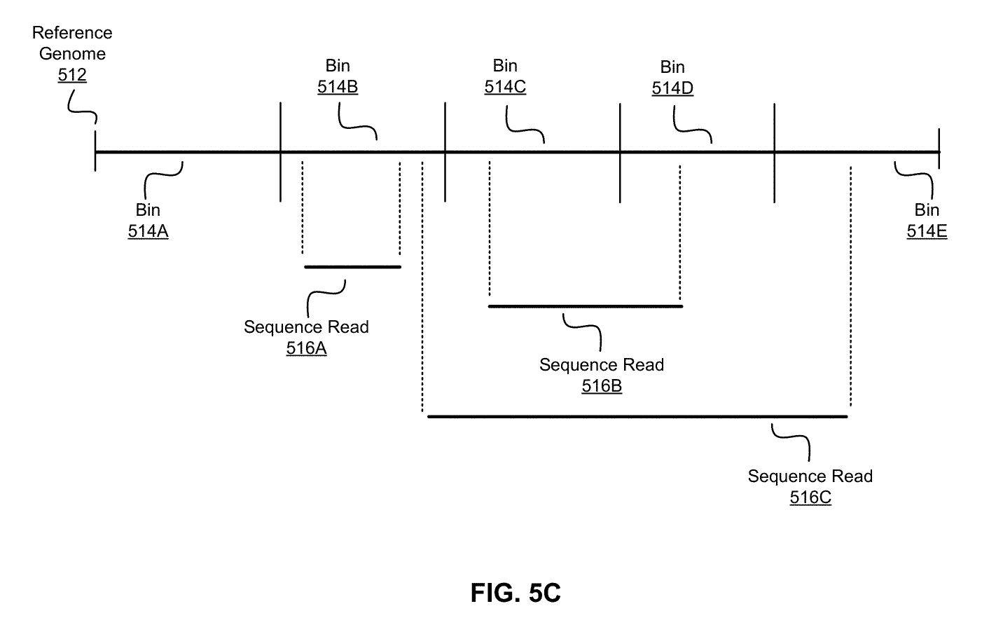

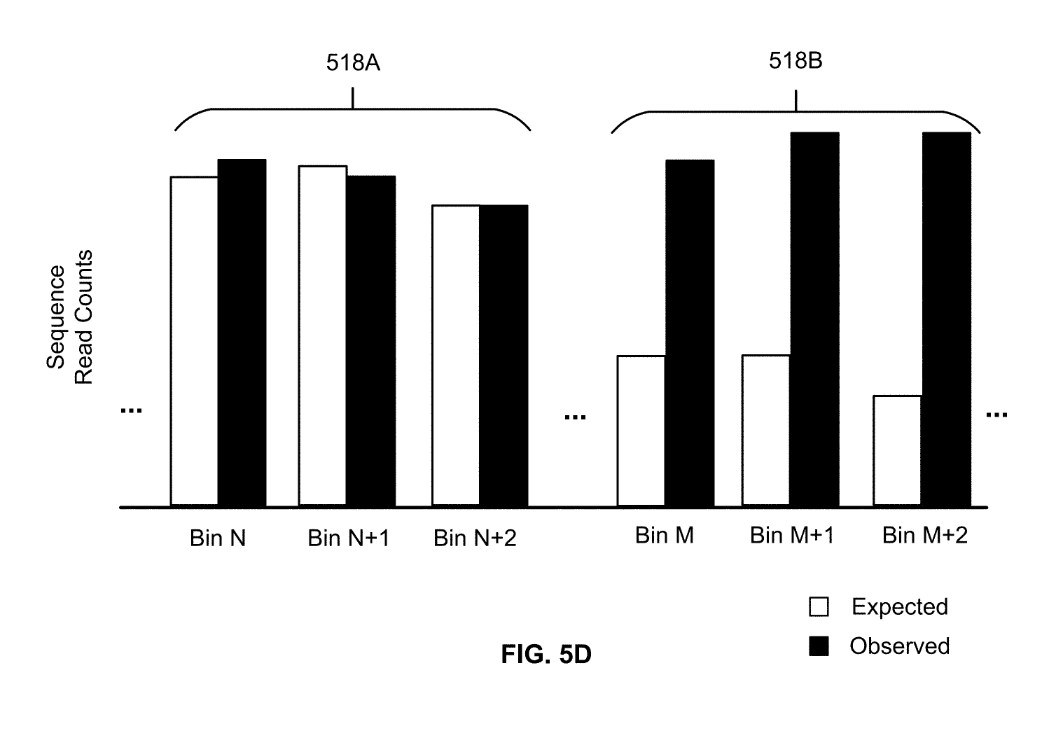

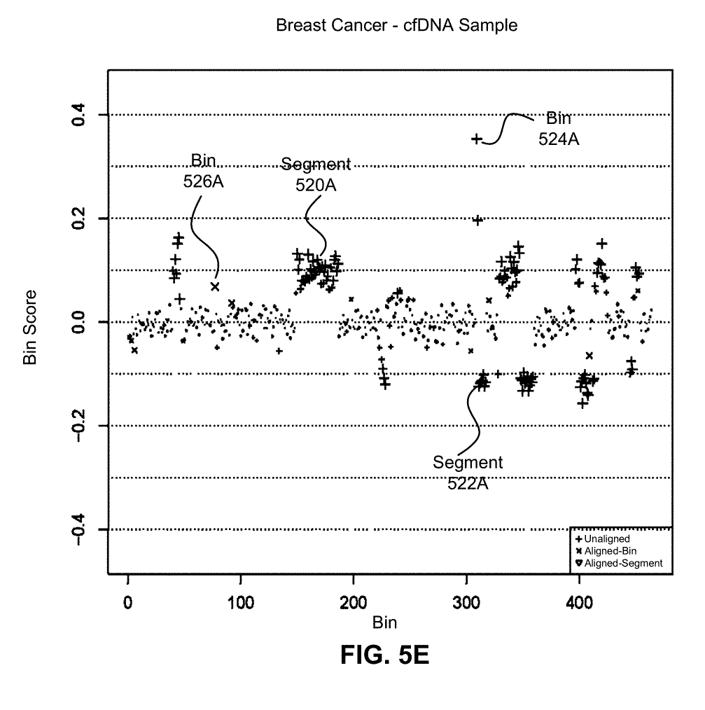

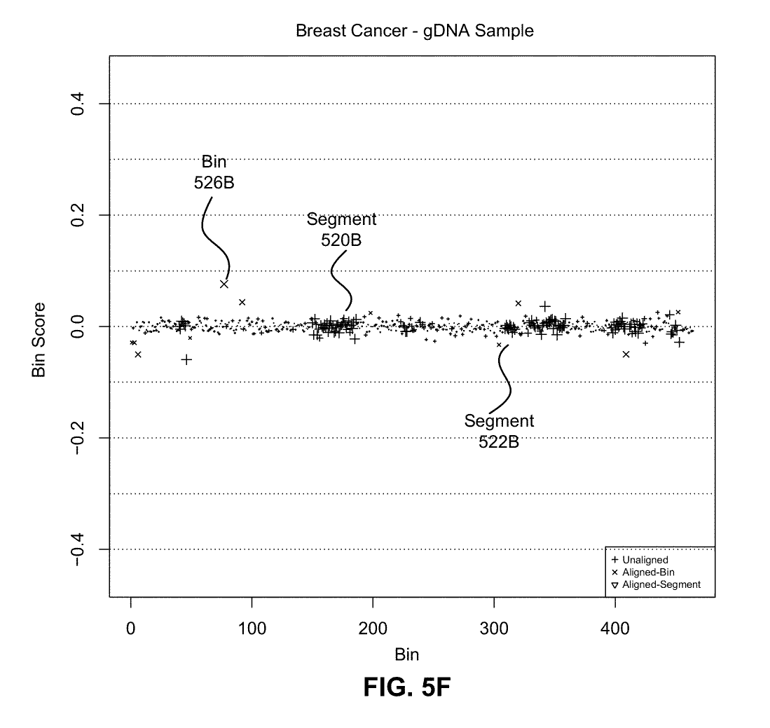

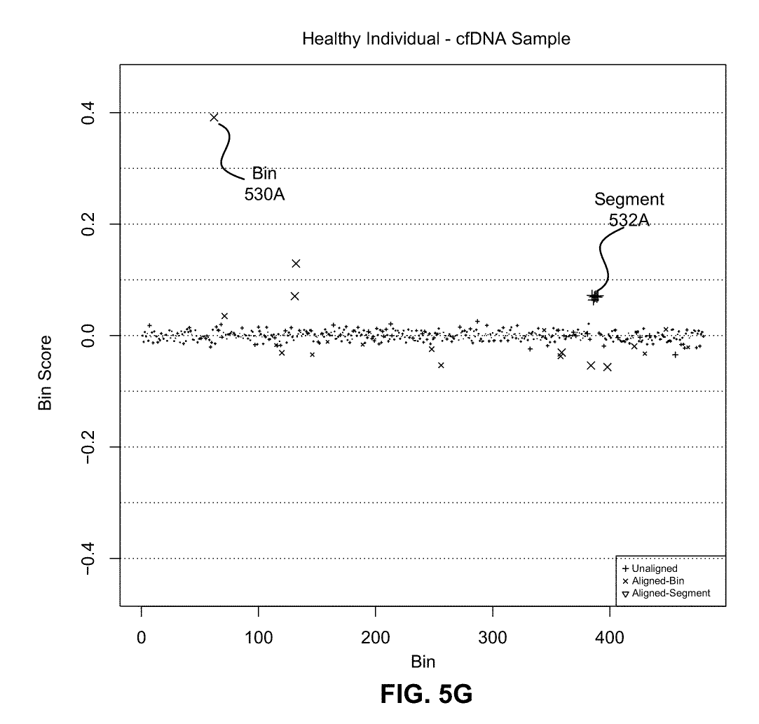

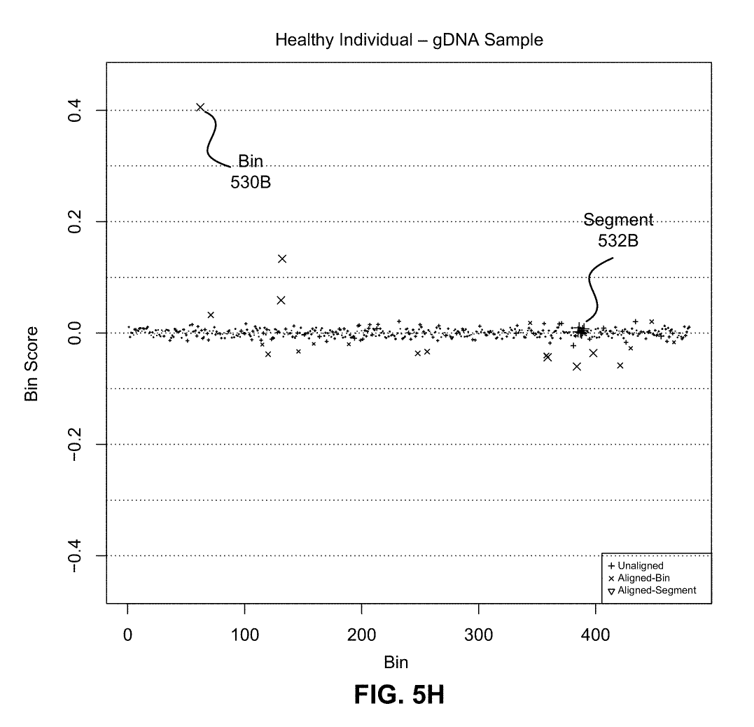

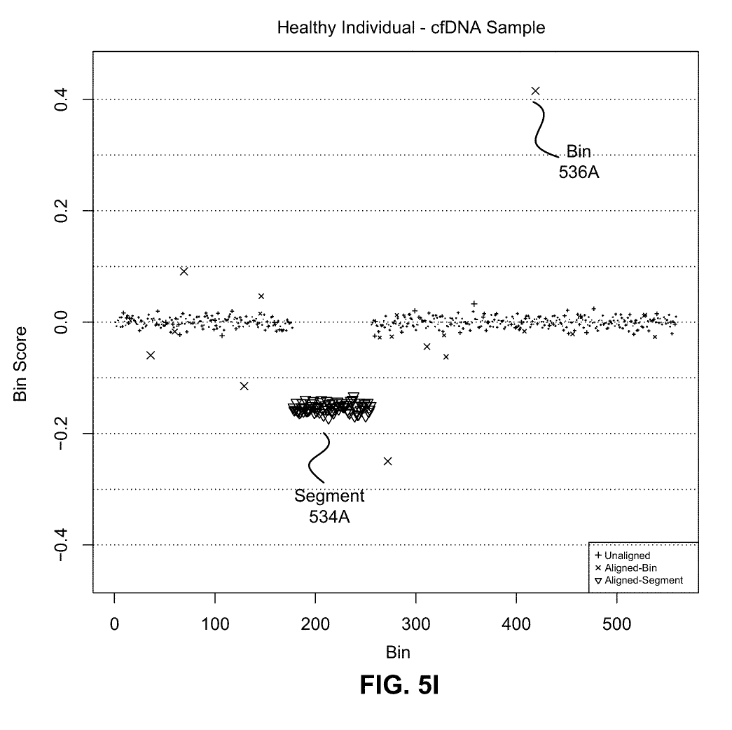

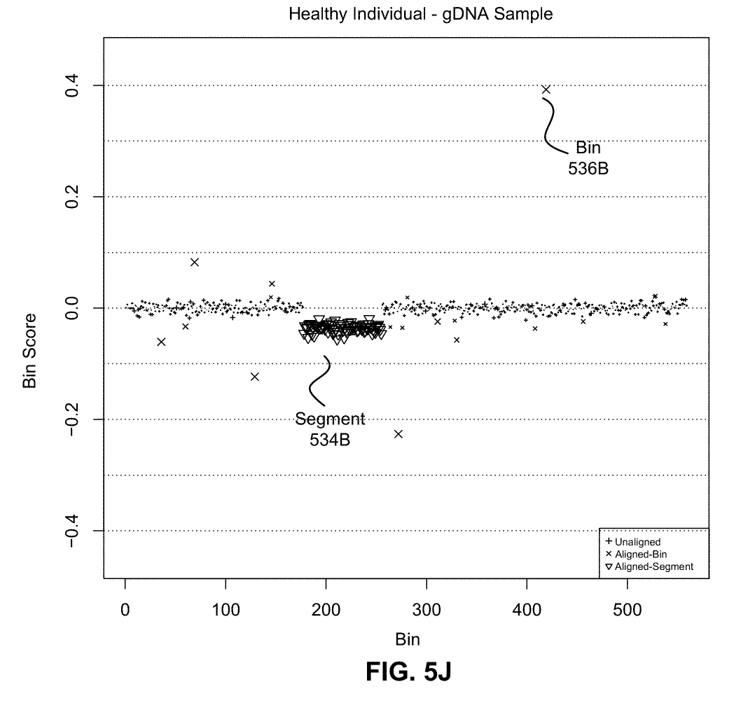

Generally, feature values of the aforementioned whole genome features 152 are determined through one of two workflows. FIG. 5A depicts an example flow process of two different workflows for determining whole genome features, in accordance with an embodiment. Steps 505 and 506A-C will hereafter be referred to as the copy number aberration detection workflow and steps 505 and 508A-C will be hereafter referred to as the dimensionality reduction workflow.

3.2. Copy Number Aberration Detection Workflow

Referring first to the copy number aberration detection workflow, the features of the characteristics of bins across the genome, the characteristics of segments across the genome, and the presence of copy number aberrations per chromosome can be determined through the copy number aberration detection workflow. Rather than the described workflow, a copy number aberration workflow known in the art may also be used (see, e.g., U.S. application Ser. No. 16/352,214, which is incorporated by reference herein).

For example, as shown in FIG. 5A, at step 505, sequence reads derived from a cfDNA sample are obtained, and optionally, at step 506A, sequence reads derived from a gDNA sample are obtained. As described above in relation to FIGS. 1B-1D, the sequence reads derived from cfDNA 115 and/or from gDNA (e.g., WBC DNA 120) can be received from a whole genome sequencing assay 132. In general, any whole genome sequencing assay known in the art can be used (see, e.g., US 2013/0040824, and US 2013/0325360, which are incorporated herein by reference). In another embodiment, sequence reads derived from a whole genome bisulfite sequencing assay or from a targeted panel can be used to determine values of whole genome features, as is known in the art.

At step 506B, sequence reads derived from each of cfDNA and gDNA are analyzed to identify characteristics of bins and segments across a genome. Generally, a bin includes a range of nucleotide bases of a genome. A segment refers to one or more bins. Therefore, each sequence read is categorized in bins and/or segments that include a range of nucleotide bases that corresponds to the sequence read. Each statistically significant bin or segment of the genome includes a total number of sequence reads (or normalized sequence reads) categorized in the bin or segment that is indicative of a copy number event. Generally, a statistically significant bin or segment includes a sequence read count that significantly differs from an expected sequence read count for the bin or segment even when accounting for possibly confounding factors, examples of which includes processing biases, variance in the bin or segment, or an overall level of noise in the sample (e.g., cfDNA sample or gDNA sample). Therefore, the sequence read count of a statistically significant bin and/or a statistically significant segment likely indicates a biological anomaly such as a presence of a copy number event in the sample.

Generally, the analysis of cfDNA sequence reads and the analysis of gDNA sequence reads are conducted independent of one another. In various embodiments, the analysis of cfDNA sequence reads and gDNA sequence reads are conducted in parallel. In some embodiments, the analysis of cfDNA sequence reads and gDNA sequence reads are conducted at separate times depending on when the sequence reads are obtained.

Reference is now made to FIG. 5B, which is an example flow process that describes the analysis for identifying characteristics of bins and segments derived from cfDNA and gDNA samples, in accordance with an embodiment. Specifically, FIG. 5B depicts additional steps included in step 506B shown in FIG. 5A. Therefore, steps 510A-510I can be performed for a cfDNA sample and similarly, steps 510A-510I can be separately performed for a gDNA sample.

At step 510A, a bin sequence read count is determined for each bin of a reference genome. Generally, each bin represents a number of contiguous nucleotide bases of the genome. A genome can be composed of numerous bins (e.g., hundreds or even thousands). In some embodiments, the number of nucleotide bases in each bin is constant across all bins in the genome. In some embodiments, the number of nucleotide bases in each bin differs for each bin in the genome. In one embodiment, the number of nucleotide bases in each bin is between 10 kilobases (kb) and 1 megabase (mb). In one embodiment, the number of nucleotide bases in each bin is between 25 kilobases (kb) and 200 kb. In one embodiment, the number of nucleotide bases in each bin is between 40 kb and 100 kb. In one embodiment, the number of nucleotide bases in each bin is between 45 kb and 75 kb. In one embodiment, the number of nucleotide bases in each bin is 50 kb. In practice, other bin sizes may be used as well.

The sequence read count of a bin represents a total number of sequence reads that are categorized in the bin. A sequence read is categorized in a bin if the sequence read spans a threshold number of nucleotide bases that are included in the bin (i.e., align or map to a bin). In one embodiment, each sequence read categorized in a bin spans at least one nucleotide base that is included in the bin. Reference is now made to FIG. 5C, which is an example depiction of sequence reads 516 in relation to bins 514 of a reference genome 512, in accordance with an embodiment. Sequence read 516A, sequence read 516B, and sequence read 516C can each include a different number of nucleotide bases and can span one or more of the bins 514.

As shown in FIG. 5C, sequence read 516A includes fewer nucleotide bases in comparison to the number of nucleotide bases in a bin (e.g., bin 514B). Here, sequence read 516A is categorized in bin 514B. Sequence read 516B spans nucleotide bases that are included in both bin 514C and bin 514D. Therefore, sequence read 516B is categorized in both bin 514C and bin 514D. Sequence read 516C spans nucleotide bases that are included in bin 514B, bin 514C, and bin 514D. Therefore, sequence read 516C is categorized in each of bin 514B, bin 514C, and bin 514D.

To determine the bin sequence read count for each bin, the sequence reads categorized in each bin are quantified. Therefore, bin 514A shown in FIG. 5C has a bin sequence read count of zero, bin 514B has a bin sequence read count of two (e.g., sequence read 516A and sequence read 516B), bin 514C has a bin sequence read count of two (e.g., sequence read 516B and sequence read 516C), bin 514D has a bin sequence read count of two (e.g., sequence read 516B and sequence read 516C), and bin 514E has a bin sequence read count of one (e.g., sequence read 516C).

Returning to FIG. 5B, at step 510B, the bin sequence read count for each bin is normalized to remove one or more different processing biases. Generally, the bin sequence read count for a bin is normalized based on processing biases that were previously determined for the same bin. In one embodiment, normalizing the bin sequence read count involves dividing the bin sequence read count by a value representing the processing bias. In one embodiment, normalizing the bin sequence read count involves subtracting a value representing the processing bias from the bin sequence read count. Examples of a processing bias for a bin can include guanine-cytosine (GC) content bias, mappability bias, or other forms of bias captured through a principal component analysis, as is known in the art. Processing biases for a bin can be determined using prior training data with, for example, sequence reads obtained from healthy individuals.

For example, in one embodiment, at step 510C, a bin score for each bin is determined by modifying the bin sequence read count for the bin by the expected bin sequence read count for the bin. Step 510C serves to normalize the observed bin sequence read count such that if the particular bin consistently has a high sequence read count (e.g., high expected bin sequence read counts) across many samples, then the normalization of the observed bin sequence read count accounts for that trend. The expected sequence read count for the bin can be determined from training data (e.g., sequence reads from healthy individuals). The generation of the expected sequence read count for each bin is described in further detail below.

In one embodiment, a bin score for a bin can be represented as the log of the ratio of the observed sequence read count for the bin and the expected sequence read count for the bin. For example, bin score bᵢ for bin i can be expressed as:

$${b}_{i} = log ( \frac{observed bin sequence read count}{expected bin sequence read count} )$$

In other embodiments, the bin score for the bin can be represented as the ratio between the observed sequence read count for the bin and the expected sequence read count for the bin (e.g.,

$$\frac{observed}{expected} ) ,$$

the square root of the ratio (e.g.,

$$\sqrt{\frac{observed}{expected} )} ,$$

a generalized log transformation (glog) expected expected of the ratio (e.g., log(observed+{square root over (observed²+expected))}) or other variance stabilizing transforms of the ratio.

Reference is now made to FIG. 5D, which is an example chart depicting expected and observed sequence read counts across different bins of a reference genome, in accordance with an embodiment. Specifically, FIG. 5D depicts observed and expected sequence read counts for a first set 518A of bins (e.g., Bin N, Bin N+1, Bin N+2) and for a second set 518B of bins (e.g., Bin M, Bin M+1, Bin M+2). In various embodiments, bins in the first set 518A may be from a first segment of the reference genome whereas bins in the second set 518B may be from a second segment of the reference genome. In some embodiments, bins in the first set 518A may be from a first chromosome whereas bins in second set 518B are from a different chromosome.

Here, the observed sequence read counts and expected sequence read counts for bins in the first set 518A may not differ significantly. However, the observed sequence read counts for bins in the second set 518B may be significantly higher than the corresponding expected read counts for the bins. Therefore, the bin scores for each of the bins in the second set 518B are higher than the bin scores for each of the bins in the first set 518A. The higher bin scores of the bins in the second set 518B indicate a higher likelihood that the observed sequence read counts in bin M, bin M+1, and bin M+2 are a result of a copy number event.

The differing bin scores for the first set 518A and second set 518B of bins illustrates the benefit of normalizing the observed sequence read counts for each bin by the corresponding expected sequence read counts for the bin. Specifically, in the example shown in FIG. 5D, the observed sequence read counts for bins in the first set 518A and the observed sequence read counts for bins in the second set 518B may not significantly differ from each other.

Here, bin scores across bins can be informative for identifying a copy number aberration. Moreover, as described above, the bin scores for different bins can serve as a whole genome feature.

Returning to FIG. 5B, at step 510D, a bin variance estimate is determined for each bin. Here, the bin variance estimate represents an expected variance for the bin that is further adjusted by an inflation factor that represents a level of variance in the sample. Put another way, the bin variance estimate represents a combination of the expected variance of the bin that is determined from prior training samples as well as an inflation factor of the current sample (e.g., cfDNA or gDNA sample) which is not accounted for in the expected variance of the bin. The bin variance can influence whether a bin score is indicative of a copy number aberration. As discussed above, the bin variance can serve as a whole genome feature.

To provide an example, a bin variance estimate (varᵢ) for a bin i can be expressed as:

varᵢ=varₑₓₚ_{ᵢ}*Iₛₐₘₚₗₑ

where varₑₓₚ_{ᵢ} represents the expected variance of bin i determined from prior training samples and Iₛₐₘₚₗₑ represents the inflation factor of the current sample. Generally, the expected variance of a bin (e.g., varₑₓₚ) is obtained from training data, such as sequence reads obtained from healthy individuals.

To determine the inflation factor Iₛₐₘₚₗₑ of the sample, a deviation of the sample is determined and combined with sample variation factors. Sample variation factors are coefficient values that are previously derived by performing a fit across data derived from multiple training samples. For example, if a linear fit is performed, sample variation factors can include a slope coefficient and an intercept coefficient. If higher order fits are performed, sample variation factors can include additional coefficient values.

More specifically, to determine coefficient values, for each training sample, sequence reads from the training sample can be used to determine z-scores for each bin of the reference genome. A z-score for bin i can be expressed as:

$${z}_{i} = \frac{{b}_{i}}{{var}_{i}}$$

where bᵢ is the bin score for bin i and varᵢ is the bin variance estimate for the bin.

A first curve fit is performed between the bin z-scores of each training sample and the theoretical distribution of z-scores. Here, an example theoretical distribution of z-scores is a normal distribution. In one embodiment, the first curve fit is a linear robust regression fit which yields a slope value. Therefore, performing the first curve fit between bin z-scores of a training sample and the theoretical distribution of z-scores yields a slope value. The first curve fit is performed multiple times for multiple training samples to calculate multiple slope values.

A second curve fit is performed between slope values and deviations of training samples. As an example, the deviation of a training sample can be a median absolute pairwise deviation (MAPD), which represents the median of absolute value differences between bin scores of adjacent bins across the training sample. In one embodiment, the second curve fit is a linear robust regression fit. In another embodiment, the second curve fit can be a higher order polynomial fit. The second curve fit yields coefficient values which, in the embodiment where the second curve fit is a linear robust regression fit, includes a slope coefficient and an intercept coefficient. The coefficient values yielded by the second curve fit are stored as sample variation factors.

The deviation of the sample represents a measure of variability of sequence read counts in bins across the sample. In one embodiment, the deviation of the sample is a median absolute pairwise deviation (MAPD) and can be calculated by analyzing sequence read counts of adjacent bins. Specifically, the MAPD represents the median of absolute value differences between bin scores of adjacent bins across the sample. Mathematically, the MAPD can be expressed as:

∀(binᵢ,binᵢ₊₁),MAPD=median{|(bᵢ)−(bᵢ₊₁)|}

where bᵢ and bᵢ₊₁ are the bin scores for bin i and bin i+1 respectively.

The inflation factor Iₛₐₘₚₗₑ is determined by combining the sample variation factors and the deviation of the sample (e.g., MAPD). As an example, the inflation factor Iₛₐₘₚₗₑ of a sample can be expressed as:

Iₛₐₘₚₗₑ=slope*σₛₐₘₚₗₑ+intercept.

Here, each of the “slope” and “intercept” coefficients are sample variation factors whereas σₛₐₘₚₗₑ represents the deviation of the sample.

At step 510E, each bin is analyzed to determine whether the bin is statistically significant based on the bin score and bin variance estimate for the bin. For each bin i, the bin score (bᵢ) and the bin variance estimate (varᵢ) of the bin can be combined to generate a z-score for the bin. An example of the z-score (zᵢ) of bin i can be expressed as:

$${z}_{i} = \frac{{b}_{i}}{{var}_{i}}$$

To determine whether a bin is a statistically significant bin, the z-score of the bin is compared to a threshold value. If the z-score of the bin is greater than the threshold value, the bin is deemed a statistically significant bin. Conversely, if the z-score of the bin is less than the threshold value, the bin is not deemed a statistically significant bin. In one embodiment, a bin is determined to be statistically significant if the z-score of the bin is greater than 2. In other embodiments, a bin is determined to be statistically significant if the z-score of the bin is greater than 2.5, 3, 3.5, or 4. In one embodiment, a bin is determined to be statistically significant if the z-score of the bin is less than −2. In other embodiments, a bin is determined to be statistically significant if the z-score of the bin is less than −2.5, −3, −3.5, or −4. The statistically significant bins can be indicative of one or more copy number events that are present in a sample (e.g., cfDNA or gDNA sample).

At step 510F, segments of the reference genome are generated. Each segment is composed of one or more bins of the reference genome and has a statistical sequence read count. Examples of a statistical sequence read count can be an average bin sequence read count, a median bin sequence read count, and the like. Generally, each generated segment of the reference genome possesses a statistical sequence read count that differs from a statistical sequence read count of an adjacent segment. Therefore, a first segment may have an average bin sequence read count that significantly differs from an average bin sequence read count of a second, adjacent segment.

In various embodiments, the generation of segments of the reference genome can include two separate phases. A first phase can include an initial segmentation of the reference genome into initial segments based on the difference in bin sequence read counts of the bins in each segment. The second phase can include a re-segmentation process that involves recombining one or more of the initial segments into larger segments. Here, the second phase considers the lengths of the segments created through the initial segmentation process to combine false-positive segments that were a result of over-segmentation that occurred during the initial segmentation process.

Referring more specifically to the initial segmentation process, one example of the initial segmentation process includes performing a circular binary segmentation algorithm to recursively break up portions of the reference genome into segments based on the bin sequence read counts of bins within the segments. In other embodiments, other algorithms can be used to perform an initial segmentation of the reference genome. As an example of the circular binary segmentation process, the algorithm identifies a break point within the reference genome such that a first segment formed by the break point includes a statistical bin sequence read count of bins in the first segment that significantly differs from the statistical bin sequence read count of bins in the second segment formed by the break point. Therefore, the circular binary segmentation process yields numerous segments, where the statistical bin sequence read count of bins within a first segment is significantly different from the statistical bin sequence read count of bins within a second, adjacent segment.

The initial segmentation process can further consider the bin variance estimate for each bin when generating initial segments. For example, when calculating a statistical bin sequence read count of bins in a segment, each bin i can be assigned a weight that is dependent on the bin variance estimate (e.g., varᵢ) for the bin. In one embodiment, the weight assigned to a bin is inversely related to the magnitude of the bin variance estimate for the bin. A bin that has a higher bin variance estimate is assigned a lower weight, thereby lessening the impact of the bin's sequence read count on the statistical bin sequence read count of bins in the segment. Conversely, a bin that has a lower bin variance estimate is assigned a higher weight, which increases the impact of the bin's sequence read count on the statistical bin sequence read count of bins in the segment.

Referring now to the re-segmenting process, it analyzes the segments created by the initial segmentation process and identifies pairs of falsely separated segments that are to be recombined. The re-segmentation process may account for a characteristic of segments not considered in the initial segmentation process. As an example, a characteristic of a segment may be the length of the segment. Therefore, a pair of falsely separated segments can refer to adjacent segments that, when considered in view of the lengths of the pair of segments, do not have significantly differing statistical bin sequence read counts. Longer segments are generally correlated with a higher variation of the statistical bin sequence read count. As such, adjacent segments that were initially determined to each have statistical bin sequence read counts that differed from the other can be deemed as a pair of falsely separated segments by considering the length of each segment.

Falsely separated segments in the pair are combined. Thus, performing the initial segmentation and re-segmenting processes results in generated segments of a reference genome that takes into consideration variance that arises from differing lengths of each segment.

At step 510G, a segment score is determined for each segment based on an observed segment sequence read count for the segment and an expected segment sequence read count for the segment. An observed segment sequence read count for the segment represents the total number of observed sequence reads that are categorized in the segment. Therefore, an observed segment read count for the segment can be determined by summating the observed bin read counts of bins that are included in the segment. Similarly, the expected segment sequence read count represents the expected sequence read counts across the bins included in the segment. Therefore, the expected segment sequence read count for a segment can be calculated by quantifying the expected bin sequence read counts of bins included in the segment. The expected read counts of bins included in the segment can be determined from prior training data, such as sequence reads from healthy individuals. As described above, the segment score can be indicative of a copy number aberration. Therefore, the segment score can serve as a whole genome feature.

The segment score for a segment can be expressed as the ratio of the segment sequence read count and the expected segment sequence read count for the segment. In one embodiment, the segment score for a segment can be represented as the log of the ratio of the observed sequence read count for the segment and the expected sequence read count for the segment. Segment score sₖ for segment k can be expressed as:

$${s}_{k} = log ( \frac{observed segment sequence read count}{expected segment sequence read count} )$$

In other embodiments, the segment score for the segment can be represented as one of the square root of the ratio (e.g.,

$$\sqrt{\frac{observed}{expected} )} ,$$

a generalized log transformation of the ratio (e.g., log(observed+{square root over (observed²+expected))}) or other variance stabilizing transforms of the ratio.

At step 510H, a segment variance estimate is determined for each segment. Generally, the segment variance estimate represents how deviant the sequence read count of the segment is. The segment variance can influence whether a segment score is indicative of a copy number aberration. As discussed above, the segment variance can serve as a whole genome feature.

In one embodiment, the segment variance estimate can be determined by using the bin variance estimates of bins included in the segment and further adjusting the bin variance estimates by a segment inflation factor (I_{segment}). To provide an example, the segment variance estimate for a segment k can be expressed as:

varₖ=mean(varᵢ)*I_{segment}

where mean(varᵢ) represents the mean of the bin variance estimates of bins i that are included in segment k. The bin variance estimates of bins can be obtained from training data e.g., sequence reads from healthy individuals.

The segment inflation factor accounts for the increased deviation at the segment level that is typically higher in comparison to the deviation at the bin level. In various embodiments, the segment inflation factor may scale according to the size of the segment. For example, a larger segment composed of a large number of bins would be assigned a segment inflation factor that is larger than a segment inflation factor assigned to a smaller segment composed of fewer bins. Thus, the segment inflation factor accounts for higher levels of deviation that arises in longer segments. In various embodiments, the segment inflation factor assigned to a segment for a first sample differs from the segment inflation factor assigned to the same segment for a second sample. In various embodiments, the segment inflation factor I_{segment} for a segment with a particular length can be empirically determined in advance.

In various embodiments, the segment variance estimate for each segment can be determined by analyzing training samples. For example, once the segments are generated in step 510F, sequence reads from training samples are analyzed to determine an expected segment sequence read count for each generated segment and an expected segment variance estimate for each segment.

The segment variance estimate for each segment can be represented as the expected segment variance estimate for each segment determined using the training samples adjusted by the sample inflation factor. For example, the segment variance estimate (varₖ) for a segment k can be expressed as:

varₖ=varₑₓₚ_{ₖ}*Iₛₐₘₚₗₑ

where varₑₓₚ_{ₖ} is the expected segment variance estimate for segment k and Iₛₐₘₚₗₑ is the sample inflation factor described above in relation to 510D.

At step 510I, each segment is analyzed to determine whether the segment is statistically significant based on the segment score and segment variance estimate for the segment. For each segment k, the segment score (Sₖ) and the segment variance estimate (varₖ) of the segment can be combined to generate a z-score for the segment. An example of the z-score (Zₖ) of segment k can be expressed as:

$${z}_{k} = \frac{{s}_{k}}{{var}_{k}}$$

To determine whether a segment is a statistically significant segment, the z-score of the segment is compared to a threshold value. If the z-score of the segment is greater than the threshold value, the segment is deemed a statistically significant segment. Conversely, if the z-score of the segment is less than the threshold value, the segment is not deemed a statistically significant segment. In one embodiment, a segment is determined to be statistically significant if the z-score of the segment is greater than 2. In other embodiments, a segment is determined to be statistically significant if the z-score of the segment is greater than 2.5, 3, 3.5, or 4. In some embodiments, a segment is determined to be statistically significant if the z-score of the segment is less than −2. In other embodiments, a segment is determined to be statistically significant if the z-score of the segment is less than −2.5, −3, −3.5, or −4. The statistically significant segments can be indicative of one or more copy number events that are present in a sample (e.g., cfDNA or gDNA sample).

Returning to FIG. 5A, at step 506C, copy number aberrations in the cfDNA are identified according to statistically significant bins (e.g., determined at step 510E) and/or statistically significant segments (e.g., determined at step 510I). Specifically, statistically significant bins/segments of the cfDNA sample are compared to corresponding bins of the gDNA sample. The comparison between statistically significant segments and bins of the cfDNA sample and corresponding segments and bins of the gDNA sample yields a determination as to whether the statistically significant segments and bins of the cfDNA sample align with the corresponding segments and bins of the gDNA sample. As used hereafter, aligned segments or bins refers to the fact that the segments or bins are statistically significant in both the cfDNA sample and the gDNA sample. On the contrary, unaligned or not aligned segments or bins refers to the fact that the segments or bins are statistically significant in one sample (e.g., cfDNA sample), but is not statistically significant in another sample (e.g., gDNA sample).

Generally, if statistically significant bins and statistically significant segments of the cfDNA sample are aligned with corresponding bins and segments of the gDNA sample that are also statistically significant, this indicates that the same copy number event is present in both the cfDNA sample and the gDNA sample. Therefore, the source of the copy number event is likely to be due to a non-tumor event (e.g., either a germline or somatic non-tumor event) and the copy number event is likely a copy number variation.

Conversely, if statistically significant bins and statistically significant segments of the cfDNA sample are aligned with corresponding bins and segments of the gDNA sample that are not statistically significant, this indicates that the copy number event is present in the cfDNA sample but is absent from the gDNA sample. In this scenario, the source of the copy number event in the cfDNA sample is due to a somatic tumor event and the copy number event is a copy number aberration.

Identifying the source of a copy number event that is detected in the cfDNA sample is beneficial in filtering out copy number events that are due to a germline or somatic non-tumor event. This improves the ability to correctly identify copy number aberrations that are due to the presence of a solid tumor.

Here, the presence of the identified copy number aberrations across the genome for a cfDNA sample can serve as a whole genome feature. In various embodiments, the locations of the copy number aberrations are further considered. For example, a whole genome feature can be the number of copy number aberrations in a particular region of a chromosome.

3.2.1. Example 1: Copy Number Aberrations Originate from Somatic Tumor Source in a Cancer Sample

FIG. 5E and FIG. 5F depicts bin scores across a plurality of bins of a genome for a cfDNA sample and a gDNA sample, respectively, that are obtained from a cancer subject. Here, the cancer patient has been clinically diagnosed with stage I breast cancer. A blood test sample was obtained through a blood draw from the cancer patient and collected in a blood collection tube. The blood sample tube was centrifuged at 1600 g, the plasma and buffy coat components extracted, respectively, and stored at minus 20° C. cfDNA was extracted from plasma using QIAAMP Circulating Nucleic Acid kit (Qiagen, Germantown, Md.) and pooled. White blood cells in the buffy coat were lysed and gDNA extracted using a DNEASY Blood and Tissue kit (Qiagen, Germantown, Md.). Sequencing libraries were prepared from both the extracted cfDNA sample and the gDNA sample using TRUSEQ Nano DNA reagents (Illumina, San Diego, Calif.). After library preparation the cfDNA sequencing library and gDNA sequencing library were sequenced using a HiSeqX sequencer (Illumina, San Diego, Calif.) to obtain sequence reads from both the cfDNA and gDNA samples. Specifically, cfDNA sequence reads and gDNA sequence reads were obtained by performing whole genome sequencing at a depth of coverage of 35×.

Referring specifically to the data shown in FIGS. 5E and 5F, each indicator in each of the graphs of FIGS. 5E and 5F represents a bin score for a bin of the reference genome. The select bins shown on the x-axis represent nucleotide sequences from chromosomes 1-22 of the cancer patient. The bin score for each bin is normalized relative to the number of sequence read counts expected for the bin and therefore, a cfDNA sample or a gDNA sample that is devoid of a copy number event would depict bin scores that minimally deviate from zero.

Unaligned indicators (e.g., marked as “+” in FIGS. 5E and 5F) refer to bins and/or segments of the cfDNA sample that are different from corresponding bins and/or segments of the gDNA sample. For example, a statistically significant bin of the cfDNA sample is depicted as an unaligned indicator in FIG. 5E if the corresponding bin of the gDNA sample is not statistically significant. Similarly, a non-statistically significant bin of the cfDNA sample is depicted as an unaligned indicator in FIG. 5E if the corresponding bin of the gDNA sample is statistically significant. Additionally, all bins within a segment of a cfDNA sample are depicted using unaligned indicators if the segment of the cfDNA sample is different (e.g., statistically significant vs non-statistically significant) from the corresponding segment of the gDNA sample.

Aligned bin indicators (e.g., marked as “x” in FIGS. 5E and 5F) refer to bins in the cfDNA sample and the gDNA sample that align. For example, a statistically significant bin of the cfDNA sample is depicted as an aligned bin indicator if the corresponding bin of the gDNA sample is also statistically significant. Similarly, a non-statistically significant bin of the cfDNA sample is depicted as an aligned bin indicator if the corresponding bin of the gDNA sample is also non-statistically significant.

Aligned segment indicators (e.g., marked as “V” in FIGS. 5E and 5F) refer to bins in the cfDNA sample and the gDNA sample that are included in aligned segments. Specifically, the bins in a statistically significant segment of the cfDNA sample are depicted using aligned segment indicators if the corresponding segment of the gDNA sample is also statistically significant. Here, the bins in the corresponding segment of the gDNA sample are also depicted using aligned segment indicators.

Referring to FIG. 5E, the cfDNA sample includes a statistically significant segment 520A that includes bins with bin scores above zero. Additionally, the cfDNA sample includes a statistically significant segment 522A that includes bins with bin scores below zero. Furthermore, the cfDNA sample includes bins 524A and 526A that are statistically significant as they each have a bin score that is above zero. Each statistically significant segment (e.g., 520A and 522A) and statistically significant bin (e.g., 524A and 526A) are indicative of a copy number event.

Referring to FIG. 5F, the gDNA sample includes segment 520B and segment 522B that each includes bins with bin scores that are not significantly different from a value of zero. Here, segment 520B of the gDNA sample is the corresponding segment of segment 520A of the cfDNA sample. Additionally, segment 522B of the gDNA sample is the corresponding segment of segment 522A of the cfDNA sample. The gDNA sample also includes statistically significant bin 526B that is the corresponding bin for bin 526A of the cfDNA sample.

Here, the statistically significant segments (e.g., segment 520A and 520B) in the cfDNA sample are unaligned with the corresponding segments (e.g., segment 520B and 522B) in the gDNA sample. Specifically, statistically significant segment 520A of the cfDNA sample is unaligned with segment 520B of the gDNA sample. Additionally, segment 522A of the cfDNA sample is unaligned with segment 522B of the gDNA sample. This indicates that the copy number events represented by each of the statistically significant segment 520A and 522A are likely due to a somatic tumor event.

Additionally, bin 526A of the cfDNA sample aligns with bin 526B of the gDNA sample. Thus, the copy number event represented by bin 526A of the cfDNA sample is likely due to either a germline or somatic non-tumor event.

3.2.2. Example 2: Potential Copy Number Aberration Originates from Somatic Tumor Source in a Non-Cancer Sample

FIG. 5G and FIG. 5I depicts bin scores across bins of a genome determined from a cfDNA sample and a gDNA sample, respectively, that are obtained from a non-cancer individual. Here, as the individual has not been diagnosed with cancer, the individual can be a candidate for early detection of cancer. A blood test sample was obtained through a blood draw from the non-cancer individual and cfDNA and gDNA was extracted. Extraction and sequencing of cfDNA and gDNA samples to generate sequence reads for analysis was performed according to the process described above in Example 1.

As shown in FIG. 5G, the cfDNA sample includes a statistically significant segment 532A that includes bins with bin scores above zero. Additionally, the cfDNA sample includes a statistically significant bin 530A that includes a bin score above zero. The statistically significant segment 532A and statistically significant bin 530A are indicative of copy number events. As shown in FIG. 5H, the gDNA sample includes segment 532B that includes bins with bin scores that are not significantly different from a value of zero. Segment 532B of the gDNA sample is the corresponding segment of segment 532A of the cfDNA sample. Additionally, the gDNA sample also includes statistically significant bin 530B that is the corresponding bin for bin 530A of the cfDNA sample.

Bin 530A of the cfDNA sample aligns with bin 530B of the gDNA sample. Thus, the copy number event represented by bin 530A of the cfDNA sample is likely due to either a germline or somatic non-tumor event. The statistically significant segment 532A in the cfDNA sample is unaligned with the corresponding segment 532B in the gDNA sample. This indicates that the copy number event represented by the statistically significant segment 532A is possibly due to a somatic tumor event. This demonstrates that a healthy individual (e.g., not diagnosed for cancer) can potentially be screened for early detection of cancer by identifying possible copy number aberrations using cfDNA and gDNA samples obtained from the individual.

3.2.3. Example 3: Copy Number Variations Originate from a Germline or Somatic Non-tumor Source in a Non-Cancer Sample

FIG. 5I and FIG. 5J depicts bin scores across bins of a genome determined from a cfDNA sample and a gDNA sample, respectively, that are obtained from a non-cancer individual. Here, as the individual has not been diagnosed with cancer, the individual can be a candidate for early detection of cancer. A blood test sample was obtained through a blood draw from the non-cancer individual and cfDNA and gDNA was extracted. Extraction and sequencing of cfDNA and gDNA samples to generate sequence reads for analysis was performed according to the process described above in Example 1.

As shown in FIG. 5I, the cfDNA sample includes a statistically significant segment 534A that includes bins with bin scores below zero. Additionally, the cfDNA sample includes a statistically significant bin 536A that includes a bin score above zero. The statistically significant segment 534A and statistically significant bin 536A are indicative of copy number events. As shown in FIG. 5J, the gDNA sample includes segment 534B. Segment 534B of the gDNA sample is the corresponding segment of segment 534A of the cfDNA sample. Here, the statistically significant segment 534B includes at least a subset of bins with bin scores that do not deviate significantly from zero. In other words, the segment-level analysis enables the identification of a statistically significant segment 534B that includes a subset of bins that, individually, would not have been identified as statistically significant bins. This demonstrates the benefit of performing a segment-level analysis, in addition to performing a bin-level analysis, in order to identify copy number events. The gDNA sample additionally includes statistically significant bin 536B that is the corresponding bin for bin 536A of the cfDNA sample.

Here, the statistically significant segment 534A in the cfDNA sample aligns with the corresponding statistically significant segment 534B in the gDNA sample. This indicates that the copy number event represented by the statistically significant segment 534A is likely due to either a germline or somatic non-tumor event. Additionally, bin 536A of the cfDNA sample aligns with bin 536B of the gDNA sample. Thus, the copy number event represented by bin 536A of the cfDNA sample is also likely due to either a germline or somatic non-tumor event.

3.3. Dimensionality Reduction Workflow

Returning again to FIG. 5A, the dimensionality reduction workflow (e.g., steps 505, 508A-C) generally analyzes high dimensionality data and performs a dimensionality reduction to obtain a lower dimensionality data that represents the main characteristics of the original sequence reads. Such lower dimensionality data may be reduced dimensionality features that serve as whole genome features. As an example, high dimensionality data may be bin scores of bins across the genome which, in some embodiments, may include thousands of values. Therefore, lower dimensionality data (e.g., reduced dimensionality features) can be a smaller number of values that are reduced from the thousands of bin scores of bins across the genome.

The dimensionality reduction workflow begins at step 505 where sequence reads derived from a cfDNA sample are obtained. The sequence reads derived from cfDNA 115 can be received from a whole genome sequencing assay 132.

At step 508A, genomic regions across the genome that exhibit low variability in sequence read counts are identified through the use of one or more criteria. In various embodiments, genomic regions can refer to bins of the genome that were discussed above in relation to the copy number aberration detection workflow.

The criteria are formulated to improve the quality of data of high dimensionality by identifying and eliminating systematic errors or other types of non-disease related noises during data collection. In other words, by applying the criteria, bins that are typically noisy which are unsuitable for subsequent analysis can be eliminated. In some embodiments, sequence reads subject to the analysis at step 508A have been pre-processed to correct biases or errors using one or more methods such as normalization, correction of GC biases, correction of biases due to PCR over-amplification, and etc.

In some embodiments, one or more criteria are established to exclude nucleic acid sequencing data that likely contain systematic errors or other types of non-disease related noises during data collection. As disclosed herein, sequence data can include sequence reads of any biological samples, including but not limited to cell-free nucleic acid samples.

In some embodiments, data from only healthy subjects are used to establish the one or more criteria to avoid interferences from data associated with one or more disease conditions. In some embodiments, a criterion as disclosed herein can be established with respect to genomic or chromosomal regions. For example, nucleic acid sequence reads can be aligned to regions of a reference genome and one or more characteristics of the sequence reads can be used to determine whether data associated with a particular genomic region is associated with a baseline noise that confounds the information from the genomic region less. Thus, the particular genomic region can be excluded from subsequent analyses. Exemplary characteristics include but are not limited to, for example, number of reads, mappability of the reads, and etc.

In some embodiments, the genomic regions have the same size. In some embodiments, the genomic regions can have different sizes. In some embodiments, a genomic region can be defined by the number of nucleic acid residues within the region. In some embodiments, a genomic region can be defined by its location and the number of nucleic acids residues within the region. Any suitable size can be used to define genomic regions. For example, a genomic region can include 10 kb or fewer, 20 kb or fewer, 30 kb or fewer, 40 kb or fewer, 50 kb or fewer, 60 kb or fewer, 70 kb or fewer, 80 kb or fewer, 90 kb or fewer, 100 kb or fewer, 110 kb or fewer, 120 kb or fewer, 130 kb or fewer, 140 kb or fewer, 150 kb or fewer, 160 kb or fewer, 170 kb or fewer, 180 kb or fewer, 190 kb or fewer, 200 kb or fewer, or 250 kb or fewer.

Regions that are possibly associated with systematic errors are identified. In some embodiments, one or more criteria can be defined to reduce or eliminate data corresponding to these noisier regions. For example, a high variability filter can be created to allow one to discard data corresponding to all regions with data variations above a threshold value. In other embodiments, a low variability filter can be created to focus subsequent analysis on data with data variations below a threshold.

As an illustration, a human haploid reference genome includes over three billion bases that can be divided into about 30,000 regions (or bins). If an experimental value is observed for each bin, for example, a total number of sequence reads that align to the particular region or bin, each subject can correspond to over 30,000 measurements. After a low or high variability filter is applied, the number of measurements corresponding to a subject can be reduced by a significant portion; for example, including but not limited to about 50% or less, about 45% or less, about 40% or less, about 35% or less, about 30% or less, about 25% or less, 20% or less, 15% or less, 10% or less, or 5% or less. In some embodiments, the number of measurements corresponding to a subject can be reduced by 500 or more such as about 55%, 600, 65%, or 70% or more. For example, a subject, which originally has over 30,000 corresponding measurements, can have over 30% fewer measurements (e.g., about 20,000) after a high or low variability filter is applied.

At step 508B, the one or more criteria established from the previous step can be applied to a biological dataset of a training group (also referred to as “training data”). As disclosed herein, the training group includes both healthy subjects and subjects known to have one or more medical conditions (also referred to as “diseased subjects”). For example, for sequencing data, the one or more criteria previously determined in step 508A (e.g., a low or high variability filter) is applied to data of the training group to completely remove the portion of the data that are associated with the chromosomal regions defined in the filter. In some embodiments, the presumably noisy data are only partially removed. In some embodiments, the presumably noisy data that are not removed can be assigned a weighting factor to reduce their significance in the overall dataset.

Once data selection is performed for the biological dataset for the training group, the remaining training data, also referred to as the “selected training data” or “filtered training data,” are subject to further analysis to extract features that reflect differences between healthy subjects and subjects known to have one or more medical conditions. As noted previously, the original training data include data from both healthy subjects and diseased subjects. The filtered training data constitute a part of the original training data and thus also include data from both healthy subjects and subjects known to have a medical condition. It is assumed that the largest variations among the filtered training data come from differences between data from the healthy subjects and data from the diseased subjects. In essence, it is assumed that data associated with a heathy subject should be more similar to data of another healthy subject than the data from any diseased subject; and vice versa.

Like the original training data, the filtered training data are also of high dimensionality. In some embodiments, the filtered training data are subject to further analysis to reduce data dimensionality and differences between the healthy and diseased subjects are defined based on the reduced dimensionalities. For a given subject, about 20,000 filtered measurements can be further reduced to a handful of data points. For example, the about 20,000 filtered measurements can be transformed based on a few extracted features (e.g., a number of principal components) to render a number of data points. In some embodiments, after reduction of dimensionality, there are 5 or fewer features; 6 or fewer features; 7 or fewer features; 8 or fewer features; 9 or fewer features; 10 or fewer features; 12 or fewer features; 15 or fewer features; or 20 or fewer features. In some embodiments, the filtered measurements can have more than 20 features. The filtered measurements can then be transformed based on the selected features. For example, a sample having two 20,000 filtered measurements can be transformed and reduced to five or fewer data points. In some embodiments, a sample having two 20,000 filtered measurements can be transformed and reduced to more than five data points, such as 10, 15, 20, and etc.

As disclosed herein, the transformed data points from all subjects in the filtered training dataset are subject to further analysis to extract relations or patterns that reflect the differences between the sub-groups in the filtered training dataset. In some embodiments, further analysis includes a binomial logistic regression process; for example, for determining the likelihood of a subject having cancer versus not having cancer. In some embodiments, further analysis includes a multinomial logistic regression process; for example, for determining the type of cancer in addition to the likelihood of a subject having cancer.

At step 508C, optionally, scores are calculated for each subject. In some embodiments, a score is a cancer prediction (e.g., cancer prediction 190B shown in FIG. 1C) that is output by a predictive cancer model 170B.

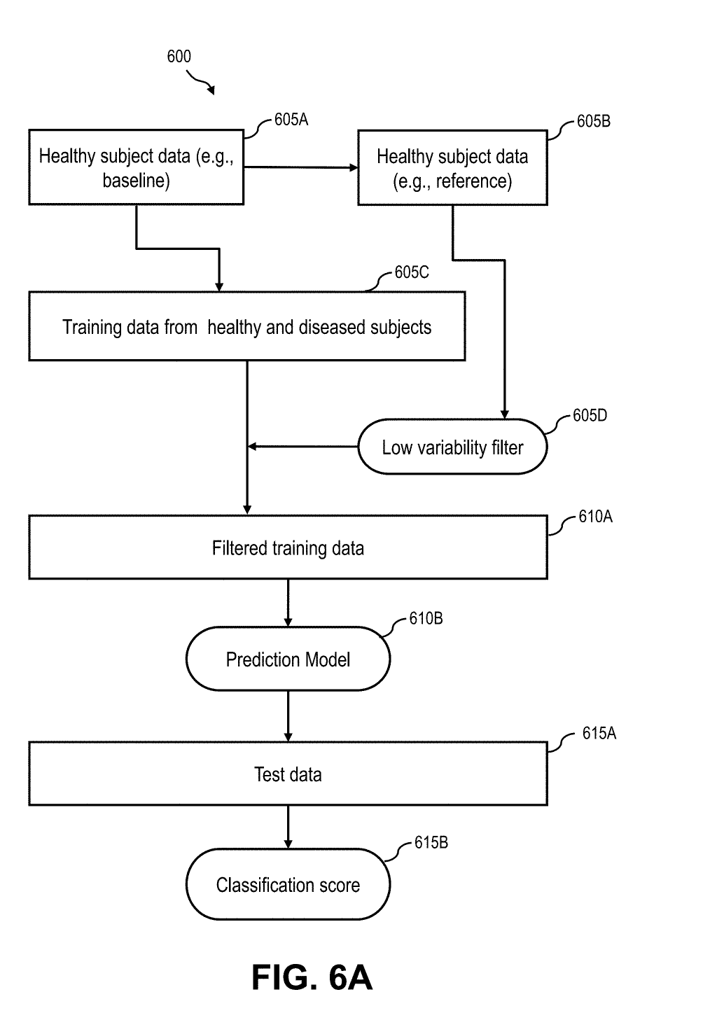

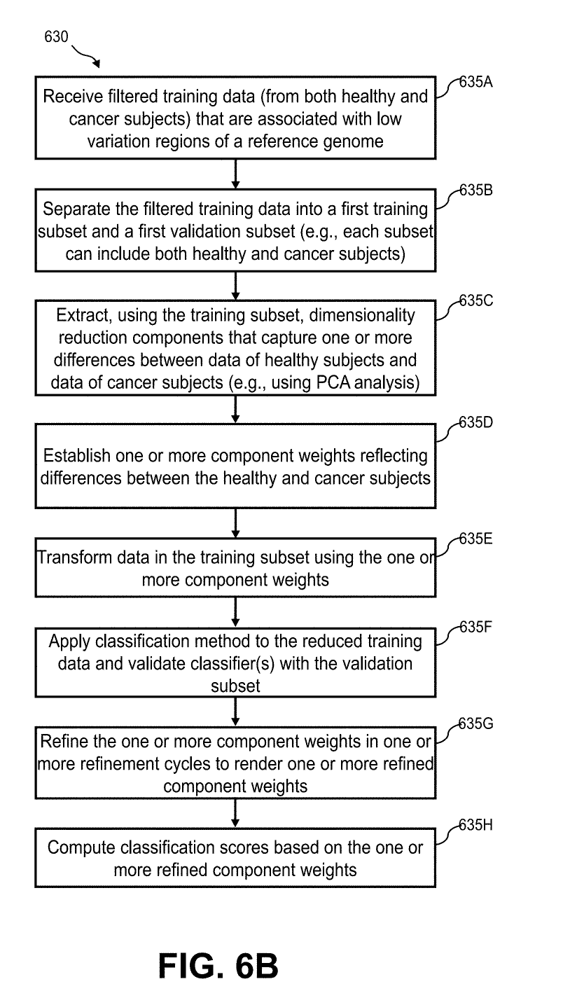

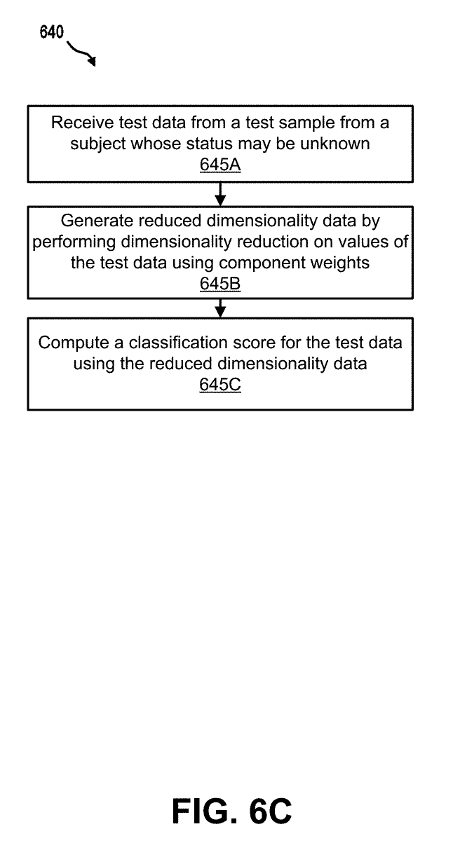

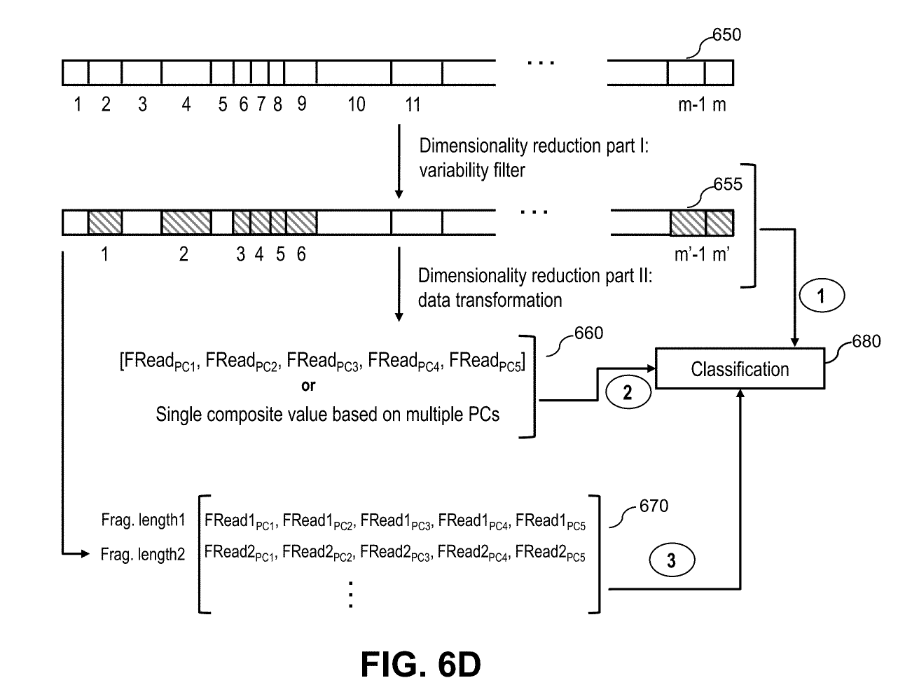

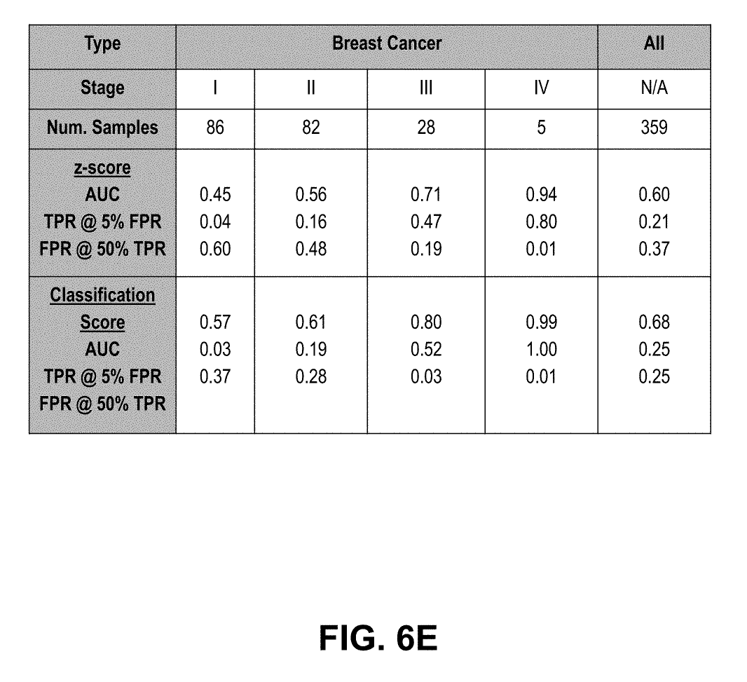

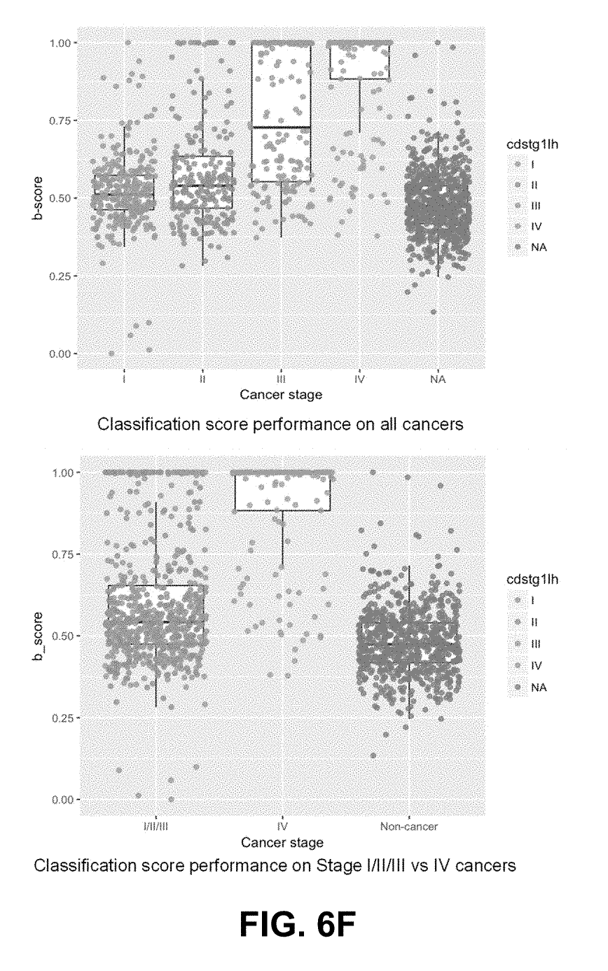

Referring now to FIG. 6A, it illustrates a flow process 600 for determining a classification score by reducing the dimensionality of high dimensionality data, in accordance with an embodiment.

During the data selection portion, data of high dimensionality are initially processed to improve quality. In some embodiments, the number of sequence reads that align to a particular region of a reference genome is normalized. For example, healthy subject data 605A can include sequence reads from a group of healthy subjects (also referred to as baseline subjects) and data from the baseline subjects can be used to establish the normalization standards. In some embodiments, sequence reads from the baseline subjects are aligned to a reference genome that is already divided into a plurality of regions. Assuming that there are no significant biases during the sequencing process, different regions in the genomes should be covered at roughly the same level. Consequently, the number of sequence reads that align to a particular region should be the same as those sequence reads that align to another region of the same size.

In one example, the number of sequence reads from a baseline subject across different genomic regions can be written as Readᵢ^{j}, where integer i denotes a subject and is 1 through n while integer j denotes a genomic region and has a value of 1 through m. As disclosed, a reference genome can be divided into any number of genomic regions, or genomic regions of any sizes. A reference genome can be divided into up to 1,000 regions, 2,000 regions, 4,000 regions, 6,000 regions, 8,000 regions, 10,000 regions, 12,000 regions, 14,000 regions, 16,000 regions, 18,000 regions, 20,000 regions, 22,000 regions, 24,000 regions, 26,000 regions, 28,000 regions, 30,000 regions, 32.000 regions, 34,000 regions, 36,000 regions, 38,000 regions, 40,000 regions, 42,000 regions, 44,000 regions, 46,000 regions, 48,000 regions, 50,000 regions, 55,000 regions, 60,000 regions, 65,000 regions, 70,000 regions, 80,000 regions, 90,000 regions, or up to 100,000 regions. As such, m can be an integer corresponding to the number of genomic regions. In some embodiments, m can be an integer larger than 100,000.

In some embodiments, sequence reads of a subject can be normalized to the average read count across all chromosomal regions for the subject. When i remains constant, sequence reads from genomic regions 1 through m and the corresponding sizes of the regions can be used to compute an average expected number of sequence reads for subject i, for example, based on the equation:

R̅e̅a̅d̅ᵢ=Σⱼ₌₁^{j=m}(Readᵢ^{j}/SizeRegionᵢ^{j})/m,

where SizeRegionᵢ^{j} represents the size of the particular chromosomal region (e.g., in bases or kilobases) to which the sequence reads (Readᵢ^{j}) are aligned. Here, Readᵢ^{j}/Size Regionᵢ^{j} is a sequence read density value. As such, for a subject i, the expected number of sequence reads that would align to a given chromosomal region j having a size of SizeRegionᵢ^{j} can be calculated using the following:

R̅e̅a̅d̅ᵢ̅×SizeRegionᵢ^{j}.

As disclosed herein, data for any subject across different genomic regions can be used as a control to normalize the sequence reads of a genomic region. Here, an average read, which is used as the basis for data normalization, can be computed for a healthy control subject, a group of control subjects, or a test subject itself.

In some embodiments, sequence reads of a subject can be normalized against an overall average count from a group of subjects (e.g., a group of n healthy subjects).

In some embodiments, sequence reads for a subject corresponding to a particular region can be normalized using multiple approaches, utilizing both data from different regions for the subject itself and cross different control subjects.

In one aspect, disclosed herein are methods for establishing a template for selecting data for further analysis, based on patterns gleaned from data from healthy subjects (e.g., baseline healthy subjects 605A and reference healthy subjects 605B). In preferred embodiments, reference healthy subjects 605B do not or only have minimum overlap with baseline healthy subjects 605A. Sequencing data of cell-free nucleic acids are used to illustrate the concepts. However, one of skill in the art would understand that the current method can be applied to sequencing data of other materials or non-sequencing data as well.

In some embodiments, the number of healthy subjects in a baseline or reference healthy subject group can be varied. In some embodiments, the selection criteria for healthy subjects in the baseline and reference healthy subject groups are the same. In some embodiments, the selection criteria for healthy subjects in the baseline and reference healthy subject groups are different.

In some embodiments, a high or low variability filter is established using data from healthy reference subjects 605B. As disclosed herein, the data from healthy reference subjects 605B can be pre-processed (e.g., undergoing various normalization steps); for example, based on baseline control data from healthy subjects 605A. For example, training data from both healthy and cancer subjects can be pre-processed. In some embodiments, raw sequence read data can be directly used to set up a high or low variability filter.

In some embodiments, sequence reads of each healthy subject (e.g., from healthy subject data 605A) can be aligned to a plurality of chromosomal regions of reference genome. The variability of reach genomic region can be evaluated; for example, by comparing numbers of sequence reads for a particular genomic region across all healthy subjects in the control group. As an illustration, healthy subjects who are not expected to have cancers can be included as reference controls. The healthy subjects include but are not limited to subjects who do not have family histories of cancer or who are healthy and young (e.g. under 35 or 30-year-old). In some embodiments, healthy subjects in the reference control group may satisfy other conditions; for example, only healthy women will be included in a control group for breast cancer analysis. Only men will be included in a control group for prostate cancer analysis. In some embodiments, for diseases that are found predominantly or only in a particular ethnic group, only people from the same ethnic group are used to establish the reference control group.

For example, for a group of control healthy subjects (n), if we count the number of sequence reads that align to a genomic region, there will be n values for each genomic region. Parameters, such as a mean or medium count, standard deviation (SD), median absolute deviation (MAD), or the interquartile range (IQR), can be computed based on the n count values and used to determine whether a genomic region is considered of low or high variability. Any method for computing these parameters can be used.

For example, the sequence read numbers for region j in subjects 1 through n can be represented as Readᵢ^{j}, where j is an integer and i is an integer between 1 and n. An average read count of region j R̅e̅a̅d̅^̅{̅J̅}̅ can be calculated using R̅e̅a̅d̅^̅{̅J̅}̅=(Σᵢ₌₁ⁱ⁼ⁿReadᵢ^{j}/n). In some embodiments, IQR can be computed and compared with R̅e̅a̅d̅^̅{̅J̅}̅. If the difference between IQR and R̅e̅a̅d̅^̅{̅J̅}̅ is above a pre-determined threshold, data from region j may be considered of high variability and will be discarded before subsequent analysis. By repeating the process for all regions in a reference genome, a genome-wide high or low variability filter (e.g., element 250) can be established. For example, for any sequencing data associated with a subject (who is preferably not in the reference control group), sequence reads that align to regions corresponding to the high variability filter will be discarded. A low variability filter would include regions whose difference between IQR and R̅e̅a̅d̅^̅{̅J̅}̅ that are below a pre-determined threshold.

In some embodiments, high or low variability filters can be created for only a portion of the genome; for example, for only a particular chromosome or a portion thereof.

In some embodiments, training data 605C includes biological data (e.g., sequencing data) from both healthy subjects and subjects known to have a medical condition (also known as diseased subjects). In some embodiments, data associated healthy subjects who have previously been included in the baseline control group or reference control group will be excluded from training data 605C to possibly avoid certain biases.

In some embodiments, normalization parameters obtained using healthy subject data 605A and the low or high variability filter 605D can be applied to training data 605C to render new and filtered training data 610A for subsequent analysis.

In some embodiments, filtered training data 610A comprise balanced data for healthy and diseased subjects; for example, the numbers of healthy and diseased subjects are within about 5 to 10% of each other. In some embodiments, filtered training data 610A comprise unbalanced data for healthy and diseased subjects; for example, the numbers of healthy and diseased subjects differ more than 10% from each other. In the latter situation, methods can be applied to reduce the impact of unbalanced data.

In some embodiments, filtered training data 610A are subject to further analysis to create prediction model 610B. Prediction model 610B is used to predict whether a subject has a certain medical condition.

In some embodiments, prediction model 610B reflects differences between healthy and diseased subjects. In some embodiments, the differences used in prediction model 610B can be obtained by applying, for example, logistic regression to filtered training data 610A. In some embodiments, filtered training data 610A (e.g., numbers of sequence read that align to certain regions of a reference genome) can be directly used in logistic regression analysis. In some embodiments, filtered training data 610A undergoes a dimensionality reduction to reduce and possibly transform the dataset to a much smaller size. For example, Principal Component Analysis (PCA) can be used to reduce the size of a data set by about 100,000-fold or less, about 90,000-fold or less, about 80,000-fold or less, about 70,000-fold or less, about 60,000-fold or less, about 50,000-fold or less, about 40,000-fold or less, about 30,000-fold or less, about 20,000-fold or less, about 10,000-fold or less, about 9,000-fold or less, about 8,000-fold or less, about 7,000-fold or less, about 6,000-fold or less, about 5,000-fold or less, about 4,000-fold or less, about 3,000-fold or less, about 2,000-fold or less, about 1,000-fold or less, or about 500 fold or less. In some embodiments, the size of a data set can be reduced by more than 100,000-fold. In some embodiments, the size of a data set can be reduced by a couple of hundred folds or less. As disclosed herein, although the size of a data set is reduced, the number of samples can be retained. For example, after PCA, a data set of 1,000 samples can still retain 1,000 samples but the complexity of each sample is reduced (e.g., from corresponding to 25,000 features to 5 or fewer features). As such, the methods disclosed herein can improve efficiency and accuracy of data processing while greatly reduce computer storage space required.

Once a prediction model is established, it can be applied to test data 615A. Test data 615A can be taken from a test subject whose status is unknown with respect to a medical condition. In some embodiments, data from test subjects of known statuses can also be used for validation purposes. In some embodiments, test data 615A will be pre-processed such as going through normalization, GC content correction, and etc. In some embodiments, a high or low variability filter 605D is applied to test data 615A to remove data in chromosomal regions that likely correspond to systematic errors. In some embodiments, both pre-processing and a high or low filter can be applied to test data 615A to render filtered test data for further processing.

In some embodiments, when prediction model 610B is applied to filtered test data 610A, a classification score can be computed as a probability score to represent the likelihood for the particular medical condition to be present in the test subject being analyzed. In such embodiments, the prediction model 610B shown in FIG. 6A may be the predictive cancer model 170B shown in FIG. 1C. Therefore, the classification score can be a cancer prediction 190B. The classification score can be a binomial classification score; for example, non-cancer versus cancer. In some embodiments, the classification score can be a multinomial classification score; for example, non-cancer, liver cancer, lung cancer, breast cancer, prostate cancer, and etc. As discussed above, the classification score can represent a whole genome feature.

FIG. 6B depicts a sample process 630 for analyzing data to reduce data dimensionality, in accordance with an embodiment. Specifically, the sample process 630 refers to steps 610A and 610B (shown in FIG. 6A) in further detail. The sample data analysis process 630 starts step 635A with filtered training data. For example, for sequencing data, only data corresponding to the presumably low variability genomic regions are received at step 635A. As disclosed herein, training data include data from healthy and diseased subjects. After a high or low variability filter has been applied the filtered training data should still include biological data from both healthy and diseased subjects. In some embodiments, the diseased subjects are patients who have been diagnosed with at least one type of cancer.

At step 635B, the filtered training data are separated using cross-validation methods. In some embodiments, the cross validation methods include but are not limited to exhaustive methods like leave-p-out cross-validation (LpO CV) where p can have any value that would create a valid partition or leave-one-out cross validation (LOOCV) where p=1. In some embodiments, the cross validation methods include but are not limited to non-exhaustive methods such as the holdout method, repeated random sub-sampling validation method, or a stratified or non-stratified k-fold cross-validation method where k can have any value that would create a valid partitioning. As disclosed herein, a cross validation procedure partitions the filtered training data into different pairs of a training subset and a validation subset at a predetermined percentage split. For example, the first training subset and first validation subset depicted at step 635B represent an 80:20 split during one fold of a k-fold cross-validation experiment. In another fold of the same k-fold cross-validation experiment, the filtered training data will be split into a different pair of training and validation subsets at the same percentage ratio. In some embodiments, multiple cross-validation experiments are applied, where the split ratio of a pair of training and validation subsets can be varied in each experiment. As disclosed herein, the subsets can be created randomly. In some embodiments, the subsets are created such that each subset include data from both healthy and diseased subjects. In some embodiments, only one of the subsets include data from both healthy and diseased subjects. For example, it is essential that the training subset include both healthy and diseased subjects.

In some embodiments, a training subset constitutes a majority of the filtered training data, for example, up to 60%, up to 65%, up to 70%, up to 75%, up to 80%, up to 85%, up to 90%, or up to 95% of the filtered training data. In some embodiments, more than 95% of a very large set of filtered training data can be used as the training subset. To avoid training biases, it is usually good practice to save at least 5% of untouched data as a test subset, i.e., as this subset will never be used as training data and will only be used to validate the resulting model.

At step 635C, data from the first training subset (e.g., counts of sequence reads or quantities derived therefrom) can be used to derive dimensionality reduction components that capture one or more differences between the data of healthy and diseased subjects. For example, samples that have been identified to have about 10,000 to about 20,000 of low variability regions can have 10,000 to 20,000 corresponding count values (or derived quantities such as a relative count value, a logCount value, and etc.). By using a dimensionality reduction method such as principal component analysis (PCA), it is possible to identify and select dimensionality reduction components, an example of which are principal components (PCs), that represent the largest variations among data in the first training subset. These dimensionality reduction components can be used to reduce the 10,000 to 20,000 values to a lower dimensionality space where each value in the reduced dimensionality space corresponds to one of the selected PCs. As an example, the higher dimensionality values may be N bin scores across N bins of the genome. Therefore, the dimensionality reduction process reduces the N bin scores down to a reduced number of values that each corresponds to a dimensionality reduction component. In some embodiments, 5 or fewer dimensionality reduction components are selected. In some embodiments, 10 or fewer dimensionality reduction components are selected. In some embodiments, 15 or fewer dimensionality reduction components are selected. In some embodiments, 20 or fewer dimensionality reduction components are selected. In some embodiments, 25 or fewer dimensionality reduction components are selected. In some embodiments, 30 or fewer dimensionality reduction components are selected. In some embodiments, 35 or fewer dimensionality reduction components are selected. In some embodiments, 40 or fewer dimensionality reduction components are selected. In some embodiments, 45 or fewer dimensionality reduction components are selected. In some embodiments, 50 or fewer dimensionality reduction components are selected. In some embodiments, 60 or fewer dimensionality reduction components are selected. In some embodiments, 70 or fewer dimensionality reduction components are selected. In some embodiments, 80 or fewer dimensionality reduction components are selected. In some embodiments, 90 or fewer dimensionality reduction components are selected. In some embodiments, 100 or fewer dimensionality reduction components are selected. In some embodiments, more than 100 dimensionality reduction components are selected.

These dimensionality reduction components identified through the dimensionality reduction method (e.g., through PCA) can be used during deployment (e.g., in the steps shown in FIG. 6D) to reduce data of high dimensionality down to data of lower dimensionality (e.g., reduced dimensionality features).

At step 635D, one or more component weights can be derived that reflect the relative contribution of each of the dimensionality reduction components. For example, for each dimensionality reduction component, a component weight is assigned to each value from each low variability region to reflect the respective importance of the data. A region contributing more to the observed differences will be assigned a larger component weight, and vice versa. In some embodiments, the component weight is specific for a component and is also region-specific, but the same for all subjects, for example, wₖ can represent the component weight associated for region j in connection with PC k, where j is an integer from 1 to m′, and m′ is an integer smaller than m, the original number of genomic regions designated in the reference genome. This component weight value is the same for different subjects. In some embodiments, more individualized component weight values can be formulated to reflect differences between the subjects. For example, the component weight may differ from one cancer type to another cancer type. For the same region and same PC, the component weight for people of different ethnic origins can differ. These component weights can be used during deployment (e.g., in the steps shown in FIG. 6D) to reduce values of high dimensionality to a smaller number of values of lower dimensionality (e.g., reduced dimensionality features).

At step 635E, data in the first training subset are transformed based on the extracted dimensionality reduction components (e.g., a few selected PCs). In some embodiments, dimensionality of the transformed data is much smaller than those of the filtered training data, whose dimensionality is already reduced from the original un-filtered data. The concepts are illustrated as follows.

Subject₁(Read)=[Read₁¹,Read₁²,Read₁³, . . . , Read₁^{m}]

illustrates the data of subject 1 before a low variability filter is applied, where m is the total number of regions.

Subject₁(FRead)=[FRead₁¹,FRead₁²,FRead₁³, . . . , FRead₁^{m′}]

illustrates the data of subject 1 before a low variability filter is applied, where m′ is the total number of regions. After a low variability filter is applied, the total number of genomic regions is reduced to m′, which can be significantly smaller than m. For example, un-filtered data for a subject can include 30,000 components or more, each associated with a genomic region. After a low variability filter is applied, a significant portion of the genomic regions can be excluded as being having high variability; for example, filtered data for the same subject can include 20,000 components or fewer, each associated with a low variability genomic region.

At step 635E, data dimensionality of the filtered data can be further reduced based on the number of extracted dimensionality reduction components. For example, if k principal components are selected, the dimensionality of the filtered data can be reduced to k. The number of selected PCs can be much smaller than the dimensionality of the filtered data. For example, when only 5 PCs are selected, the data dimensionality of filtered read data (FRead) for subject 1 can be further reduced to 5, such as the expression below:

$$\begin{matrix}{FRead}_{PC 1} = {\sum }_{j = 1}^{j = {m}^{′}} ( {w}_{PC 1}^{j} \times {FRead}_{1}^{j} ) & \\ {FRead}_{PC 2} = {\sum }_{j = 1}^{j = {m}^{′}} ( {w}_{PC 2}^{j} \times {FRead}_{1}^{j} ) & \\ {FRead}_{PC 3} = {\sum }_{j = 1}^{j = {m}^{′}} ( {w}_{PC 3}^{j} \times {FRead}_{1}^{j} ) & \\ {FRead}_{PC 4} = {\sum }_{j = 1}^{j = {m}^{′}} ( {w}_{PC 4}^{j} \times {FRead}_{1}^{j} ) & \\ {FRead}_{PC 5} = {\sum }_{j = 1}^{j = {m}^{′}} ( {w}_{PC 5}^{j} \times {FRead}_{1}^{j} ) & \end{matrix}$$

As such, quantity data (e.g., read numbers) associated with a large number of low variability regions can be reduced and transformed to a handful of numeric values. In some embodiments, a component weight (e.g., w_{PC}^{j}) can be assigned to each PC. In some embodiments, a single value can be computed based on the values associated with multiple PCs.

At step 635F, a classification method is applied to the transformed data of each subject to provide a classification score. In some embodiments, the classification score can be a binomial or multinomial probability score. For example, in a binomial classification for cancer, logistic regression can be applied to compute a probability score, where 0 represents no likelihood of cancer while I represents the highest certainty of having cancer. A score of over 0.5 indicates that the subject is more likely to have cancer than not having cancer. Logistic regression generates the coefficients (and its standard errors and significance levels) of a formula to predict a logit transformation of the probability of presence of the characteristic of interest. Using the same example to illustrate probability determination by logistic regression, the probability (p) of a subject having cancer can be written as the following

logit(p)=b₀+b₁×FRead_{PC1}+b₂×FRead_{PC2}+b₃×FRead_{PC3}+b₄×FRead_{PC4}+b₅×FRead_{PC5}

where each transformed and reduced data derived from PCI is assigned a weight. The logit transformation is defined as the logged odds:

$$odds = \frac{p}{1 - p} = \frac{probability of cancer being present}{probability of cancer being absent}$$

and p probability

$$p = \frac{1}{1 + {e}^{- logit ( p )}}$$

The value of p can be computed by plugging the logit(p) value. In some embodiments, it is possible to look up values in a logit table.

In some embodiments, a multinomial classification approach can be taken to classify subjects into different cancer types. For example, existing multinomial classification techniques can be categorized into (i) transformation to binary (ii) extension from binary and (iii) hierarchical classification. In a transformation to binary approach, a multi-class problem can be transformed into multiple binary problems based on a one-vs-rest or one-vs-one approach. Exemplary extension from binary algorithms include but are not limited to neural networks, decision trees, k-nearest neighbors, naive Bayes, support vector machines and Extreme Learning Machines, and etc. Hierarchical classification tackles the multinomial classification problem by dividing the output space i.e. into a tree. Each parent node is divided into multiple child nodes and the process is continued until each child node represents only one class. Several methods have been proposed based on hierarchical classification. In some embodiments, multinomial logistic regression can be applied. It is used to predict the probabilities of the different possible outcomes of a categorically distributed dependent variable, given a set of independent variables (which may be real-valued, binary-valued, categorical-valued, etc.).

At step 635G, the filtered training data are partitioned into a second training subset and a second test/validation subset and the steps of 635B through 635F are repeated in one or more refinement cycles (also referred to as “one or more cross-validation cycles”). As disclosed herein, in the cross-validation procedure the validation subsets themselves have little (e.g., in repeated random sampling) or no overlap at all (LOOCV, LpO CV, k-fold) over different folds.

During a refinement cycle, a predetermined condition (e.g., a cost function) can be applied to optimize the classification results. In some embodiments, one or more parameters in a classification function are refined using training data subset and validated by validation or held out subset during each fold of the cross-validation procedure. In some embodiments, PC-specific weights and/or region-specific weights can be refined to optimize classification results.

In some embodiments, a small portion of the filtered training data can be kept aside, not as a part of a training subset during any fold of the cross-validation procedure to better estimate overfitting.

At step 635H, the refined parameters are used to compute classification scores. As disclosed herein, the refined parameters can function as a prediction model for cancer as well as cancer types. It is possible to construct a prediction model using multiple types of biological data; including but not limited to, for example, nucleic acid sequencing data (cell-free versus non cell-free, whole genome sequencing data, whole genome methylation sequencing data, RNA sequencing data, targeted panel sequencing data), protein sequencing data, tissue pathology data, family history data, epidemiology data, and etc.

In one aspect, disclosed herein are method for classifying a subject as having a certain medical condition, based on the dimensionality reduction components and the component weights (or refined component weights) established using training data. FIG. 6C depicts a process for analyzing data from a test sample based on information learned from data with reduced dimensionality, in accordance with an embodiment. Process 640 illustrates how test data from a subject, whose status with respect to a medical condition is unknown, can be used to compute a classification score and serve as a basis for diagnosing whether the subject is likely to have the condition.

At step 645A, test data is received from a test sample from the subject who status is unknown. In some embodiments, the test data are of the same type as those from the baseline healthy subjects including. In some embodiments, the test data are of the same type as those from the reference healthy subjects. Sample data type includes but not limited to sequencing data for detecting targeted mutations, whole genome sequencing data, RNA sequencing data, and whole genome sequencing data for detecting methylation. In some embodiments, the test data can be calibrated and adjusted for improved quality (e.g., normalization, GC content correction and etc.).

At step 645B, high dimensionality values of the test data are reduced to a lower dimensional space using component weights that are determined during training. For example, the high dimensionality values may be read data, such as a bin score (e.g., the normalized number of sequence reads that are categorized in bins across the genome). First, a data selection can be performed using a previously defined low variability filter. Advantageously, the filter-based approach is straight-forward and can be easily adjusted by changing the threshold value of the reference quantities computed for genomic regions in a reference genome. Therefore, high dimensionality values that are highly variable (e.g., due to presence of noise in certain bins) can be filtered and removed. Next, the high dimensionality values that remain following the data selection step can be reduced in dimensionality using a dimensionality reduction process such as PCA. The number of dimensionality reduction components resulting from the dimensionality reduction process can be based on the number of dimensionality reduction components that were determined during training. For example, if the dimensionality reduction process generated 5 principal components, then the dimensionality reduction process for values of the test sample also generates 5 principal components. Each dimensionality reduction component is weighted by a corresponding component weight that was determined during training. Therefore the lower dimensionality data can be calculated.

As an example, with 5 dimensionality reduction components, then 5 lower dimensionality data can be generated. Specifically, the data dimensionality of filtered read data (FRead) can be reduced to 5 lower dimensionality data (FRead_{PC1}, FRead_{PC2}, FRead_{PC3}, FRead_{PC4}, FRead_{PC5}), which can be expressed as:

$$\begin{matrix}{FRead}_{PC 1} = {\sum }_{j = 1}^{j = {m}^{′}} ( {w}_{PC 1}^{j} \times {FRead}_{1}^{j} ) \\ {FRead}_{PC 2} = {\sum }_{j = 1}^{j = {m}^{′}} ( {w}_{PC 2}^{j} \times {FRead}_{1}^{j} ) \\ {FRead}_{PC 3} = {\sum }_{j = 1}^{j = {m}^{′}} ( {w}_{PC 3}^{j} \times {FRead}_{1}^{j} ) \\ {FRead}_{PC 4} = {\sum }_{j = 1}^{j = {m}^{′}} ( {w}_{PC 4}^{j} \times {FRead}_{1}^{j} ) \\ {FRead}_{PC 5} = {\sum }_{j = 1}^{j = {m}^{′}} ( {w}_{PC 5}^{j} \times {FRead}_{1}^{j} )\end{matrix}$$

where w_{PC}^{j} represents the component weight assigned to each dimensionality reduction component. Here, the reduced dimensionality data represented as FRead_{PC} can serve as whole genome features, as described above.

At step 645C, a classification score can be computed for the test subject using the reduced dimensionality data. In various embodiments, the classification score can be a cancer prediction outputted by a prediction model, such as predictive cancer model 170C shown in FIG. 1C. In other words, the prediction model can serve as a single-assay prediction model that outputs a cancer prediction 190B based only on whole genome features 152.

FIG. 6D depicts a sample process for data analysis in accordance with an embodiment. As described in detail in connection with FIG. 6B, reduction of dimensionality can take place during multiple points of the data analysis.

In some embodiments, a certain level of data selection can occur during initial data processing: e.g., during normalization, GC content correction, and other initial data calibration steps, it is possible to rejects sequence reads that are clearly defective and thus reduce the number of data. As illustrated in FIG. 6D, data dimensionality reduction can take place with the application of a low or high variability filter. For example, a reference genome can be divided into a number of regions, examples of which are numbered in element 610 as 1, 2, 3, . . . , m−1, and m. The regions can be equal or non-equal in size.

As disclosed herein, a low variability filter specifies a subset of the genomic regions that will be selected for further processing. For example, the regions indicated with a hatched shading (e.g., regions indicated as 1, 2, 3, 4, 5, 6 . . . m′−1, m′) are selected regions. The filter allows categorical selection or rejection of data based on established analysis of possible systematic errors using reference healthy subjects.

The selected data are then transformed to further reduce data dimensionality to reduced dimensionality data (e.g., element 660). In some embodiments, transformed data 660 can be generated using data from all the selected genomic regions, but the dimensionality of data can be greatly reduced. For example, data from over 20,000 different genomic regions can be transformed into a handful of values. In some embodiments, a single value can be generated. Here, the reduced dimensionality data in the form of a handful of values or a single value are the reduced dimensionality features described above that can serve as whole genome features.

In some embodiments, selected sequencing data from element 655 can be sorted in subgroups according to the fragment size represented by the sequencing data. For example, instead of a single count for all sequence reads bound to a particular region, multiple quantiles can be derived each corresponding to a size or size range. For example, sequence reads corresponding to fragments of 140-150 bases will be separately grouped from sequence reads corresponding to fragments of 150-160 bases, as illustrated in element 670. As such, additional detail and fine tuning can be made before the data are used for classification.

As illustrated in FIG. 6D, multiple types of data can be used for classification 680, including but not limited to data from selected/filtered genomic regions without dimensionality reduction, reduced data, reduced and sorted data, and etc.

The current method and system offer advantages over previously known methods. For example, classification is done using quantities that can be easily derived from raw sequencing data. The current does not require building chromosome-specific segmentation maps, thus eliminating the time-consuming process for generating those maps. Also, the current method permits more efficient use of computer storage space because it no longer requires storage for the large segmentation maps.

3.3.1. Example 1: Comparison of Classification Score and Z-Score

FIG. 6E depicts a table comparing the current method (classification score) with a previous known segmentation method (z-score). The data showed that overall the predictive power of the classification score is consistently higher than that of the z-scores across all stages of breast cancer samples.

FIG. 6F depicts the improved predictive power of using the classification score method can be observed for all types of cancer (top). The predictive power for early-stage cancer is improved and for late-stage cancer is especially good (bottom).

### 4. Methylation Computational Analysis

4.1. Methylation Features

Referring briefly again to FIGS. 1B-1D, the methylation computational analysis 140D receives sequence reads generated by the methylation sequencing assay 136 and determines values of methylation features 156 based on the sequence reads. In general, any known methylation-based sequencing approach known in the art may be used to assess methylation status of a plurality of CpG sites across the genome or across a gene panel (e.g., see US 2014/0080715 and U.S. Ser. No. 16/352,602, which are incorporated herein by reference). Examples of methylation features 156 include any of: quantity of hypomethylated counts, quantity of hypermethylated counts, hypomethylation score per CpG site, hypermethylation score per CpG site, rankings based on hypermethylation scores, rankings based on hypomethylation scores, and presence or absence of abnormally methylated (e.g., hypomethylated or hypermethylated) fragments at one or more CpG sites. Methylation features can be a fragment methylation pattern, which can be determined, e.g., by counting fragments satisfying a set of criteria, or by a separate machine learning model that may be trained separately or in conjunction with this model.

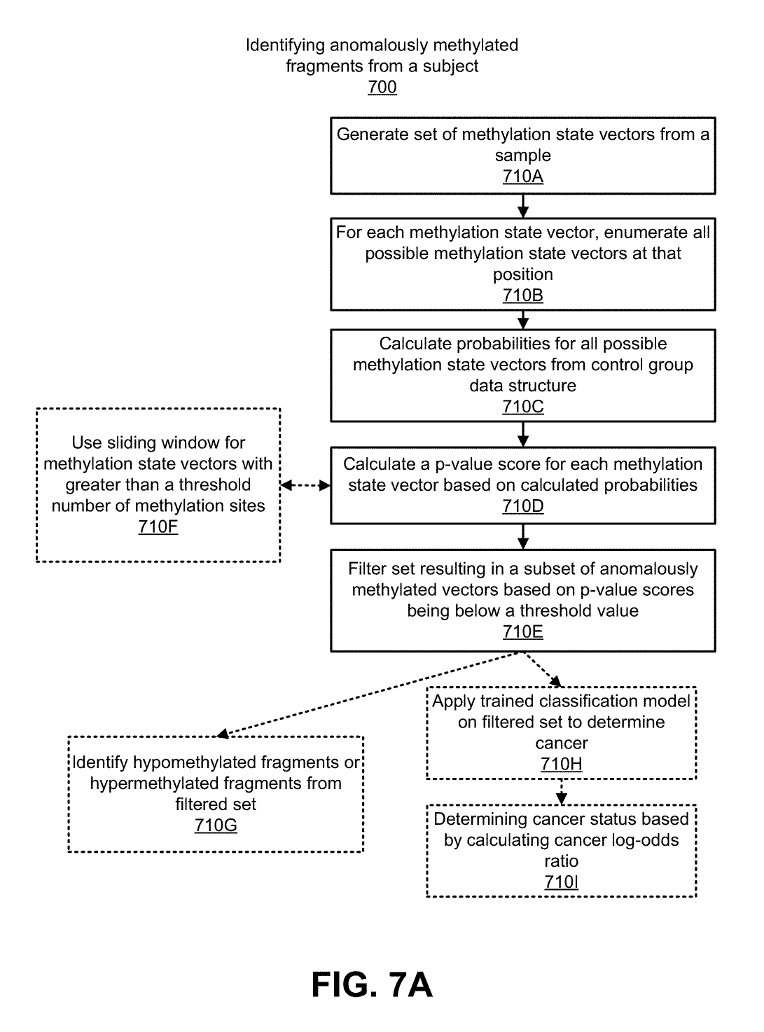

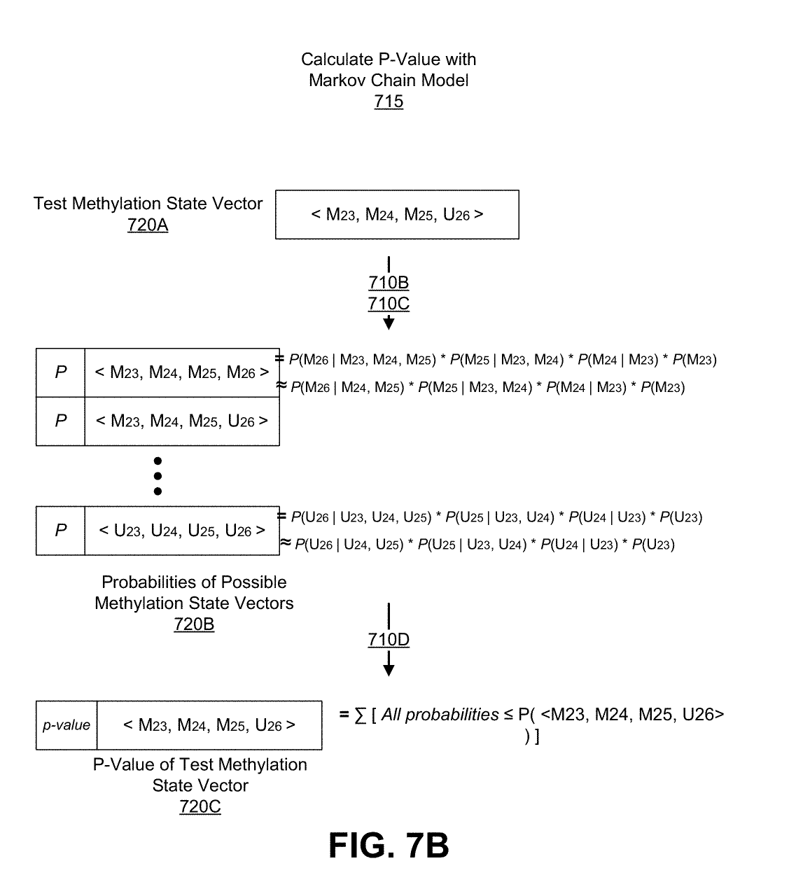

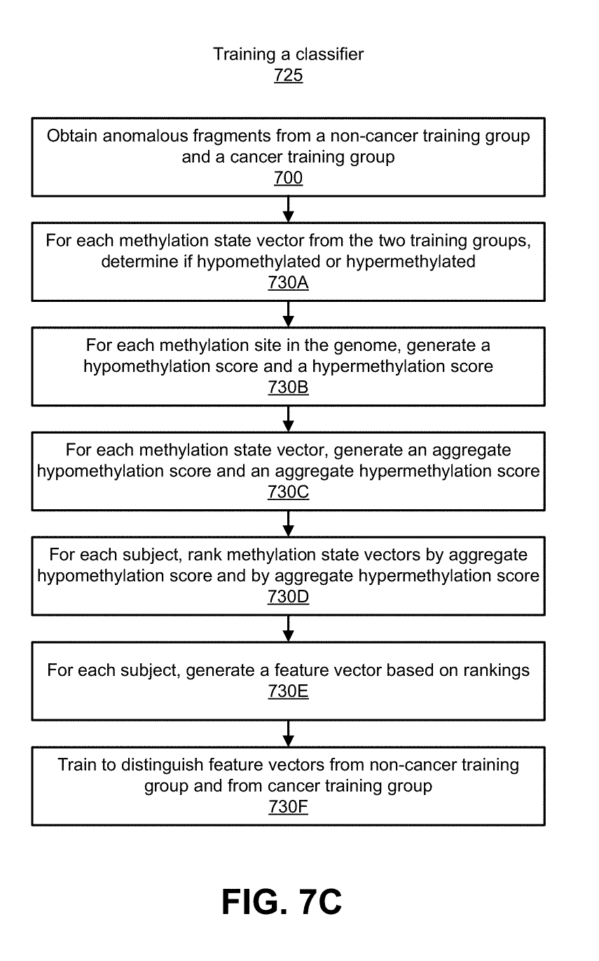

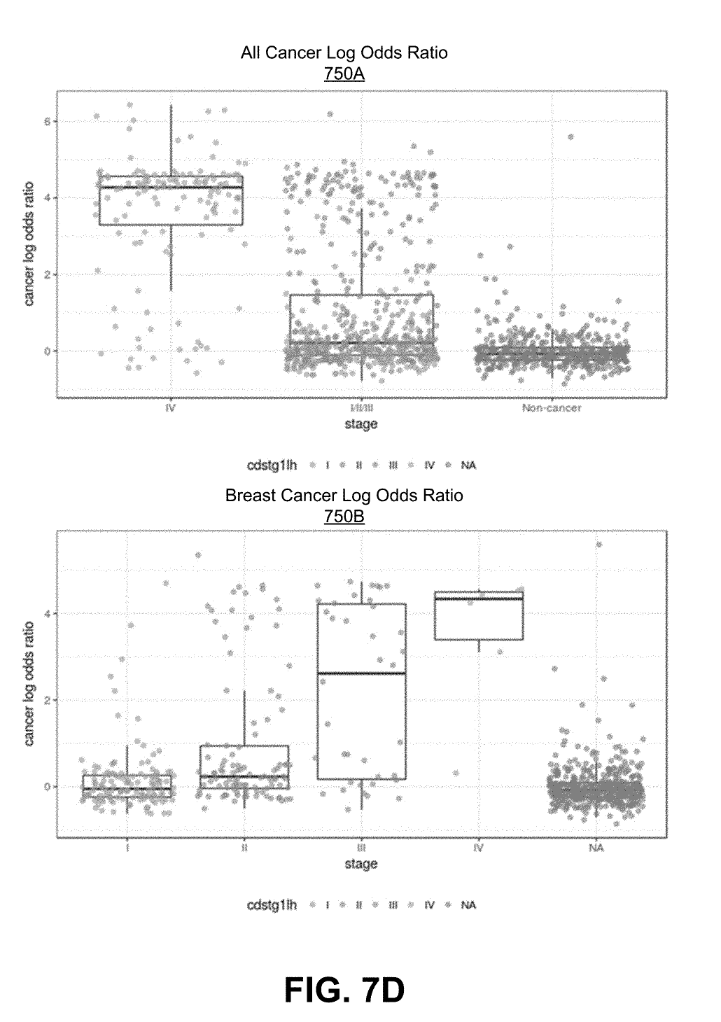

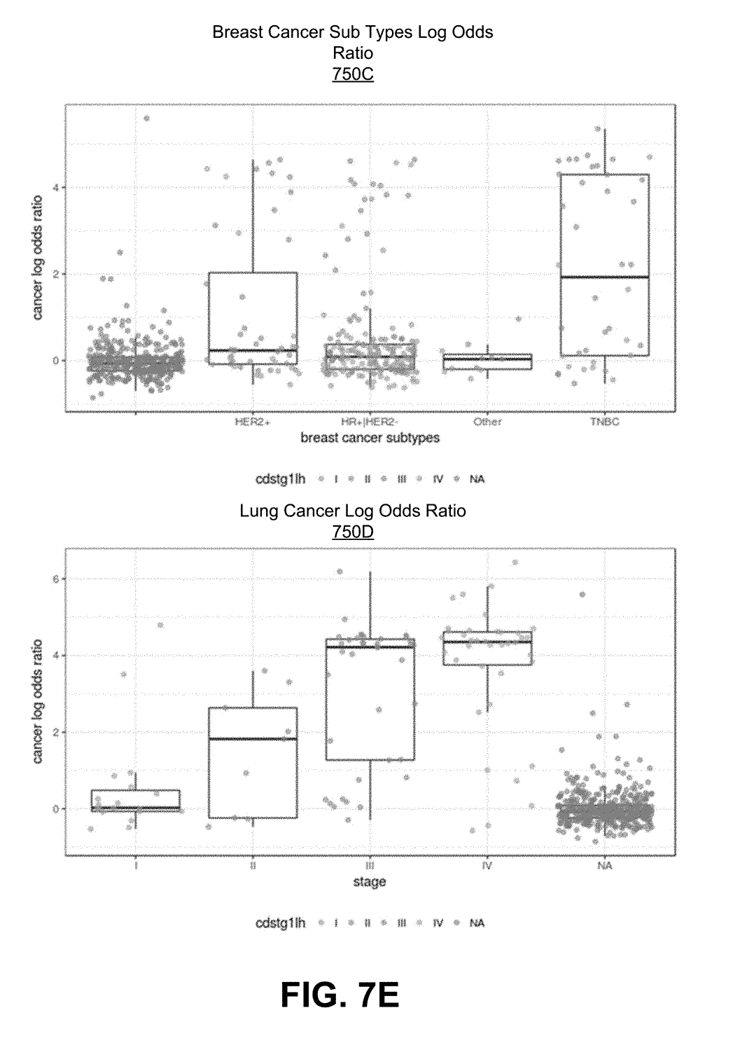

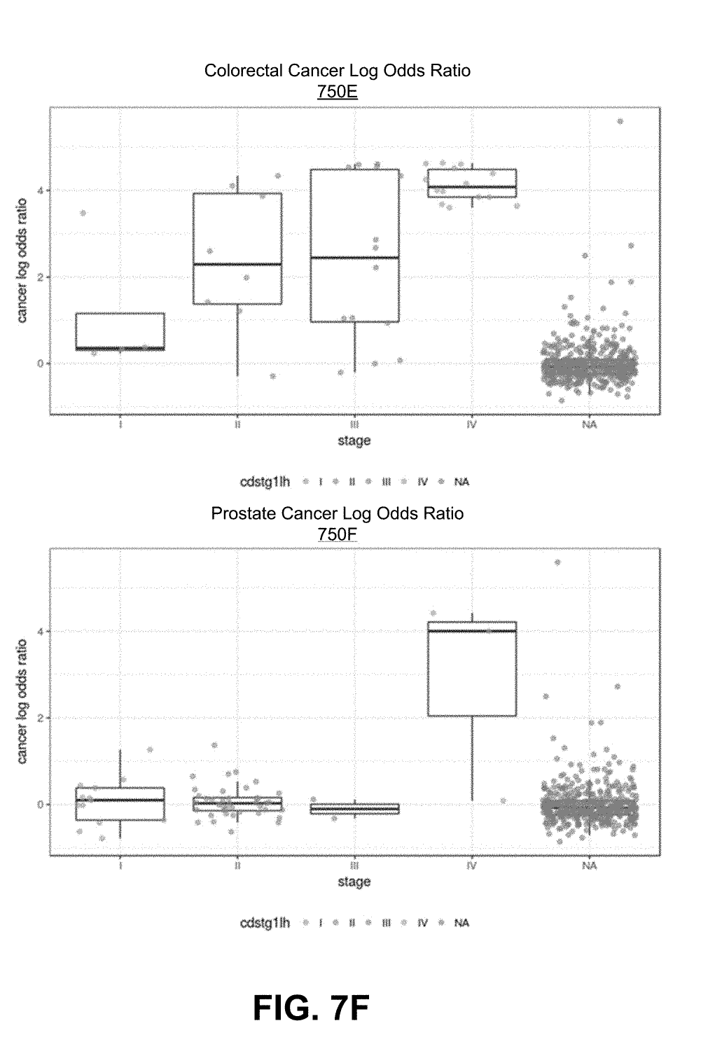

The quantity of hypomethylated counts and quantity of hypermethylated counts refer to the total number of cfDNA fragments that are hypomethylated and hypermethylated, respectively. Hypomethylated and hypermethylated cfDNA fragments are identified using methylation state vectors, as is described below in relation to process 700 in FIG. 7A.

The hypomethylation score per CpG site and hypermethylation score per CpG site relate to an estimate of cancer probability given the presence of hypomethylation or hypermethylation of fragments. Generally, the hypomethylation score per CpG site and hypermethylation score per CpG site is based on counts of methylation state vectors that overlap the CpG site that are either hypomethylated or hypermethylated.

The rankings based on hypermethylation scores and rankings based on hypomethylation scores can refer to rankings of methylation state vectors based on their associated hypomethylation/hypermethylation scores. In one embodiment, the rankings refer to rankings of fragments based on a maximum hypomethylation/hypermethylation score of methylation state vectors in each fragment.

The presence or absence of an abnormally methylated (e.g., hypomethylated or hypermethylated) fragment at a CpG site can be expressed as a 0 (absent) or 1 (present) value for each CpG site. In some aspects, the presence/absence of an abnormally methylated fragment is determined for CpG sites that provide the most information gain. For example, in some scenarios, ˜3000 CpG sites are identified as providing information gain and therefore, there are ˜3000 feature values for this particular feature.

4.2. Methylation Computational Analysis Overview

FIG. 7A is a flowchart describing a process 700 for identifying anomalously methylated fragments from a subject, according to an embodiment. An example of process 700 is visually illustrated in FIG. 7B, and is further described below in reference to FIG. 7A. In process 700, a processing system generates 710A methylation state vectors from cfDNA fragments of the subject. A methylation state vector comprises the CpG sites in the cfDNA fragments as well as their methylation state—methylated or unmethylated. For example, a methylation state vector can be expressed as <M₂₃, U₂₄, M₂₅>, thereby indicating that position 23 is methylated, position 24 is unmethylated, and position 25 is methylated. The processing system handles each methylation state vector as follows.

For a given methylation state vector, the processing system enumerates 710B all possibilities of methylation state vectors having the same starting CpG site and same length (i.e., set of CpG sites) in the methylation state vector. As each methylation state may be methylated or unmethylated there are only two possible states at each CpG site, and thus the count of distinct possibilities of methylation state vectors depends on a power of 2, such that a methylation state vector of length n would be associated with 2ⁿ possibilities of methylation state vectors.

The processing system calculates 710C the probability of observing each possibility of methylation state vector for the identified starting CpG site/methylation state vector length by accessing a healthy control group data structure. In one embodiment, calculating the probability of observing a given possibility uses a Markov chain probability to model the joint probability calculation which will be described in greater detail with respect to FIG. 7B below. In other embodiments, calculation methods other than Markov chain probabilities are used to determine the probability of observing each possibility of methylation state vector.

To generate a healthy control group data structure, the processing system subdivides methylation state vectors derived from cfDNA of individuals in the healthy control group into strings of CpG sites. In one embodiment, the processing system subdivides the methylation state vector such that the resulting strings are all less than a given length. For example, a methylation state vector of length 11 may be subdivided into strings of length less than or equal to 3 would result in 9 strings of length 3, 10 strings of length 2, and 11 strings of length 1. In another example, a methylation state vector of length 7 being subdivided into strings of length less than or equal to 4 would result in 4 strings of length 4, 5 strings of length 3, 6 strings of length 2, and 7 strings of length l. If a methylation state vector is shorter than or the same length as the specified string length, then the methylation state vector may be converted into a single string containing all of the CpG sites of the vector.

The processing system tallies the strings by counting, for each possible CpG site and possibility of methylation states in the vector, the number of strings present in the control group having the specified CpG site as the first CpG site in the string and having that possibility of methylation states. For example, at a given CpG site and considering string lengths of 3, there are 2\hat{ }3 or 8 possible string configurations. At that given CpG site, for each of the 8 possible string configurations, the processing system tallies how many occurrences of each methylation state vector possibility come up in the control group. Continuing this example, this may involve tallying the following quantities: <Mₓ, Mₓ₊₁, Mₓ₊₂, <Mₓ, Mₓ₊₁, Uₓ₊₂>, . . . , <Uₓ, Uₓ₊₁, Uₓ₊₂> for each starting CpG site x in the reference genome. The processing system creates the data structure storing the tallied counts for each starting CpG site and string possibility. Thus, this data structure can be used to determine the probability of observing each possibility of a methylation state vector.

The processing system calculates 710D a p-value score for the methylation state vector using the calculated probabilities for each possibility. In one embodiment, this includes identifying the calculated probability corresponding to the possibility that matches the methylation state vector in question. Specifically, this is the possibility having the same set of CpG sites, or similarly the same starting CpG site and length as the methylation state vector. The processing system sums the calculated probabilities of any possibilities having probabilities less than or equal to the identified probability to generate the p-value score.

This p-value represents the probability of observing the methylation state vector of the fragment or other methylation state vectors even less probable in the healthy control group. A low p-value score, thereby, generally corresponds to a methylation state vector which is rare in a healthy subject, and which causes the fragment to be labeled anomalously methylated, relative to the healthy control group. A high p-value score generally relates to a methylation state vector is expected to be present, in a relative sense, in a healthy subject. If the healthy control group is a non-cancerous group, for example, a low p-value indicates that the fragment is anomalously methylated relative to the non-cancer group, and therefore possibly indicative of the presence of cancer in the test subject.

As above, the processing system calculates p-value scores for each of a plurality of methylation state vectors, each representing a cfDNA fragment in the test sample. To identify which of the fragments are anomalously methylated, the processing system may filter 710E the set of methylation state vectors based on their p-value scores. In one embodiment, filtering is performed by comparing the p-values scores against a threshold and keeping only those fragments below the threshold. This threshold p-value score could be on the order of 0.1, 0.01, 0.001, 0.0001, or similar.

FIG. 7B is an illustration 715 of an example p-value score calculation, according to an embodiment. To calculate a p-value score given a test methylation state vector 720A, the processing system takes that test methylation state vector 720A and enumerates 710B possibilities of methylation state vectors. In this illustrative example, the test methylation state vector 720A is <M₂₃, M₂₄, M₂₅, U₂₆>. As the length of the test methylation state vector 720A is 4, there are 2∝possibilities of methylation state vectors encompassing CpG sites 23-26. In a generic example, the number of possibilities of methylation state vectors is 2\hat{ }n, where n is the length of the test methylation state vector or alternatively the length of the sliding window (described further below).

The processing system calculates 710C probabilities 720B for the enumerated possibilities of methylation state vectors. As methylation is conditionally dependent on methylation status of nearby CpG sites, one way to calculate the probability of observing a given methylation state vector possibility is to use Markov chain model. Generally a methylation state vector such as <S₁, S₂, . . . , Sₙ>, where S denotes the methylation state whether methylated (denoted as M), unmethylated (denoted as U), or indeterminate (denoted as I), has a joint probability that can be expanded using the chain rule of probabilities as:

P(<S₁,S₂, . . . , Sₙ>)=P(Sₙ|S₁, . . . , Sₙ₋₁)*P(Sₙ₋₁|S₁, . . . , Sₙ₋₂)* . . . *P(S₂|S₁)*P(S₁).

Markov chain model can be used to make the calculation of the conditional probabilities of each possibility more efficient. In one embodiment, the processing system selects a Markov chain order k which corresponds to how many prior CpG sites in the vector (or window) to consider in the conditional probability calculation, such that the conditional probability is modeled as P(Sₙ|S₁, . . . , Sₙ₋₁)˜P(Sₙ|Sₙ₋ₖ₋₂, . . . , Sₙ₋₁).

To calculate each Markov modeled probability for a possibility of methylation state vector, the processing system accesses the control group's data structure, specifically the counts of various strings of CpG sites and states. To calculate P(Mₙ|Sₙ₋ₖ₋₂, . . . , Sₙ₋₁), the processing system takes a ratio of the stored count of the number of strings from the data structure matching <Sₙ₋ₖ₋₂, . . . , Sₙ₋₁, Mₙ> divided by the sum of the stored count of the number of strings from the data structure matching <Sₙ₋ₖ₋₂, . . . , Sₙ₋₁, Mₙ> and <Sₙ₋ₖ₋₂, . . . , Sₙ₋₁, Uₙ>. Thus, P(Mₙ|Sₙ₋ₖ₋₂, . . . , Sₙ₋₁), is calculated ratio having the form:

[# of <Sₙ₋ₖ₋₂, . . . , Sₙ₋₁,Mₙ>]/[# of <Sₙ₋ₖ₋₂, . . . , Sₙ₋₁,Mₙ>+# of <Sₙ₋ₖ₋₂, . . . , Sₙ₋₁,Uₙ>].

The calculation may additionally implement a smoothing of the counts by applying a prior distribution. In one embodiment, the prior distribution is a uniform prior as in Laplace smoothing. As an example of this, a constant is added to the numerator and another constant (e.g., twice the constant in the numerator) is added to the denominator of the above equation. In other embodiments, an algorithmic technique such as Knesser-Ney smoothing is used.

In the illustration, the above denoted formulas are applied to the test methylation state vector 720A covering sites 23-26. Once the calculated probabilities 720B are completed, the processing system calculates 710D a p-value score 720C that sums the probabilities that are less than or equal to the probability of possibility of methylation state vector matching the test methylation state vector 720A.

In one embodiment, the computational burden of calculating probabilities and/or p-value scores may be further reduced by caching at least some calculations. For example, the analytic system may cache in transitory or persistent memory calculations of probabilities for possibilities of methylation state vectors (or windows thereof). If other fragments have the same CpG sites, caching the possibility probabilities allows for efficient calculation of p-score values without needing to re-calculate the underlying possibility probabilities. Equivalently, the processing system may calculate p-value scores for each of the possibilities of methylation state vectors associated with a set of CpG sites from vector (or window thereof). The processing system may cache the p-value scores for use in determining the p-value scores of other fragments including the same CpG sites. Generally, the p-value scores of possibilities of methylation state vectors having the same CpG sites may be used to determine the p-value score of a different one of the possibilities from the same set of CpG sites.

Referring again to FIG. 7A, in one embodiment, the processing system uses 710F a sliding window to determine possibilities of methylation state vectors and calculate p-values. Rather than enumerating possibilities and calculating p-values for entire methylation state vectors, the processing system enumerates possibilities and calculates p-values for only a window of sequential CpG sites, where the window is shorter in length (of CpG sites) than at least some fragments (otherwise, the window would serve no purpose). The window length may be static, user determined, dynamic, or otherwise selected.

In calculating p-values for a methylation state vector larger than the window, the window identifies the sequential set of CpG sites from the vector within the window starting from the first CpG site in the vector. The analytic system calculates a p-value score for the window including the first CpG site. The processing system then “slides” the window to the second CpG site in the vector, and calculates another p-value score for the second window. Thus, for a window size I and methylation vector length m, each methylation state vector will generate m−l+1 p-value scores. After completing the p-value calculations for each portion of the vector, the lowest p-value score from all sliding windows is taken as the overall p-value score for the methylation state vector. In another embodiment, the processing system aggregates the p-value scores for the methylation state vectors to generate an overall p-value score.

Using the sliding window helps to reduce the number of enumerated possibilities of methylation state vectors and their corresponding probability calculations that would otherwise need to be performed. Example probability calculations are shown in FIG. 7B, but generally the number of possibilities of methylation state vectors increases exponentially by a factor of 2 with the size of the methylation state vector. To give a realistic example, it is possible for fragments to have upwards of 54 CpG sites. Instead of computing probabilities for 2\hat{ }54 (˜1.8×10\hat{ }16) possibilities to generate a single p-score, the processing system can instead use a window of size 5 (for example) which results in 50 p-value calculations for each of the 50 windows of the methylation state vector for that fragment. Each of the 50 calculations enumerates 2\hat{ }5 (32) possibilities of methylation state vectors, which total results in 50×2\hat{ }5 (1.6×10\hat{ }3) probability calculations. This results in a vast reduction of calculations to be performed, with no meaningful hit to the accurate identification of anomalous fragments.

The processing system may perform any variety and/or possibility of additional analyses with the set of anomalous fragments. One additional analysis identifies 710G hypomethylated fragments or hypermethylated fragments from the filtered set. Fragments that are hypomethylated or hypermethylated may be defined as fragments of a certain length of CpG sites (e.g., more than 3, 4, 5, 6, 7, 8, 9, 10, etc.) with a high percentage of methylated CpG sites (e.g., more than 80%, 85%, 90%, or 95%, or any other percentage within the range of 50%-100%) or a high percentage of unmethylated CpG sites (e.g., more than 80%, 85%, 90%, or 95%, or any other percentage within the range of 50%-100%), respectively. FIG. 7C, described below, illustrates an example process for identifying these anomalously methylated portions of a genome based on the set of anomalously methylated fragments. Although the steps of FIG. 7C describe the process of training a classifier using anomalously methylated fragments from training groups, the particular steps (e.g., steps 730A-730E) can also refer to the generating of features, such as methylation features, for the set of anomalously methylated fragments identified in Step 710G.

In various embodiments, the processing system may identify a presence or absence of an anomalously methylated fragment that overlaps a particular CpG site. As described above, the presence/absence of an anomalously methylated fragment of a CpG site can serve as a methylation feature. As one example, if a hypermethylated or hypomethylated fragment overlaps a particular CpG site, then the CpG site can be assigned a value of 1, indicating the presence of an anomalously methylated fragment. Conversely, the CpG site can be assigned a value of 0 which indicates the lack of an anomalously methylated fragment at the CpG site. In various embodiments, the processing system identifies CpG sites that provide the most information gain and determines the presence/absence of an anomalously methylated fragment at the CpG sites that provide the most information gain. For example, for each CpG site, the processing system computes information gain for the label (cancer vs. non-cancer) given knowledge of whether a highly methylated or unmethylated fragment overlaps that CpG site. The processing system selects the top k CpG sites based on the information gain of each CpG site and can determine the presence/absence of an anomalously methylated fragment at each of the most informative CpG sites.

An alternate analysis applies 710H a trained classification model on the set of anomalous fragments. The trained classification model described hereafter may refer to the predictive cancer model 170B shown in FIG. 1C. In other words, the trained classification model can be a single-assay prediction model that outputs a cancer prediction 190B based on whole genome features 152.

The trained classification model can be trained to identify any condition of interest that can be identified from the methylation state vectors. Generally, the trained classification model can employ any one of a number of classification techniques. In one embodiment the classifier is a non-linear classifier. In a specific embodiment, the classifier is a non-linear classifier utilizing a L2-regularized kernel logistic regression with a Gaussian radial basis function (RBF) kernel.

In one embodiment, the trained classification model is a binary classifier trained based on methylation states for cfDNA fragments obtained from a subject cohort with cancer, and optionally based on methylation states for cfDNA fragments obtained from a healthy subject cohort without cancer, and is then used to classify a test subject probability of having cancer, or not having cancer, based on anomalously methylation state vectors. In further embodiments, different classifiers may be trained using subject cohorts known to have particular cancer (e.g., breast, lung, prostrate, etc.) to predict whether a test subject has those specific cancers.

In one embodiment, the classifier is trained based on information about hyper/hypo methylated regions from the process 710G and as described with respect to FIG. 7C below.

Another additional analysis calculates the log-odds ratio that the anomalous fragments from a subject are indicative of cancer generally, or of particular types of cancer. The log-odds ratio can be calculated by taking the log of a ratio of a probability of being cancerous over a probability of being non-cancerous (i.e., one minus the probability of being cancerous), both as determined by the applied classification model.

FIGS. 7D-F show graphs of various cancers from various subjects across different stages, plotting the log-odds ratio of the anomalous fragments identified according to the process described with respect to FIG. 7A above. This data was obtained through testing of more than 1700 clinically evaluable subjects with over 1400 subjects filtered including nearly 600 subjects without cancer and just over 800 subjects with cancer. The first graph 750A in FIG. 7D shows all cancer cases across three different levels—non-cancer; stage I/II and stage IV. The cancer log-odds ratio for stage IV is significantly larger than those for stage VI/II and non-cancer. The second graph 750B in FIG. 7D shows breast cancer cases across all stages of cancer and non-cancer, with a similar progression in log-odds ratio increasing through the progressive stages of cancer. The third graph 750C in FIG. 7E shows breast cancer sub-types. Noticeably sub-types HER2+ and TNBC are more spread out, whereas HR+/HER2− is concentrated closer to ˜1. The fourth graph 750D in FIG. 7E shows lung cancer cases across all stages of cancer and non-cancer with steady progression through progressive stages of the lung cancer. The fifth graph 750E shows colorectal cancer cases across all stages of cancer and non-cancer, again showing steady progression through progressive stages of the colorectal cancer. The sixth graph 750F in FIG. 7F shows prostate cancer cases across all stages of cancer and non-cancer. This example is different than most of the previously illustrated, only stage IV is significantly different compared to other stages I/II/II and non-cancer.

4.3. Hyper/Hypo Methylated Regions and a Classifier

FIG. 7C is a flowchart describing a process 725 of training a classifier based on methylation status of cfDNA fragments, according to an embodiment. The process accesses two training groups of samples—a non-cancer group and a cancer group—and obtains 700 a non-cancer set of methylation state vectors and a cancer set of methylation state vectors comprising the anomalous fragments of the samples in each group. The anomalous fragments may be identified according to the process of FIG. 7A, for example.

The process determines 730A, for each methylation state vector, whether the methylation state vector is hypomethylated or hypermethylated. Here, the hypermethylated or hypomethylated label is assigned if at least some number of CpG sites have a particular state (methylated or unmethylated, respectively) and/or have a threshold percentage of sites that are the particular state (again, methylated or unmethylated, respectively). As defined above, cfDNA fragments are identifed as hypomethylated or hypermethylated, respectively, if the fragment has at least five CpG sites that are either unmethylated or methylated and (logical AND) above 80% of the fragments CpG sites being unmethylated or methylated. The total fragments that are hypomethylated (e.g., quantity of hypomethylated counts) and the total fragments that are hypermethylated (e.g., quantity of hypermethylated counts) can serve as methylation features.

In an alternate embodiment, the process considers portions of the methylation state vector and determines whether the portion is hypomethylated or hypermethylated, and may distinguish that portion to be hypomethylated or hypermethylated. This alternative resolves missing methylation state vectors which are large in size but contain at least one region of dense hypomethylation or hypermethylation. This process of defining hypomethylation and hypermethylation can be applied in step 710G of FIG. 7A.

The process generates 730B a hypomethylation score and a hypermethylation score per CpG site in the genome. As discussed above, the hypomethylation score and hypermethylation score for each CpG site in the genome can serve as a methylation feature. To generate either score at a given CpG site, the classifier takes four counts at that CpG site—(1) count of (methylations state) vectors of the cancer set labeled hypomethylated that overlap the CpG site; (2) count of vectors of the cancer set labeled hypermethylated that overlap the CpG site; (3) count of vectors of the non-cancer set labeled hypomethylated that overlap the CpG site; and (4) count of vectors of the non-cancer set labeled hypermethylated that overlap the CpG site. Additionally the process may normalize these counts for each group to account for variance in group size between the non-cancer group and the cancer group.

To generate 730C the hypomethylation score at a given CpG site, the process takes a ratio of (1) over (1) summed with (3). Similarly the hypermethylation score is calculated by taking a ratio of (2) over (2) and (4). Additionally these ratios may be calculated with an additional smoothing technique as discussed above. The hypomethylation score and the hypermethylation score relate to an estimate of cancer probability given the presence of hypomethylation or hypermethylation of fragments from the cancer set.

The process generates 730C an aggregate hypomethylation score and an aggregate hypermethylation score for each anomalous methylation state vector. The aggregate hyper and hypo methylation scores, are determined based on the hyper and hypo methylation scores of the CpG sites in the methylation state vector. In one embodiment, the aggregate hyper and hypo methylation scores are assigned as the largest hyper and hypo methylation scores of the sites in each state vector, respectively. However, in alternate embodiments, the aggregate scores could be based on means, medians, maximum, or other calculations that use the hyper/hypo methylation scores of the sites in each vector.

The process then ranks 730D all of that subject's methylation state vectors by their aggregate hypomethylation score and by their aggregate hypermethylation score, resulting in two rankings per subject. In particular embodiments, the process ranks fragments based on the maximum hypomethylation score or maximum hypermethylation score, thereby resulting in two rankings per subject. These two rankings, which are the rankings based on hypermethylation scores and the rankings based on hypomethylation scores, can serve as methylation features. The process selects aggregate hypomethylation scores from the hypomethylation ranking and aggregate hypermethylation scores from the hypermethylation ranking. With the selected scores, the classifier generates 730E a single feature vector for each subject. In one embodiment, the scores selected from either ranking are selected with a fixed order that is the same for each generated feature vector for each subject in each of the training groups. As an example, in one embodiment the classifier selects the first, the second, the fourth, the eighth, and the sixteenth aggregate hyper methylation score, and similarly for each aggregate hypo methylation score, from each ranking and writes those scores in the feature vector for that subject. At step 730F, the process trains 730F a binary classifier to distinguish feature vectors between the cancer and non-cancer training groups. The trained classifier can then be applied when needed at, for example, step 710H of FIG. 7A.

### 5. Common Assay Features

Referring again to FIGS. 1B-1D, in various embodiments, one or more of the whole genome features 152, small variant features 154, and methylation features 156 can also include one or more common assay features. Common assay features refer to features that can be determined regardless of the physical assay (e.g., whole genome sequencing assay 132, small variant sequencing assay 134, methylation sequencing assay 136) that is performed.

Common assay features can be informative for predicting cancer. As one example, common assay features can be a characteristic of the cfDNA 115. As another example, common assay features can be a characteristic of the gDNA (e.g., WBC DNA 120). For example, a characteristic of cfDNA or gDNA can be a total quantity of cfDNA or gDNA in a sample. Tumors in cancerous individuals often produce higher levels of DNA and therefore, the total quantity of DNA (e.g., cfDNA) can be informative for generating a cancer prediction. As another example, a characteristic of cfDNA can be a total concentration of tumor-derived nucleic acid. As yet another example, a characteristic of cfDNA can be a mean or median fragment length across DNA fragments obtained from the cfDNA sample.

### 6. Baseline Analysis and Baseline Computational Analysis

6.1. Baseline Features

Referring again to FIGS. 1B-1D, the baseline analysis 130 and the computational analysis 140A determines values of baseline features 150. Examples of baseline features 150 include clinical features of the individual 110 such as age, weight, body mass index (BMI), patient behavior (e.g., smoker/non-smoker, pack years smoked, alcohol intake, lack of physical activity), family history (e.g., history that is obtained through a questionnaire), physical symptoms (e.g., coughing, blood in stool, etc.), anatomical observations (e.g., breast density), and carrier of a penetrant germline cancer. In various embodiments, clinical features of the individual 110 can be directly obtained through a baseline analysis 130. In other words, the baseline analysis 130 can directly generate baseline features 150 without a baseline computational analysis 140A.

Examples of baseline features 150 can also include a polygenic risk score derived from germline mutations. Germline mutations can include known germline mutations that are associated with cancer, such as one of ATM, BRCA1, BRCA2, CHEK2, PALB2, PTEN, STK11, or TP53. Here, the baseline computational analysis 140A can perform the steps to determine a feature value represented by the polygenic risk score.

6.1. Baseline Analysis and Computational Analysis Workflow

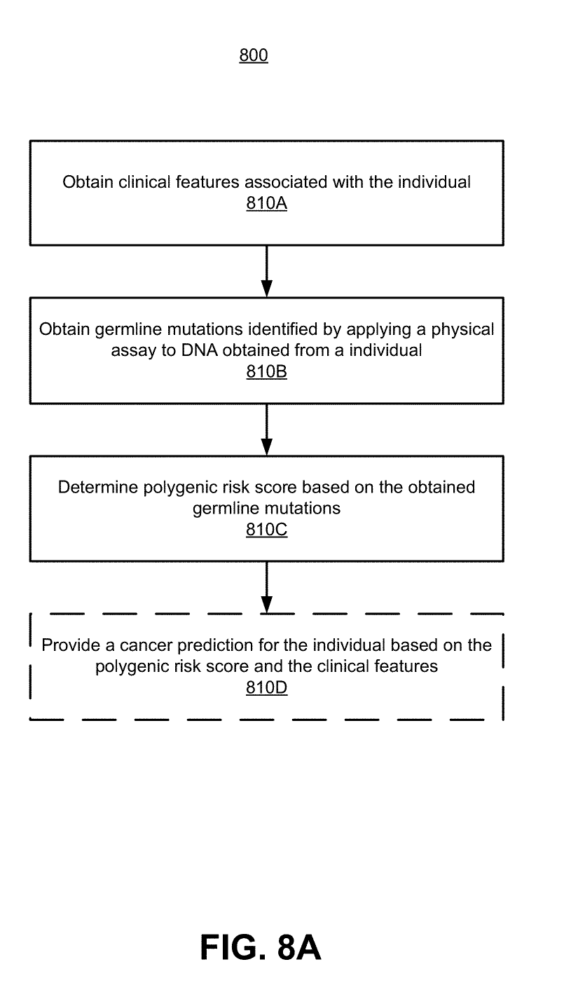

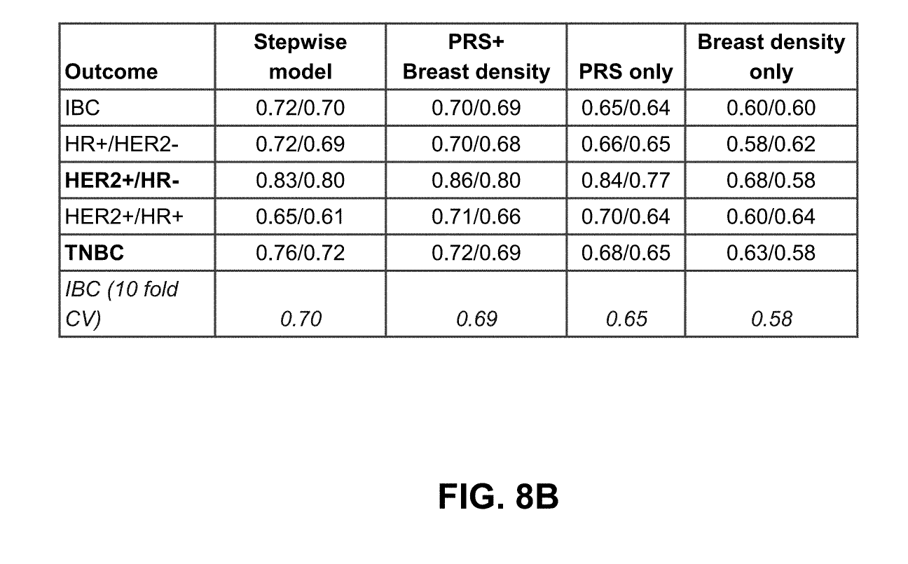

FIG. 8A depicts a flow process 800 for determining baseline features that can be used to stratify a patient, in accordance with an embodiment. At step 810A, clinical features associated with the individual 110 are obtained. As an example, clinical features can be obtained as a result of a medical examination of the individual 110 (e.g., an examination conducted by a medical professional or a physician).

At step 810B, germline mutations of the individual are obtained. In various embodiments, identified germline mutations are obtained from the application of a physical assay to DNA obtained from the individual. As one example, the germline mutations are previously identified through application of the whole genome sequencing assay 132 and the whole genome computational analysis 140B. Specifically, the whole genome sequencing assay 132 can be applied to gDNA (e.g., WBC DNA 120) to generate sequence reads and the whole genome computational analysis 140B can be applied to identify the germline mutations that are present. As an example, a germline mutation can be a copy number variation that is jointly found in both gDNA and cfDNA, as discussed above in relation to the copy number aberration detection workflow (e.g., Section 3.2).

At step 810C, a polygenic risk score (PRS) is determined for the individual based on the germline mutations that were obtained. As an example, a PRS can be represented as the weighted summation of the identified germline mutations. Each assigned weight to a germline mutation can be previously determined through training data. As one example, the PRS for a is calculated as the weighted sum of log(odds ratio) across all risk loci for each individual i:
genetic log(odds ratio) for a given cancer: log(OR)=(Σₖ₌₁^{#loci}βₖ·allele count) The β weights for each germline mutation were obtained from the external large scale genome-wide association studies and applied to each participant's allele count for that germline mutation and summed across all risk loci into a single polygenic score.

At step 810D, optionally, a cancer prediction for the individual can be provided based on the baseline features including the clinical features and the polygenic risk score. In one embodiment, a cancer prediction can include the stratification of the individual in a particular category based on the baseline features. Stratification of the individual can be useful because a cancer prediction for the individual can be tailored for the particular stratification that the individual is categorized in. An example of a stratification can be a risk of developing cancer (e.g., high risk, medium risk, low risk).

In one embodiment, criteria of the baseline features used to stratify the individual into a category. As an example, a criterion for a PRS can be a threshold PRS percentile (e.g. top 5, 10, 25, 50% of individuals). If the individual is assigned a PRS that is above the threshold PRS percentile, then the individual can be stratified in a first group. Conversely, if the individual is assigned a PRS that is below the threshold PRS percentile, then the individual can be stratified in a second group. As another example, a criterion for a clinical feature can be a presence or absence of cancer in an individual's family history. Thus, if the individual's family history includes the presence of cancer, then the individual is assigned in a first category. Conversely, the individual is categorized in a second category if the individual's family history is lacking a presence of cancer. Various criteria can be jointly used to further stratify individuals into different categories.

In an embodiment, the cancer prediction can be a presence or absence of cancer based on the baseline features. For example, given the PRS and one or more clinical features, a prediction as to whether the individual has cancer can be provided.

6.2. Example 1: Cancer Prediction Based on Baseline Features

FIG. 8B depicts the performance of logistic regression predictive models across different types of breast cancer based on different combinations of baseline features. The breast cancers of interest include overall invasive breast cancer (IBC) and 4 subtypes of breast cancers based on hormone receptor status: (HR+/HER2− breast cancer, HER2+/HR− breast cancer, HER2+/HR+ breast cancer, and triple negative breast cancer (TNBC).

Many baseline features were predictive of breast cancer. Different predictive models were tested for their ability to predict presence of the different types of breast cancer. The first model was based on the variable selection resulting from stepwise logistic regression, a second model that receives baseline features including a PRS and breast density, a third model that receives PRS only as the baseline feature, and a fourth model that receives breast density as the baseline feature. The performance of each model is documented as both the area under the curve (AUC) and the leave one out cross validation (LOOCV) result. In FIG. 8B, the performance is expressed as AUC/LOOCV. PRS and breast density strongly predict breast cancer. The model that receives PRS+breast density as baseline features generally showed similar performance as the first model that is based on stepwise logistic regression and outperformed both the model that receives PRS only as a feature and the model that receives breast density only as a feature. For some cancers (e.g., HER2+/HR− and HER2+/HR+), the model that receives PRS+breast density as baseline features also outperformed the stepwise model.

### 7. Examples of Predictive Cancer Models

Each of the predictive cancer models described within this subjection that generate a cancer prediction based on one or more types of features (e.g., small variant features, whole genome features, methylation features, and/or baseline features) through a logistic regression model, unless stated otherwise. Each predictive cancer model is trained using a set of training data derived from a first subset of patients of a circulating cell-free genome atlas (CCGA) study (NCT02889978) and then subsequently tested using a set of testing data derived from a second subset of patients from the CCGA study.

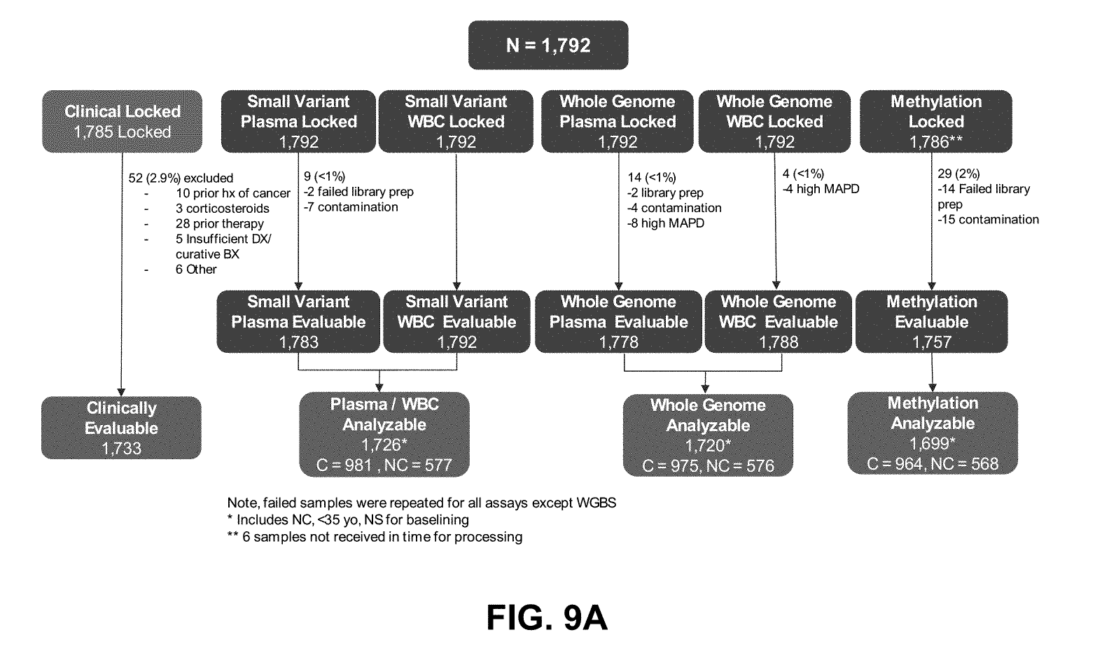

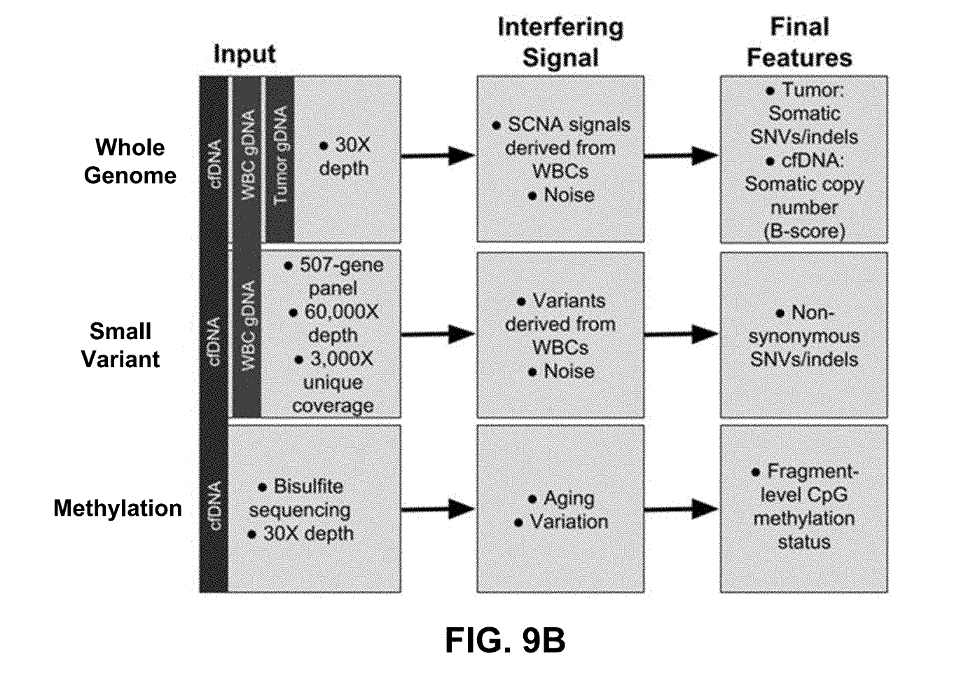

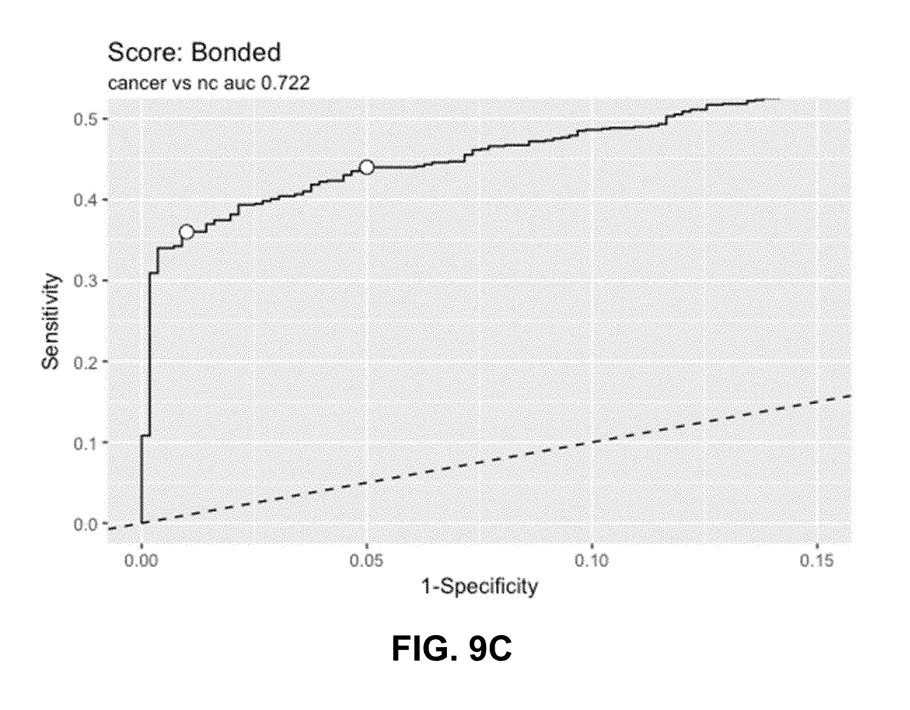

FIG. 9A depicts the experimental parameters of the CCGA study. 1792 patients were categorized in the training set to train the models in the different computational analyses and the predictive cancer models whereas the other 1008 patients were held out of the training as a test set. The 1792 patients were evaluated against eligibility criteria and ineligible patients were removed from training. Altogether, data of 1726 patients (981 cancerous and 577 noncancerous) were used to train the models in the small variant workflow, data of 1720 patients (975 cancerous and 576 noncancerous) were used to train the models for the whole genome workflow, and data of 1699 patients (964 cancerous and 568 noncancerous) were used to train the models for the methylation workflow.

Of the 1792 patients in the training set, 1399 patients passed the eligibility criteria. The 1399 patients, of which 841 were cancerous (with 437 of the 841 presenting with tumor tissue) and 558 were non-cancerous, were used to train the models using a 10-fold cross validation process. The 1008 held out patients were subsequently used to test the performance of the models. FIG. 9B depicts the experimental details (e.g., gene panel, sequencing depth, etc.) used to determine values of features for each respective predictive cancer model. Unless indicated otherwise, the predictive cancer models described below were trained using a plurality of known cancer types from the circulating cell-free genome atlas (CCGA) study. As described above, the CCGA sample set included the following cancer types: breast, lung, prostate, colorectal, renal, uterine, pancreas, esophageal, lymphoma, head and neck, ovarian, hepatobiliary, melanoma, cervical, multiple myeloma, leukemia, thyroid, bladder, gastric, and anorectal. As such, each model described below, unless indicated otherwise, is a multi-cancer model (or a multi-cancer classifier) for detecting of one or more, two or more, three or more, four or more, five or more, ten or more, or 20 or more different types of cancer. In some embodiments, the one or more cancer can be a “high-signal” cancer (defined as cancers with greater than 50% 5-year cancer-specific mortality), such as anorectal, colorectal, esophageal, head & neck, hepatobiliary, lung, ovarian, and pancreatic cancers, as well as lymphoma and multiple myeloma. High-signal cancers tend to be more aggressive and typically have an above-average cell-free nucleic acid concentration in test samples obtained from a patient.

7.1. Multi-Assay Predictive Cancer Model in Accordance with FIG. 1B

FIG. 9C depicts a receiver operating characteristic (ROC) curve of the specificity and sensitivity of a predictive cancer model that predicts the presence of cancer using small variant features, whole genomes features, and methylation features, in accordance with the embodiment shown in FIG. 1B. Here, the predictive cancer model is a penalized logistic regression model and outputs a score (e.g., a Bonded score). The Bonded score is then used to predict the presence or absence of cancer. Specifically, the small variant features of the predictive cancer model include a presence/absence of somatic variants per gene in a gene panel (a total of 469 gene vectors), the whole genome features of the predictive cancer model include reduced dimensionality features (a total of 5 reduced dimensionality features), and the methylation features of the predictive cancer model include rankings based on hypermethylation scores and rankings based on hypomethylation scores. In particular, the rankings based on hypermethylation scores includes 7 hypermethylated fragments (e.g., the rank 1, rank 2, rank 4, rank 8, rank 16, rank 32, and rank 64 hypermethylated fragments). Similarly, the rankings based on hypomethylation scores includes 7 hypomethylated fragments (e.g., the rank 1, rank 2, rank 4, rank 8, rank 16, rank 32, and rank 64 hypomethylated fragments). The total AUC of the ROC curve is 0.722. FIG. 9C depicts the performance of the predictive cancer model in a 85%-100% specificity range. In this example, the ROC curve indicates a ˜44% sensitivity at a 95% specificity and a ˜36% sensitivity at a 99% specificity.

7.2. Single-Assay Predictive Cancer Model in accordance with FIG. 1C

7.2.1. Small Variant Assay Predictive Cancer Model

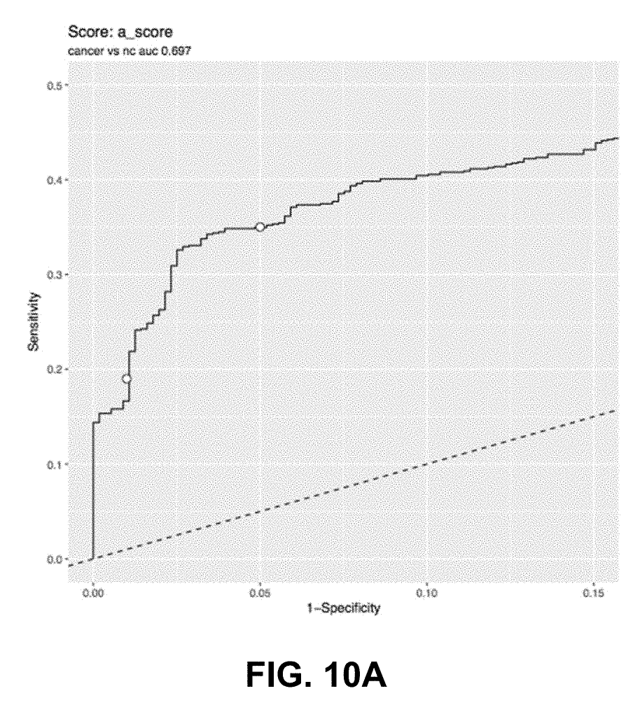

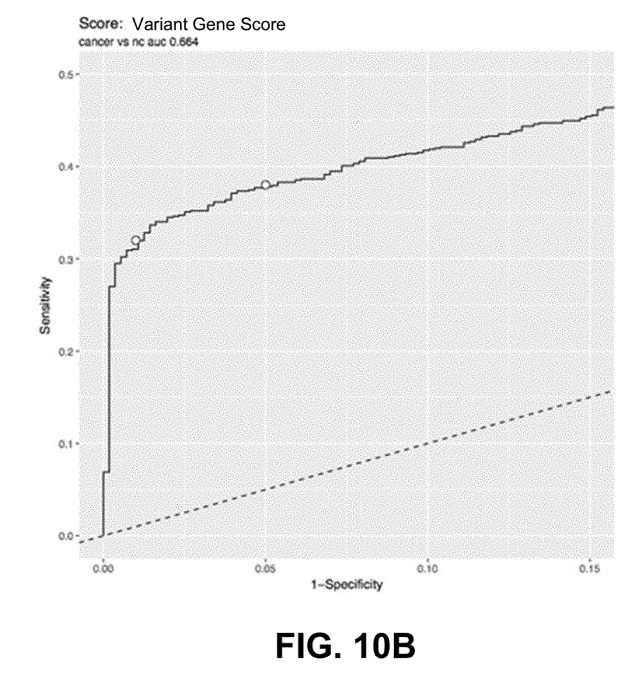

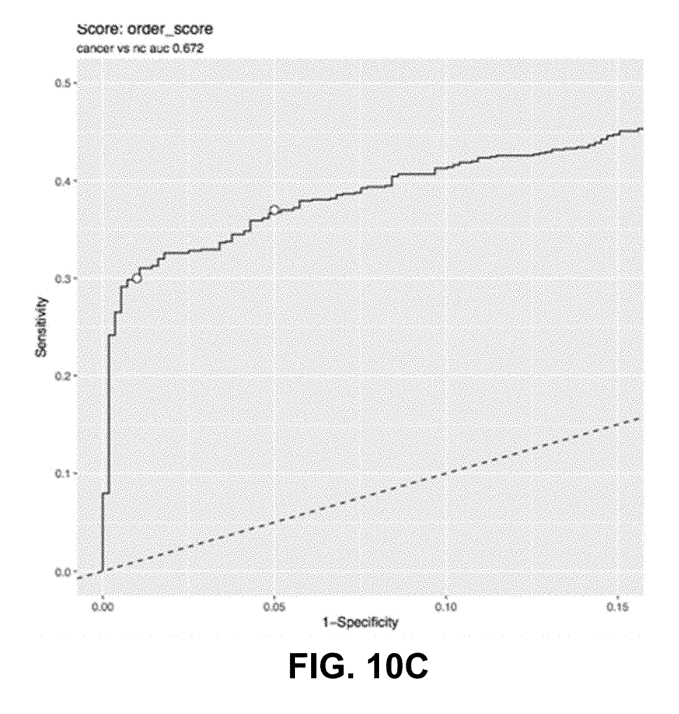

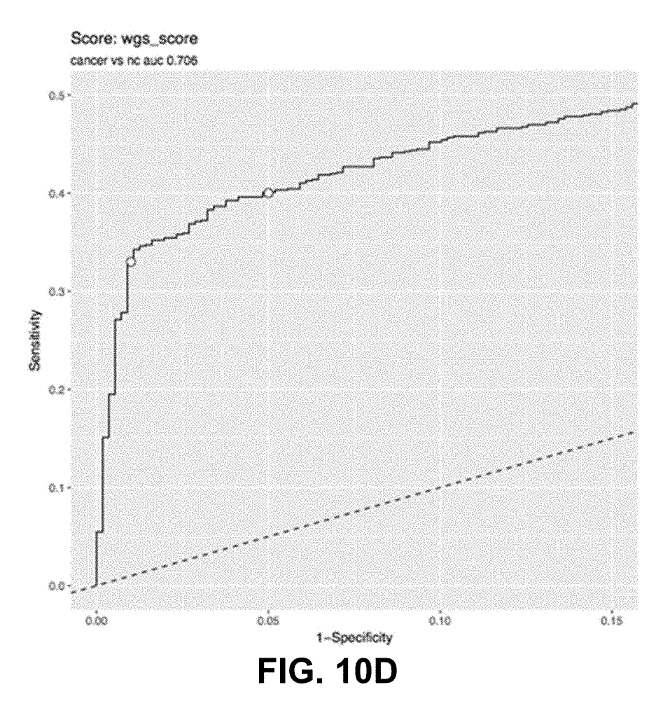

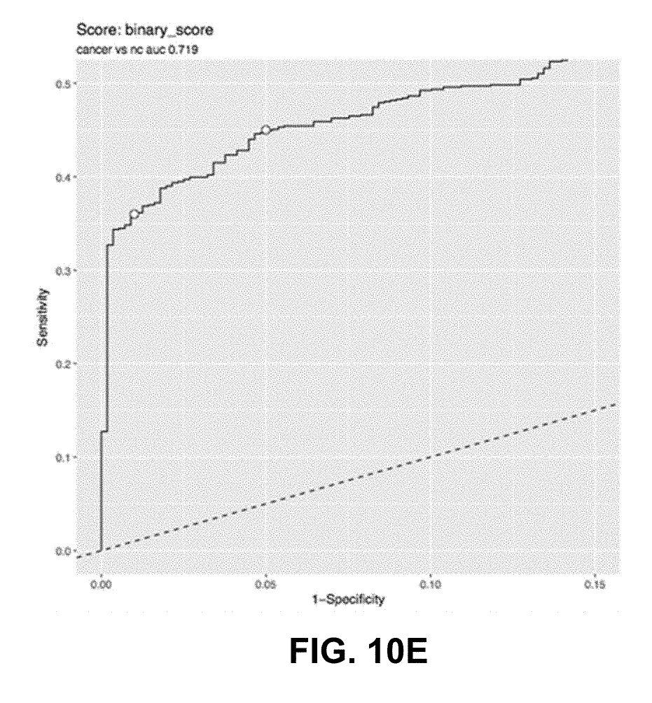

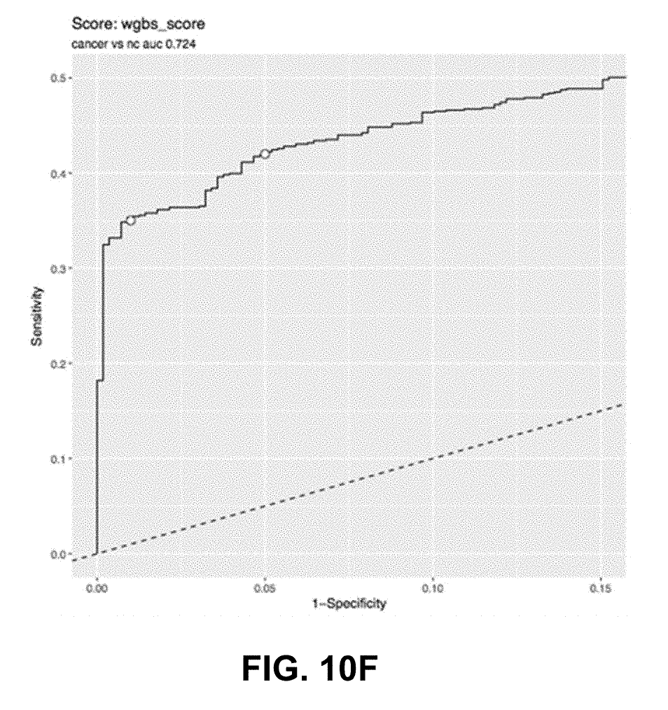

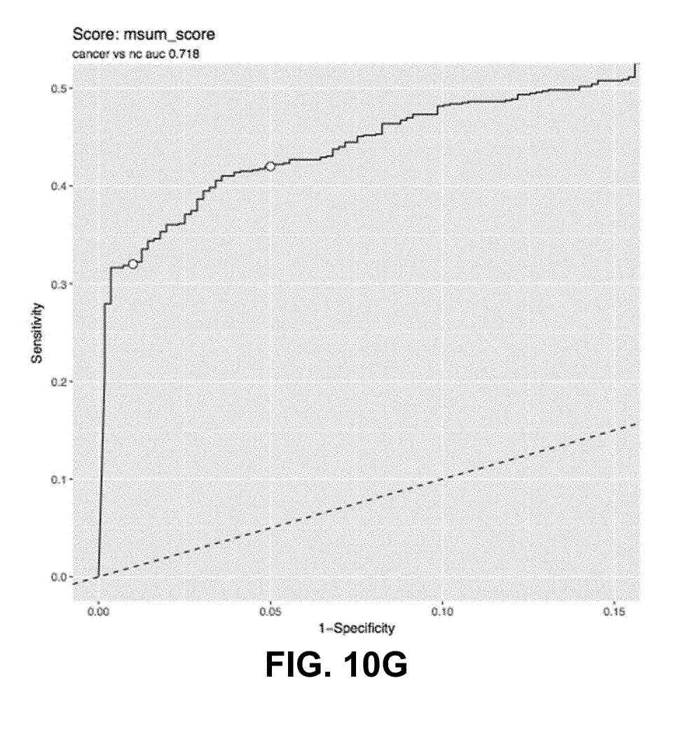

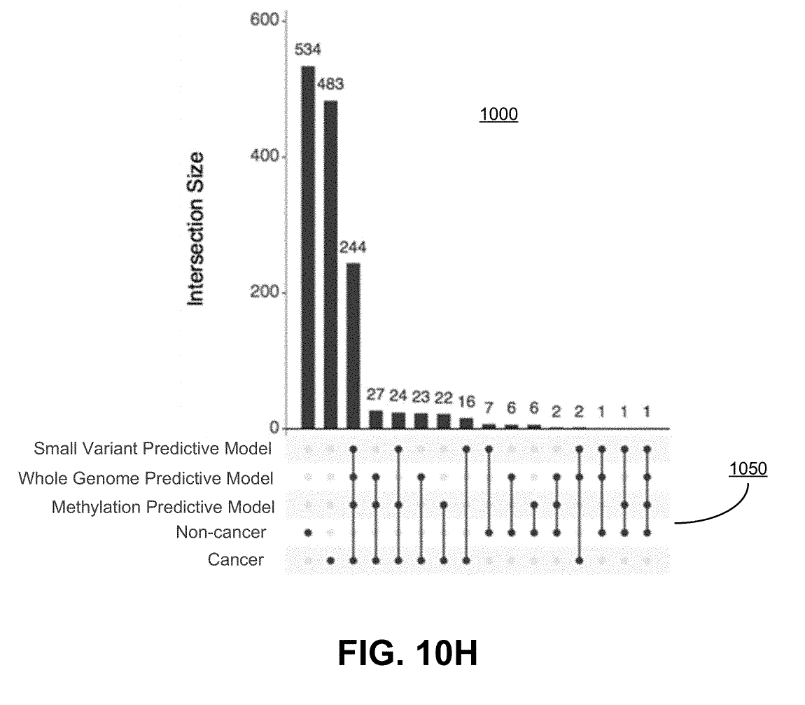

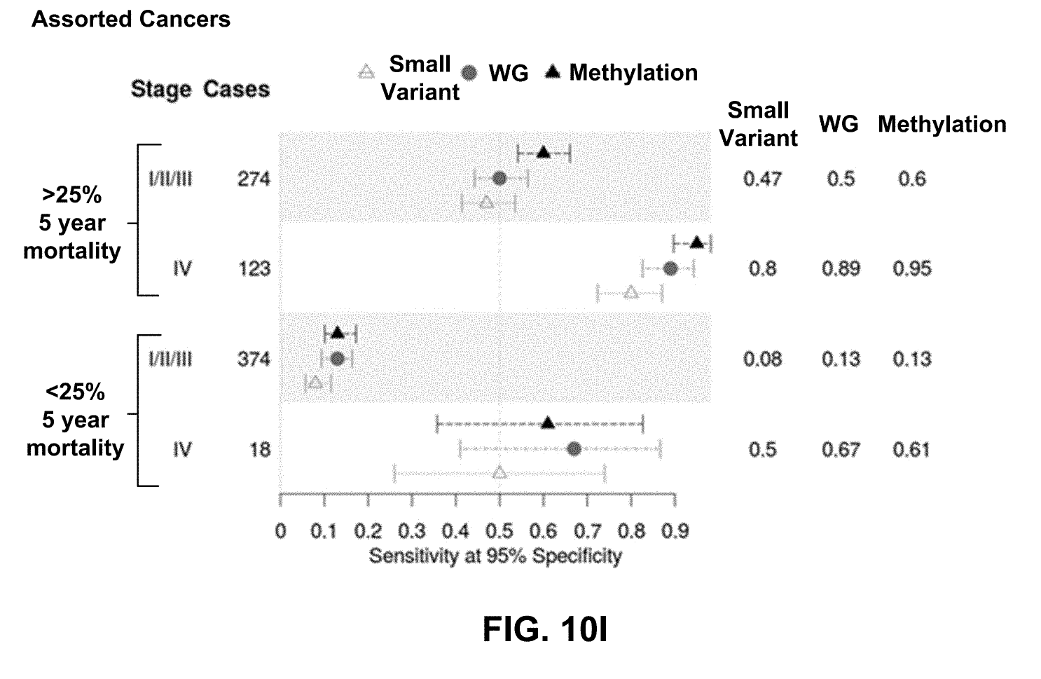

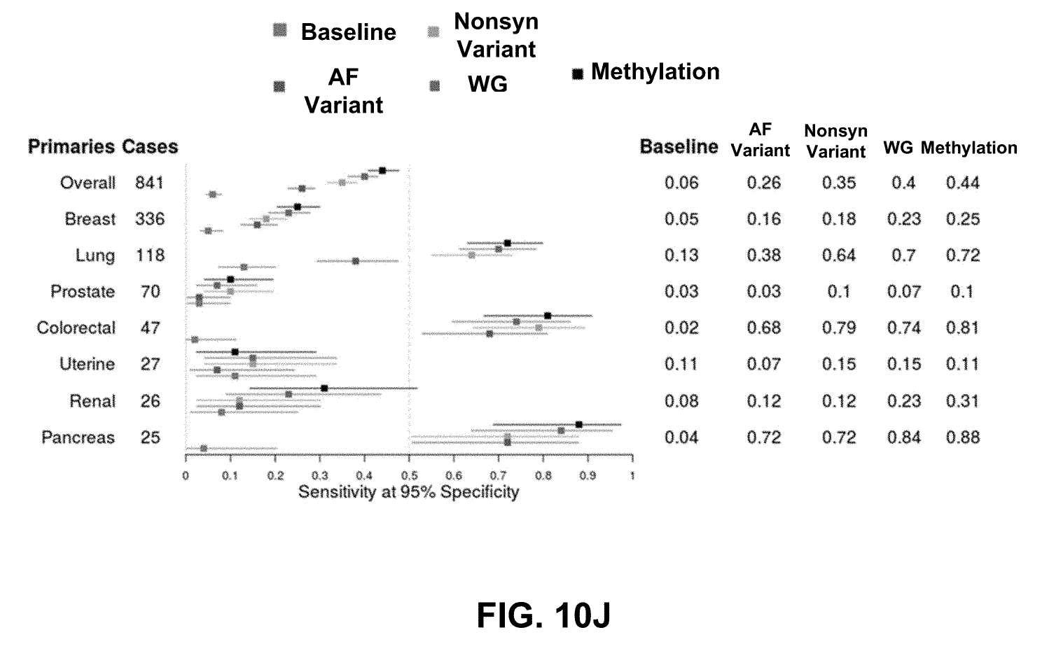

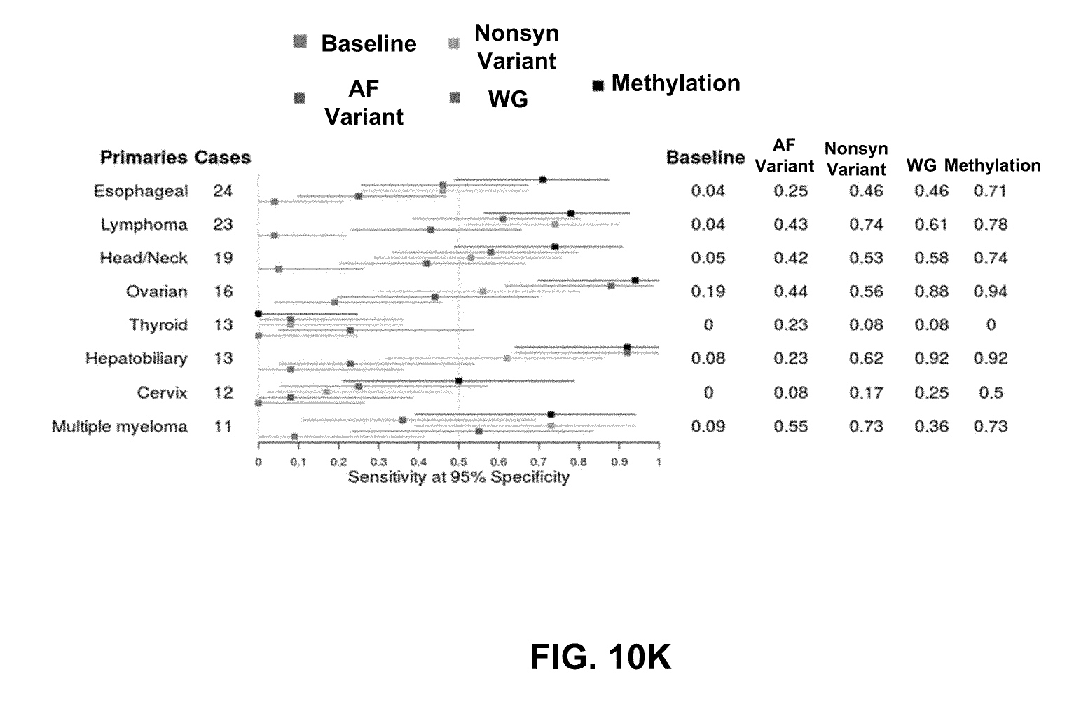

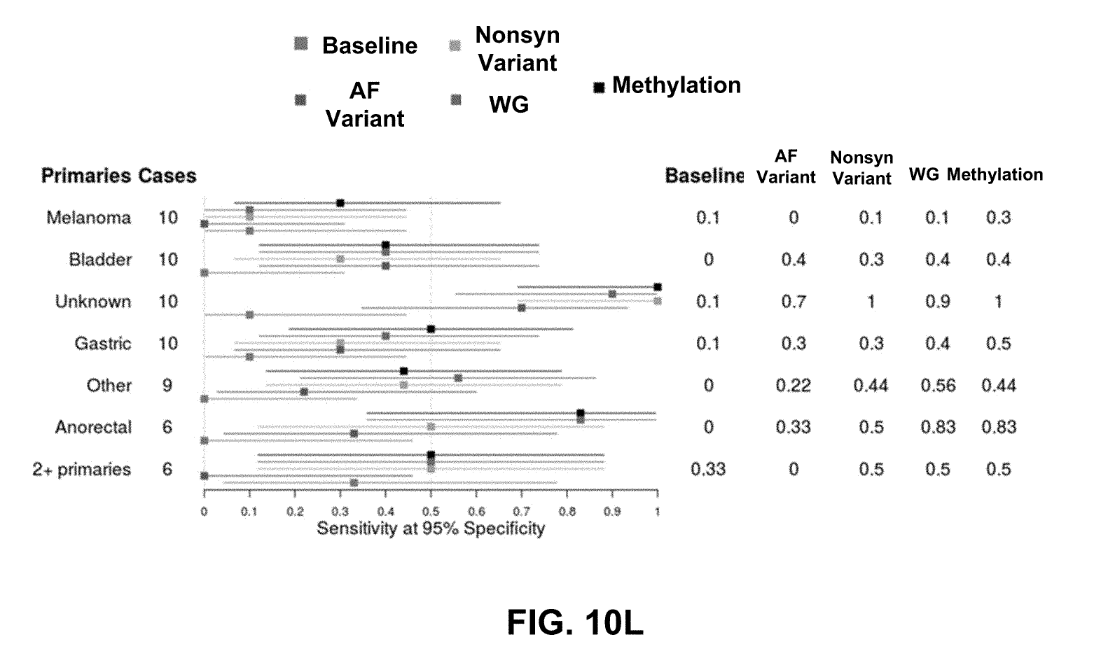

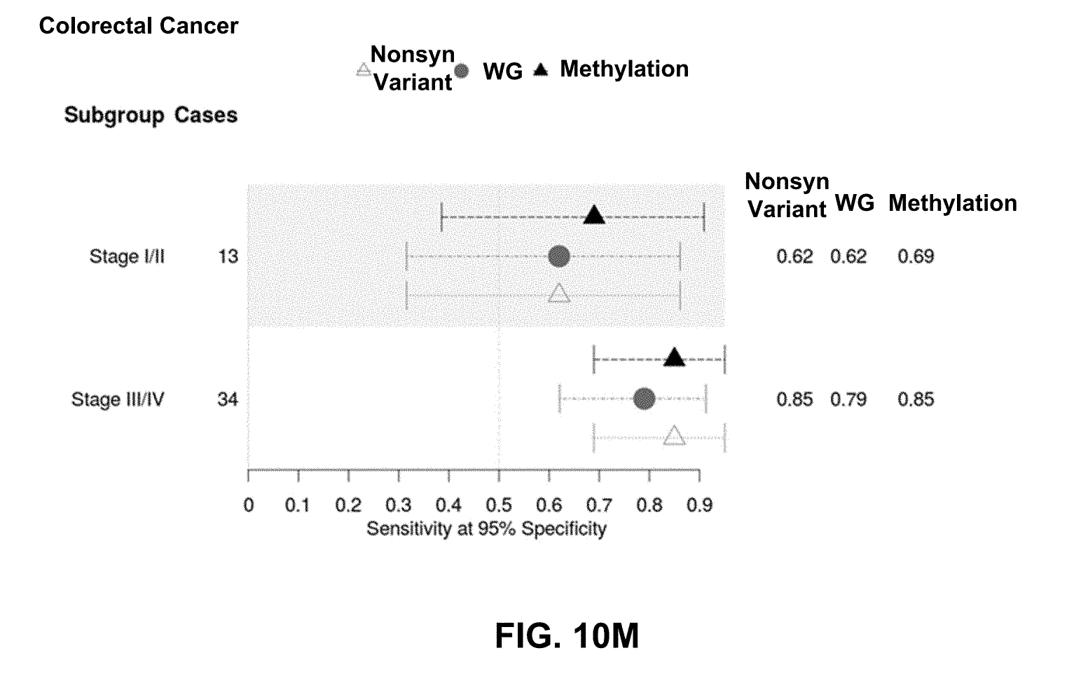

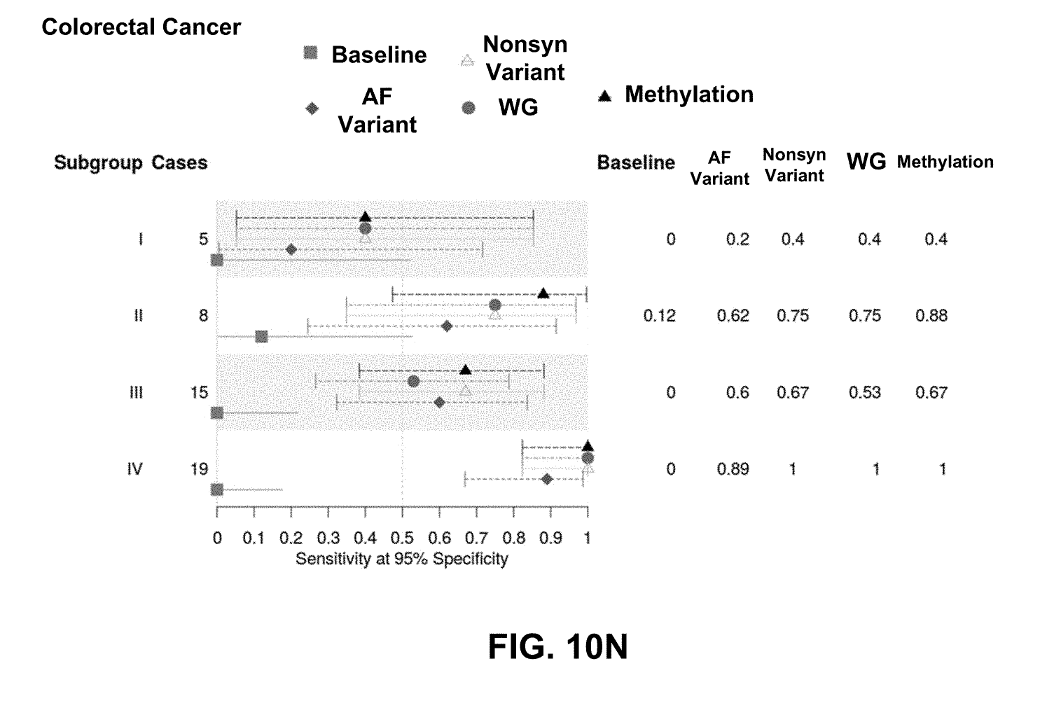

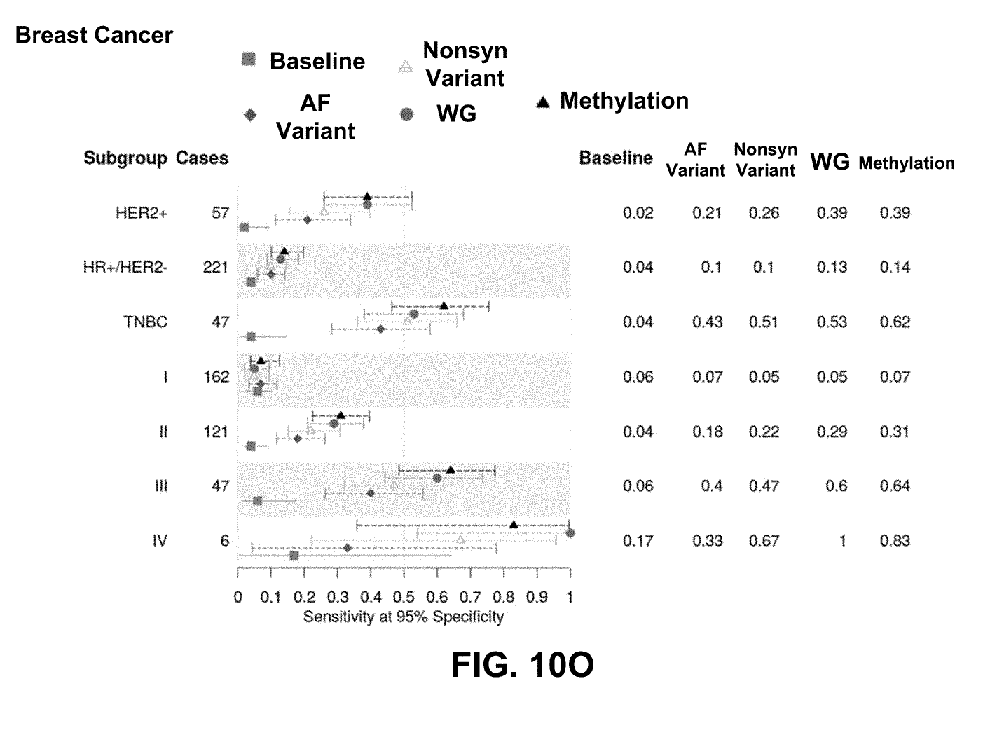

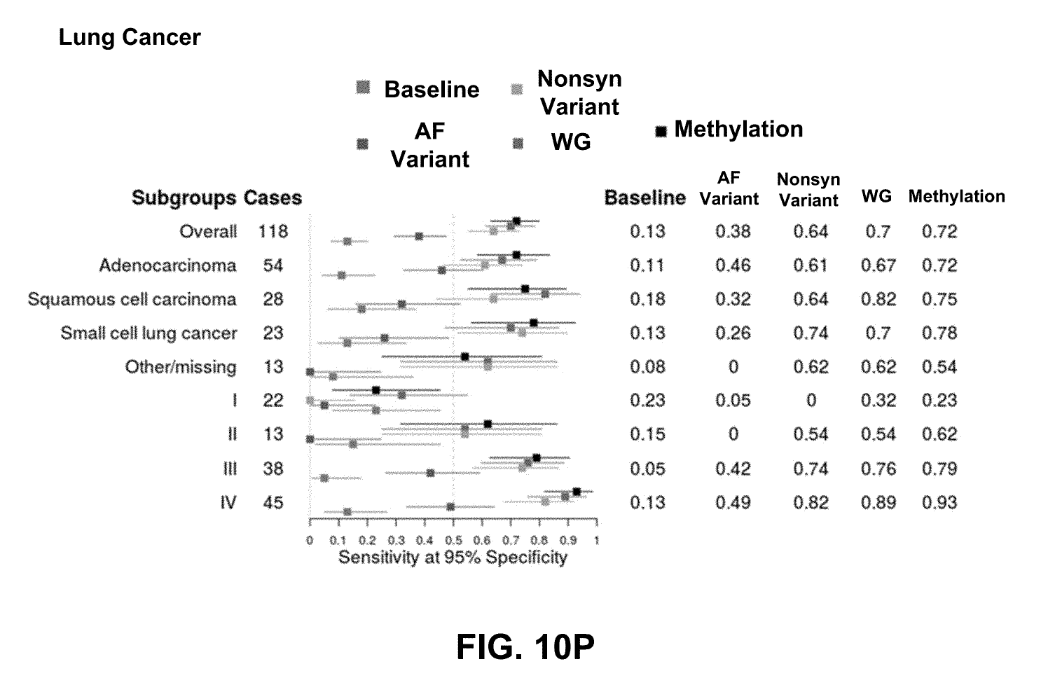

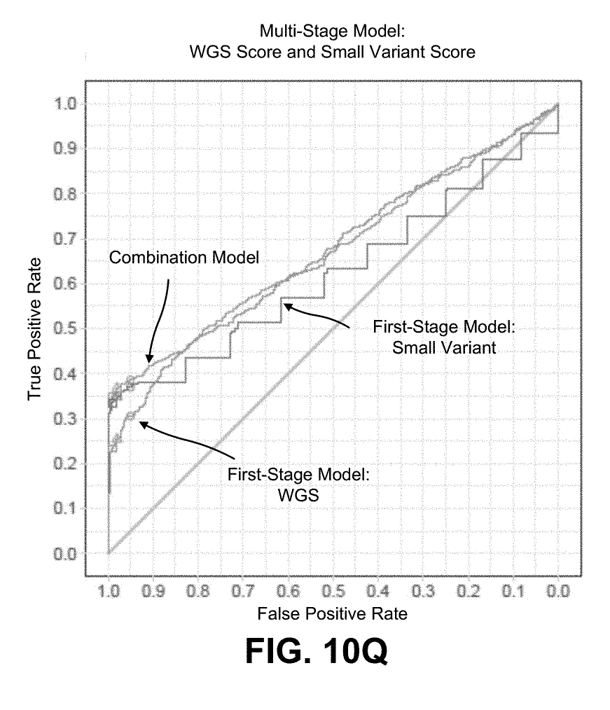

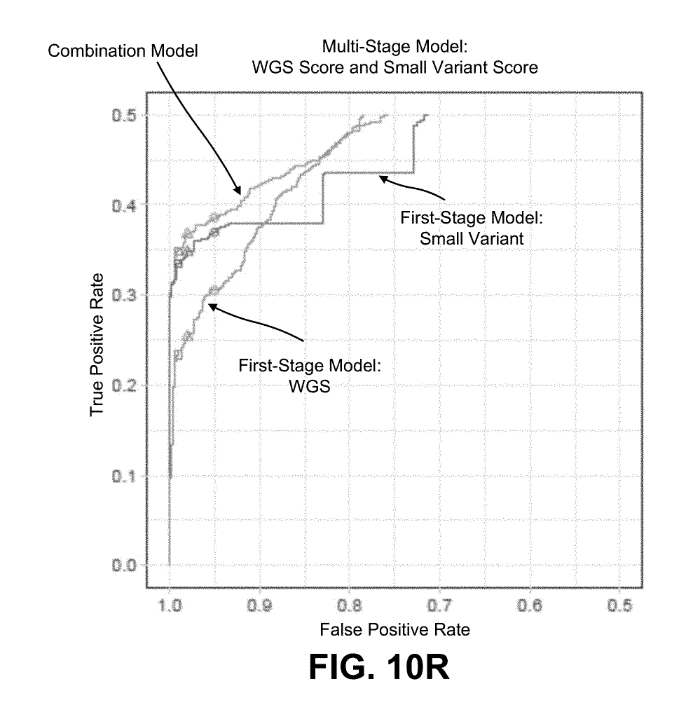

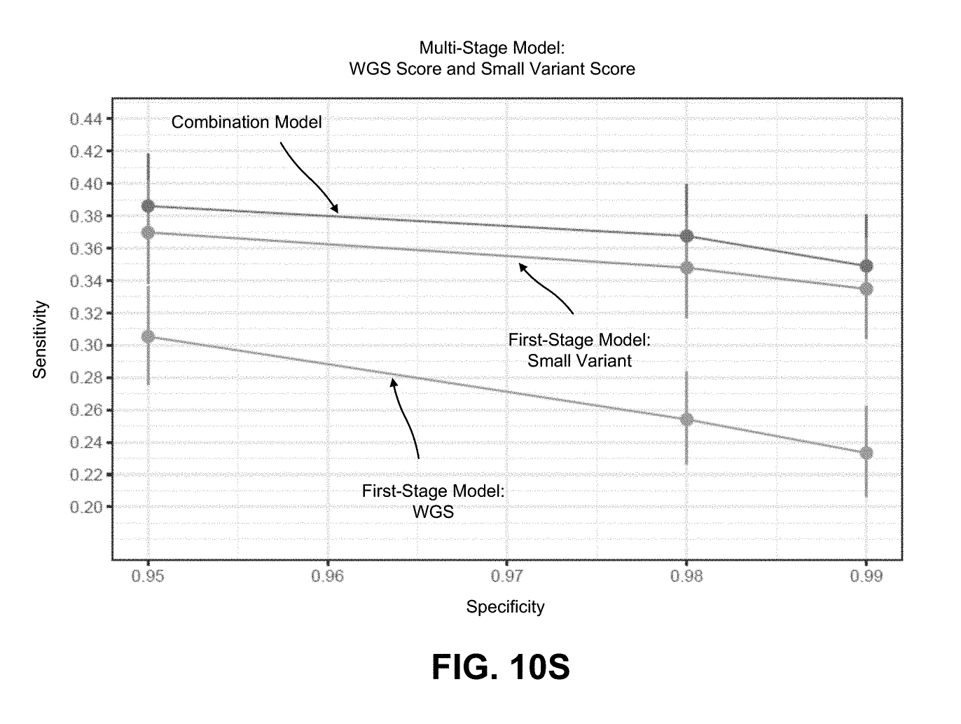

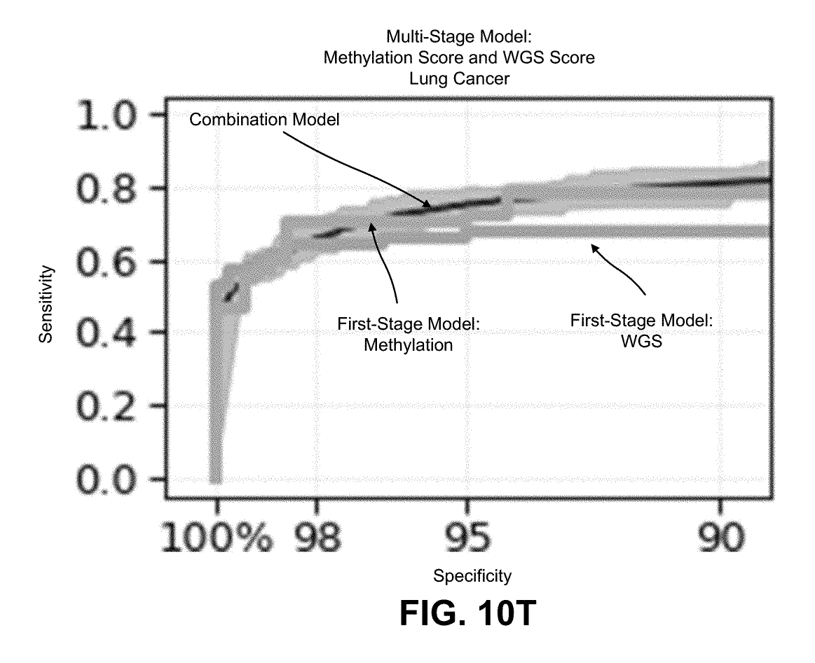

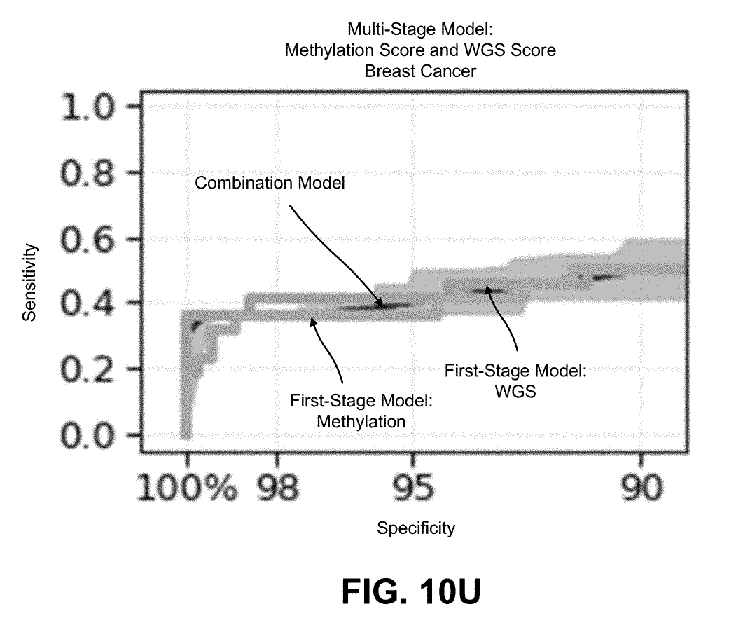

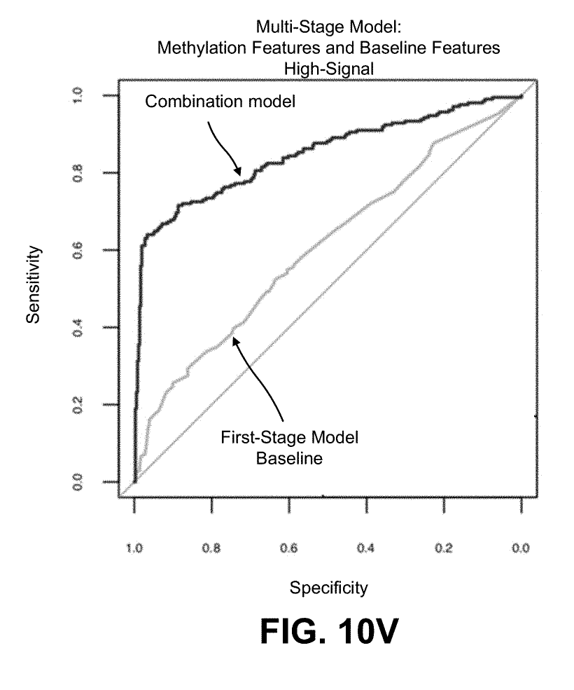

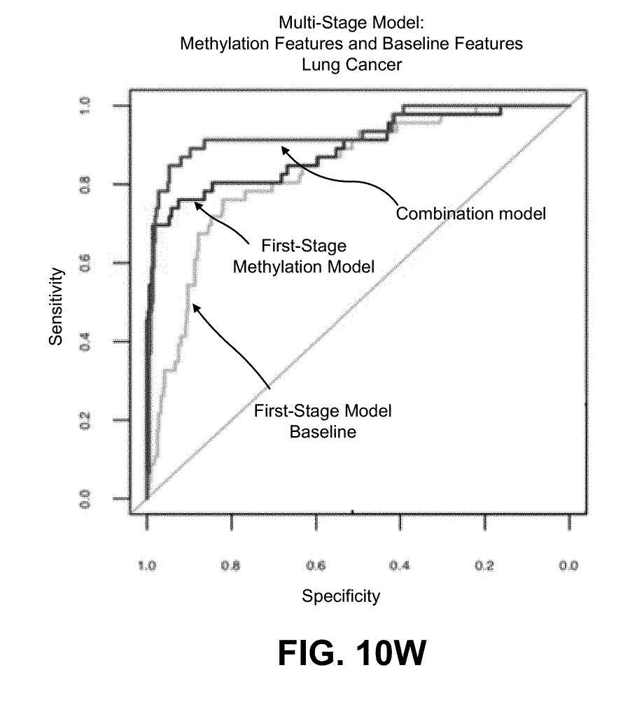

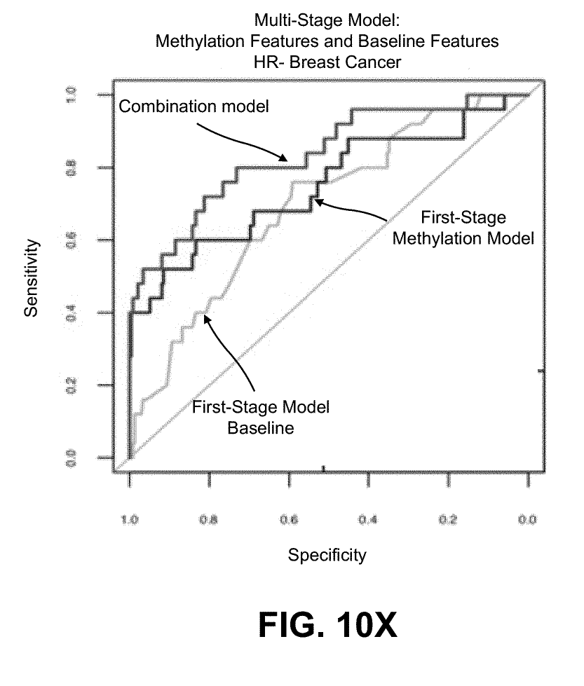

FIG. 10A depicts a receiver operating characteristic (ROC) curve of the specificity and sensitivity of a predictive cancer model that predicts the presence of cancer using a first set of small variant features. Specifically the predictive cancer model outputs a score, hereafter referred to as an “A_score” that indicates the presence or absence of cancer. The total area under the curve (AUC) of the ROC curve is 0.697. Given that the goal is to achieve a sensitivity given a set specificity (e.g., 95% or 99% specificity), FIG. 10A depicts the performance of a predictive cancer model in a 85%-100% specificity range. In this example, the small variant features provided to the predictive cancer model include: a total number of somatic variants and a total number of nonsynonymous variants. The ROC curve indicates a 35% sensitivity at a 95% specificity and a ˜19% sensitivity at a 99% specificity. Proceeding from 99% specificity to 95% specificity, the ROC curve increases non-linearly, thereby indicating that there likely true positives detected in this sensitivity/specificity tradeoff.

FIG. 10B depicts a ROC curve of the specificity and sensitivity of a predictive cancer model that predicts the presence of cancer using a second set of small variant features. Specifically the predictive cancer model outputs a score, hereafter referred to as a variant gene score that indicates the presence or absence of cancer. The total AUC of the ROC curve is 0.664. FIG. 10B depicts the performance of the predictive cancer model in a 85%-100% specificity range. In this example, the small variant features provided to the predictive cancer model include an AF of somatic variants per gene. Here, the AF of somatic variants per gene represents the maximum AF of a somatic variant in each gene. Therefore, a total of 500 values of the maximum AF of somatic variants per gene (corresponding to 500 genes) were provided as feature values to the predictive cancer model. The ROC curve indicates a ˜38% sensitivity at a 95% specificity and a ˜31% sensitivity at a 99% specificity. This represents an improvement in comparison to the results of the predictive cancer model shown in FIG. 10A.

FIG. 10C depicts a ROC curve of the specificity and sensitivity of a predictive cancer model that predicts the presence of cancer using a third set of small variant features. Specifically the predictive cancer model outputs a score, hereafter referred to as an Order score that indicates the presence or absence of cancer. The total AUC of the ROC curve is 0.672. FIG. 10C depicts the performance of the predictive cancer model in a 85%-100% specificity range. In this example, the small variant features of the predictive cancer model include the top 6 ranked order according to AF of somatic variants. The ROC curve indicates a ˜37% sensitivity at a 95% specificity and a ˜30% sensitivity at a 99% specificity. Again, this represents an improvement in comparison to the results of the predictive cancer model shown in FIG. 10A.

7.2.2. Whole Genome Predictive Cancer Model

FIG. 10D depicts a ROC curve of the specificity and sensitivity of a predictive cancer model that predicts the presence of cancer using a set of whole genome features. Specifically the predictive cancer model outputs a score, hereafter referred to as a WGS score that indicates the presence or absence of cancer. The total AUC of the ROC curve is 0.706. FIG. 10D depicts the performance of the predictive cancer model in a 85%-100% specificity range. In this example, the whole genome features of the predictive cancer model include bin scores of bins across the genome and reduced dimensionality features (e.g., values derived based on PCA). In totality, 37,000 feature values representing bin scores of bins across the genome whereas features values for 5-200 lower dimensionality components were provided as input to the cancer predictive model. The ROC curve indicates a ˜40% sensitivity at a 95% specificity and a ˜33% sensitivity at a 99% specificity.

7.2.3. Methylation Predictive Cancer Model

FIG. 10E depicts a ROC curve of the specificity and sensitivity of a predictive cancer model that predicts the presence of cancer using a first set of methylation features. Here, the predictive cancer model is a penalized logistic regression model. Specifically the predictive cancer model outputs a score, hereafter referred to as a Binary score that indicates the presence or absence of cancer. The total AUC of the ROC curve is 0.719. FIG. 10E depicts the performance of the predictive cancer model in a 85%-100% specificity range. In this example, the methylation features of the predictive cancer model include rankings based on hypermethylation scores and rankings based on hypomethylation scores. In particular, the rankings based on hypermethylation scores includes 7 hypermethylated fragments (e.g., the rank 1, rank 2, rank 4, rank 8, rank 16, rank 32, and rank 64 hypermethylated fragments). Similarly, the rankings based on hypomethylation scores includes 7 hypomethylated fragments (e.g., the rank 1, rank 2, rank 4, rank 8, rank 16, rank 32, and rank 64 hypomethylated fragments). The ROC curve indicates a ˜45% sensitivity at a 95% specificity and a ˜36% sensitivity at a 99% specificity.

FIG. 10F depicts a ROC curve of the specificity and sensitivity of a predictive cancer model that predicts the presence of cancer using a second set of methylation features. Specifically the predictive cancer model outputs a score, hereafter referred to as a WGBS score that indicates the presence or absence of cancer. The total AUC of the ROC curve is 0.724. FIG. 10F depicts the performance of the predictive cancer model in a 85%-100% specificity range. In this example, the methylation features of the predictive cancer model include the presence or absence of abnormally methylated (e.g., hypomethylated or hypermethylated) fragment at CpG sites. In this particular example, the CpG sites that provide the most information gain (e.g., ˜3000 CpG sites) were identified. Therefore, there are a total of ˜3000 feature values for this feature, each feature value being either 0 or 1 that indicates whether there is an abnormally methylated fragment at an identified CpG site. The ROC curve indicates a ˜42% sensitivity at a 95% specificity and a ˜35% sensitivity at a 99% specificity.

FIG. 10G depicts a ROC curve of the specificity and sensitivity of a predictive cancer model that predicts the presence of cancer using values of a third set of methylation features. Specifically the predictive cancer model outputs a score, hereafter referred to as a MSUM score that indicates the presence or absence of cancer. The total AUC of the ROC curve is 0.718. FIG. 10G depicts the performance of the predictive cancer model in a 85%-100% specificity range. In this example, the methylation features of the predictive cancer model include the mean value across the features of the presence or absence of abnormally methylated (e.g., hypomethylated or hypermethylated) fragment at CpG sites. In this example, there is one mean value that represents the average of a total of the ˜3000 values described above in relation to FIG. 10F. Here, the values are either 0 or 1 and indicate whether there is an abnormally methylated fragment at a CpG site of interest. The ROC curve indicates a ˜42% sensitivity at a 95% specificity and a ˜32% sensitivity at a 99% specificity.

7.2.4. Summary of Results of Single-Assay Predictive Cancer Models

FIG. 10H depicts the performance of each of the single-assay predictive cancer models (e.g., predictive cancer models applied for features derived from sequence reads generated from each of the small variant sequencing assay, whole genome sequencing assay, and methylation sequencing assay). In particular, the predictive cancer model that includes features derived from sequence reads generated by the small variant sequencing assay refers to the predictive cancer model that outputs a variant gene score, as described above in FIG. 10B. The predictive cancer model that includes features derived from sequence reads generated by the whole genome sequencing assay refers to the predictive cancer model that outputs a WGS score, as described above in FIG. 10D. The predictive cancer model for features derived from sequence reads generated from the methylation sequencing assay refers to the predictive cancer model that outputs a WGBS score, as described above in FIG. 10F.

FIG. 10H includes a graph 1000 that depicts the quantitative intersection between the different predictive models and non-cancer/cancer. Additionally, FIG. 10H depicts a matrix 1050 below the graph that denotes the identification (e.g., solid dot) or non-identification (e.g., empty dot) of cancer by a predictive model.

As shown in FIG. 10H, 534 known non-cancerous patients and 483 known cancer patients were analyzed using each of the predictive cancer models. Of the 483 known cancer patients, each of the three predictive cancer models successfully called the presence of cancer in 244 of those patients. Additionally, the whole genome predictive model and the methylation predictive model, but not the small variant predictive model, successfully called the presence of cancer in 27 additional patients. Further intersections of successfully called cancer between one or more predictive models is shown in the matrix 1050.

Of note, there are five patients that are clinically non-cancerous, but at least two of the predictive models called a presence of cancer. Of these five patients, one of the patients was subsequently clinically diagnosed with cancer. The additional four patients may be false positives.

7.3. Additional Single Assay Predictive Cancer Models

FIG. 10I depicts the performance of predictive cancer models for different types of cancer across different stages. Specifically, FIG. 10I depicts the sensitivity of each single-assay predictive cancer model (e.g., small variant predictive cancer model, whole genome (WG) predictive cancer model, and methylation predictive cancer model) at a 95% specificity. Here, the small variant predictive cancer model refers to the predictive cancer model that includes a feature of the total number of nonsynonymous variants and outputs an A score. An example of this small variant predictive cancer model is described above in relation to FIG. 10A. The WG predictive cancer model refers to a predictive cancer model that includes whole genome features including bin scores of bins across the genome and reduced dimensionality features (e.g., values derived based on PCA). The WG predictive cancer model outputs a WGS score. An example of this whole genome predictive cancer model is described above in relation to FIG. 10D. The methylation predictive cancer model refers to the predictive cancer model that includes methylation features including the presence or absence of abnormally methylated (e.g., hypomethylated or hypermethylated) fragment at CpG sites. The methylation predictive cancer model outputs a WGBS score and is described above in relation to FIG. 10F.

FIG. 10I further divides up the performance of predictive cancer models for cancers that have a greater than 25% 5 year mortality rate and cancers that have a less than 25% 5 year mortality rate. Cancers with a greater than 25% 5 year mortality rate include anorectal, cervical, colorectal, esophageal, gastric, head and neck, hepatobillary, lung, lymphoma, multiple myeloma, ovarian, pancreatic ductal adenocarcinoma, renal, and triple-negative breast cancer. Cancers with a less than 25% 5 year mortality rate include bladder, ER+ breast cancer, melanoma, prostate, thyroid, and uterine cancer. As depicted in FIG. 10I, the sensitivity of the prediction models is improved for late stage cancer (e.g., stage IV cancer) in comparison to the sensitivity of the prediction models at earlier stage cancers (e.g., stages I/II/III cancer). Furthermore, as depicted in FIG. 10I, the single-assay models described herein showed higher sensitivity at 95% specificity for cancers with greater than 25% 5 year mortality. For example, at 95% specificity, the methylation assay showed a 60% detection sensitivity (at stages I/II/III) in cancers with high mortality (greater than 25% 5 year mortality) versus a 13% detection sensitivity (at stages I/II/III) in cancers with low mortality (less than 25% 5 year mortality).

FIGS. 10J-10L each depicts the performance of additional predictive cancer models (in addition to the small variant (referred to in each of FIGS. 10J-10L as the nonsynonymous variant), WG, and methylation predictive cancer models shown in FIG. 10I) for different types of invasive cancers. The additional predictive cancer models include a baseline predictive model that receives, as input, values of age, smoking habits, and family history of cancer, as well as a small variant predictive model (referred to as AF Variant) that receives values of the feature of AF of somatic variants per gene. The particular small variant predictive model (e.g., AF variant) is described above in relation to FIG. 10B.

Specifically, FIG. 10J depicts the performance of predictive cancer models at 95% specificity for overall cancers, breast, lung, prostate, colorectal, uterine, renal, and pancreatic cancers. FIG. 10K depicts the performance of predictive cancer models at 95% specificity for esophageal, lymphoma, head/neck, ovarian, thyroid, hepatobiliary, cervix, and multiple myeloma cancers. FIG. 10L depicts the performance of predictive cancer models at 95% specificity for melanoma, bladder, unknown, gastric, anorectal, 2+ primary cancers, and other cancers.

FIG. 10M depicts the performance of predictive cancer models for different stages of colorectal cancer. Specifically, FIG. 10M depicts the sensitivity of each single-assay predictive cancer model at a 95% specificity. Here, each of the predictive cancer models exhibit a greater than 50% sensitivity at early stage (e.g., stages I/II) and late stage (e.g., stages Ill/IV) cancers.

FIG. 10N depicts the performance of additional predictive cancer models (in addition to the nonsynonymous variant, WG, and methylation predictive cancer models shown in FIG. 10M) for different stages of colorectal cancer. FIG. 10N depicts the sensitivity of each single-assay predictive cancer model at a 95% specificity.

FIG. 10O depicts the performance of predictive cancer models for different stages and different types of breast cancer. FIG. 10O depicts the sensitivity of each single-assay predictive cancer model at a 95% specificity. Here, FIG. 10O shows that the performance of predictive cancer models are different for different types of breast cancer. For example, each of the AF Variant, Nonsynonymous Variant, WG, and Methylation predictive cancer models are more sensitive for triple negative breast cancer (TNBC) in comparison to HER2+ and HR+/HER2− breast cancers. Additionally, the sensitivity of predictive cancer models are improved for later stage breast cancers in comparison to early stage breast cancers. Furthermore, as also shown in FIG. 10L, at 95% specificity, detection sensitivity for TNBC is higher than HER2+ or HR+/HER2−, respectively (e.g., 62% for TNBC versus 39% and 14%, respectively, using the methylation assay).

FIG. 10P depicts the performance of predictive cancer models for different stages and different types of lung cancer. FIG. 10P depicts the sensitivity of each single-assay predictive cancer model at a 95% specificity. Here, FIG. 10P shows that the performances of predictive cancer models are different for different types of lung cancer. For example, the sensitivity of predictive cancer models are improved for later stage lung cancers in comparison to early stage lung cancers. Additionally, generally, each of the Nonsynonymous Variant, WG, and Methylation predictive cancer models exhibit higher sensitivities across the different types and stages of lung cancer in comparison to the baseline and AF variant predictive models.

7.4. Multi-Stage, Multi-Assay Predictive Cancer Model in Accordance with FIG. 1D

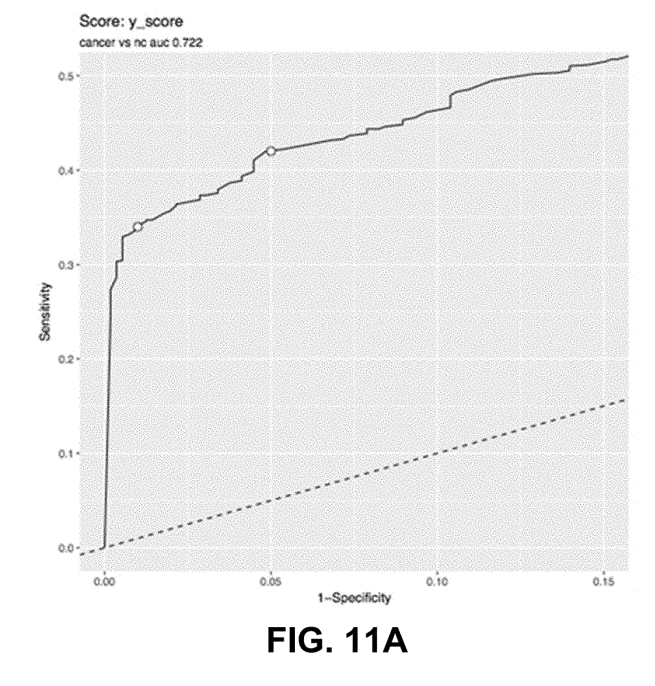

FIG. 11A depicts a ROC curve of the specificity and sensitivity of a two-stage predictive cancer model that predicts the presence of cancer. Here, the predictive cancer model includes four first stage models (e.g., predictive models 180 shown in FIG. 1D) that are each logistic regressions. The predictive cancer model further includes a second stage model (e.g., overall predictive model 185 shown in FIG. 1D), where the second stage model is a random forest model.

In accordance with FIG. 1D, a first stage of the predictive cancer model includes separate predictive models 180B, 180C, and 180D that respectively analyze whole genome features, small variant features, and methylation features. Specifically, the predictive model 180B is a predictive model that outputs a WGS score, as is described in relation to FIG. 10D. The predictive model 180C is a combination of a predictive cancer model that outputs a variant gene score, as is described above in relation to FIG. 10B, and a predictive cancer model that outputs an Order score, as is described above in relation to FIG. 10C. The predictive model 180D is a combination of a predictive cancer model that outputs a Binary score, as is described in relation to FIG. 10E, a predictive cancer model that outputs a WGBS score, as is described in relation to FIG. 10F, and a predictive cancer model that outputs a MSUM score, as is described in relation to FIG. 10G.

The second stage of the predictive cancer model is an overall predictive model is trained to output a cancer prediction using values of each of the WGS score, variant gene score, Order score, Binary score, WGBS score, and MSUM score. Therefore, during deployment, the second stage of the predictive cancer model receives, as input, the various scores and outputs a cancer prediction for an individual 110, such as a presence/absence of cancer in the individual.

The total AUC of the ROC curve is 0.722. FIG. 11A depicts the performance of the predictive cancer model in a 85%-100% specificity range. The ROC curve indicates a ˜43% sensitivity at a 95% specificity and a ˜34% sensitivity at a 99% specificity.

7.5. Example Multi-Stage, Multiple Assay Predictive Cancer Models

As described above in FIG. 1D, a predictive cancer model (e.g., predictive cancer model 195) may be a multi-stage multiple assay predictive cancer model (“multi-stage model”). A multi-stage model includes two or more first-stage predictive cancer models (e.g., predictive cancer model 180A, 180B, 180C and 180D) that each generate a cancer prediction for a subject. Each of the first-stage predictive cancer models (“first-stage model”) generates a cancer prediction from one or more features determined from a test sample obtained from an individual (e.g., individual 110).

In an embodiment, each first-stage model is configured to generate a cancer prediction from a similar type of features (e.g., features from a single sequencing assay). For example, a first first-stage model may generate a cancer prediction for the individual using small variant features (e.g., small variant features 154), a second first-stage model may generate a cancer prediction using methylation features (e.g., methylation features 156), etc. In another embodiment, each first stage model is configured to generate a cancer prediction from one or more types of features. For example, a first first-stage model may generate a cancer prediction for the individual using small variant features and methylation features, and the second first-stage model may generate a cancer prediction using whole genome features (e.g., whole genome features 152) and baseline features (e.g., baseline features 150).

There are many possible configurations of first-stage models for a multi-stage model. For example, there may be two, three, four, five, etc. first-stage models included in a multi-stage model. As another example, there may be one, two, three, four, five, etc. types of features used as an input to a first stage model.

A multi-stage model includes an overall predictive model (e.g., overall predictive model 185) that generates a cancer prediction (e.g., cancer prediction 190). The overall predictive model (“second-stage model”) inputs the cancer predictions of the first-stage models and generates the cancer prediction. The second-stage model may be trained using any of the various methods described herein. As an example, the second-stage model may be a random-forest model trained to determine a cancer prediction using features derived from test sequences with a known cancer diagnosis.

There are many possible configurations of second-stage models for a multi-stage model. For example, the multi-stage model may be trained to determine a cancer prediction using two, three, four, five, etc. cancer predictions generated from their corresponding first-stage models. As another example, a multi-stage model may have more than two stages. To illustrate, the results of two second-stage models may be input into a cancer prediction model (“third-stage model) trained to determine a cancer prediction for an individual.

7.5.1. Example 1: First-Stage Models Using WGS and Small Variant Features

As described above, one example of a multi-stage model includes two first-stage models and a single second-stage model. In this example, the first first-stage model (“first model”) is a cancer prediction model configured to determine a cancer prediction for an individual using small variant features. The features input into the first model may be derived using techniques similar to those described in Section 2, “Small Variant Computation Analysis.” The first model may output a score (e.g., an A score) indicating a cancer prediction for the individual. Section 7.2.1, “Small Variant Assay Predictive Caner Model,” describes a predictive cancer model that may be used as the first model. FIGS. 10A-IOC illustrate examples of the predictive capability of a first model. Further, in this example, the second first-stage model (“second model”) is a cancer prediction model configured to determine a cancer prediction for an individual using WGS features. The features input into the second model may be derived using techniques similar to those described in Section 3, “Whole Genome Computational Analysis.” The second model may output a score (e.g., a WGS score) indicating a cancer prediction for the individual. Section 7.2.2, “Whole Genome Predictive Cancer Model,” describes a predictive cancer model that may be used as the second model. FIG. 10D illustrates an example of the predictive capability of the second model. Finally, in this example, the second-stage model (“combined model”) is configured to generate a cancer prediction using the cancer predictions of the first model and second model. Here, the second-stage model employs a second layer regularized logistic regression algorithm to determine a cancer prediction, but could employ other algorithms.

FIG. 10Q illustrates a ROC curve plot for a multi-stage model, according to a first example embodiment. FIG. 10R illustrates the ROC curve plot for the multi-stage model shown in FIG. 10Q with a different view, according to one example embodiment. The multi-stage model was configured in the manner described above. In particular, the multi-stage model included (i) a first model to generate a cancer prediction using small variant features (maximum allele frequency for each gene in a targeted gene panel comprising genes associated with cancer (GRAIL's proprietary targeted small variant panel)) as an input, (ii) a second model to generate a cancer prediction using WGS features (per gene copy number z-score across a targeted gene panel comprising genes associated with cancer (GRAIL's proprietary targeted small variant panel)), and (iii) a combined model to generate a cancer prediction using the cancer predictions of the first model and the second model (using second layer regularized logistic regression). The multi-stage model is applied to test sequences obtained from a group of individuals to determine a cancer prediction. The test sequences include liquid cancer sequences, non-cancer sequences, and solid cancer sequences.

The ROC curve plots illustrate the true positive rate as a function of the false positive rate for the first model, the second model, and the combined model. Curves on the ROC curve plots provide a representation of the predictive ability of each of the cancer prediction models (FIG. 10Q includes sensitivity estimates at 99% specificity (shown as a square), 98% specificity (shown as a triangle), and 95% specificity (shown as a circle). Notably, the predictive capability of the combined model is better than either of the first model or the second model. In other words, using the predictive results of two cancer prediction models in a multi-stage cancer prediction model provided better predictive capability than individual cancer prediction models.

Table 7.5.1.1. provides quantifications of the predictive capability of the multi-stage model illustrated in FIGS. 10Q and 10R. The table indicates the specificity, sensitivity for the first model, the second model, and the combination model. The lower and upper bound for the 95% confidence intervals of the sensitivity are also shown in the table. Here, the combination model outperforms the first model and second model at all specificities.

TABLE 7.5.1.1
Cancer predictions for a multi-stage model.

| Specificity | Model | Lower CI | Sensitivity | Upper CI |
|---|---|---|---|---|
| 0.95 | Second | 0.2757 | 0.3053 | 0.3363 |
| 0.95 | Combination | 0.3544 | 0.3860 | 0.4184 |
| 0.95 | First | 0.3384 | 0.3697 | 0.4019 |
| 0.98 | Second | 0.2262 | 0.2541 | 0.2836 |
| 0.98 | Combination | 0.3362 | 0.3675 | 0.3996 |
| 0.98 | First | 0.3170 | 0.3479 | 0.3797 |
| 0.99 | Second | 0.2063 | 0.2334 | 0.2621 |
| 0.99 | Combination | 0.3181 | 0.3490 | 0.3808 |
| 0.99 | First | 0.3043 | 0.3348 | 0.3364 |

FIG. 10S illustrates a comparison of the sensitivity as a function of sensitivity for the first model, the second model, and the combination model, according to one example embodiment. FIG. 10S is a visualization of the data included in Table 7.5.1.1.

Table 7.5.1.2 provides another quantification of the predictive ability of the first model and the combination model. In this example, the test sequences include 27 liquid cancer test sequences, 574 non-cancer test sequences, and 890 solid cancer test sequences. Within the table, “True” indicates that a model determined a positive cancer prediction and “False” indicates that a model determined a negative cancer prediction. A number under a cancer type indicates the number of test-sequences having the indicated predictions. So, for example, the combination model correctly called three liquid cancer test sequences “True” while the first model incorrectly called those same three liquid cancer test sequences “False.”

TABLE 7.5.1.2
Classification comparison for liquid/non liquid cancers.

| Combination | First | Liquid | Non-Cancer | Solid |
|---|---|---|---|---|
| False | False | 12 | 522 | 532 |
| False | True | 0 | 22 | 18 |
| True | False | 3 | 23 | 31 |
| True | True | 12 | 7 | 30 |

7.5.2. Example 2: First-Stage Models Using WGS and Methylation Features

Example 2 is another multi-stage model having a first model, a second model, and a combination model. In this example, the first model is configured to determine a cancer prediction for an individual using WGS features. The features input into the first model may derived using techniques similar to those described in Section 3, “Whole Genome Computational Analysis.” The first model may output a score (e.g., a WGS score) indicating a cancer prediction for the individual. Section 7.2.2, “Whole Genome Predictive Cancer Model,” describes a predictive cancer model that may be used as the first model. FIG. 10D illustrates an example of the predictive capability of the second model. Further, in this example, the second model is configured to determine a cancer prediction for an individual using methylation features. The features input into the second model may derived using techniques similar to those described in Section 4, “Methylation Computational Analysis.” The second model may output a score (e.g., an M score) indicating a cancer prediction for the individual. Section 7.2.3, “Methylation Predictive Cancer Model,” describes a predictive cancer model that may be used as the second model. FIGS. 10E-10F illustrate examples of the predictive capability of the second model. Finally, in this example, the combined model is configured to generate a cancer prediction using the cancer predictions of the first model and second model.

FIG. 10T and FIG. 10U illustrate ROC curve plots for a multi-stage model, according to a first example embodiment. FIG. 10T demonstrates the capability of a multi-stage model in determining a lung cancer prediction for an individual, and FIG. 10U demonstrates the capability of the multi-stage model, the first model, and the second model in determining a breast cancer prediction for the individual.

The multi-stage model was configured in the manner described above. In particular, the multi-stage model included (i) a first model to generate a cancer prediction using WGS features (principal component projects on the first 5 principal components of the read counts as described in section 3.1) as an input, (ii) a second model to generate a cancer prediction using methylation features (hyper-methylation counts and/or hypo-methylation counts as described in section 4.3), and (iii) a combined model generating a cancer prediction using the cancer predictions of the first model and the second model. The multi-stage model was applied to test sequences obtained from a group of individuals to determine a cancer prediction.

The ROC curve plots illustrate the sensitivity as a function of specificity for the first model, the second model, and the combined model. Curves on the ROC curve plots provide a representation of the predictive ability of each of the cancer prediction models. In this case, the improvement of the combined model is more marginal than the multi-stage model shown in Example 1. As such, it is useful to analyze the area under curve (“AUC”) for each of the lines on the ROC curve plots. The AUC is a measure of the total predictive capability of each of the models, with a higher AUC representing, generally, a more sensitive and specific model.

Table 7.5.2.1. provides the AUC for the multi-stage model for determining a cancer prediction for different types of cancer. The table includes a column indicating the type of cancer and the AUC for the combined model, the first model, and the second model. Here, marginal improvement in cancer prediction is seen in lung cancer and colorectal cancer.

TABLE 7.5.2.1
Classification comparison for liquid/non liquid cancers.

| Cancer Type | Comb. Model | First Model | Second Model |
|---|---|---|---|
| Lung | 0.913 | 0.836 | 0.879 |
| Colorectal | 0.858 | 0.817 | 0.842 |
| Pancreas | 0.915 | 0.938 | 0.932 |
| Breast | 0.761 | 0.762 | 0.730 |
| Lymphoma | 0.873 | 0.769 | 0.910 |

7.5.3. Example 3: First-Stage Models Using Baseline and Methylation Features

Example 3 is another multi-stage model having a first model, a second model, and a combination model. In this example, the first model is configured to determine a cancer prediction for an individual using methylation features. The features input into the first model may be derived using techniques similar to those described in Section 4, “Methylation Computational Analysis.” The first model may output a score (e.g., an M score) indicating a cancer prediction for the individual. Section 7.2.3, “Methylation Predictive Cancer Model,” describes a predictive cancer model that may be used as the second model. FIGS. 10E-10F illustrate examples of the predictive capability of the second model. Further, in this example, the second model is configured to determine a cancer prediction for an individual using baseline features. The features input into the second model may derived using techniques similar to those described in Section 6, “Baseline Analysis and Baseline Computational Analysis.” The second model may output a score indicating a cancer prediction for the individual. FIG. 8B illustrates examples of the predictive capability of the second model when applied to breast cancer. Finally, in this example, the combined model is configured to generate a cancer prediction using the cancer predictions of the first model and second model.

FIG. 10V, FIG. 10W and FIG. 10X illustrate ROC curve plots for a multi-stage model, according to an example embodiment. FIG. 10V demonstrates the capability of a multi-stage model in determining a cancer prediction for an individual having a high signal cancers, FIG. 10OW demonstrates the capability of the multi-stage model in determining a lung cancer prediction for the individual, and FIG. 10X demonstrates the capability of the multi-stage model in determining HR− breast cancer prediction for the individual.

The multi-stage model was configured in the manner described above. In particular, the multi-stage model included (i) a first model to generate a cancer prediction using methylation features (hyper-methylation counts and/or hypo-methylation counts as described in section 4.3), (ii) a second model to generate a cancer prediction using baseline features (including, sex, race, body mass index (BMI), alcohol intake, breastfeeding history, BiRAD breast density, smoking history, second hand smoking, and/or immediate family history of lung cancer), and (iii) a combined model generating a cancer prediction using the cancer predictions of the first model and the second model (using logistic regression). The multi-stage model was applied to test sequences obtained from a group of individuals to determine a cancer prediction.

The ROC curve plots illustrate the sensitivity as a function of specificity for the first model, and the combined model. Curves on the ROC curve plots provide a representation of the predictive ability of each of the cancer prediction models. Second models are not illustrated but perform worse than the combination model. For FIG. 10V (high signal cancers), the ROC curve had an area under the curve (AUC) of 0.65 for the first-stage model (baseline features), and an AUC of 0.85 for the combination model (baseline and methylation features). For FIG. 10W (lung cancer), the ROC curve had an area under the curve (AUC) of 0.83 for the first-stage baseline model, an AUC of 0.89 for the first-stage methylation model, and an AUC of 0.93 for the combination model (baseline and methylation features). And for FIG. 10X (HR− breast cancer), the ROC curve had an area under the curve (AUC) of 0.69 for the first-stage baseline model, an AUC of 0.75 for the first-stage methylation model, and an AUC of 0.83 for the combination model (baseline and methylation features). Notably, the predictive capability of the combined model was better than either of the first model or the second model. In other words, using the predictive results of two cancer prediction models in a multi-stage cancer prediction model provides better predictive capability than individual cancer prediction models.

7.6. Example Single-Stage, Multiple Assay Predictive Cancer Models

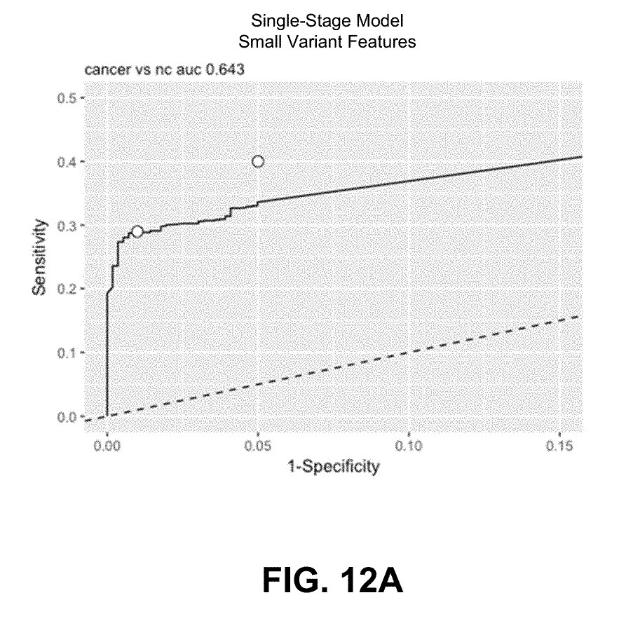

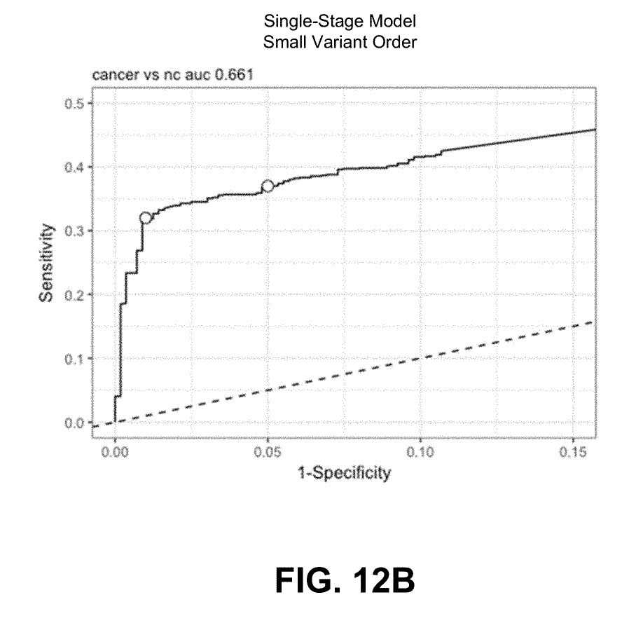

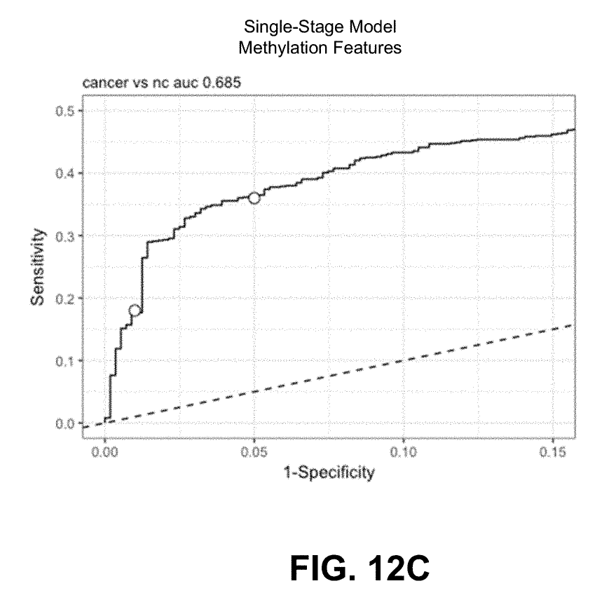

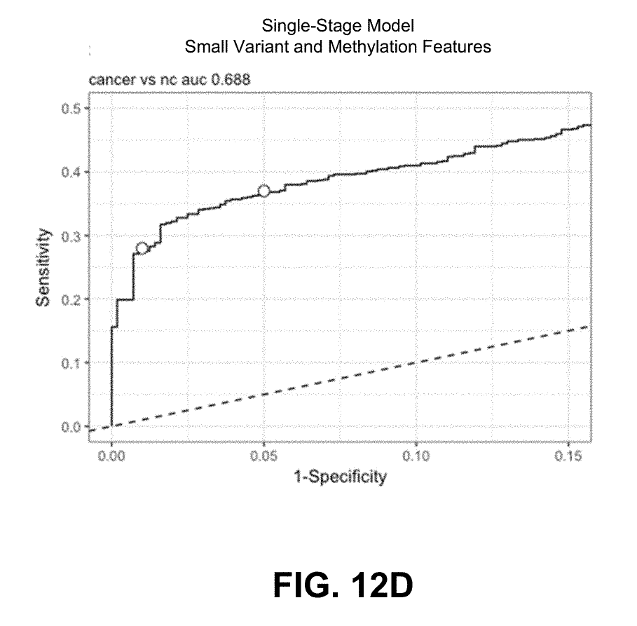

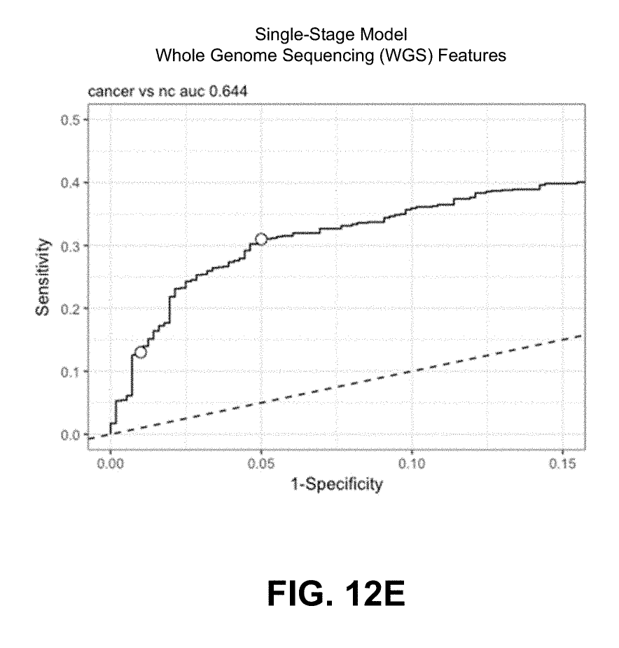

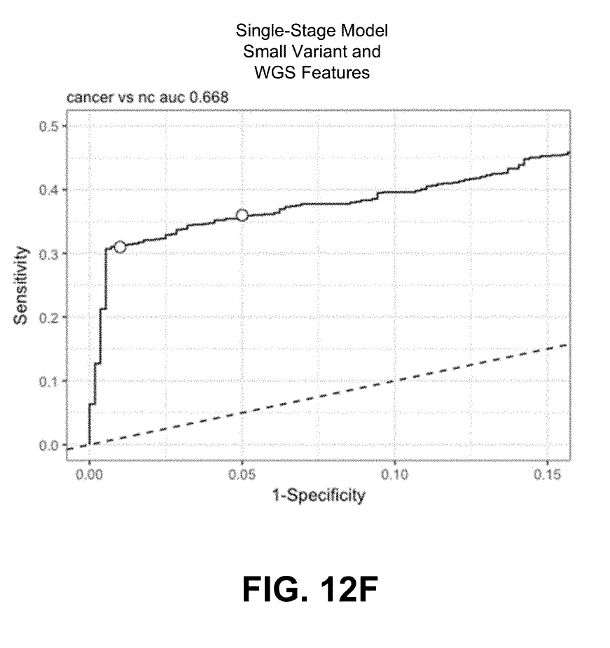

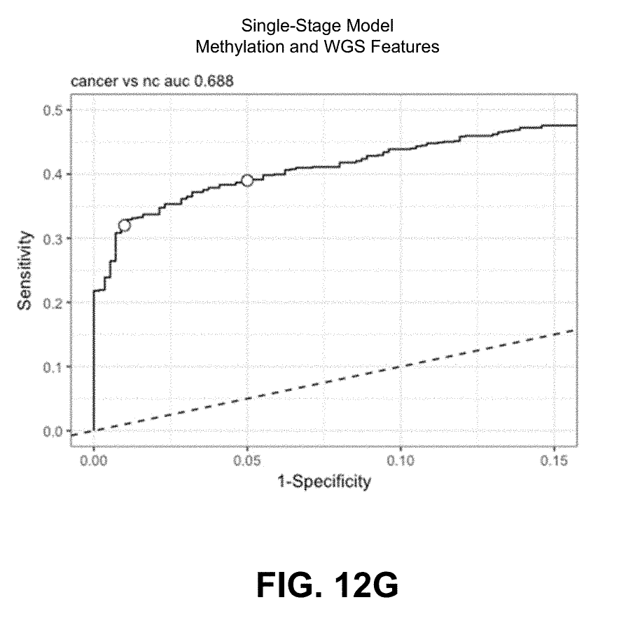

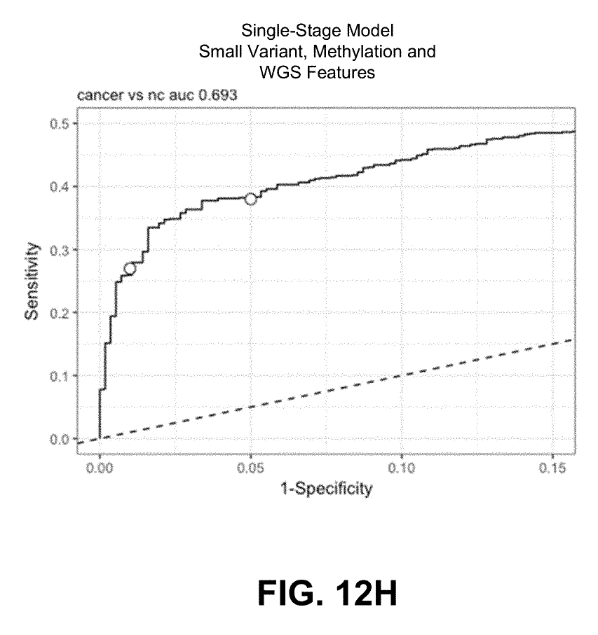

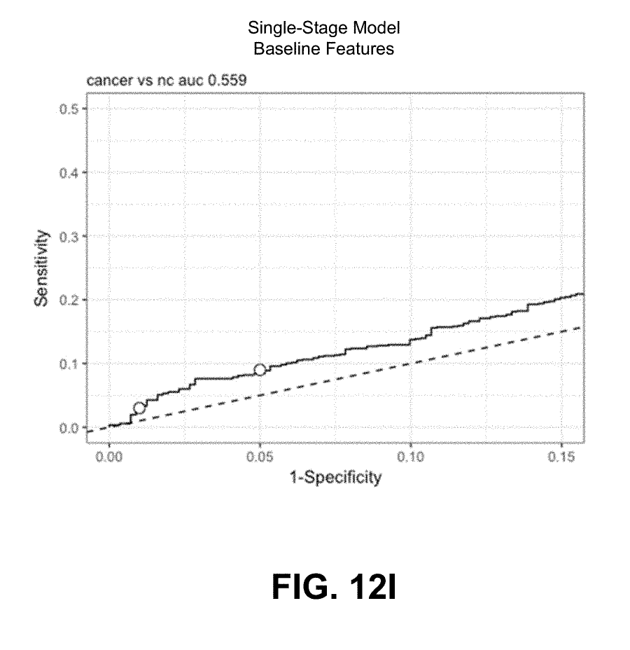

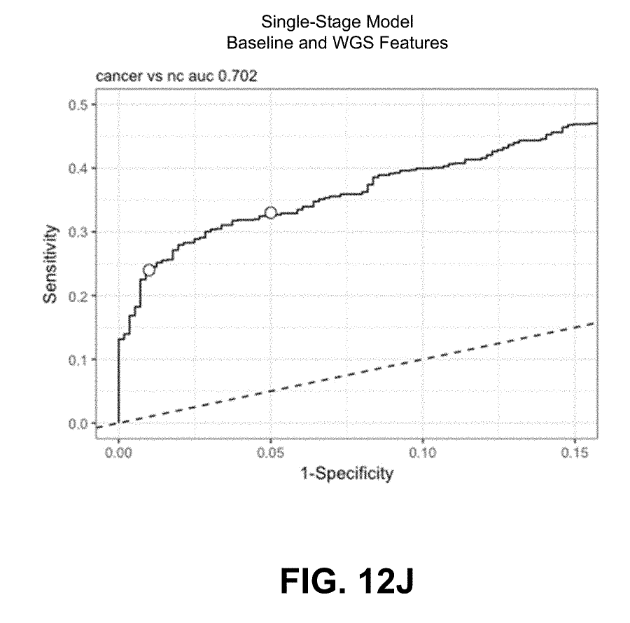

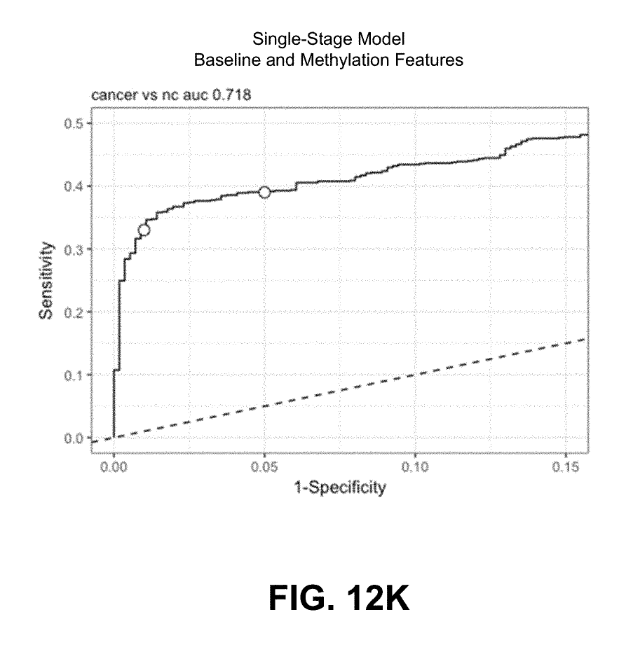

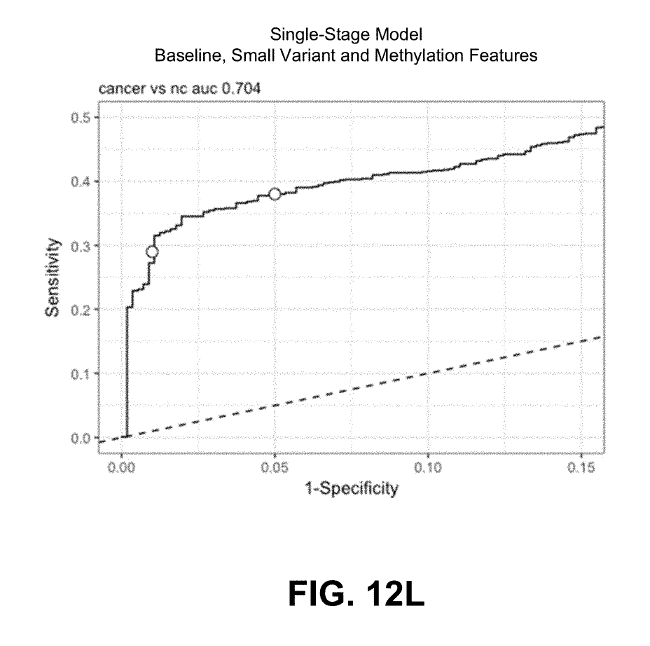

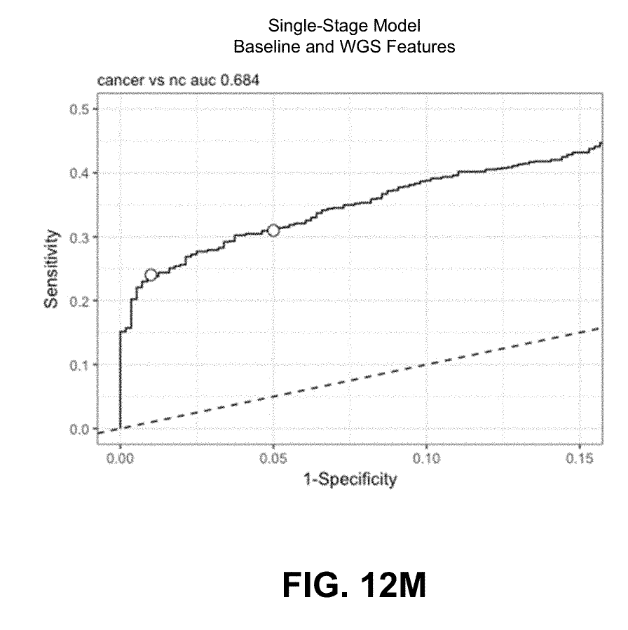

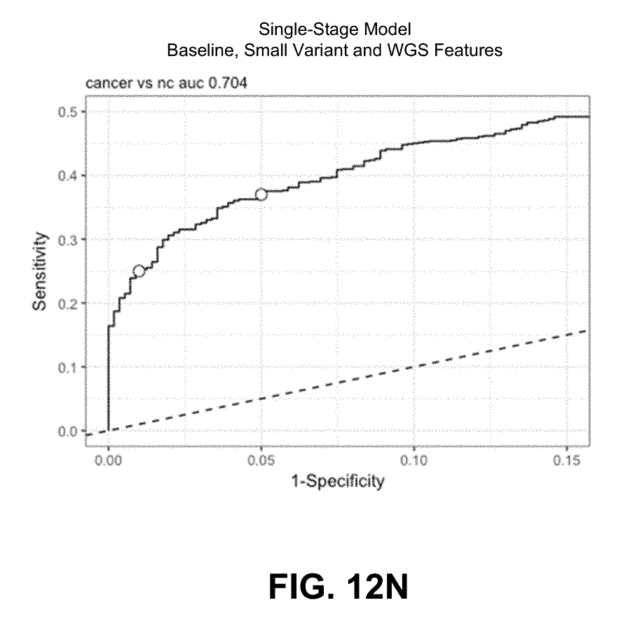

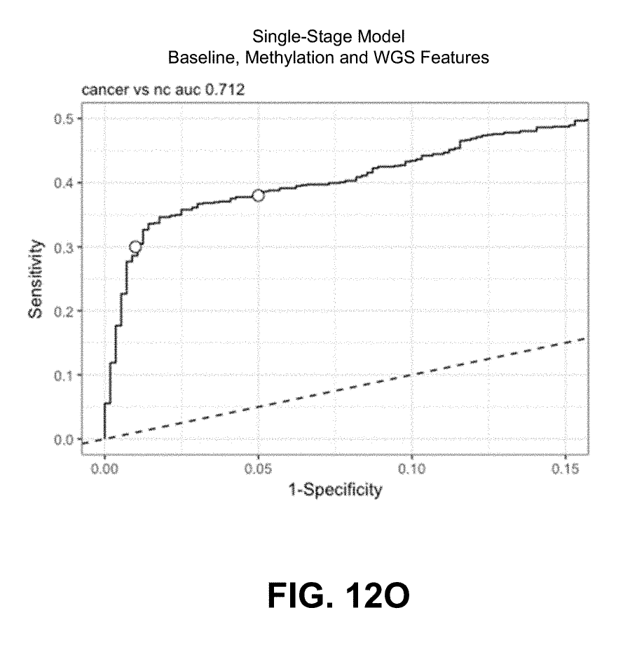

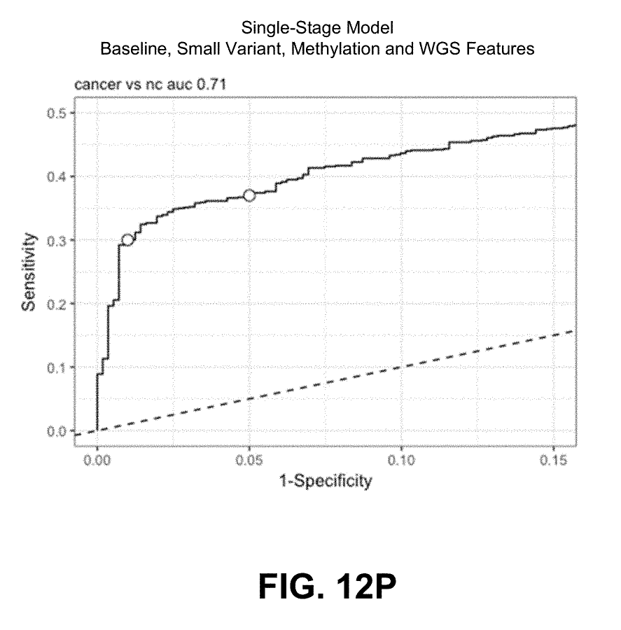

As described above in FIG. 1B, FIGS. 12A through 12P illustrate a number of single-stage predictive cancer models (e.g., predictive cancer model 160) utilizing one or more features from one or more sequencing based assays (e.g., baseline features 150, whole genome features 152, small variant features 154, and/or methylation features 156) to generate a cancer prediction for a subject.

Each of FIGS. 12A through 12P depict a receiver operating characteristic (ROC) curve, for all cancers in the CCGA study (previously described), of the specificity and sensitivity of a number of single-stage predictive cancer models that predicts the presence of cancer using one or more small variant features, whole genomes features, methylation features, and or baseline features in accordance with the embodiment shown in FIG. 1B. Here, each of the predictive cancer models exemplified comprises a XGBoost model, but could be other machine learning models. Specifically, the small variant features of the predictive cancer model include one of two features: (1) the maximum allele frequency (MAF) of non-synonymous somatic variant for each of a plurality of genes in a gene panel (GRAIL's proprietary 507-cancer associated gene panel) (see, e.g., section 2.1; see also FIG. 12A), or (2) the order statistics of somatic variants across all the genes of a cancer gene panel (GRAIL's proprietary 507-cancer associated gene panel), but could include other features. The range for these features are between 0-1, with most values being exactly zero, and typical non-zero values being below 0.1 (see, e.g., section 2.1; see also FIGS. 12B, 12D, 12F, 12H, 12J, 12L, 12N, and 12P). The whole genome features of the predictive cancer model include reduced dimensionality features (a total of 5 reduced dimensionality features) (as described in section 3.1), but could include other features. The methylation features of the predictive cancer model include rankings based on hypermethylation scores and rankings based on hypomethylation scores (as described in sections 4.1 and 4.3), but could include other features. In particular, the rankings based on hypermethylation scores includes 7 hypermethylated fragments (e.g., the rank 1, rank 2, rank 4, rank 8, rank 16, rank 32, and rank 64 hypermethylated fragments). Similarly, the rankings based on hypomethylation scores includes 7 hypomethylated fragments (e.g., the rank 1, rank 2, rank 4, rank 8, rank 16, rank 32, and rank 64 hypomethylated fragments). The baseline analysis included analysis of clinical features for: continuous variables for BMI, age, smoking duration, and indicator variables for breast tissue density, self-reported general health, self-reported exercise intensity, but could include other features. Indicator variables were coded as 0 or 1, and continuous variables were used in the range they are reported (see, e.g., section 6.1).

As described above, FIG. 12A illustrates a receiver operating characteristic (ROC) curve, for all cancers in the previously described CCGA study, of the specificity and sensitivity of a single-stage predictive cancer model that predicts the presence of cancer utilizing small variant features (e.g., MAF) from a targeted panel of genes known to be associated with cancer risk. The ROC curve indicates a ˜29 sensitivity at a 98% specificity, a ˜33% sensitivity at a 98% specificity, and an area under the curve of 0.643.

FIG. 12B illustrates a ROC curve, for all cancers in the previously described CCGA study, of the specificity and sensitivity of a single-stage predictive cancer model that predicts the presence of cancer utilizing small variant features (e.g., rank order) from a targeted panel of genes known to be associated with cancer risk. The ROC curve indicates a ˜32% sensitivity at a 37% specificity, a ˜33% sensitivity at a 98% specificity, and an area under the curve of 0.661.

FIG. 12C illustrates a ROC curve, for all cancers in the previously described CCGA study, of the specificity and sensitivity of a single-stage predictive cancer model that predicts the presence of cancer utilizing methylation features from a methylation assay. The ROC curve indicates a ˜18% sensitivity at a 98% specificity, a ˜36% sensitivity at a 98% specificity, and an area under the curve of 0.685.

FIG. 12D illustrates a ROC curve, for all cancers in the previously described CCGA study, of the specificity and sensitivity of a single-stage predictive cancer model that predicts the presence of cancer utilizing small variant features and methylation features. The ROC curve indicates a ˜28% sensitivity at a 98% specificity, a ˜37% sensitivity at a 98% specificity, and an area under the curve of 0.688.

FIG. 12E illustrates a ROC curve, for all cancers in the previously described CCGA study, of the specificity and sensitivity of a single-stage predictive cancer model that predicts the presence of cancer utilizing whole genome sequencing (e.g., WGS) features. The ROC curve indicates a ˜13% sensitivity at a 98% specificity, a ˜31% sensitivity at a 98% specificity, and an area under the curve of 0.644.

FIG. 12F illustrates a ROC curve, for all cancers in the previously described CCGA study, of the specificity and sensitivity of a single-stage predictive cancer model that predicts the presence of cancer utilizing small variant features and whole genome sequencing (WGS) features. The ROC curve indicates a ˜31% sensitivity at a 98% specificity, a ˜36% sensitivity at a 98% specificity, and an area under the curve of 0.668.

FIG. 12G illustrates a ROC curve, for all cancers in the previously described CCGA study, of the specificity and sensitivity of a single-stage predictive cancer model that predicts the presence of cancer utilizing WGS features and methylation features. The ROC curve indicates a ˜32% sensitivity at a 98% specificity, a ˜39% sensitivity at a 98% specificity, and an area under the curve of 0.688.

FIG. 12H illustrates a ROC curve, for all cancers in the previously described CCGA study, of the specificity and sensitivity of a single-stage predictive cancer model that predicts the presence of cancer utilizing small variant features, WGS features and methylation features. The ROC curve indicates a ˜27% sensitivity at a 98% specificity, a ˜38% sensitivity at a 98% specificity, and an area under the curve of 0.693.

FIG. 12I illustrates a ROC curve, for all cancers in the previously described CCGA study, of the specificity and sensitivity of a single-stage predictive cancer model that predicts the presence of cancer utilizing baseline features. The ROC curve indicates a ˜3% sensitivity at a 98% specificity, a ˜90% sensitivity at a 98% specificity, and an area under the curve of 0.559.

FIG. 12J illustrates a ROC curve, for all cancers in the previously described CCGA study, of the specificity and sensitivity of a single-stage predictive cancer model that predicts the presence of cancer utilizing small variant features and baseline features. The ROC curve indicates a ˜9% sensitivity at a 98% specificity, a ˜33% sensitivity at a 98% specificity, and an area under the curve of 0.702.

FIG. 12K illustrates a ROC curve, for all cancers in the previously described CCGA study, of the specificity and sensitivity of a single-stage predictive cancer model that predicts the presence of cancer utilizing methylation features and baseline features. The ROC curve indicates a ˜33% sensitivity at a 98% specificity, a ˜39% sensitivity at a 98% specificity, and an area under the curve of 0.718.

FIG. 12L illustrates a ROC curve, for all cancers in the previously described CCGA study, of the specificity and sensitivity of a single-stage predictive cancer model that predicts the presence of cancer utilizing small variant features, methylation features and baseline features. The ROC curve indicates a ˜29% sensitivity at a 98% specificity, a ˜38% sensitivity at a 98% specificity, and an area under the curve of 0.704.

FIG. 12M illustrates a ROC curve, for all cancers in the previously described CCGA study, of the specificity and sensitivity of a single-stage predictive cancer model that predicts the presence of cancer utilizing WGS features and baseline features. The ROC curve indicates a ˜24% sensitivity at a 98% specificity, a ˜31% sensitivity at a 98% specificity, and an area under the curve of 0.684.

FIG. 12N illustrates a ROC curve, for all cancers in the previously described CCGA study, of the specificity and sensitivity of a single-stage predictive cancer model that predicts the presence of cancer utilizing small variant features, WGS features and baseline features. The ROC curve indicates a ˜25% sensitivity at a 98% specificity, a ˜37% sensitivity at a 98% specificity, and an area under the curve of 0.704.

FIG. 12O illustrates a ROC curve, for all cancers in the previously described CCGA study, of the specificity and sensitivity of a single-stage predictive cancer model that predicts the presence of cancer utilizing WGS features, methylation features, and baseline features. The ROC curve indicates a ˜30% sensitivity at a 98% specificity, a ˜38% sensitivity at a 98% specificity, and an area under the curve of 0.712.

FIG. 12P illustrates a ROC curve, for all cancers in the previously described CCGA study, of the specificity and sensitivity of a single-stage predictive cancer model that predicts the presence of cancer utilizing small variant features, WGS features, methylation features, and baseline features. The ROC curve indicates a ˜30% sensitivity at a 98% specificity, a ˜37% sensitivity at a 98% specificity, and an area under the curve of 0.710.

The above-illustrated ROC curve plots illustrate the true positive rate as a function of the false positive rate for the each of the above cancer predictive models utilizing one or more features as described. Each of the curves of the ROC curve plots provide a representation of the predictive ability of each of the cancer prediction models. Notably, the predictive capability of the cancer predictive models including two or more, three or more, or four features is better than the cancer predictive models based on a single feature. In other words, using two or more sequencing features in the cancer predictive models described herein results in a model with better cancer predictive capability than models based on a single feature.

### ADDITIONAL CONSIDERATIONS

The foregoing detailed description of embodiments refers to the accompanying drawings, which illustrate specific embodiments of the present disclosure. Other embodiments having different structures and operations do not depart from the scope of the present disclosure. The term “the invention” or the like is used with reference to certain specific examples of the many alternative aspects or embodiments of the applicants' invention set forth in this specification, and neither its use nor its absence is intended to limit the scope of the applicants' invention or the scope of the claims. This specification is divided into sections for the convenience of the reader only. Headings should not be construed as limiting of the scope of the invention. The definitions are intended as a part of the description of the invention. It will be understood that various details of the present invention may be changed without departing from the scope of the present invention. Furthermore, the foregoing description is for the purpose of illustration only, and not for the purpose of limitation.

What is claimed is:

1. A method for determining a cancer prediction for a subject, the method comprising:
obtaining a dataset associated with cell-free nucleic acids in a test sample obtained from the subject, the dataset comprising sequence reads generated from one or more sequencing assays on the cell-free nucleic acids;
performing or having performed a computational analysis on the sequence reads to generate values for one or more features derived from the sequence reads;
applying a cancer prediction model to the values for the one or more features to generate a cancer prediction for the subject, the cancer prediction model comprising a function that computes the cancer prediction using learned weights;
providing the cancer prediction for the subject.

2. The method of claim 1, wherein applying the cancer prediction model to generate the cancer prediction comprises executing the function using two of:
one or more methylation features derived from a methylation sequencing assay on the cell-free nucleic acids in the test sample,
one or more whole genome features derived from a whole genome sequencing assay on the nucleic acids in the test sample,
one or more small variant features derived from a small variant sequencing assay on the nucleic acids in the test sample, and
one or more baseline features derived from a baseline analysis.

3. The method of claim 2, wherein the one or more methylation features comprise one of:
a quantity of hypomethylated counts,
a quantity of hypermethylated counts,
a presence or an absence of abnormally methylated fragments at a plurality of CpG sites,
a hypomethylation score at each of a plurality of CpG sites,
a hypermethylation score at each of a plurality of CpG sites,
a set of rankings based on hypermethylation scores, and
a set of rankings based on hypomethylation scores.

4. The method of claim 3, wherein the one or more the one or more methylation features further comprises one of:
a characteristic for each of a plurality of bins across the genome from a cfDNA test sample,
a characteristic for each of a plurality of bins across the genome from a gDNA sample,
a characteristic for each of a plurality of segments across the genome from a cfDNA sample,
a characteristic for each of a plurality of segments across the genome from a gDNA sample,
a presence of one or more copy number aberrations, and
a set of reduced dimensionality features.

5. The method of claim 2, wherein the one or more whole genome sequencing features comprise one of:
a characteristic for each of a plurality of bins across the genome from a cfDNA test sample,
a characteristic for each of a plurality of bins across the genome from a gDNA sample,
a characteristic for each of a plurality of segments across the genome from a cfDNA sample,
a characteristic for each of a plurality of segments across the genome from a gDNA sample,
a presence of one or more copy number aberrations, and
a set of reduced dimensionality features.

6. The method of claim 2, wherein the one or more small variant features comprise one of:
a total number of somatic variants,
a total number of nonsynonymous variants,
a total number of synonymous variants,
a presence or absence of a somatic variant for each of a plurality of genes in a gene panel,
a presence or absence of a somatic variant for each of a plurality of genes known to be associated with cancer,
an allele frequency of a somatic variant for each of a plurality of genes in a gene panel,
a ranked order according to AF of a somatic variant for each of a plurality of genes in a gene panel, and
an allele frequency of a somatic variant per category.

7. The method of claim 6, wherein the one or more small variant features further comprises one of:
a characteristic for each of a plurality of bins across the genome from a cfDNA test sample,
a characteristic for each of a plurality of bins across the genome from a gDNA sample,
a characteristic for each of a plurality of segments across the genome from a cfDNA sample,
a characteristic for each of a plurality of segments across the genome from a gDNA sample,
a presence of one or more copy number aberrations, and
a set of reduced dimensionality features.

8. The method of claim 2, wherein the one or more baseline features comprise any of:
a polygenic risk score or clinical features of an individual,
a clinical feature comprising any of age, body mass index (BMI), behavior, smoking history, alcohol intake, family history, symptoms, anatomical observations, breast density, and
a penetrant germline cancer carrier.

9. The method of claim 2, wherein applying the cancer prediction model to generate the cancer prediction further comprises applying the cancer prediction model to a value of a common assay feature, wherein the common assay feature comprises any of:
a quantity of nucleic acids,
a tumor-derived nucleic acid concentration of a sample,
a mean length of nucleic acid fragments, and
a median length of nucleic acid fragments.

10. The method of claim 1, wherein performing or having performed a computational analysis on the sequence reads to generate values for the set of features comprises performing a methylation computational analysis on the sequence reads.

11. The method of claim 1, wherein performing or having performed a computational analysis on the sequence reads to generate values for the set of features comprises performing a whole genome computational analysis on the sequence reads.

12. The method of any claim 1, wherein performing or having performed a computational analysis on the sequence reads to generate values for the set of features comprises performing a small variant computational analysis on the sequence reads.

13. The method of claim 1, further comprising:
performing or having performed a baseline analysis on the subject to generate values for a set of baseline features describing symptoms exhibited by the subject.

14. The method of claim 13, wherein applying the cancer prediction model to generate the cancer prediction for the subject further comprises applying the cancer prediction model to the values of the baseline features.

15. The method of claim 1, wherein performance of the cancer prediction model is characterized by a 30% sensitivity at a 95% specificity.

16. The method of claim 1, wherein a performance of the predictive cancer model is characterized by an area under the curve (AUC) of a receiver operating characteristic (ROC) for the presence of cancer is greater than 0.60.

17. The method of claim 1, wherein the subject is asymptomatic.

18. The method of claim 1, wherein the method determines two or more different types of cancer selected from: breast cancer, lung cancer, prostate cancer, colorectal cancer, renal cancer, uterine cancer, pancreas cancer, esophageal cancer, lymphoma, head and neck cancer, ovarian cancer, hepatobiliary cancer, melanoma, cervical cancer, multiple myeloma, leukemia, thyroid cancer, bladder cancer, gastric cancer, anorectal cancer.

19. The method of claim 1, wherein
the computational analysis detects a presence of a viral-derived nucleic acid in the test sample, and
applying the cancer prediction model to generate the cancer prediction is based, in part, on the detected viral nucleic acid.

20. The method of claim 19, wherein the viral-derived nucleic acid is derived from one of a human papillomavirus, an Epstein-Barr virus, a hepatitis B virus, or a hepatitis C virus.

21. The method of claim 1, wherein the sample is selected from the group consisting of blood, plasma, serum, urine, fecal, saliva, whole blood, a blood fraction, a tissue biopsy, pleural fluid, pericardial fluid, cerebral spinal fluid, and peritoneal fluid sample.

22. The method of claim 1, wherein the cell-free nucleic acids comprise cell-free DNA (cfDNA).

23. The method of claim 1, wherein the sequence reads are generated from a next generation sequencing (NGS) procedure.

24. The method of claim 1, wherein the sequence reads are generated from a massively parallel sequencing procedure using sequencing-by-synthesis.

25. The method of claim 1, wherein the nucleic acids in the sample includes DNA from white blood cells.

26. The method of claim 1, wherein the predictive cancer model is one of a logistic regression predictor, a random forest predictor, a gradient boosting machine, Naïve Bayes classifier, a neural network, or a XGBoost model.

27. A system for determining a cancer prediction for a subject, the system comprising:
a processor; and
a non-transitory computer-readable storage medium with encoded instructions that, when executed by the processor, cause the processor to accomplish steps of:
accessing a dataset associated with cell-free nucleic acids in a test sample obtained from the subject, the dataset comprising sequence reads generated from one or more sequencing assays on the cell-free nucleic acids;
performing or having performed a computational analysis on the sequence reads to generate values for one or more features derived from the sequence reads;
applying a cancer prediction model to the values for the one or more features to generate a cancer prediction for the subject, the cancer prediction model comprising a function that computes the cancer prediction using learned weights;
providing the cancer prediction for the subject.

28. A non-transitory computer readable storage medium storing executable instructions for determining a cancer prediction for a subject that, when executed by a hardware processor, causes the hardware processor to perform steps comprising:
accessing a dataset associated with cell-free nucleic acids in a test sample obtained from the subject, the dataset comprising sequence reads generated from one or more sequencing assays on the cell-free nucleic acids;
performing or having performed a computational analysis on the sequence reads to generate values for one or more features derived from the sequence reads;
applying a cancer prediction model to the values for the one or more features to generate a cancer prediction for the subject, the cancer prediction model comprising a function that computes the cancer prediction using learned weights;
providing the cancer prediction for the subject.

29. A method for determining a cancer prediction for a subject, the method comprising:
obtaining a dataset associated with cell-free nucleic acids in a test sample obtained from the subject, the dataset comprising sequence reads generated from one or more sequencing assays on the cell-free nucleic acids;
performing or having performed a computational analysis on the sequence reads to generate a first set and a second set of values from a first set and a second set of features derived from the sequence reads;
applying a first model to the first set of values from the first set of features to generate a first score,
applying a second model to the second set of values from the second set of features to generate a second score,
the first model comprising a first function that computes the first score and the second model comprising a second function that computes the second score such that each of the first score and the second score are computed based on different features;
applying a cancer prediction model to the first score and the second score to generate a cancer prediction; and
providing the cancer prediction for the subject.

30. The method of claim 29, wherein, the score for each set of features is weighted according to any of:
a type of the feature,
a tissue of origin for the feature,
a significance value of the feature,
a characteristic of the feature, and
a predetermined value for the feature.

31. The method of claim 29, wherein the first and/or second score represents one of:
a presence or an absence of cancer in the subject,
a severity or a grade of cancer in the subject,
a type of cancer,
a likelihood of a presence or and absence of cancer in the subject,
a likelihood of a severity or a grade of cancer in the subject,
a likelihood that the feature originated from a cancerous tissue, and
a likelihood that the feature originated from a particular type of tissue.

32. The method of claim 31, wherein applying the first and/or second model to the values of the first and/or second set of features to generate the first and/or second scores comprises executing the function using two of:
one or more methylation features derived from a methylation sequencing assay on the cell-free nucleic acids in the test sample,
one or more whole genome features derived from a whole genome sequencing assay on the nucleic acids in the test sample,
one or more small variant features derived from a small variant sequencing assay on the nucleic acids in the test sample, and
one or more baseline features derived from a baseline analysis.

33. The method of claim 29, wherein the one or more methylation features comprise one of:
a quantity of hypomethylated counts,
a quantity of hypermethylated counts,
a presence or an absence of abnormally methylated fragments at a plurality of CpG sites,
a hypomethylation score at each of a plurality of CpG sites,
a hypermethylation score at each of a plurality of CpG sites,
a set of rankings based on hypermethylation scores, and
a set of rankings based on hypomethylation scores.

34. The method of claim 33, wherein the one or more the one or more methylation features further comprises one of:
a characteristic for each of a plurality of bins across the genome from a cfDNA test sample,
a characteristic for each of a plurality of bins across the genome from a gDNA sample,
a characteristic for each of a plurality of segments across the genome from a cfDNA sample,
a characteristic for each of a plurality of segments across the genome from a gDNA sample,
a presence of one or more copy number aberrations, and
a set of reduced dimensionality features.

35. The method of claim 29, wherein the one or more whole genome sequencing features comprise one of:
a characteristic for each of a plurality of bins across the genome from a cfDNA test sample,
a characteristic for each of a plurality of bins across the genome from a gDNA sample,
a characteristic for each of a plurality of segments across the genome from a cfDNA sample,
a characteristics for each of a plurality of segments across the genome from a gDNA sample,
a presence of one or more copy number aberrations, and
a set of reduced dimensionality features.

36. The method of claim 29, wherein the one or more small variant features comprise one of
a total number of somatic variants,
a total number of nonsynonymous variants,
a total number of synonymous variants,
a presence or absence of a somatic variant for each of a plurality of genes in a gene panel,
a presence or absence of a somatic variant for each of a plurality of genes known to be associated with cancer,
an allele frequency of a somatic variant for each of a plurality of genes in a gene panel,
a ranked order according to AF of a somatic variant for each of a plurality of genes in a gene panel, and
an allele frequency of a somatic variant per category.

37. The method of claim 36, wherein the one or more small variant features further comprises one of:
a characteristic for each of a plurality of bins across the genome from a cfDNA test sample,
a characteristic for each of a plurality of bins across the genome from a gDNA sample, a characteristic for each of a plurality of segments across the genome from a cfDNA sample,
a characteristic for each of a plurality of segments across the genome from a gDNA sample,
a presence of one or more copy number aberrations, and
a set of reduced dimensionality features.

38. The method of claim 29, wherein the one or more baseline features comprise any of:
a polygenic risk score or clinical features of an individual,
a clinical feature comprising any of age, body mass index (BMI), behavior, smoking history, alcohol intake, family history, symptoms, anatomical observations, breast density, and
a penetrant germline cancer carrier.

39. The method of claim 29, wherein applying the cancer prediction model to generate the cancer prediction further comprises applying the cancer prediction model to a value of a common assay feature, wherein the common assay feature comprises any of:
a quantity of nucleic acids,
a tumor-derived nucleic acid concentration of a sample,
a mean length of nucleic acid fragments, and
a median length of nucleic acid fragments.

40. The method of claim 29, wherein performing or having performed a computational analysis on the sequence reads to generate values for the set of features comprises performing a methylation computational analysis on the sequence reads.

41. The method of claim 29, wherein performing or having performed a computational analysis on the sequence reads to generate values for the set of features comprises performing a whole genome computational analysis on the sequence reads.

42. The method of any claim 29, wherein performing or having performed a computational analysis on the sequence reads to generate values for the set of features comprises performing a small variant computational analysis on the sequence reads.

43. The method of claim 29, further comprising:
performing or having performed a baseline analysis on the subject to generate values for a set of baseline features describing symptoms exhibited by the subject.

44. The method of claim 43, wherein applying the cancer prediction model to generate the cancer prediction for the subject further comprises applying the cancer prediction model to the values of the set of baseline features.

45. The method of claim 29, wherein a performance of the cancer prediction model is characterized by a 30% sensitivity at a 95% specificity.

46. The method of claim 29, wherein a performance of the predictive cancer model is characterized by an area under the curve (AUC) of a receiver operating characteristic (ROC) for the presence of cancer is greater than 0.60.

47. The method of claim 29, wherein the cancer prediction the subject is asymptomatic.

48. The method of claim 29, wherein the method determines two or more different types of cancer selected from: breast cancer, lung cancer, prostate cancer, colorectal cancer, renal cancer, uterine cancer, pancreas cancer, esophageal cancer, lymphoma, head and neck cancer, ovarian cancer, hepatobiliary cancer, melanoma, cervical cancer, multiple myeloma, leukemia, thyroid cancer, bladder cancer, gastric cancer, anorectal cancer.

49. The method of claim 29, wherein
the computational analysis detects a presence of a viral-derived nucleic acid in the test sample, and
applying the cancer prediction model to generate the cancer prediction is based, in part, on the detected viral nucleic acid.

50. The method of claim 49, wherein the viral-derived nucleic acid is derived from one of a human papillomavirus, an Epstein-Barr virus, a hepatitis B virus, or a hepatitis C virus.

51. The method of claim 29, wherein the sample is selected from the group consisting of blood, plasma, serum, urine, fecal, saliva, whole blood, a blood fraction, a tissue biopsy, pleural fluid, pericardial fluid, cerebral spinal fluid, and peritoneal fluid sample.

52. The method of claim 29, wherein the cell-free nucleic acids comprise cell-free DNA (cfDNA).

53. The method of claim 29, wherein the sequence reads are generated from a next generation sequencing (NGS) procedure.

54. The method of claim 29, wherein the sequence reads are generated from a massively parallel sequencing procedure using sequencing-by-synthesis.

55. The method of claim 29, wherein the nucleic acids in the sample includes DNA from white blood cells.

56. The method of claim 29, wherein the predictive cancer model is one of a logistic regression predictor, a random forest predictor, a gradient boosting machine, Naïve Bayes classifier, a neural network, or a XGBoost model.

57. A system for determining a cancer prediction for a subject, the system comprising:
a processor; and
a non-transitory computer-readable storage medium with encoded instructions that, when executed by the processor, cause the processor to accomplish steps of
accessing a dataset associated with cell-free nucleic acids in a test sample obtained from the subject, the dataset comprising sequence reads generated from one or more sequencing assays on the cell-free nucleic acids;
performing or having performed a computational analysis on the sequence reads to generate a first set and a second set of values from a first set and a second set of features derived from the sequence reads;
applying a first model to the first set of values from the first set of features to generate a first score,
applying a second model to the second set of values from the second set of features to generate a second score,
the first model comprising a first function that computes the first score and the second model comprising a second function that computes the second score such that each of the first score and the second score are computed based on different features;
applying a cancer prediction model to the first score and the second score to generate a cancer prediction; and
providing the cancer prediction for the subject.

58. A non-transitory computer readable storage medium storing executable instructions for determining a cancer prediction for a subject that, when executed by a hardware processor, cause the hardware processor to perform steps comprising:
accessing a dataset associated with cell-free nucleic acids in a test sample obtained from the subject, the dataset comprising sequence reads generated from one or more sequencing assays on the cell-free nucleic acids;
performing or having performed a computational analysis on the sequence reads to generate a first set and a second set of values from a first set and a second set of features derived from the sequence reads;
applying a first model to the first set of values from the first set of features to generate a first score,
applying a second model to the second set of values from the second set of features to generate a second score,
the first model comprising a first function that computes the first score and the second model comprising a second function that computes the second score such that each of the first score and the second score are computed based on different features;
applying a cancer prediction model to the first score and the second score to generate a cancer prediction; and
providing the cancer prediction for the subject.

59. A method for determining a cancer prediction for a subject, the method comprising:
obtaining a dataset associated with cell-free nucleic acids in a test sample obtained from the subject, the dataset comprising sequence reads generated from one or more sequencing assays on the cell-free nucleic acids;
performing or having performed a computational analysis on the dataset to generate values for two or more features describing the cell-free nucleic acids in the test sample, the features including:
a first set of methylation features derived from sequence reads from a methylation sequencing assay on nucleic acids in the test sample, and
a second set of non-methylation features derived from sequence reads from a sequencing assay on nucleic acids in the test sample;
applying a cancer prediction model to the values from the first set of methylation features and the values from the second set of non-methylation features to generate a cancer prediction for the subject, the cancer prediction model comprising a first function that computes the cancer prediction using learned weights; and
providing the cancer prediction for the subject.

60. The method of claim 59, wherein applying the cancer prediction model further comprises:
applying the cancer prediction model to the first set of methylation features to generate a first score,
applying the cancer prediction model to the second set of non-methylation features to generate a second score,
the cancer prediction model comprising a first function that computes the first score and a second function that computes a second score such that each of the first score and the second score are computed based on different features; and
wherein the first function computes the cancer prediction using the first and second scores.

61. The method of claim 59, wherein, the score for each set of features is weighted according to any of:
a type of the feature,
a tissue of origin for the feature,
a significance value of the feature,
a characteristic of the feature, and
a predetermined value for the feature.

62. The method of claim 59, wherein, the first and/or second score represents one of:
a presence or an absence of cancer in the subject,
a severity or a grade of cancer in the subject,
a type of cancer,
a likelihood of a presence or and absence of cancer in the subject,
a likelihood of a severity or a grade of cancer in the subject,
a likelihood that the feature originated from a cancerous tissue, and
a likelihood that the feature originated from a particular type of tissue.

63. The method of claim 59, wherein the first set of methylation features comprises one of:
a quantity of hypomethylated counts,
a quantity of hypermethylated counts,
a presence or an absence of abnormally methylated fragments at each of a plurality of CpG sites,
a hypomethylation score at each of a plurality of CpG sites,
a hypermethylation score at each of a plurality of CpG sites,
a set of rankings based on hypermethylation scores, and
a set of rankings based on hypomethylation scores.

64. The method of claim 59, wherein applying the cancer prediction model further comprises inputting, into the first function, the values of the non-methylation features, the non-methylation features comprising any of:
one or more whole genome features derived from a sequencing assay on the nucleic acids in the test sample,
one or more small variant features derived from a small variant sequencing assay on the nucleic acids in the test sample, and
one or more baseline features derived from a baseline analysis.

65. The method of claim 64, wherein the one or more whole genome features are derived from the sequence reads from the methylation assay.

66. The method of claim 64, wherein the one or more whole genome features are derived from sequence reads from a whole genome sequencing assay.

67. The method of and one of claim 64, wherein the one or more whole genome sequencing feature comprises one of:
a characteristic for each of a plurality of bins across the genome from a cfDNA test sample,
a characteristic for each of a plurality of bins across the genome from a gDNA sample,
a characteristic for each of a plurality of segments across the genome from a cfDNA sample,
a characteristics for each of a plurality of segments across the genome from a gDNA sample,
a presence of one or more copy number aberrations, and
a set of reduced dimensionality features.

68. The method of claim 64, wherein the one or more small variant features comprises one of
a total number of somatic variants,
a total number of nonsynonymous variants,
a total number of synonymous variants,
a presence or absence of a somatic variant for each of a plurality of genes in a gene panel,
a presence or absence of a somatic variants for each of a plurality of genes known to be associated with cancer,
an allele frequency of a somatic variant for each of a plurality of genes in a gene panel,
a ranked order according to AF of a somatic variant for each of a plurality of genes in a gene panel, and
an allele frequency of a somatic variant per category.

69. The method of claim 64, wherein the one or more baseline feature comprises any of:
a polygenic risk score or clinical features of an individual,
a clinical feature comprising any of age, body mass index (BMI), behavior, smoking history, alcohol intake, family history, symptoms, anatomical observations, breast density, and
a penetrant germline cancer carrier.

70. The method of claim 64, wherein applying the cancer prediction model to generate the cancer prediction further comprises applying the cancer prediction model to a value of a common assay feature, wherein the common assay feature comprises any of:
a quantity of nucleic acids,
a tumor-derived nucleic acid concentration of a sample,
a mean length of nucleic acid fragments, and
a median length of nucleic acid fragments.

71. The method of claim 59, wherein performing or having performed a computational analysis on the sequence reads to generate values for the first set of methylation features comprises performing a methylation computational analysis on the sequence reads.

72. The method of claim 59, wherein performing or having performed a computational analysis on the sequence reads to generate values for the second set of non-methylation features comprises performing a whole genome computational analysis on the sequence reads.

73. The method of any claim 59, wherein performing or having performed a computational analysis on the sequence reads to generate values for the second set of non-methylation features comprises performing a small variant computational analysis on the sequence reads.

74. The method of claim 59, further comprising:
performing or having performed a baseline analysis on the subject to generate values for a set of baseline features describing symptoms exhibited by the subject.

75. The method of claim 74, wherein applying the cancer prediction model to generate the cancer prediction for the subject further comprises applying the cancer prediction model to the values of the baseline features.

76. The method of claim 59, wherein performance of the cancer prediction model is characterized by a 30% sensitivity at a 95% specificity.

77. The method of any one of claims 59-77, wherein performance of the predictive cancer model is characterized by an area under the curve (AUC) of a receiver operating characteristic (ROC) for the presence of cancer is greater than 0.60.

78. The method of claim 59, wherein the cancer prediction the subject is asymptomatic.

79. The method of claim 59, wherein the method determines two or more different types of cancer selected from: breast cancer, lung cancer, prostate cancer, colorectal cancer, renal cancer, uterine cancer, pancreas cancer, esophageal cancer, lymphoma, head and neck cancer, ovarian cancer, hepatobiliary cancer, melanoma, cervical cancer, multiple myeloma, leukemia, thyroid cancer, bladder cancer, gastric cancer, anorectal cancer.

80. The method of claim 59, wherein
the computational analysis detects a presence of a viral-derived nucleic acid in the test sample, and
applying the cancer prediction model to generate the cancer prediction is based, in part, on the detected viral nucleic acid.

81. The method of claim 80, wherein the viral-derived nucleic acid is derived from one of a human papillomavirus, an Epstein-Barr virus, a hepatitis B virus, or a hepatitis C virus.

82. The method of claim 59, wherein the sample is selected from the group consisting of blood, plasma, serum, urine, fecal, saliva, whole blood, a blood fraction, a tissue biopsy, pleural fluid, pericardial fluid, cerebral spinal fluid, and peritoneal fluid sample.

83. The method of claim 59, wherein the cell-free nucleic acids comprise cell-free DNA (cfDNA).

84. The method of claim 59, wherein the sequence reads are generated from a next generation sequencing (NGS) procedure.

85. The method of claim 59, wherein the sequence reads are generated from a massively parallel sequencing procedure using sequencing-by-synthesis.

86. The method of claim 59, wherein the nucleic acids in the sample includes DNA from white blood cells.

87. The method of claim 59, wherein the predictive cancer model is one of a logistic regression predictor, a random forest predictor, a gradient boosting machine, Naïve Bayes classifier, a neural network, or a XGBoost model.

88. A system for determining a cancer prediction for a subject, the system comprising:
a processor; and
a non-transitory computer-readable storage medium with encoded instructions that, when executed by the processor, cause the processor to accomplish steps of
accessing a dataset associated with cell-free nucleic acids in a test sample obtained from the subject, the dataset comprising sequence reads generated from one or more sequencing assays on the cell-free nucleic acids;
performing or having performed a computational analysis on the dataset to generate values for two or more features describing the cell-free nucleic acids in the test sample, the features including:
a first set of methylation features derived from sequence reads from a methylation sequencing assay on nucleic acids in the test sample, and
a second set of non-methylation features derived from sequence reads from a sequencing assay on nucleic acids in the test sample;
applying a cancer prediction model to the values from the first set of methylation features and the values from the second set of non-methylation features to generate a cancer prediction for the subject, the cancer prediction model comprising a first function that computes the cancer prediction using learned weights; and
providing the cancer prediction for the subject.

89. A non-transitory computer readable storage medium storing executable instructions for determining a cancer prediction for a subject that, when executed by a hardware processor, cause the hardware processor to perform steps comprising:
obtaining a dataset associated with cell-free nucleic acids in a test sample obtained from the subject, the dataset comprising sequence reads generated from one or more sequencing assays on the cell-free nucleic acids;
performing or having performed a computational analysis on the dataset to generate values for two or more features describing the cell-free nucleic acids in the test sample, the features including:
a first set of methylation features derived from sequence reads from a methylation sequencing assay on nucleic acids in the test sample, and
a second set of non-methylation features derived from sequence reads from a sequencing assay on nucleic acids in the test sample;
applying a cancer prediction model to the values from the first set of methylation features and the values from the second set of non-methylation features to generate a cancer prediction for the subject, the cancer prediction model comprising a first function that computes the cancer prediction using learned weights; and
providing the cancer prediction for the subject.
